(19) United States
(12) Patent Application Publication
(10) Pub. No.: US 2017/0088902 A1
(43) Pub. Date: Mar. 30, 2017

(54) EXPRESSION PROFILING FOR CANCERS TREATED WITH ANTI-ANGIOGENIC THERAPY

(71) Applicant: ALMAC DIAGNOSTICS LIMITED, Craigavon (GB)

(72) Inventors: Denis Paul HARKIN, Dromore (GB); Richard KENNEDY, Belfast (GB); Andrena MCCAVIGAN, Derryadd (GB); Katherine KEATING, Magherafelt (GB); Laura HILL, Lisburn (GB); Steve DEHARO, Belfast (GB); Timothy DAVISON, Hillsborough (GB); Fionnuala PATTERSON, Hauxton (GB); Sinead DONEGAN, Belfast (GB); Gera JELLEMA, Portadown (GB); Charlie GOURLEY, Dumfermline (GB)

(73) Assignee: ALMAC DIAGNOSTICS LIMITED, Craigavon (GB)

(21) Appl. No.: 15/311,618

(22) PCT Filed: May 28, 2015
(86) PCT No.: PCT/GB2015/051557

§ 371 Date: Nov. 16, 2016

(30) Foreign Application Priority Data
May 28, 2014 (GB) 1409476.7

(51) Int. Cl. C12Q 1/68

(52) U.S. Cl. CPC C12Q 1/6886; C12Q 2600/106; C12Q 2600/118; C12Q 2600/158

(57) ABSTRACT

The present invention relates to a cancer sub-type. Provided are methods for determining clinical prognosis of a subject with cancer, selecting whether to administer an anti-angiogenic therapeutic agent to a subject with cancer and predicting responsiveness of a subject with cancer to an anti-angiogenic therapeutic agent. The methods are based on assessing from the expression level of biomarkers disclosed herein whether the cancer belongs to the sub-type. Companion methods of treating cancer and agents for use in treating cancer are also provided.

### FIELD OF THE INVENTION

The present invention relates to a cancer sub-type. Provided are methods for determining clinical prognosis and selecting whether to administer an anti-angiogenic therapeutic agent based on assessing from the expression level of biomarkers whether the cancer belongs to the sub-type.

### BACKGROUND OF THE INVENTION

Individualisation of therapy for cancer patients is desirable in order to ensure the most effective treatment for a particular patient. Currently, it is often difficult for healthcare professionals to identify cancer patients who will benefit from a given therapy regime. Thus, patients often needlessly undergo ineffective, toxic drug therapy. The advent of microarrays and molecular genomics has the potential to aid in the prediction of the response of an individual patient to a defined therapeutic regimen.

Angiogenesis is a key area for therapeutic intervention. This has promoted the development of a number of agents that target angiogenesis related processes and pathways, including the market leader and first FDA-approved anti-angiogenic, bevacizumab (Avastin), produced by Genentech/Roche.

Treatment regimens that include bevacizumab have demonstrated broad clinical activity ¹⁻¹⁰. However, no overall survival (OS) benefit has been shown after the addition of bevacizumab to cytotoxic chemotherapy in most cancers ^{8, 12-13}. This suggests that a substantial proportion of tumours are either initially resistant or quickly develop resistance to VEGF blockade (the mechanism of action of bevacizumab). In fact, 21% of ovarian, 10% of renal and 33% of rectal cancer patients show partial regression when receiving bevacizumab monotherapy, suggesting that bevacizumab may be active in small subgroups of patients, but that such incremental benefits do not reach significance in unselected patients¹⁵⁻¹⁸. As such, the availability of biomarkers of response to bevacizumab would improve assessment of treatment outcomes and thus enable the identification of patient subgroups that would receive the most clinical benefit from bevacizumab treatment.

Thus, there is a need for a test that would facilitate the stratification of patients based upon their predicted response to anti-angiogenic therapeutics, either in combination with standard of care or as a single-agent therapeutic. This would allow for the rapid identification of those patients who should receive alternative therapies.

### DESCRIPTION OF THE INVENTION

A cancer with a given histopathological diagnosis may represent multiple diseases at a molecular level.

The present inventors have identified a molecular sub-type of high grade serous ovarian cancer (HGSOC) that has an improved prognosis and where the addition of bevacizumab to the treatment regimen significantly reduces overall survival and progression free survival. The sub-type is associated with an up-regulation in molecular signaling related to immune response and a down-regulation in molecular signaling related to angiogenesis and vasculature development, referred to herein as a “non-angiogenesis” or “immune” subtype. The inventors have found that this sub-type can be reliably identified using a range of biomarker expression signatures.

Thus, in a first aspect the invention provides a method for selecting whether to administer an anti-angiogenic therapeutic agent to a subject with cancer, comprising:

measuring the expression levels of at least 3 biomarkers in a sample from the subject,

wherein at least two of the biomarkers are from Table A and at least one of the biomarkers is from Table B and assessing from the expression levels of the biomarkers whether the cancer belongs to a cancer sub-type

wherein the cancer sub-type is defined by the expression levels of a set of biomarkers associated with angiogenesis and a set of biomarkers associated with immune response

wherein if the cancer belongs to the sub-type an anti-angiogenic therapeutic agent is contraindicated

wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

TABLE A
Angiogenesis PS

| Cluster | | SEQ ID | Core | Gene | | mean expression | |
|---|---|---|---|---|---|---|---|
| # | Probeset ID | NO | (Yes/No) | Symbol | Orientation | in Immune Group | bias |
| 2 | OCHP.1836_s_at | 1 | Yes | GJA1 | Sense (Fully Exonic) | −0.4998 | −0.4190 |
| 2 | OC3P.1987.C1_x_at | 2 | Yes | IGFBP5 | Sense (Fully Exonic) | −0.5447 | −0.3128 |
| 2 | OCADNP.7251_s_at | 3 | Yes | MMP2 | Sense (Fully Exonic) | −0.7734 | −0.6010 |
| 2 | OC3P.4984.C1-787a_s_at | 4 | Yes | COL5A1 | Sense (Fully Exonic) | −0.7237 | −0.6844 |
| 2 | OCRS2.11009_x_at | 5 | Yes | TAGLN | Sense (Fully Exonic) | −0.4502 | −0.2326 |
| 2 | OC3P.89.C6_s_at | 6 | Yes | ELN | Sense (Fully Exonic) | −0.4746 | −0.4273 |
| 2 | OC3P.694.CB1-490a_s_at | 7 | Yes | DCN | Sense (Fully Exonic) | −0.6608 | −0.4760 |
| 2 | OCADNP.9526_s_at | 8 | Yes | CTGF | Sense (Fully Exonic) | −0.5069 | −0.5898 |
| 2 | OCADNP.3131_x_at | 9 | Yes | IGFBP7 | Sense (Includes Intronic) | −0.3405 | −0.4693 |
| 2 | OC3SNG.461-892a_s_at | 10 | Yes | DCN | Sense (Fully Exonic) | −0.6436 | −0.4910 |
| 2 | OC3P.12939.C1_s_at | 11 | Yes | IGF1 | Sense (Fully Exonic) | −0.3956 | −0.3536 |
| 2 | OC3SNG.6042-23a_x_at | 12 | Yes | FGFR1 | Sense (Fully Exonic) | −0.4413 | −0.5410 |
| 2 | OC3P.2790.C1_s_at | 13 | Yes | THY1 | Sense (Fully Exonic) | −0.6144 | −0.4273 |
| 2 | OC3SNGnh.5811_at | 14 | Yes | DMD | Sense (Includes Intronic) | −0.3557 | −0.2186 |
| 2 | OC3P.1178.C1_x_at | 15 | Yes | CTGF | Sense (Fully Exonic) | −0.5309 | −0.5211 |
| 2 | OC3SNGn.1211-6a_s_at | 16 | Yes | COL3A1 | Sense (Fully Exonic) | −0.7527 | −0.6400 |
| 2 | OCHP.1216_s_at | 17 | Yes | ACTA2 | Sense (Fully Exonic) | −0.5347 | −0.5909 |
| 2 | OCMXSNG.274_s_at | 18 | Yes | NFKBIZ | AntiSense | 0.0554 | 0.0891 |
| 2 | OC3SNGn.1637-35a_s_at | 19 | Yes | ZFP36 | Sense (Fully Exonic) | 0.0280 | −0.0911 |
| 2 | OC3P.1910.C1_s_at | 20 | Yes | EGR1 | Sense (Fully Exonic) | −0.1851 | −0.1155 |
| 2 | OC3P.564.C1-358a_s_at | 21 | Yes | VMP1 | Sense (Fully Exonic) | −0.1199 | −0.0070 |
| 2 | OC3P.3499.C1_s_at | 22 | Yes | FAT1 | Sense (Fully Exonic) | −0.4657 | −0.3351 |
| 2 | OC3P.7845.C1_s_at | 23 | Yes | COL14A1 | Sense (Fully Exonic) | −0.3109 | −0.2482 |
| 2 | OC3SNGnh.3734_s_at | 24 | Yes | TGFB2 | Sense (Fully Exonic) | −0.2690 | −0.1760 |
| 2 | OC3P.4123.C1_x_at | 25 | Yes | MMP14 | Sense (Fully Exonic) | −0.7302 | −0.6159 |
| 2 | OCADNP.2432_s_at | 26 | Yes | EGR1 | AntiSense | −0.1490 | −0.0805 |
| 2 | OC3P.1987.CB1_x_at | 27 | Yes | IGFBP5 | Sense (Fully Exonic) | −0.5509 | −0.2866 |
| 2 | OC3P.14073.C1_s_at | 28 | Yes | COL12A1 | Sense (Fully Exonic) | −0.4360 | −0.4135 |
| 2 | OC3P.2409.C1_s_at | 29 | Yes | MIR21 | Sense (Fully Exonic) | −0.0786 | 0.0052 |
| 2 | OC3SNGnh.14507_x_at | 30 | Yes | RORA | Sense (Includes Intronic) | −0.4118 | −0.3526 |
| 2 | OC3P.354.CB1_s_at | 31 | Yes | COL1A1 | Sense (Fully Exonic) | −0.7565 | −0.6116 |
| 2 | OC3P.3100.C1_s_at | 32 | Yes | RGS2 | Sense (Fully Exonic) | −0.3108 | −0.2359 |
| 2 | OC3SNGnh.14507_at | 33 | Yes | RORA | Sense (Includes Intronic) | −0.3389 | −0.2364 |
| 2 | OCMXSNG.5052_s_at | 34 | Yes | FN1 | AntiSense | −0.4764 | −0.5067 |
| 2 | OC3SNGn.2375-26a_s_at | 35 | Yes | MMP11 | Sense (Fully Exonic) | −0.4921 | −0.7305 |
| 2 | OC3P.2679.C1_s_at | 36 | Yes | ANGPTL2 | Sense (Fully Exonic) | −0.6830 | −0.5973 |
| 2 | OCADA.11214_s_at | 37 | Yes | SPHK2 | Sense (Fully Exonic) | −0.1649 | −0.3022 |
| 2 | OCRS2.11542_s_at | 38 | Yes | TWIST1 | Sense (Fully Exonic) | −0.6596 | −0.4253 |
| 2 | OCMX.15173.C1_s_at | 39 | Yes | VCAN | Sense (Fully Exonic) | −0.7228 | −0.6443 |
| 2 | OC3SNGn.2538-539a_x_at | 40 | Yes | COL1A2 | Sense (Fully Exonic) | −0.8816 | −0.6950 |
| 2 | OC3SNGn.8705-760a_x_at | 41 | Yes | MGP | Sense (Fully Exonic) | −0.1183 | −0.2157 |
| 2 | OC3SNG.1640-14a_s_at | 42 | Yes | SMARCA1 | Sense (Fully Exonic) | −0.5240 | −0.2337 |
| 2 | OC3SNG.5134-22a_s_at | 43 | Yes | IGFBP4 | Sense (Fully Exonic) | −0.6135 | −0.6133 |
| 2 | OCADA.9921_s_at | 44 | Yes | FOS | Sense (Fully Exonic) | −0.1143 | −0.1006 |
| 2 | OC3P.5101.C1_s_at | 45 | Yes | NR2F1 | Sense (Fully Exonic) | −0.5796 | −0.5018 |
| 2 | OC3P.3764.C1_s_at | 46 | Yes | MMP11 | Sense (Fully Exonic) | −0.5294 | −0.6942 |
| 2 | OC3SNG.2502-79a_s_at | 47 | Yes | IGFBP5 | Sense (Fully Exonic) | −0.5301 | −0.3920 |
| 2 | OCHP.1534_s_at | 48 | Yes | LUM | Sense (Fully Exonic) | −0.5754 | −0.4696 |
| 2 | OC3P.10470.C1_s_at | 49 | Yes | TIMP3 | Sense (Fully Exonic) | −0.5642 | −0.5616 |
| 2 | OC3SNGnh.19479_s_at | 50 | Yes | EGR1 | AntiSense | −0.1970 | −0.1264 |
| 2 | OC3P.13634.C1_s_at | 51 | Yes | IRS2 | Sense (Fully Exonic) | −0.4432 | −0.4892 |
| 2 | OC3P.373.C1-533a_s_at | 52 | Yes | RHOB | Sense (Fully Exonic) | −0.4433 | −0.2783 |
| 2 | OCMX.8.C2_s_at | 53 | Yes | EGR1 | AntiSense | 0.0109 | −0.0248 |
| 2 | OC3SNGnh.985_s_at | 54 | Yes | ABLIM1 | Sense (Fully Exonic) | −0.3497 | −0.2860 |
| 2 | OC3P.3458.C1_s_at | 55 | Yes | AEBP1 | Sense (Fully Exonic) | −0.6274 | −0.5348 |
| 2 | OC3SNGn.8474-50a_x_at | 56 | Yes | COL1A2 | Sense (Fully Exonic) | −0.8915 | −0.7965 |
| 2 | OC3P.81.CB2_s_at | 57 | Yes | COL3A1 | Sense (Fully Exonic) | −0.7728 | −0.6448 |
| 2 | OC3P.564.C1_s_at | 58 | Yes | VMP1 | Sense (Fully Exonic) | −0.0002 | −0.0165 |
| 2 | OCHP.148_s_at | 59 | Yes | CDH11 | Sense (Fully Exonic) | −0.6261 | −0.6122 |
| 2 | OC3P.4001.C1_s_at | 60 | Yes | GADD45B | Sense (Fully Exonic) | −0.3177 | −0.1886 |
| 2 | OC3P.1200.C1_s_at | 61 | Yes | VCAN | Sense (Fully Exonic) | −0.7519 | −0.6159 |
| 2 | OCMXSNG.5132_s_at | 62 | Yes | COL1A1 | AntiSense | −0.8073 | −0.6347 |
| 2 | OC3P.13652.C1_s_at | 63 | Yes | COL8A1 | Sense (Fully Exonic) | −0.6009 | −0.6239 |
| 2 | OC3P.1292.C1_s_at | 64 | Yes | EMP1 | Sense (Fully Exonic) | −0.5022 | −0.3751 |
| 2 | OC3P.543.CB1-699a_s_at | 65 | Yes | TIMP2 | Sense (Fully Exonic) | −0.7411 | −0.6593 |
| 2 | OC3P.2713.C1_s_at | 66 | Yes | COL5A2 | Sense (Fully Exonic) | −0.7083 | −0.7010 |
| 2 | OCHP.769_s_at | 67 | Yes | PDGFRA | Sense (Fully Exonic) | −0.5769 | −0.4759 |
| 2 | OC3SNGn.484-1a_s_at | 68 | Yes | HOXC6 | Sense (Fully Exonic) | −0.1743 | −0.2252 |
| 2 | OCADNP.830_s_at | 69 | Yes | IGFBP5 | Sense (Fully Exonic) | −0.4702 | −0.3184 |
| 2 | OC3SNGn.2801-166a_s_at | 70 | Yes | TWIST1 | Sense (Fully Exonic) | −0.6419 | −0.7108 |
| 2 | OCMXSNG.2027_x_at | 71 | Yes | TWIST1 | AntiSense | −0.6599 | −0.5645 |
| 2 | OCADA.8344_s_at | 72 | Yes | TPM1 | Sense (Includes Intronic) | −0.2574 | −0.2064 |
| 2 | OCHPRC.15_s_at | 73 | Yes | MSX1 | Sense (Fully Exonic) | −0.0635 | −0.2121 |
| 2 | OC3P.11485.C1_s_at | 74 | Yes | PSD3 | Sense (Fully Exonic) | −0.5048 | −0.3704 |
| 2 | OC3P.11604.C1_s_at | 75 | Yes | THBS1 | Sense (Fully Exonic) | −0.4353 | −0.2976 |
| 2 | OC3SNGn.793-57a_s_at | 76 | Yes | STMN3 | Sense (Fully Exonic) | −0.1961 | −0.1494 |
| 2 | OC3P.5893.C1_s_at | 77 | Yes | IRS1 | Sense (Fully Exonic) | −0.5374 | −0.4238 |
| 2 | OC3P.13061.C1_s_at | 78 | Yes | ROBO1 | Sense (Fully Exonic) | −0.4637 | −0.3727 |
| 2 | OCMXSNG.2027_at | 79 | Yes | TWIST1 | AntiSense | −0.6848 | −0.6751 |
| 2 | OC3P.10233.C1_s_at | 80 | Yes | TGFB3 | Sense (Fully Exonic) | −0.4452 | −0.3709 |
| 2 | OCMX.11138.C1_x_at | 81 | Yes | IGF1 | AntiSense | −0.2342 | −0.3079 |
| 2 | OCADA.6468_s_at | 82 | Yes | MSN | Sense (Includes Intronic) | 0.0426 | 0.2319 |
| 2 | OC3P.7062.C1_s_at | 83 | Yes | SGCB | Sense (Fully Exonic) | −0.3278 | −0.2089 |
| 2 | OC3SNG.1705-33a_s_at | 84 | Yes | WNT7A | Sense (Fully Exonic) | −0.5164 | −0.7588 |
| 2 | OC3P.164.C1_s_at | 85 | Yes | NID2 | Sense (Fully Exonic) | −0.4941 | −0.3889 |
| 2 | OC3SNGnh.6980_s_at | 86 | Yes | IGFBP5 | AntiSense | −0.4812 | −0.2969 |
| 2 | OC3SNGn.469-921a_s_at | 87 | Yes | EGR1 | Sense (Fully Exonic) | −0.0509 | −0.1453 |
| 2 | OCMX.493.C1_s_at | 88 | Yes | FN1 | Sense (Fully Exonic) | −0.3423 | −0.1453 |
| 2 | OC3P.10127.C1_s_at | 89 | Yes | HOXC6 | Sense (Fully Exonic) | −0.1116 | −0.1512 |
| 2 | OC3P.2278.C1_x_at | 90 | Yes | CERCAM | Sense (Fully Exonic) | −0.7347 | −0.7399 |
| 2 | OC3P.2179.C1_s_at | 91 | Yes | SULF2 | Sense (Fully Exonic) | −0.6395 | −0.5969 |
| 2 | OC3P.8087.C1_s_at | 92 | Yes | GAS7 | Sense (Fully Exonic) | −0.4776 | −0.4086 |
| 2 | OC3P.3034.C1_s_at | 93 | Yes | NDN | Sense (Fully Exonic) | −0.5590 | −0.5346 |
| 2 | OC3P.1178.C1_at | 94 | Yes | CTGF | Sense (Fully Exonic) | −0.4900 | −0.4178 |
| 2 | OC3P.10040.C1_s_at | 95 | Yes | PDGFC | Sense (Fully Exonic) | −0.4219 | −0.3349 |
| 2 | OC3SNGnh.11427_x_at | 96 | Yes | COL12A1 | Sense (Includes Intronic) | −0.3941 | −0.3794 |
| 2 | OCADA.1904_s_at | 97 | Yes | PDGFC | Sense (Includes Intronic) | −0.3168 | −0.1160 |
| 2 | OC3SNGnh.11631_s_at | 98 | Yes | SDK1 | Sense (Includes Intronic) | −0.6334 | −0.4632 |
| 2 | OCADNP.13759_s_at | 99 | Yes | DPYSL3 | Sense (Includes Intronic) | −0.3283 | −0.1273 |
| 2 | OC3SNG.5645-98a_x_at | 100 | Yes | CCDC80 | Sense (Fully Exonic) | −0.5288 | −0.3665 |
| 2 | OC3SNGnh.487_at | 101 | Yes | TPM1 | Sense (Fully Exonic) | −0.2798 | −0.1964 |
| 2 | OC3SNG.3829-22a_s_at | 102 | Yes | CSRNP1 | Sense (Fully Exonic) | −0.0370 | −0.1197 |
| 2 | OCHP.164_s_at | 103 | Yes | PROCR | Sense (Fully Exonic) | −0.2058 | −0.3175 |
| 2 | OC3P.10157.C1_s_at | 104 | Yes | COL15A1 | Sense (Fully Exonic) | −0.3492 | −0.3688 |
| 2 | OCMX.11138.C1_at | 105 | Yes | IGF1 | AntiSense | −0.2100 | −0.3583 |
| 2 | OC3SNGnh.11427_at | 106 | Yes | COL12A1 | Sense (Includes Intronic) | −0.3071 | −0.1665 |
| 2 | OCHP.1423_s_at | 107 | Yes | APCDD1 | Sense (Fully Exonic) | −0.4017 | −0.3332 |
| 2 | OCADNP.8535_s_at | 108 | Yes | FGFR1 | Sense (Fully Exonic) | −0.2415 | −0.2793 |
| 2 | OC3P.13517.C1_s_at | 109 | Yes | EDA2R | Sense (Fully Exonic) | −0.4296 | −0.2344 |
| 2 | OC3SNGnh.1613_at | 110 | Yes | ACSL4 | Sense (Includes Intronic) | −0.1428 | −0.0762 |
| 2 | OCMX.2061.C1_s_at | 111 | Yes | ENC1 | Sense (Fully Exonic) | −0.3510 | −0.3167 |
| 2 | OC3P.560.C1_s_at | 112 | Yes | JAM3 | Sense (Fully Exonic) | −0.5978 | −0.7041 |
| 2 | OC3SNG.1834-947a_s_at | 113 | Yes | COL10A1 | Sense (Fully Exonic) | −0.5399 | −0.4784 |
| 2 | OC3P.6769.C1_s_at | 114 | Yes | HOPX | Sense (Fully Exonic) | −0.3602 | −0.3815 |
| 2 | OC3SNGn.2612-800a_s_at | 115 | Yes | ARL4A | Sense (Fully Exonic) | −0.2482 | −0.1542 |
| 2 | OCADNP.2893_s_at | 116 | Yes | ASH2L | Sense (Includes Intronic) | −0.0048 | 0.0555 |
| 2 | OCRS.320_s_at | 117 | Yes | NOX4 | Sense (Fully Exonic) | −0.1853 | −0.1276 |
| 2 | OC3SNGn.6594-7a_s_at | 118 | Yes | COL14A1 | Sense (Fully Exonic) | −0.0757 | 0.0013 |
| 2 | OC3P.5849.C1_s_at | 119 | Yes | TYRO3 | Sense (Fully Exonic) | −0.0297 | −0.0889 |
| 2 | OC3P.10562.C1_s_at | 120 | Yes | COL8A1 | Sense (Fully Exonic) | −0.5165 | −0.4212 |
| 2 | OC3SNGnh.5170_x_at | 121 | Yes | RORA | Sense (Includes Intronic) | −0.1302 | −0.3066 |
| 2 | OC3P.6842.C1_s_at | 122 | Yes | NPAS2 | Sense (Fully Exonic) | −0.1420 | 0.0132 |
| 2 | OC3P.5913.C1_s_at | 123 | Yes | PRICKLE2 | Sense (Fully Exonic) | −0.4466 | −0.4348 |
| 2 | OC3SNGnh.14944_at | 124 | Yes | PLA2R1 | Sense (Includes Intronic) | −0.1046 | −0.1698 |
| 2 | OCADA.7782_s_at | 125 | Yes | GSN | Sense (Includes Intronic) | −0.2917 | −0.2583 |
| 2 | OC3P.12692.C1_s_at | 126 | Yes | ADH5 | Sense (Fully Exonic) | −0.3531 | −0.2766 |
| 2 | OCHP.1016_s_at | 127 | Yes | APOD | Sense (Fully Exonic) | −0.2923 | −0.3323 |
| 2 | OCHP.739_s_at | 128 | Yes | PLAU | Sense (Fully Exonic) | −0.2212 | −0.1977 |
| 2 | OC3P.8445.C1_s_at | 129 | Yes | NRP1 | Sense (Fully Exonic) | −0.2833 | −0.2569 |
| 2 | OC3SNGn.7890-859a_x_at | 130 | Yes | WNT4 | Sense (Fully Exonic) | −0.2175 | −0.3361 |
| 2 | OC3SNGnh.3154_s_at | 131 | Yes | CHN1 | Sense (Fully Exonic) | −0.5802 | −0.5367 |
| 2 | OC3P.305.C1_at | 132 | Yes | BTG2 | Sense (Fully Exonic) | 0.1127 | −0.0791 |
| 2 | OC3SNGn.6036-20a_x_at | 133 | Yes | FGFR1 | Sense (Fully Exonic) | −0.3997 | −0.3814 |
| 2 | OC3P.697.C1_s_at | 134 | Yes | NFKBIZ | Sense (Fully Exonic) | 0.0166 | 0.0547 |
| 2 | OCMXSNG.5132_x_at | 135 | Yes | COL1A1 | AntiSense | −0.8603 | −0.6216 |
| 2 | OC3P.1878.C1_s_at | 136 | Yes | TNC | Sense (Fully Exonic) | −0.2603 | −0.2119 |
| 2 | OC3SNGnh.5090_at | 137 | Yes | TPM1 | Sense (Fully Exonic) | −0.2257 | −0.1486 |
| 2 | OC3P.13621.C1_s_at | 138 | Yes | SFRP2 | Sense (Fully Exonic) | −0.2070 | −0.2520 |
| 2 | OC3SNGnh.8739_s_at | 139 | Yes | DUSP4 | Sense (Fully Exonic) | −0.1419 | −0.2038 |
| 2 | OCHP.1881_s_at | 140 | Yes | KIT | Sense (Fully Exonic) | −0.4643 | −0.5299 |
| 2 | OCHP.1072_s_at | 141 | Yes | CXCL14 | Sense (Fully Exonic) | −0.7036 | −0.7096 |
| 2 | OCRS.383_s_at | 142 | Yes | COL10A1 | Sense (Fully Exonic) | −0.3721 | −0.4865 |
| 2 | OCHPRC.106_s_at | 143 | Yes | ADAMTS2 | Sense (Fully Exonic) | −0.5826 | −0.6585 |
| 2 | OCHP.1005_s_at | 144 | Yes | COL5A1 | Sense (Fully Exonic) | −0.6231 | −0.5273 |
| 2 | OC3P.925.C1_s_at | 145 | Yes | ANTXR1 | Sense (Fully Exonic) | −0.7671 | −0.6024 |
| 2 | OC3P.9910.C1_s_at | 146 | Yes | FBLIM1 | Sense (Fully Exonic) | −0.7257 | −0.4521 |
| 2 | OCRS2.9432_s_at | 147 | Yes | SPAG16 | Sense (Fully Exonic) | −0.1089 | 0.0000 |
| 2 | OC3SNGnh.16119_at | 148 | Yes | PDGFD | Sense (Includes Intronic) | −0.1329 | −0.2945 |
| 2 | OCADNP.7019_s_at | 149 | Yes | PLXNA4 | Sense (Fully Exonic) | −0.1474 | −0.3637 |
| 2 | OC3P.8373.C1_s_at | 150 | Yes | SDC2 | Sense (Fully Exonic) | −0.4106 | −0.4582 |
| 2 | OC3P.13498.C1_s_at | 151 | Yes | NAV1 | Sense (Fully Exonic) | −0.5696 | −0.4732 |
| 2 | OC3SNGnh.19238_s_at | 152 | Yes | TIMP2 | Sense (Fully Exonic) | −0.7506 | −0.8010 |
| 2 | OC3P.2537.CB1_s_at | 153 | Yes | MYL9 | Sense (Fully Exonic) | −0.3703 | −0.2316 |
| 2 | OCADA.6829_s_at | 154 | Yes | MAP3K1 | Sense (Includes Intronic) | −0.1712 | −0.0706 |
| 2 | OC3P.5230.C1_s_at | 155 | Yes | EPDR1 | Sense (Fully Exonic) | −0.4676 | −0.3070 |
| 2 | OCADA.3572_s_at | 156 | Yes | TRIM13 | Sense (Fully Exonic) | −0.3381 | −0.2101 |
| 2 | OCADA.7893_s_at | 157 | Yes | EFNA5 | Sense (Fully Exonic) | −0.1234 | −0.1621 |
| 2 | OC3SNG.1306-60a_s_at | 158 | Yes | DDR2 | Sense (Fully Exonic) | −0.2990 | −0.4008 |
| 2 | OC3P.850.C1-1145a_s_at | 159 | Yes | COL4A1 | Sense (Fully Exonic) | −0.8270 | −0.8242 |
| 2 | OC3SNGnh.9087_at | 160 | Yes | EFNA5 | AntiSense | −0.2111 | −0.0308 |
| 2 | OC3SNGnh.12139_at | 161 | Yes | FYN | Sense (Fully Exonic) | −0.1142 | −0.1521 |

TABLE B
Immune PS

| Cluster | | SEQ ID | Core | Gene | | mean expression | |
|---|---|---|---|---|---|---|---|
| # | Probeset ID | NO | (Yes/No) | Symbol | Orientation | in Immune Group | bias |
| 1 | OC3P.141.C13_s_at | 162 | Yes | HLA-F | Sense (Fully Exonic) | 0.3512 | 0.4146 |
| 1 | OC3SNGn.2735-12a_s_at | 163 | Yes | HLA-DPA1 | Sense (Fully Exonic) | 0.4004 | 0.4337 |
| 1 | OC3P.5227.C1_s_at | 164 | Yes | HCLS1 | Sense (Fully Exonic) | 0.0636 | 0.0808 |
| 1 | OCHP.345_s_at | 165 | Yes | SFN | Sense (Fully Exonic) | 0.1115 | 0.1967 |
| 1 | OCMXSNG.5067_s_at | 166 | Yes | B2M | Sense (Fully Exonic) | 0.3440 | 0.4275 |
| 1 | OC3P.7557.C1_s_at | 167 | Yes | NLRC5 | Sense (Fully Exonic) | 0.3322 | 0.4390 |
| 1 | OCRS2.2571_s_at | 168 | Yes | HCLS1 | Sense (Fully Exonic) | 0.0461 | 0.0695 |
| 1 | OCMXSNG.5608_at | 169 | Yes | APOL1 | AntiSense | 0.2376 | 0.2180 |
| 1 | OCRS2.4310_s_at | 170 | Yes | ITGB2 | Sense (Fully Exonic) | 0.0740 | 0.1774 |
| 1 | OCHP.1588_s_at | 171 | Yes | STAT1 | Sense (Fully Exonic) | 0.3506 | 0.4967 |
| 1 | OCHP.1640_s_at | 172 | Yes | NNMT | Sense (Fully Exonic) | −0.1648 | −0.2533 |
| 1 | OC3P.7284.C1_s_at | 173 | Yes | VCAM1 | Sense (Fully Exonic) | −0.2257 | −0.2808 |
| 1 | OC3SNG.2605-236a_x_at | 174 | Yes | XAF1 | Sense (Fully Exonic) | 0.4106 | 0.5108 |
| 1 | OC3P.805.C1_s_at | 175 | Yes | CIITA | Sense (Fully Exonic) | 0.4992 | 0.5791 |
| 1 | OCRS2.731_x_at | 176 | Yes | HLA-B | Sense (Fully Exonic) | 0.3616 | 0.5702 |
| 1 | OC3P.2460.C1_s_at | 177 | Yes | IFIT2 | Sense (Fully Exonic) | 0.3214 | 0.3687 |
| 1 | OC3P.3169.C1_s_at | 178 | Yes | GBP2 | Sense (Fully Exonic) | 0.1979 | 0.2353 |
| 1 | OC3SNGn.6880-3840a_x_at | 179 | Yes | HLA-A | Sense (Fully Exonic) | 0.3232 | 0.4391 |
| 1 | OC3SNGn.1244-62a_x_at | 180 | Yes | HLA-A | Sense (Fully Exonic) | 0.0343 | 0.3472 |
| 1 | OCRS2.4548_s_at | 181 | Yes | PML | Sense (Fully Exonic) | 0.2464 | 0.1236 |
| 1 | OCMXSNG.5528_s_at | 182 | Yes | C1QC | AntiSense | 0.0561 | 0.1424 |
| 1 | OC3P.4435.C1-401a_s_at | 183 | Yes | IRF1 | Sense (Fully Exonic) | 0.4130 | 0.5033 |
| 1 | OC3P.8722.C1_s_at | 184 | Yes | ITGB2 | Sense (Fully Exonic) | 0.1402 | 0.2332 |
| 1 | OC3P.1164.C1_s_at | 185 | Yes | HLA-DPB1 | Sense (Fully Exonic) | 0.0267 | 0.1437 |
| 1 | OC3SNGn.6460-38a_x_at | 186 | Yes | HLA-A | Sense (Fully Exonic) | 0.0686 | 0.3330 |
| 1 | OC3P.141.C12_x_at | 187 | Yes | HLA-B | Sense (Fully Exonic) | 0.3578 | 0.5488 |
| 1 | OC3P.5468.C1_s_at | 188 | Yes | C1QB | Sense (Fully Exonic) | 0.1769 | 0.1907 |
| 1 | OC3P.1177.C1_x_at | 189 | Yes | APOL1 | Sense (Fully Exonic) | 0.2318 | 0.1271 |
| 1 | OC3SNG.1495-79a_s_at | 190 | Yes | BST2 | Sense (Fully Exonic) | 0.1831 | 0.2477 |
| 1 | OCMX.670.CB2_at | 191 | Yes | CD74 | AntiSense | 0.4907 | 0.5318 |
| 1 | OC3SNG.4002-20a_s_at | 192 | Yes | RASGRP2 | Sense (Fully Exonic) | 0.0743 | 0.0246 |
| 1 | OC3SNGnh.19645_s_at | 193 | Yes | MX1 | Sense (Fully Exonic) | 0.3941 | 0.5564 |
| 1 | OCHP.366_s_at | 194 | Yes | CTSB | Sense (Fully Exonic) | −0.0673 | 0.0000 |
| 1 | OCMX.125.C1_s_at | 195 | Yes | GBP1 | AntiSense | 0.4669 | 0.5520 |
| 1 | OC3P.4873.C1_s_at | 196 | Yes | XAF1 | Sense (Fully Exonic) | 0.3593 | 0.5107 |
| 1 | OCADNP.3105_s_at | 197 | Yes | B2M | Sense (Includes Intronic) | 0.3000 | 0.3888 |
| 1 | OCRS2.2819_x_at | 198 | Yes | HLA-F | Sense (Fully Exonic) | 0.3840 | 0.4789 |
| 1 | OC3P.6011.C1_s_at | 199 | Yes | PLCG2 | Sense (Fully Exonic) | 0.0644 | −0.0159 |
| 1 | OC3SNG.856-35a_x_at | 200 | Yes | C1QC | Sense (Fully Exonic) | 0.1522 | 0.1744 |
| 1 | OC3SNGn.3058-31a_s_at | 201 | Yes | GBP5 | Sense (Fully Exonic) | 0.4388 | 0.2827 |
| 1 | OC3P.14483.C1_s_at | 202 | Yes | SOD2 | Sense (Fully Exonic) | 0.1843 | 0.2792 |
| 1 | OC3SNGn.2005-402a_s_at | 203 | Yes | CD163 | Sense (Fully Exonic) | 0.0270 | 0.1413 |
| 1 | OC3SNGnh.10611_x_at | 204 | Yes | BST2 | Sense (Fully Exonic) | −0.0402 | 0.1233 |
| 1 | OC3SNG.2053-58a_s_at | 205 | Yes | FBP1 | Sense (Fully Exonic) | 0.2508 | 0.2776 |
| 1 | OC3P.4732.C1_s_at | 206 | Yes | CD44 | Sense (Fully Exonic) | 0.1702 | 0.1368 |
| 1 | OCRS2.2819_at | 207 | Yes | HLA-F | Sense (Fully Exonic) | 0.4535 | 0.5759 |
| 1 | OC3SNG.3064-21a_x_at | 208 | Yes | CD74 | Sense (Fully Exonic) | 0.3467 | 0.3885 |
| 1 | Adx-Hs-ISGF3A-300-3_x_at | 209 | Yes | STAT1 | Sense (Fully Exonic) | 0.2161 | 0.3869 |
| 1 | OC3SNGn.6006-1022a_s_at | 210 | Yes | C1S | Sense (Fully Exonic) | −0.2849 | −0.2531 |
| 1 | OCADA.10565_s_at | 211 | Yes | GBP1 | Sense (Fully Exonic) | 0.3837 | 0.4946 |
| 1 | OC3P.530.C1-561a_s_at | 212 | Yes | XBP1 | Sense (Fully Exonic) | 0.2479 | 0.1445 |
| 1 | OC3P.4729.C1_s_at | 213 | Yes | HLA-DMB | Sense (Fully Exonic) | 0.4402 | 0.5053 |
| 1 | OC3P.9869.C1_s_at | 214 | Yes | MAFB | Sense (Fully Exonic) | −0.2958 | −0.2942 |
| 1 | OCADA.3339_s_at | 215 | Yes | DERL3 | Sense (Fully Exonic) | −0.0282 | 0.0194 |
| 1 | OC3SNG.3595-3338a_s_at | 216 | Yes | CYLD | Sense (Fully Exonic) | 0.1067 | 0.1572 |
| 1 | Adx-Hs-ISGF3A-400-3_x_at | 217 | Yes | STAT1 | Sense (Fully Exonic) | 0.2313 | 0.3557 |
| 1 | OC3SNGn.883-5a_s_at | 218 | Yes | TREM2 | Sense (Fully Exonic) | 0.2394 | −0.0068 |
| 1 | OC3SNGnh.2550_s_at | 219 | Yes | FCER1G | Sense (Fully Exonic) | 0.0441 | 0.1559 |
| 1 | OC3P.1033.C1_s_at | 220 | Yes | LGALS9 | Sense (Fully Exonic) | 0.3702 | 0.4531 |
| 1 | OC3P.7068.C1_s_at | 221 | Yes | UBE2L6 | Sense (Fully Exonic) | 0.4012 | 0.4545 |
| 1 | OCHP.1827_s_at | 222 | Yes | SIGLEC1 | Sense (Fully Exonic) | 0.3244 | 0.2161 |
| 1 | OC3SNGn.5100-4676a_s_at | 223 | Yes | MMP7 | Sense (Fully Exonic) | 0.0456 | 0.1118 |
| 1 | OCADA.10811_s_at | 224 | Yes | SLAMF7 | Sense (Fully Exonic) | 0.3653 | 0.3153 |
| 1 | OC3P.5930.C1_at | 225 | Yes | LITAF | Sense (Fully Exonic) | 0.0439 | 0.1634 |
| 1 | OC3P.10280.C1_s_at | 226 | Yes | IFIH1 | Sense (Fully Exonic) | 0.4117 | 0.5086 |
| 1 | OC3SNG.2984-24a_s_at | 227 | Yes | TYROBP | Sense (Fully Exonic) | 0.1072 | 0.0052 |
| 1 | OC3P.10546.C1_s_at | 228 | Yes | ALOX5 | Sense (Fully Exonic) | −0.0189 | −0.0037 |
| 1 | OCHP.489_s_at | 229 | Yes | IL1RN | Sense (Fully Exonic) | 0.1878 | 0.1202 |
| 1 | OC3P.7013.C1_s_at | 230 | Yes | ADAM8 | Sense (Fully Exonic) | 0.1126 | 0.1079 |
| 1 | OC3P.1545.CB1_x_at | 231 | Yes | BST2 | Sense (Fully Exonic) | −0.0202 | 0.1606 |
| 1 | OCADNP.7474_s_at | 232 | Yes | CTSS | Sense (Fully Exonic) | 0.3166 | 0.4647 |
| 1 | OC3P.13144.C1-468a_s_at | 233 | Yes | HMHA1 | Sense (Fully Exonic) | 0.3289 | 0.3302 |
| 1 | OCADNP.3111_s_at | 234 | Yes | STAT1 | Sense (Includes Intronic) | 0.4544 | 0.5399 |
| 1 | OCRS2.2290_s_at | 235 | Yes | DGKA | Sense (Fully Exonic) | −0.0641 | −0.0057 |
| 1 | OC3P.77.C1_s_at | 236 | Yes | CTSB | Sense (Fully Exonic) | 0.0202 | 0.1147 |
| 1 | OCMX.2432.C4_s_at | 237 | Yes | CTSB | Sense (Fully Exonic) | 0.1490 | 0.1090 |
| 1 | OC3P.9251.C1_s_at | 238 | Yes | CD4 | Sense (Fully Exonic) | 0.0415 | 0.1595 |

The cancer sub-type may be defined by the probesets listed in Tables A and B and by the expression levels of the corresponding genes in Tables A and B, which may be measured using the probesets. Negative values are indicative of decreased (mean) expression levels and positive values of increased (mean) expression levels.

In a further aspect the invention provides a method for selecting whether to administer an anti-angiogenic therapeutic agent to a subject with cancer, comprising:

measuring the expression levels of at least 3 biomarkers in a sample from the subject,

wherein at least two of the biomarkers are from Table A and at least one of the biomarkers is from Table B and assessing from the expression levels of the biomarkers whether the cancer belongs to a cancer sub-type

(optionally) wherein the cancer sub-type is defined by the expression levels of the genes in Tables A and B

wherein if the cancer belongs to the sub-type an anti-angiogenic therapeutic agent is contraindicated

wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

According to a further aspect of the invention there is provided a method for selecting whether to administer an anti-angiogenic therapeutic agent to a subject with cancer, comprising:

measuring the expression levels of at least 3 biomarkers in a sample from the subject,

wherein at least two of the biomarkers are from Table A and at least one of the biomarkers is from Table B and assessing from the expression levels of the biomarkers whether the cancer belongs to a cancer sub-type, wherein if the cancer belongs to the subtype the expression levels of the at least two biomarkers from Table A and the at least one biomarker from Table B are increased or decreased as defined for each biomarker in Table A and Table B (optionally) wherein the cancer sub-type is defined by the expression levels of the genes in Tables A and B

wherein if the cancer belongs to the sub-type an anti-angiogenic therapeutic agent is contraindicated.

In yet a further aspect, the present invention relates to a method for predicting the responsiveness of a subject with cancer to an anti-angiogenic therapeutic agent comprising:

allocating the cancer to a cancer sub-type by measuring the expression levels of at least 3 biomarkers in a sample from the subject, wherein at least two of the biomarkers are from Table A and at least one of the biomarkers is from Table B [IMMUNE LIST] and assessing from the expression levels of the biomarkers whether the cancer belongs to the sub-type (optionally) wherein the cancer sub-type is defined by the expression levels of the genes in Tables A and B

classifying the subject as responsive or non-responsive to the anti-angiogenic therapeutic agent on the basis of allocation to the subtype, wherein if the cancer belongs to the sub-type it is predicted to be non-responsive to the anti-angiogenic therapeutic agent

wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

The invention also relates to a method for predicting the responsiveness of a subject with cancer to an anti-angiogenic therapeutic agent comprising:

allocating the cancer to a cancer sub-type by measuring the expression level of at least 3 biomarkers in a sample from the subject, wherein at least two of the biomarkers are from Table A and at least one of the biomarkers is from Table B and assessing from the expression levels of the biomarkers whether the cancer belongs to the sub-type wherein if the cancer belongs to the subtype the expression levels of the at least two biomarkers from Table A and the at least one biomarker from Table B are increased or decreased as defined for each biomarker in Table A and Table B

(optionally) wherein the cancer sub-type is defined by the expression levels of the genes in Tables A and B

classifying the subject as responsive or non-responsive to the anti-angiogenic therapeutic agent on the basis of allocation to the subtype, wherein if the cancer belongs to the sub-type it is predicted to be non-responsive to the anti-angiogenic therapeutic agent.

In a further aspect, the present invention relates to a method of determining clinical prognosis of a subject with cancer comprising:

measuring the expression level of at least 3 biomarkers in a sample from the subject,

wherein at least two of the biomarkers are from Table A and at least one of the biomarkers is from Table B and assessing from the expression levels of the biomarkers whether the cancer belongs to a cancer sub-type

(optionally) wherein the cancer sub-type is defined by the expression levels of the genes in Tables A and B

classifying the subject as having a good prognosis if the cancer belongs to the sub-type wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

The invention also relates to a method of determining clinical prognosis of a subject with cancer comprising:

measuring the expression level of at least 3 biomarkers in a sample from the subject,

wherein at least two of the biomarkers are from Table A and at least one of the biomarkers is from Table B and assessing from the expression levels of the biomarkers whether the cancer belongs to a cancer sub-type wherein if the cancer belongs to the subtype the expression levels of the at least two biomarkers from Table A and the at least one biomarker from Table B are increased or decreased as defined for each biomarker in Table A and Table B

(optionally) wherein the cancer sub-type is defined by the expression levels of the genes in Tables A and B

classifying the subject as having a good prognosis if the cancer belongs to the sub-type.

In yet a further aspect, the present invention relates to a method for selecting whether to administer an anti-angiogenic therapeutic agent to a subject with cancer, comprising:

measuring the expression levels of at least 2 biomarkers in a sample from the subject and assessing from the expression levels of the biomarkers whether the cancer belongs to a cancer sub-type

(optionally) wherein the cancer sub-type is defined by the expression levels of the genes in Tables A and B

wherein if the cancer belongs to the sub-type an anti-angiogenic therapeutic agent is contraindicated

wherein the at least 2 biomarkers do not consist of from 1 to 63 of the biomarkers shown in Table C.

TABLE C

| GeneSymbol | GeneWeights | GeneBias |
|---|---|---|
| IGF2 | −0.01737 | 9.8884 |
| SOX11 | −0.01457 | 4.5276 |
| INS | −0.01409 | 7.0637 |
| CXCL17 | 0.012568 | 4.8478 |
| SLC5A1 | 0.012426 | 4.892 |
| TMEM45A | −0.0124 | 6.1307 |
| CXCR2P1 | 0.011427 | 3.1478 |
| MFAP2 | −0.01039 | 9.0516 |
| MATN3 | −0.01028 | 3.7313 |
| RTP4 | 0.010052 | 4.9852 |
| COL3A1 | −0.01002 | 8.413 |
| CDR1 | −0.00916 | 8.1778 |
| RARRES3 | 0.009056 | 6.8964 |
| TNFSF10 | 0.008876 | 6.2325 |
| NUAK1 | −0.0087 | 6.6771 |
| SNORD114-14 | −0.00864 | 5.6385 |
| SRPX | −0.00862 | 5.085 |
| SPARC | −0.00848 | 6.0135 |
| GJB1 | 0.008445 | 5.8142 |
| TIMP3 | −0.00823 | 6.5937 |
| ISLR | −0.0079 | 8.9876 |
| TUBA1A | −0.00754 | 9.654 |
| DEXI | 0.007271 | 5.5913 |
| BASP1 | −0.00724 | 8.4396 |
| PXDN | −0.00724 | 7.757 |
| GBP4 | 0.007226 | 3.1119 |
| SLC28A3 | 0.007201 | 4.2125 |
| HLA-DRA | 0.007197 | 8.3089 |
| TAP2 | 0.007189 | 4.8464 |
| ACSL5 | 0.007155 | 6.8703 |
| CDH11 | −0.00708 | 4.9925 |
| PSMB9 | 0.006962 | 4.1122 |
| MMP14 | −0.00683 | 10.1689 |
| CD74 | 0.006825 | 9.2707 |
| LOXL1 | −0.00676 | 9.6429 |
| CIITA | 0.006623 | 5.5396 |
| ZNF697 | −0.00658 | 7.0319 |
| SH3RF2 | 0.006549 | 5.0029 |
| MIR198 | −0.00654 | 5.1935 |
| COL1A2 | −0.00645 | 6.0427 |
| TNFRSF14 | 0.006421 | 9.0366 |
| COL8A1 | −0.00642 | 6.4565 |
| C21orf63 | 0.006261 | 5.9811 |
| TAP1 | 0.006215 | 8.6458 |
| PDPN | −0.00612 | 5.3198 |
| RHOBTB3 | −0.00597 | 3.5609 |
| BCL11A | 0.005943 | 4.3818 |
| HLA-DOB | 0.005851 | 4.6075 |
| XAF1 | 0.005742 | 7.9229 |
| ARHGAP26 | 0.005632 | 4.3991 |
| POLD2 | −0.00558 | 9.4183 |
| DPYSL2 | −0.00533 | 8.3469 |
| COL4A1 | −0.0052 | 7.0317 |
| ID3 | −0.00516 | 7.5673 |
| CFB | 0.005077 | 5.7951 |
| NID1 | −0.00494 | 4.7186 |
| FKBP7 | −0.00489 | 2.9437 |
| TIMP2 | −0.00468 | 7.5253 |
| RCBTB1 | −0.00458 | 7.4491 |
| ANGPTL2 | −0.00448 | 5.6807 |
| ENTPD7 | −0.00442 | 7.3772 |
| SHISA4 | −0.00403 | 6.0601 |
| HINT1 | 0.003651 | 6.0724 |

The genes from Table C are shown ranked in Table D and probesets that can be used to detect these genes are shown in Table E.

TABLE D

| Gene | Total Delta HR | Rank |
|---|---|---|
| IGF2 | 0.048910407 | 1 |
| CDR1 | 0.045335288 | 2 |
| COL3A1 | 0.044869217 | 3 |
| SPARC | 0.043434096 | 4 |
| TIMP3 | 0.042053053 | 5 |
| INS | 0.04013658 | 6 |
| COL8A1 | 0.026780907 | 7 |
| NUAK1 | 0.026752491 | 8 |
| MATN3 | 0.02402318 | 9 |
| TMEM45A | 0.016999761 | 10 |
| SRPX | 0.016372168 | 11 |
| CDH11 | 0.015604812 | 12 |
| MMP14 | 0.014583388 | 13 |
| LOXL1 | 0.010315358 | 14 |
| PXDN | 0.009728534 | 15 |
| COL1A2 | 0.009267887 | 16 |
| ANGPTL2 | 0.006071504 | 17 |
| POLD2 | 0.004297935 | 18 |
| NID1 | 0.00408724 | 19 |
| ISLR | 0.003014488 | 20 |
| SNORD114-14 | 0.002992636 | 21 |
| CXCR2P1 | 0.002804432 | 22 |
| MIR198 | 0.002173041 | 23 |
| BCL11A | 0.001258286 | 24 |
| PDPN | 0.000989109 | 25 |
| TNFRSF14 | 0.000132838 | 26 |
| ENTPD7 | 6.25143E−05 | 27 |
| HINT1 | −0.000113156 | 28 |
| TAP1 | −0.000379242 | 29 |
| ID3 | −0.000452476 | 30 |
| RCBTB1 | −0.000695459 | 31 |
| SOX11 | −0.001068812 | 32 |
| SHISA4 | −0.001470801 | 33 |
| COL4A1 | −0.001714442 | 34 |
| TUBA1A | −0.001817696 | 35 |
| TIMP2 | −0.004079263 | 36 |
| FKBP7 | −0.004575097 | 37 |
| TAP2 | −0.004597761 | 38 |
| TNFSF10 | −0.005307314 | 39 |
| ZNF697 | −0.007733496 | 40 |
| CIITA | −0.008785689 | 41 |
| BASP1 | −0.009340492 | 42 |
| XAF1 | −0.009760794 | 43 |
| DEXI | −0.009798099 | 44 |
| SH3RF2 | −0.009856754 | 45 |
| HLA-DOB | −0.009987248 | 46 |
| RHOBTB3 | −0.010264542 | 47 |
| GBP4 | −0.010747831 | 48 |
| DPYSL2 | −0.012042179 | 49 |
| ARHGAP26 | −0.012380203 | 50 |
| MFAP2 | −0.013981916 | 51 |
| CD74 | −0.016415304 | 52 |
| ACSL5 | −0.016912224 | 53 |
| SLC28A3 | −0.016996213 | 54 |
| GJB1 | −0.018395345 | 55 |
| C21orf63 | −0.019853038 | 56 |
| PSMB9 | −0.020314379 | 57 |
| HLA-DRA | −0.020436677 | 58 |
| CFB | −0.022202886 | 59 |
| RARRES3 | −0.034723666 | 60 |
| CXCL17 | −0.038523986 | 61 |
| SLC5A1 | −0.042034346 | 62 |
| RTP4 | −0.045259104 | 63 |

TABLE E

| Probeset | Gene | SEQ ID No. |
|---|---|---|
| OC3P.6916.C1_s_at | ACSL5 | 239 |
| OC3P.5381.C1_s_at | ACSL5 | 240 |
| OC3P.2679.C1_s_at | ANGPTL2 | 241 |
| ADXStrongB12_at | ANGPTL2 | N/A |
| OC3P.9834.C1_s_at | ANGPTL2 | 242 |
| OCMX.9546.C1_x_at | ANGPTL2 | 243 |
| OCADA.8226_s_at | ANGPTL2 | 244 |
| OCADNP.8811_s_at | ANGPTL2 | 245 |
| OCADA.3065_s_at | ARHGAP26 | 246 |
| OCADA.1272_s_at | ARHGAP26 | 247 |
| OC3SNGnh.16379_x_at | ARHGAP26 | 248 |
| OCMX.11710.C1_at | ARHGAP26 | 249 |
| OCADA.4396_s_at | ARHGAP26 | 250 |
| OC3P.15451.C1_at | ARHGAP26 | 251 |
| OC3SNGnh.16379_at | ARHGAP26 | 252 |
| OC3SNGnh.17316_s_at | ARHGAP26 | 253 |
| OCADA.964_s_at | ARHGAP26 | 254 |
| OC3SNGnh.6403_s_at | ARHGAP26 | 255 |
| OC3P.3912.C1_s_at | ARHGAP26 | 256 |
| OC3P.2419.C1_s_at | BASP1 | 257 |
| OCRS2.9952_s_at | BASP1 | 258 |
| OCRS2.9952_x_at | BASP1 | 259 |
| OCRS.854_s_at | BCL11A | 260 |
| OC3P.14938.C1_s_at | BCL11A | 261 |
| OCMX.12290.C1_at | BCL11A | 262 |
| OCADA.10230_s_at | BCL11A | 263 |
| OC3SNGnh.4343_at | BCL11A | 264 |
| OC3SNGnh.16766_x_at | BCL11A | 265 |
| OCMX.1680.C1_s_at | BCL11A | 266 |
| OC3P.14938.C1-334a_s_at | BCL11A | 267 |
| OCMX.12290.C1_x_at | BCL11A | 268 |
| OCADA.2850_s_at | BCL11A | 269 |
| OCADA.1135_s_at | C21orf63 | 270 |
| OCMX.14248.C1_s_at | C21orf63 | 271 |
| OC3P.14091.C1_s_at | C21orf63 | 272 |
| OC3P.14431.C1_s_at | C21orf63 | 273 |
| OCADA.8368_x_at | CD74 | 274 |
| OC3SNGnh.19144_s_at | CD74 | 275 |
| OC3P.104.CB1_x_at | CD74 | 276 |
| OCADNP.1805_s_at | CD74 | 277 |
| OC3SNG.3064-21a_x_at | CD74 | 278 |
| OC3P.14147.C1_s_at | CDH11 | 279 |
| OCADNP.10024_s_at | CDH11 | 280 |
| OCHP.148_s_at | CDH11 | 281 |
| OCADA.6210_s_at | CDH11 | 282 |
| OC3SNGnh.5056_x_at | CDH11 | 283 |
| OC3SNGnh.4032_s_at | CDH11 | 284 |
| OCHPRC.58_s_at | CDH11 | 285 |
| OCMX.1718.C1_s_at | CDH11 | 286 |
| OCADA.8067_x_at | CDH11 | 287 |
| OCADNP.8007_s_at | CDR1 | 288 |
| OC3P.295.C1_s_at | CFB | 289 |
| ADXStrongB56_at | CFB | N/A |
| OC3P.295.C2_x_at | CFB | 290 |
| OC3SNGnh.14167_at | CFB | 291 |
| OC3SNGn.5914-165a_s_at | CFB | 292 |
| OC3SNGn.970-10a_s_at | CFB | 293 |
| OCADNP.9683_s_at | CFB | 294 |
| OC3P.295.C2_at | CFB | 295 |
| OC3SNGnh.14167_s_at | CFB | 296 |
| OCADNP.17538_s_at | CIITA | 297 |
| OC3P.805.C1_s_at | CIITA | 298 |
| OCEM.1780_s_at | CIITA | 299 |
| OC3SNGnh.16892_s_at | CIITA | 300 |
| OCADA.6540_s_at | CIITA | 301 |
| OCHP.1927_s_at | CIITA | 302 |
| OC3SNGn.354-123a_s_at | CIITA | 303 |
| OC3SNGnh.4794_at | CIITA | 304 |
| OC3SNGn.8474-50a_x_at | COL1A2 | 305 |
| OCMX.184.C11_s_at | COL1A2 | 306 |
| OC3SNG.115-2502a_at | COL1A2 | 307 |
| OC3SNG.116-9169a_s_at | COL1A2 | 308 |
| OC3P.60.CB2_x_at | COL1A2 | 309 |
| OC3P.6454.C1_s_at | COL1A2 | 310 |
| OC3SNG.115-2502a_x_at | COL1A2 | 311 |
| OCMX.184.C16_x_at | COL1A2 | 312 |
| OCHP.173_x_at | COL1A2 | 313 |
| OC3P.60.CB1_x_at | COL1A2 | 314 |
| OC3SNGn.2538-539a_x_at | COL1A2 | 315 |
| OCMX.184.C16_s_at | COL1A2 | 316 |
| OCADNP.4048_s_at | COL3A1 | 317 |
| OC3P.81.CB2_s_at | COL3A1 | 318 |
| OC3SNGnh.19127_s_at | COL3A1 | 319 |
| OC3SNGn.1211-6a_s_at | COL3A1 | 320 |
| OCADNP.11975_s_at | COL4A1 | 321 |
| OC3P.850.C1-1145a_s_at | COL4A1 | 322 |
| OCHPRC.29_s_at | COL4A1 | 323 |
| OC3SNGnh.276_x_at | COL4A1 | 324 |
| OC3SNGnh.18844_at | COL8A1 | 325 |
| OC3P.1087.C1_s_at | COL8A1 | 326 |
| OC3P.13652.C1_s_at | COL8A1 | 327 |
| OCADNP.14932_s_at | COL8A1 | 328 |
| OC3P.10562.C1_s_at | COL8A1 | 329 |
| OCHPRC.94_s_at | CXCL17 | 330 |
| OC3SNG.3604-23a_at | CXCR2P1 | 331 |
| OC3SNG.3604-23a_x_at | CXCR2P1 | 332 |
| OC3SNGnh.13095_at | DEXI | 333 |
| OC3P.7366.C1_s_at | DEXI | 334 |
| OCADA.2531_s_at | DEXI | 335 |
| OC3SNGnh.3527_at | DEXI | 336 |
| OC3P.10489.C1_s_at | DEXI | 337 |
| OCADNP.10600_s_at | DEXI | 338 |
| OCADA.1911_s_at | DPYSL2 | 339 |
| OC3P.7322.C1_s_at | DPYSL2 | 340 |
| OC3SNG.366-35a_s_at | ENTPD7 | 341 |
| OC3SNGnh.5644_s_at | FKBP7 | 342 |
| OC3SNGnh.17831_at | FKBP7 | 343 |
| OCADNP.7326_s_at | FKBP7 | 344 |
| OC3P.12003.C1_x_at | FKBP7 | 345 |
| OC3P.4378.C1_s_at | GBP4 | 346 |
| OC3SNGnh.5459_s_at | GBP4 | 347 |
| OCADNP.3694_s_at | GBP4 | 348 |
| OC3SNG.3671-13a_s_at | GJB1 | 349 |
| 2874688_at | HINT1 | N/A |
| 2874689_at | HINT1 | N/A |
| Adx-200093_s_at | HINT1 | 350 |
| OC3SNGnh.5235_x_at | HINT1 | 351 |
| 2874702_at | HINT1 | N/A |
| 2874727_at | HINT1 | N/A |
| 200093_s_at | HINT1 | 352 |
| 2874697_at | HINT1 | N/A |
| 2874725_at | HINT1 | N/A |
| 2874696_at | HINT1 | N/A |
| 2874737_at | HINT1 | N/A |
| 2874735_at | HINT1 | N/A |
| Adx-200093-up_s_at | HINT1 | 353 |
| OC3P.14829.C1_s_at | HLA-DOB | 354 |
| ADXBad55_at | HLA-DOB | N/A |
| OC3P.674.C1_s_at | HLA-DRA | 355 |
| OCADNP.8307_s_at | HLA-DRA | 356 |
| OC3P.2407.C1_s_at | ID3 | 357 |
| ADXGood100_at | IGF2 | N/A |
| OC3SNG.899-20a_s_at | IGF2 | 358 |
| OC3SNGn.5728-103a_x_at | IGF2 | 360 |
| OC3P.4645.C1_s_at | IGF2 | 363 |
| OC3SNGnh.19773_s_at | IGF2 | 364 |
| OCADNP.10122_s_at | IGF2 | 365 |
| OCADNP.7400_s_at | IGF2 | 366 |
| ADXGood100_at | INS | N/A |
| OCADNP.17017_s_at | INS | 359 |
| OC3SNGn.5728-103a_x_at | INS | 360 |
| OCEM.2174_s_at | INS | 361 |
| OCEM.2035_x_at | INS | 362 |
| OC3P.4645.C1_s_at | INS | 363 |
| OC3SNGnh.19773_s_at | INS | 364 |
| OCADNP.10122_s_at | INS | 365 |
| OCADNP.7400_s_at | INS | 366 |
| OCEM.2035_at | INS | 367 |
| OC3P.9976.C1_x_at | ISLR | 368 |
| OCHP.1306_s_at | LOXL1 | 369 |
| OCADA.10621_s_at | MATN3 | 370 |
| OC3P.2576.C1_x_at | MFAP2 | 371 |
| OCHP.1079_s_at | MFAP2 | 372 |
| OC3P.11139.C1_s_at | MIR198 | 373 |
| OC3P.211.C1_x_at | MIR198 | 374 |
| ADXBad7_at | MIR198 | N/A |
| OCHP.462_s_at | MIR198 | 375 |
| OC3SNGn.8954-766a_s_at | MIR198 | 376 |
| OCADNP.4997_s_at | MIR198 | 377 |
| OCHP.228_s_at | MMP14 | 378 |
| OC3P.4123.C1_x_at | MMP14 | 379 |
| OC3P.4123.C1_s_at | MMP14 | 380 |
| OCADA.1433_x_at | NID1 | 381 |
| OCADNP.7347_s_at | NID1 | 382 |
| OC3P.3404.C1_s_at | NID1 | 383 |
| OC3SNGn.3328-664a_s_at | NID1 | 384 |
| OCADNP.9225_s_at | NUAK1 | 385 |
| ADXStrongB87_at | NUAK1 | N/A |
| OC3SNGn.2676-391a_s_at | NUAK1 | 386 |
| OCHPRC.111_s_at | PDPN | 387 |
| OCADNP.10047_s_at | PDPN | 388 |
| OCHPRC.96_s_at | PDPN | 389 |
| OC3P.13523.C1_s_at | PDPN | 390 |
| OC3SNG.4571-22a_x_at | POLD2 | 391 |
| OCEM.1126_s_at | POLD2 | 392 |
| ADXGood4_at | POLD2 | N/A |
| OC3SNGn.890-5a_s_at | POLD2 | 393 |
| OC3P.14770.C1_s_at | PSMB9 | 394 |
| OCRS.920_s_at | PSMB9 | 395 |
| OC3P.4627.C1_s_at | PSMB9 | 396 |
| OC3SNGnh.8187_at | PSMB9 | 397 |
| OCMX.15283.C1_x_at | PSMB9 | 398 |
| OCADNP.804_s_at | PSMB9 | 399 |
| OC3SNGnh.8187_x_at | PSMB9 | 400 |
| OCMX.14440.C1_x_at | PSMB9 | 401 |
| OC3P.1307.C1_s_at | PXDN | 402 |
| OC3P.8838.C1_s_at | PXDN | 403 |
| OCHP.1891_s_at | RARRES3 | 404 |
| OC3P.8963.C1_s_at | RCBTB1 | 405 |
| OC3SNGnh.6721_x_at | RHOBTB3 | 406 |
| OC3SNGnh.6912_x_at | RHOBTB3 | 407 |
| OC3SNGnh.957_s_at | RHOBTB3 | 408 |
| OC3SNG.2402-2883a_s_at | RHOBTB3 | 409 |
| OCHPRC.1436_at | RHOBTB3 | 410 |
| OC3SNGn.5382-76a_s_at | RHOBTB3 | 411 |
| OC3SNGnh.957_x_at | RHOBTB3 | 412 |
| OC3SNGnh.957_at | RHOBTB3 | 413 |
| OC3P.12862.C1_s_at | RHOBTB3 | 414 |
| OC3SNG.2401-1265a_x_at | RHOBTB3 | 415 |
| OC3P.5737.C1_s_at | RHOBTB3 | 416 |
| OCHP.1722_s_at | RTP4 | 417 |
| OC3P.9552.C1-496a_s_at | RTP4 | 418 |
| OC3P.9552.C1_x_at | RTP4 | 419 |
| OC3P.9552.C1_at | RTP4 | 420 |
| OC3SNGnh.865_s_at | SH3RF2 | 421 |
| OC3SNGnh.16695_s_at | SH3RF2 | 422 |
| OCADNP.12161_s_at | SH3RF2 | 423 |
| OC3SNGn.439-184a_s_at | SH3RF2 | 424 |
| OCHPRC.86_s_at | SH3RF2 | 425 |
| OCADNP.2340_s_at | SHISA4 | 426 |
| OC3SNG.6118-43a_s_at | SHISA4 | 427 |
| OCADNP.8940_s_at | SLC28A3 | 428 |
| OC3SNGnh.971_s_at | SLC28A3 | 429 |
| OCADA.4025_s_at | SLC28A3 | 430 |
| OC3P.9666.C1_s_at | SLC28A3 | 431 |
| OC3P.5726.C1_s_at | SLC5A1 | 432 |
| OCADNP.7872_s_at | SLC5A1 | 433 |
| OCRS2.10331_x_at | SNORD114-14 | 434 |
| OCRS2.8538_x_at | SNORD114-14 | 435 |
| OCRS2.10331_at | SNORD114-14 | 436 |
| OC3SNGn.2110-23a_s_at | SOX11 | 437 |
| OCHP.1171_s_at | SOX11 | 438 |
| OCHP.1523_s_at | SOX11 | 439 |
| OC3SNGnh.19157_x_at | SPARC | 440 |
| OCHP.508_s_at | SPARC | 441 |
| OC3P.148.CB1-990a_s_at | SPARC | 442 |
| OCEM.2143_at | SPARC | 443 |
| OC3SNG.2614-40a_s_at | SPARC | 444 |
| OC3P.148.CB1_x_at | SPARC | 445 |
| OCEM.2143_x_at | SPARC | 446 |
| OC3SNG.1657-20a_s_at | SRPX | 447 |
| ADXGoodB4_at | TAP1 | N/A |
| OC3SNG.2665-23a_s_at | TAP1 | 448 |
| OC3P.5602.C1_s_at | TAP2 | 449 |
| OCADNP.2260_s_at | TAP2 | 450 |
| OCADNP.8242_s_at | TAP2 | 451 |
| OC3SNGnh.18127_s_at | TAP2 | 452 |
| OC3P.14195.C1_s_at | TIMP2 | 453 |
| OCHP.320_s_at | TIMP2 | 454 |
| OC3P.543.CB1_x_at | TIMP2 | 455 |
| OC3SNGnh.19238_s_at | TIMP2 | 456 |
| OC3P.543.CB1-699a_s_at | TIMP2 | 457 |
| OCADNP.14191_s_at | TIMP2 | 458 |
| OCADNP.13017_s_at | TIMP3 | 459 |
| OCADA.9324_s_at | TIMP3 | 460 |
| OCHP.1200_s_at | TIMP3 | 461 |
| ADXGood73_at | TIMP3 | N/A |
| OC3P.10470.C1_s_at | TIMP3 | 462 |
| OC3P.15327.C1_at | TIMP3 | 463 |
| OCHP.112_s_at | TIMP3 | 464 |
| OC3P.5348.C1_s_at | TMEM45A | 465 |
| OC3P.4028.C1_at | TNFRSF14 | 466 |
| OC3SNGn.2230-103a_s_at | TNFRSF14 | 467 |
| OC3P.4028.C1_x_at | TNFRSF14 | 468 |
| OC3SNG.1683-90a_s_at | TNFSF10 | 469 |
| OC3P.2087.C1_s_at | TNFSF10 | 470 |
| OCHP.318_x_at | TNFSF10 | 471 |
| OC3SNGn.6279-343a_s_at | TNFSF10 | 472 |
| OC3SNGn.5842-826a_x_at | TNFSF10 | 473 |
| OCADNP.9180_s_at | TNFSF10 | 474 |
| OCHP.1136_s_at | TUBA1A | 475 |
| OCADNP.7771_s_at | XAF1 | 476 |
| ADXStrongB9_at | XAF1 | N/A |
| OC3SNG.2606-619a_x_at | XAF1 | 477 |
| OC3SNGnh.10895_at | XAF1 | 478 |
| OC3P.4873.C1_s_at | XAF1 | 479 |
| OC3SNGnh.10895_x_at | XAF1 | 480 |
| OC3SNG.2605-236a_x_at | XAF1 | 481 |
| OC3SNG.5460-81a_x_at | XAF1 | 482 |
| OCADA.154_s_at | ZNF697 | 483 |
| OCADA.3112_s_at | ZNF697 | 484 |

According to a further aspect of the invention there is provided a method for predicting the responsiveness of a subject with cancer to an anti-angiogenic therapeutic agent comprising: allocating the cancer to a cancer sub-type by measuring the expression levels of at least 2 biomarkers in a sample from the subject and assessing from the expression levels of the biomarkers whether the cancer belongs to the sub-type

(optionally) wherein the cancer sub-type is defined by the expression levels of the genes in Tables A and B

classifying the subject as responsive or non-responsive to the anti-angiogenic therapeutic agent on the basis of allocation to the subtype, wherein if the cancer belongs to the sub-type it is predicted to be non-responsive to the anti-angiogenic therapeutic agent

wherein the at least 2 biomarkers do not consist of from 1 to 63 of the biomarkers shown in Table C.

In yet a further aspect, the present invention relates to a method of determining clinical prognosis of a subject with cancer comprising:

measuring the expression levels of at least 2 biomarkers in a sample from the subject and assessing from the expression levels of the biomarkers whether the cancer belongs to a cancer sub-type

(optionally) wherein the cancer sub-type is defined by the expression levels of the genes in Tables A and B

classifying the subject as having a good prognosis if the cancer belongs to the sub-type wherein the at least 2 biomarkers do not consist of from 1 to 63 of the biomarkers shown in Table C.

According to all relevant aspects of the invention the subject (whose clinical prognosis is determined) is receiving, has received and/or will receive a standard chemotherapeutic treatment for the subject's cancer type and/or has not, is not and/or will not receive an anti-angiogenic therapeutic agent. In certain embodiments the standard chemotherapeutic treatment comprises, consists essentially of or consists of a platinum based-chemotherapeutic agent, a mitotic inhibitor, or a combination thereof. In specific embodiments the standard chemotherapeutic treatment comprises, consists essentially of or consists of carboplatin (or cisplatin) and/or paclitaxel.

Good prognosis may indicate increased progression free survival and/or overall survival rates and/or decreased likelihood of recurrence or metastasis compared to subjects with cancers that do not belong to the sub-type. Metastasis, or metastatic disease, is the spread of a cancer from one organ or part to another non-adjacent organ or part. The new occurrences of disease thus generated are referred to as metastases.

A therapeutic agent is “contraindicated” or “detrimental” to a patient if the cancer's rate of growth is accelerated as a result of contact with the therapeutic agent, compared to its growth in the absence of contact with the therapeutic agent and/or if the therapeutic agent is toxic to a patient. Growth of a cancer can be measured in a variety of ways. For instance, the size of a tumour, or measuring the expression of tumour markers appropriate for that tumour type. A therapeutic agent can also be considered “contraindicated” or “detrimental” if the patient's overall prognosis (progression free survival and/or overall survival) is reduced by the administration of the therapeutic agent.

A cancer is “responsive” to a therapeutic agent if its rate of growth is inhibited as a result of contact with the therapeutic agent, compared to its growth in the absence of contact with the therapeutic agent. Growth of a cancer can be measured in a variety of ways. For instance, the size of a tumor or measuring the expression of tumour markers appropriate for that tumour type. A cancer can also be considered responsive to a therapeutic agent if the patient's overall prognosis (progression free survival and/or overall survival) is improved by the administration of the therapeutic agent.

A cancer is “non-responsive” to a therapeutic agent if its rate of growth is not inhibited, or inhibited to a very low degree or to a non-statistically significant degree, as a result of contact with the therapeutic agent when compared to its growth in the absence of contact with the therapeutic agent. As stated above, growth of a cancer can be measured in a variety of ways, for instance, the size of a tumour or measuring the expression of tumour markers appropriate for that tumour type. A cancer can also be considered non-responsive to a therapeutic agent if the patient's overall prognosis (progression free survival and/or overall survival) is not improved by the administration of the therapeutic agent. Still further, measures of non-responsiveness can be assessed using additional criteria beyond growth size of a tumor such as, but not limited to, patient quality of life, and degree of metastases.

In a further aspect, the present invention relates to a method of treating cancer comprising administering a chemotherapeutic agent to a subject wherein the subject is selected for treatment on the basis of a method as described herein and wherein an anti-angiogenic therapeutic agent is not administered (if the cancer is determined to belong to the subtype).

The invention also relates to a method of treating cancer comprising administering a chemotherapeutic agent and not administering an anti-angiogenic therapeutic agent to a subject, wherein the subject has a cancer that has been determined to belong to a cancer sub-type, (optionally) wherein the cancer sub-type is defined by the expression levels of the genes in Tables A and B, by either:

(i) measuring the expression levels of at least 3 biomarkers in a sample from the subject,

wherein at least two of the biomarkers are from Table A and at least one of the biomarkers is from Table B and assessing from the expression levels of the biomarkers whether the cancer belongs to the cancer sub-type

wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74; or

(ii) measuring the expression levels of at least 2 biomarkers in a sample from the subject and assessing from the expression levels of the biomarkers whether the cancer belongs to the cancer sub-type

wherein the at least 2 biomarkers do not consist of from 1 to 63 of the biomarkers shown in Table C.

According to a further aspect of the invention there is provided a chemotherapeutic agent for use in treating cancer in a subject wherein the subject is selected for treatment on the basis of a method as described herein and wherein the subject is not treated with an anti-angiogenic therapeutic agent (if the cancer is determined to belong to the subtype).

In yet a further aspect, the present invention relates to a chemotherapeutic agent for use in treating cancer in a subject wherein the subject has a cancer that has been determined to belong to a cancer sub-type, (optionally) wherein the cancer sub-type is defined by the expression levels of the genes in Tables A and B, by either:

(i) measuring the expression levels of at least 3 biomarkers in a sample from the subject, wherein at least two of the biomarkers are from Table A and at least one of the biomarkers is from Table B and assessing from the expression levels of the biomarkers whether the cancer belongs to the cancer sub-type

wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74; or

(ii) measuring the expression levels of at least 2 biomarkers in a sample from the subject and assessing from the expression levels of the biomarkers whether the cancer belongs to the cancer sub-type

wherein the at least 2 biomarkers do not consist of from 1 to 63 of the biomarkers shown in Table C

and wherein the subject is not treated with an anti-angiogenic therapeutic agent.

The invention also relates to a method of treating cancer comprising administering a chemotherapeutic agent to a subject wherein the subject has a cancer that belongs to a cancer sub-type defined by the expression levels of the genes in Tables A and B and wherein an anti-angiogenic therapeutic agent is not administered.

In a further aspect, the present invention relates to a chemotherapeutic agent for use in treating cancer in a subject wherein the subject has a cancer that belongs to a cancer sub-type defined by the expression levels of the genes in Tables A and B and wherein the subject is not treated with an anti-angiogenic therapeutic agent.

According to all aspects of the invention the chemotherapeutic agent may comprise a platinum-based chemotherapeutic agent, an alkylating agent, an anti-metabolite (such as 5FU), an anti-tumour antibiotic, a topoisomerase inhibitor, a mitotic inhibitor, or a combination thereof. In certain embodiments the chemotherapeutic agent comprises a platinum based-chemotherapeutic agent, a mitotic inhibitor, or a combination thereof. In specific embodiments the chemotherapeutic agent comprises carboplatin and/or paclitaxel. The chemotherapeutic agent may reflect the standard of care treatment for the cancer. The standard of care treatment may differ for different types of cancer—for example, carboplatin in ovarian cancer, 5FU in colorectal cancer, platinum in head and neck cancer.

According to all aspects of the invention assessing whether the cancer belongs to the sub-type may comprise the use of classification trees.

According to all aspects of the invention assessing whether the cancer belongs to the sub-type may comprise:

determining a sample expression score for the biomarkers;

comparing the sample expression score to a threshold score; and

determining whether the sample expression score is above or

equal to or below the threshold expression score,

wherein if the sample expression score is above or equal to the threshold expression score the cancer belongs to the sub-type.

The sample expression score and threshold score may also be determined such that if the sample expression score is below or equal to the threshold expression score the cancer belongs to the sub-type.

“Expression levels” of biomarkers may be numerical values or directions of expression.

In certain embodiments the expression score is calculated using a weight value and/or a bias value for each biomarker. In specific embodiments the at least two biomarkers from Table A are weighted as 1/N where N is the number of biomarkers used from Table A and the at least one biomarker from Table B is weighted as 1/M where M is the number of biomarkers used from Table B.

As used herein, the term “weight” refers to the absolute magnitude of an item in a mathematical calculation. The weight of each biomarker in a gene expression classifier may be determined on a data set of patient samples using learning methods known in the art. As used herein the term “bias” or “offset” refers to a constant term derived using the mean or median expression of the signatures genes in a training set and is used to mean- or median-center each gene analyzed in the test dataset.

By expression score is meant a compound decision score that summarizes the expression levels of the biomarkers. This may be compared to a threshold score that is mathematically derived from a training set of patient data. The threshold score is established with the purpose of maximizing the ability to separate cancers into those that belong to the sub-type and those that do not. The patient training set data is preferably derived from cancer tissue samples having been characterized by sub-type, prognosis, likelihood of recurrence, long term survival, clinical outcome, treatment response, diagnosis, cancer classification, or personalized genomics profile. Expression profiles, and corresponding decision scores from patient samples may be correlated with the characteristics of patient samples in the training set that are on the same side of the mathematically derived score decision threshold. In certain example embodiments, the threshold of the (optionally linear) classifier scalar output is optimized to maximize the sum of sensitivity and specificity under cross-validation as observed within the training dataset.

The overall expression data for a given sample may be normalized using methods known to those skilled in the art in order to correct for differing amounts of starting material, varying efficiencies of the extraction and amplification reactions, etc.

In one embodiment, the biomarker expression levels in a sample are evaluated by a linear classifier. As used herein, a linear classifier refers to a weighted sum of the individual biomarker intensities into a compound decision score (“decision function”). The decision score is then compared to a pre-defined cut-off score threshold, corresponding to a certain set-point in terms of sensitivity and specificity which indicates if a sample is equal to or above the score threshold (decision function positive) or below (decision function negative).

Using a linear classifier on the normalized data to make a call (e.g. cancer belongs to the sub-type or not) effectively means to split the data space, i.e. all possible combinations of expression values for all genes in the classifier, into two disjoint segments by means of a separating hyperplane. This split is empirically derived on a large set of training examples. Without loss of generality, one can assume a certain fixed set of values for all but one biomarker, which would automatically define a threshold value for this remaining biomarker where the decision would change from, for example, belonging to the sub-type or not. The precise value of this threshold depends on the actual measured expression profile of all other biomarkers within the classifier, but the general indication of certain biomarkers remains fixed. Therefore, in the context of the overall gene expression classifier, relative expression can indicate if either up- or down-regulation of a certain biomarker is indicative of belonging to the sub-type or not. In certain example embodiments, a sample expression score above the threshold expression score indicates the cancer belongs to the subtype. In certain other example embodiments, a sample expression score above a threshold score indicates the subject has a good clinical prognosis compared to a subject with a sample expression score below the threshold score. In certain other example embodiments, a sample expression score above the threshold score indicates the subject has an increased relative risk of experiencing a detrimental effect, or having a poor prognosis, if an anti-angiogenic therapeutic agent is administered.

In certain embodiments the biomarkers used to assess whether the cancer belongs to the cancer sub-type do not comprise or consist of any one or more of the 63 biomarkers shown in Table C.

According to all aspects of the invention the cancer sub-type may be defined by increased and/or decreased expression levels of the genes listed in Tables A and B as shown in Tables A and B.

When a biomarker indicates or is a sign of an abnormal process, disease or other condition in an individual, that biomarker may be described as being either over-expressed or under-expressed or having an increased or decreased expression level as compared to an expression level or value of the biomarker that indicates or is a sign of a normal process, an absence of a disease or other condition in an individual. “Up-regulation”, “up-regulated”, “over-expression”, “over-expressed”, “increased expression” and any variations thereof are used interchangeably to refer to a value or level of a biomarker in a biological sample that is (statistically significantly) greater than a value or level (or range of values or levels) of the biomarker that is typically detected in similar biological samples from healthy or normal individuals. The terms may also refer to a value or level of a biomarker in a biological sample that is (statistically significantly) greater than a value or level (or range of values or levels) of the biomarker that may be detected at a different stage of a particular disease. The terms may also be used to refer to a value or level of biomarker in a biological sample that is (statistically significantly) greater than the average value or level of the biomarker that may be detected for samples of the same disease as a whole. For example, the level of biomarker may be (statistically significantly) greater than the average level for ovarian cancer samples, preferably serous ovarian cancer samples, more preferably high-grade serous ovarian cancer samples.

“Down-regulation”, “down-regulated”, “under-expression”, “under-expressed”, “decreased expression” and any variations thereof are used interchangeably to refer to a value or level of a biomarker in a biological sample that is (statistically significantly) less than a value or level (or range of values or levels) of the biomarker that is typically detected in similar biological samples from healthy or normal individuals. The terms may also refer to a value or level of a biomarker in a biological sample that is (statistically significantly) less than a value or level (or range of values or levels) of the biomarker that may be detected at a different stage of a particular disease. The terms may also be used to refer to a value or level of biomarker in a biological sample that is (statistically significantly) less than the average value or level of the biomarker that may be detected for samples of the same disease as a whole. For example, the level of biomarker may be (statistically significantly) less than the average level for ovarian cancer samples, preferably serous ovarian cancer samples, more preferably high-grade serous ovarian cancer samples.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease, disease subtype, or other condition in an individual. Thus, “differential expression” of a biomarker can also be referred to as a variation from a “normal” expression level of the biomarker.

The terms “differential biomarker expression” and “differential expression” are used interchangeably to refer to a biomarker whose expression is activated to a higher or lower level in a subject suffering from a specific disease, relative to its expression in a normal subject, or relative to its expression in a patient that responds differently to a particular therapy or has a different prognosis. The terms also include biomarkers whose expression is activated to a higher or lower level at different stages of the same disease. It is also understood that a differentially expressed biomarker may be either activated or inhibited at the nucleic acid level or protein level, or may be subject to alternative splicing to result in a different polypeptide product. Such differences may be evidenced by a variety of changes including mRNA levels, miRNA levels, antisense transcript levels, or protein surface expression, secretion or other partitioning of a polypeptide. Differential biomarker expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a biomarker among, for example, normal and diseased cells, or among cells which have undergone different disease events or disease stages.

In certain embodiments the subject is receiving, has received and/or will receive (optionally together with the anti-angiogenic therapeutic agent) treatment with a chemotherapeutic agent.

According to all aspects of the invention the method may further comprise obtaining a test sample from the subject. The methods may be vitro methods performed on an isolated sample.

According to all aspects of the invention samples may be of any suitable form including any material, biological fluid, tissue, or cell obtained or otherwise derived from an individual. In specific embodiments the sample comprises, consists essentially of or consists of a formalin-fixed paraffin-embedded biopsy sample. In further embodiments the sample comprises, consists essentially of or consists of a fresh/frozen (FF) sample. The sample may comprise, consist essentially of or consist of tumour (cancer) tissue, optionally ovarian tumour (cancer) tissue. The sample may comprise, consist essentially of or consist of tumour (cancer) cells, optionally ovarian tumour (cancer) cells. The sample may be obtained by any suitable technique. Examples include a biopsy procedure, optionally a fine needle aspirate biopsy procedure. Body fluid samples may also be utilised. Suitable sample types include blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, nipple aspirate, bronchial aspirate, synovial fluid, joint aspirate, ascites, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “sample” also includes materials containing homogenized solid material, such as from a stool sample, a tissue sample, or a tissue biopsy, for example. The term “sample” also includes materials derived from a tissue culture or a cell culture, including tissue resection and biopsy samples. Example methods for obtaining a sample include, e.g., phlebotomy, swab (e.g., buccal swab). Samples can also be collected, e.g., by micro dissection (e.g., laser capture micro dissection (LCM) or laser micro dissection (LMD)), bladder wash, smear (e.g., a PAP smear), or ductal lavage. A “sample” obtained or derived from an individual includes any such sample that has been processed in any suitable manner after being obtained from the individual. The methods of the invention as defined herein may begin with an obtained sample and thus do not necessarily (although they may) incorporate the step of obtaining the sample from the patient. As used herein, the term “patient” includes human and non-human animals. The preferred patient for treatment is a human. “Patient,” “individual” and “subject” are used interchangeably herein.

According to all aspects of the invention the cancer may be ovarian cancer,

peritoneal cancer or fallopian tube cancer. In certain embodiments the ovarian cancer is high grade serous ovarian cancer. The cancer may also be leukemia, brain cancer, glioblastoma prostate cancer, liver cancer, stomach cancer, colorectal cancer, colon cancer, thyroid cancer, neuroendocrine cancer, gastrointestinal stromal tumors (GIST), gastric cancer, lymphoma, throat cancer, breast cancer, skin cancer, melanoma, multiple myeloma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, and the like. As used herein, colorectal cancer encompasses cancers that may involve cancer in tissues of both the rectum and other portions of the colon as well as cancers that may be individually classified as either colon cancer or rectal cancer.

In all aspects of the invention the anti-angiogenic therapeutic agent may be a VEGF-pathway-targeted therapeutic agent, an angiopoietin-TIE2 pathway inhibitor, an endogenous angiogenic inhibitor, or an immunomodulatory agent. In certain embodiments the VEGF pathway-targeted therapeutic agent is selected from Bevacizumab (Avastin), Aflibercept (VEGF Trap), IMC-1121B (Ramucirumab), Imatinib (Gleevec), Sorafenib (Nexavar), Gefitinib (Iressa), Sunitinib (Sutent), Erlotinib, Tivozinib, Cediranib (Recentin), Pazopanib (Votrient), BIBF 1120 (Vargatef), Dovitinib, Semaxanib (Sugen), Axitinib (AG013736), Vandetanib (Zactima), Nilotinib (Tasigna), Dasatinib (Sprycel), Vatalanib, Motesanib, ABT-869, TKI-258 or a combination thereof. The angiopoietin-TIE2 pathway inhibitor may be selected from AMG-386, PF-4856884 CVX-060, CEP-11981, CE-245677, MEDI-3617, CVX-241, Trastuzumab (Herceptin) or a combination thereof. In certain embodiments the endogenous angiogenic inhibitor is selected from Thombospondin, Endostatin, Tumstatin, Canstatin, Arrestin, Angiostatin, Vasostatin, Interferon alpha or a combination thereof. In further embodiments the immunomodulatory agent is selected from thalidomide and lenalidomide. In specific embodiments the VEGF pathway-targeted therapeutic agent is bevacizumab.

Accordingly, in a further aspect, the present invention relates to a method for selecting whether to administer Bevacizumab to a subject, comprising:

in a test sample obtained from a subject suffering from ovarian cancer, which subject is being, has been and/or will be treated using a platinum-based chemotherapeutic agent and/or a mitotic inhibitor;

measuring expression levels of at least 2 biomarkers;

determining a sample expression score for the 2 or more biomarkers;

comparing the sample expression score to a threshold score;

wherein if the sample expression score is above or equal to the threshold expression score the cancer belongs to a cancer sub-type defined by the expression levels of the genes in Tables A and B

selecting a treatment based on whether the cancer belongs to the sub-type, wherein if the cancer belongs to the sub-type Bevacizumab is contraindicated.

In certain embodiments if Bevacizumab is contraindicated the patient is and/or continues to be treated with a platinum-based chemotherapeutic agent and/or a mitotic inhibitor. In further embodiments if the cancer does not belong to the sub-type the patient is and/or continues to be treated with a platinum-based chemotherapeutic agent and/or a mitotic inhibitor together with Bevacizumab.

According to all aspects of the invention the method may comprise measuring the expression level of at least 3 biomarkers in a sample from the subject, wherein at least two of the biomarkers are from Table A and at least one of the biomarkers is from Table B.

The method may comprise measuring the expression levels of at least 4, 5, 6, 7, 8, 9, 10, 11, 12, 13, 14, 15, 16, 17, 18, 19, 20, 25, 30, 40, 50, 60, 70, 80, 90, 100, 110, 120 or each of the biomarkers from Table F. In certain embodiments the method may comprise measuring the expression levels of 4-20, preferably 4-15, more preferably 4-11 of the biomarkers from Table F. The inventors have shown that measuring the expression levels of at least 4 of the markers in Table F enables the subtype to be reliably detected.

TABLE F

| GeneSymbol | GeneWeights | GeneBias |
|---|---|---|
| UPK2 | −0.018035721 | 3.359991 |
| HLA-DPA1 | 0.015817304 | 5.777439 |
| GABRE | 0.014231336 | 4.945322 |
| KCND2 | 0.014177587 | 6.395784 |
| RPL23AP1 | 0.013258308 | 5.567101 |
| CLDN6 | −0.012995984 | 5.379913 |
| ST6GAL1 | 0.01287146 | 4.244109 |
| PKHD1L1 | 0.012741215 | 3.248153 |
| TMEM169 | −0.012606474 | 4.477176 |
| SECTM1 | 0.012507431 | 6.054561 |
| GBP3 | 0.012101898 | 5.97683 |
| HDHD1 | 0.010328046 | 5.533878 |
| APOBEC3G | 0.009738711 | 6.158638 |
| EIF2AK1 | −0.009557918 | 5.892837 |
| LRP8 | 0.009520369 | 3.493186 |
| KIF26A | −0.009387132 | 5.443061 |
| FAAH2 | 0.009074719 | 4.674146 |
| FAT4 | −0.009068276 | 3.220141 |
| RCAN2 | −0.008853666 | 4.772453 |
| IFI16 | 0.008775954 | 5.108484 |
| GBP1 | 0.00877032 | 5.336176 |
| LYRM7 | 0.008652914 | 6.816823 |
| GNAI1 | −0.008542682 | 7.209451 |
| DIS3L | 0.008481441 | 5.705728 |
| C20orf103 | −0.008457354 | 4.990673 |
| LY6E | 0.008385642 | 8.386388 |
| FIGN | −0.008364187 | 4.693932 |
| GSDMC | 0.008065541 | 4.880615 |
| LRRN4CL | −0.008011982 | 4.043768 |
| C10orf82 | −0.00786412 | 3.821355 |
| GLRX | −0.007725939 | 2.63047 |
| TXK | 0.007709943 | 3.368429 |
| SYTL4 | −0.007709867 | 4.018044 |
| C2orf88 | 0.007705706 | 5.990158 |
| PIGR | 0.00766774 | 5.910846 |
| DLL1 | −0.00765528 | 3.955139 |
| NXNL2 | 0.007564036 | 4.795136 |
| SLC44A4 | 0.007531574 | 6.082619 |
| SAMD9L | 0.007519146 | 5.679514 |
| FAM19A5 | −0.007481583 | 4.233516 |
| PARP14 | 0.007413434 | 6.95454 |
| EFNB3 | −0.007373074 | 5.0962 |
| CHI3L1 | 0.007198574 | 9.270811 |
| TCIRG1 | 0.007149493 | 7.692661 |
| WNT11 | −0.006953495 | 4.967626 |
| EHF | 0.006830876 | 6.295278 |
| CILP | −0.006827864 | 4.158272 |
| TMEM62 | 0.006801865 | 5.533521 |
| TMEM200A | −0.006757567 | 3.718522 |
| POU2F3 | 0.006721892 | 4.061305 |
| USP53 | 0.006591725 | 4.810373 |
| RDBP | 0.006481046 | 11.09852 |
| MTM1 | 0.006429026 | 5.424149 |
| PLSCR1 | 0.006420716 | 5.810762 |
| LRRN1 | −0.006346395 | 4.202345 |
| SP140L | 0.006193052 | 5.282879 |
| SNORD114-7 | −0.006137667 | 4.661787 |
| CCNJL | −0.006103292 | 5.896248 |
| LGALS9 | 0.006096398 | 7.231844 |
| LATS2 | −0.006081829 | 4.567592 |
| GPC2 | −0.006055543 | 6.943001 |
| GATA2 | −0.005830083 | 5.378733 |
| MIR1245 | −0.005762982 | 5.445651 |
| SERPINB1 | 0.005760253 | 5.612094 |
| ST6GAL2 | −0.005718803 | 3.692136 |
| P4HA1 | −0.005703193 | 6.366304 |
| FAM198B | −0.005497488 | 2.963395 |
| DLX5 | −0.005455726 | 4.488077 |
| SEMA3C | −0.005255281 | 5.740108 |
| FAM86A | 0.005123765 | 6.441416 |
| AEBP1 | −0.005066506 | 7.563053 |
| SLC26A10 | −0.005038618 | 5.723967 |
| MAT2B | 0.004967947 | 9.217941 |
| POC1B | 0.004866035 | 6.018808 |
| MYO1B | −0.004846194 | 3.763944 |
| TCF4 | −0.004810352 | 4.934118 |
| GPT | 0.004636147 | 6.287225 |
| FZD2 | −0.0046194 | 4.632028 |
| ASRGL1 | 0.004485953 | 5.341796 |
| CALU | −0.004468499 | 7.661819 |
| HTRA1 | −0.004463171 | 9.086012 |
| ENPP1 | −0.00443649 | 3.567087 |
| MRVI1 | −0.004434326 | 5.098207 |
| MEG3 | −0.004411079 | 7.374835 |
| TWIST1 | −0.00437896 | 7.413093 |
| C4orf31 | −0.00436173 | 3.646165 |
| DTX3L | 0.004098616 | 10.27099 |
| FAM101B | −0.004074778 | 4.69517 |
| APBA2 | −0.003973865 | 5.193996 |
| FAM86C | 0.003951991 | 6.177085 |
| NUDT10 | −0.003940655 | 3.632575 |
| S100A13 | 0.003886817 | 7.069111 |
| TC2N | 0.003875623 | 3.898429 |
| IGFBP4 | −0.003756434 | 7.755969 |
| PRICKLE2 | −0.003495233 | 6.465212 |
| KDM5B | −0.003484745 | 6.159924 |
| CYB5R3 | −0.003468881 | 11.07312 |
| PRKG1 | −0.003447485 | 3.123224 |
| PCOLCE | −0.003433563 | 6.611068 |
| PSME1 | 0.003417446 | 8.183136 |
| FAM101A | −0.003083221 | 5.370094 |
| UTP14A | 0.00296573 | 6.68806 |
| DACT3 | −0.002875519 | 5.333928 |
| C5orf13 | −0.002820432 | 6.823887 |
| CNPY4 | −0.002636714 | 6.331606 |
| MEIS3P1 | −0.002609561 | 6.576464 |
| COL10A1 | −0.002471957 | 6.413886 |
| BGN | −0.002395437 | 10.16321 |
| MN1 | −0.002369196 | 3.490203 |
| MMP2 | −0.002302352 | 5.442494 |
| ETV1 | −0.002266856 | 3.175207 |
| SLC22A17 | −0.002225371 | 6.628063 |
| MEIS3P2 | −0.002084583 | 5.814197 |
| FBLN2 | −0.001963851 | 6.566804 |
| LTBP2 | −0.001948347 | 8.741894 |
| COL1A1 | −0.001923836 | 10.56997 |
| MSRB3 | −0.001698388 | 3.001042 |
| NKD2 | 0.00152605 | 7.385352 |
| MFAP4 | −0.001422147 | 5.833216 |
| VCAN | −0.001290874 | 5.572734 |
| ZNF469 | −0.000451207 | 5.78573 |

The biomarkers from Table F are ranked in Table G from most important to least important based upon hazard ratio reduction when the genes are included versus when they are excluded. The genes/biomarkers may be selected for inclusion in a panel of biomarkers/a signature based on their ranking. Table H illustrates probesets that can be used to detect expression of the biomarkers.

TABLE G

| | Combined Delta | |
|---|---|---|
| Gene | HR | Rank |
| GABRE | 0.337359062 | 1 |
| HLA-DPA1 | 0.300256284 | 2 |
| CHI3L1 | 0.296360718 | 3 |
| KCND2 | 0.257226045 | 4 |
| GBP3 | 0.227046996 | 5 |
| UPK2 | 0.222007152 | 6 |
| SYTL4 | 0.211040547 | 7 |
| LRRN1 | 0.206205626 | 8 |
| USP53 | 0.154837732 | 9 |
| POU2F3 | 0.145576691 | 10 |
| IFI16 | 0.144743856 | 11 |
| GPT | 0.139488308 | 12 |
| SECTM1 | 0.131242036 | 13 |
| GBP1 | 0.127721221 | 14 |
| DLX5 | 0.116832218 | 15 |
| C4orf31 | 0.114744132 | 16 |
| DLL1 | 0.109780949 | 17 |
| EHF | 0.106293094 | 18 |
| SAMD9L | 0.104709676 | 19 |
| PLSCR1 | 0.104625768 | 20 |
| LY6E | 0.103280138 | 21 |
| EFNB3 | 0.101572355 | 22 |
| APOBEC3G | 0.087233468 | 23 |
| RPL23AP1 | 0.084711903 | 24 |
| GNAI1 | 0.081209911 | 25 |
| C20orf103 | 0.071107778 | 26 |
| DTX3L | 0.065552768 | 27 |
| MAT2B | 0.065475368 | 28 |
| CLDN6 | 0.062021901 | 29 |
| P4HA1 | 0.061878907 | 30 |
| SLC44A4 | 0.060350743 | 31 |
| FAT4 | 0.059503895 | 32 |
| LGALS9 | 0.056554956 | 33 |
| FAM19A5 | 0.056059383 | 34 |
| MTM1 | 0.050315972 | 35 |
| SLC26A10 | 0.049327133 | 36 |
| SP140L | 0.048168599 | 37 |
| SLC22A17 | 0.047816275 | 38 |
| FAM198B | 0.047192056 | 39 |
| CCNJL | 0.045558068 | 40 |
| NUDT10 | 0.044612641 | 41 |
| MEG3 | 0.044024878 | 42 |
| GATA2 | 0.043610514 | 43 |
| RDBP | 0.038861452 | 44 |
| EIF2AK1 | 0.037086703 | 45 |
| LYRM7 | 0.031769711 | 46 |
| PRICKLE2 | 0.031098441 | 47 |
| S100A13 | 0.030632337 | 48 |
| PSME1 | 0.029722311 | 49 |
| MYO1B | 0.028958889 | 50 |
| UTP14A | 0.024013078 | 51 |
| PARP14 | 0.023229799 | 52 |
| IGFBP4 | 0.021289533 | 53 |
| FZD2 | 0.021033055 | 54 |
| CALU | 0.020542261 | 55 |
| GPC2 | 0.017999692 | 56 |
| C10orf82 | 0.015198024 | 57 |
| GSDMC | 0.015070219 | 58 |
| CYB5R3 | 0.011241468 | 59 |
| TCIRG1 | 0.010154223 | 60 |
| APBA2 | 0.008802409 | 61 |
| ST6GAL1 | 0.008747796 | 62 |
| CNPY4 | 0.008020809 | 63 |
| FAM101B | 0.0055168 | 64 |
| KDM5B | 0.005118183 | 65 |
| SERPINB1 | 0.005078998 | 66 |
| PIGR | 0.004839196 | 67 |
| PKHD1L1 | 2.51362E−05 | 68 |
| POC1B | −0.00076447 | 69 |
| FAM86A | −0.010246498 | 70 |
| FIGN | −0.010303757 | 71 |
| ASRGL1 | −0.016190261 | 72 |
| FAM86C | −0.017669256 | 73 |
| SNORD114-7 | −0.018123626 | 74 |
| TXK | −0.018325835 | 75 |
| NXNL2 | −0.018378062 | 76 |
| TC2N | −0.020647383 | 77 |
| LATS2 | −0.022701806 | 78 |
| TCF4 | −0.026124482 | 79 |
| TMEM62 | −0.033738079 | 80 |
| PCOLCE | −0.034311272 | 81 |
| ETV1 | −0.037268287 | 82 |
| DIS3L | −0.038288521 | 83 |
| HTRA1 | −0.045043294 | 84 |
| MSRB3 | −0.046398147 | 85 |
| TMEM169 | −0.047281991 | 86 |
| HDHD1 | −0.055954287 | 87 |
| C5orf13 | −0.058378337 | 88 |
| MEIS3P1 | −0.059584725 | 89 |
| GLRX | −0.059644388 | 90 |
| LRRN4CL | −0.060202172 | 91 |
| LTBP2 | −0.060491887 | 92 |
| LRP8 | −0.062812677 | 93 |
| AEBP1 | −0.067344525 | 94 |
| RCAN2 | −0.076520381 | 95 |
| KIF26A | −0.077150316 | 96 |
| MEIS3P2 | −0.082183776 | 97 |
| MFAP4 | −0.087999078 | 98 |
| SEMA3C | −0.089439853 | 99 |
| FAAH2 | −0.10199233 | 100 |
| FBLN2 | −0.10238978 | 101 |
| MRVI1 | −0.104468956 | 102 |
| TWIST1 | −0.105178179 | 103 |
| DACT3 | −0.113122024 | 104 |
| PRKG1 | −0.114727895 | 105 |
| BGN | −0.123157122 | 106 |
| TMEM200A | −0.123401993 | 107 |
| ZNF469 | −0.137897067 | 108 |
| FAM101A | −0.152538637 | 109 |
| WNT11 | −0.153828906 | 110 |
| ENPP1 | −0.171279236 | 111 |
| NKD2 | −0.183893488 | 112 |
| MN1 | −0.191802042 | 113 |
| C2orf88 | −0.209518103 | 114 |
| CILP | −0.222557009 | 115 |
| COL1A1 | −0.225250378 | 116 |
| MMP2 | −0.24991078 | 117 |
| ST6GAL2 | −0.294860786 | 118 |
| COL10A1 | −0.303286192 | 119 |
| VCAN | −0.325923129 | 120 |
| MIR1245 | −0.379590501 | 121 |

TABLE H

| Probeset | Gene | SEQ ID No. |
|---|---|---|
| OCMXSNG.5475_at | AEBP1 | 485 |
| OCMXSNG.2603_at | AEBP1 | 486 |
| ADXStrongB47_at | AEBP1 | N/A |
| OCHP.1649_s_at | AEBP1 | 487 |
| OC3P.3458.C1_s_at | AEBP1 | 488 |
| ADXStrongB42_at | AEBP1 | N/A |
| OCMXSNG.5474_at | AEBP1 | 489 |
| OCMXSNG.5474_x_at | AEBP1 | 490 |
| OCHP.1147_s_at | APBA2 | 491 |
| OC3P.3328.C1_s_at | APBA2 | 492 |
| OCADA.11807_s_at | APBA2 | 493 |
| OC3SNG.5308-20a_s_at | APOBEC3G | 494 |
| OCADNP.16260_s_at | APOBEC3G | 495 |
| OCMX.6106.C2_at | ASRGL1 | 496 |
| OC3SNGnh.20113_s_at | ASRGL1 | 497 |
| OC3SNGnh.15728_x_at | ASRGL1 | 498 |
| OCHPRC.72_s_at | ASRGL1 | 499 |
| OC3P.7460.C1_s_at | ASRGL1 | 500 |
| OC3P.13249.C2_x_at | ASRGL1 | 501 |
| OC3SNGnh.20112_s_at | ASRGL1 | 502 |
| ADXGood55_at | ASRGL1 | N/A |
| OC3P.13249.C2_s_at | ASRGL1 | 503 |
| OC3SNGnh.20112_x_at | ASRGL1 | 504 |
| OCHP.937_s_at | BGN | 505 |
| OCADNP.9883_s_at | BGN | 506 |
| ADXStrong61_at | BGN | N/A |
| OCADNP.5820_s_at | C10orf82 | 507 |
| OC3SNGnh.6274_s_at | C10orf82 | 508 |
| OC3P.7546.C1_s_at | C20orf103 | 509 |
| OC3P.6691.C1_x_at | C2orf88 | 510 |
| OC3SNGn.3209-1053a_s_at | C2orf88 | 511 |
| OC3P.1793.C1_s_at | C2orf88 | 512 |
| OC3SNGnh.6041_x_at | C2orf88 | 513 |
| OCADA.11194_s_at | C2orf88 | 514 |
| OCRS2.1788_s_at | C2orf88 | 515 |
| OC3SNG.2094-40a_s_at | C4orf31 | 516 |
| OC3SNGn.377-427a_s_at | C4orf31 | 517 |
| OC3P.3548.C2_s_at | C5orf13 | 518 |
| OCADNP.9115_s_at | C5orf13 | 519 |
| OCADNP.14721_s_at | C5orf13 | 520 |
| OC3SNGn.2096-734a_s_at | C5orf13 | 521 |
| OCADA.5808_s_at | C5orf13 | 522 |
| OCADNP.11684_s_at | C5orf13 | 523 |
| ADXGood25_at | CALU | N/A |
| OC3SNGnh.9873_s_at | CALU | 524 |
| OC3SNG.123-901a_s_at | CALU | 525 |
| OCADNP.14456_x_at | CALU | 526 |
| OC3P.2001.C2-449a_s_at | CALU | 527 |
| OCADNP.7231_s_at | CALU | 528 |
| OC3SNGnh.11073_x_at | CALU | 529 |
| OC3P.13898.C1_s_at | CALU | 530 |
| OCHP.1141_s_at | CALU | 531 |
| OCADNP.3994_s_at | CALU | 532 |
| OC3P.12365.C1_s_at | CCNJL | 533 |
| OCHP.1872_s_at | CHI3L1 | 534 |
| OCRS.342_at | CILP | 535 |
| OC3P.12218.C1_s_at | CILP | 536 |
| OCHPRC.81_x_at | CLDN6 | 537 |
| OCRS2.7326_x_at | CLDN6 | 538 |
| OC3SNG.2953-20a_x_at | CLDN6 | 539 |
| OCADNP.9501_s_at | CLDN6 | 540 |
| OCRS2.3430_at | CNPY4 | 541 |
| OC3P.12351.C1_s_at | CNPY4 | 542 |
| OCRS.383_s_at | COL10A1 | 543 |
| OC3SNG.1834-947a_s_at | COL10A1 | 544 |
| OC3SNG.3967-1156a_x_at | COL1A1 | 545 |
| OC3P.162.C1_x_at | COL1A1 | 546 |
| OC3SNGnh.2873_x_at | COL1A1 | 547 |
| OCADNP.2115_s_at | COL1A1 | 548 |
| OC3P.354.CB1_s_at | COL1A1 | 549 |
| OC3P.162.C3_x_at | COL1A1 | 550 |
| OC3P.1226.C1_s_at | CYB5R3 | 551 |
| ADXStrong34_at | CYB5R3 | N/A |
| OCEM.1219_s_at | CYB5R3 | 552 |
| OC3SNG.3685-20a_s_at | DACT3 | 553 |
| OC3P.7775.C1_s_at | DIS3L | 554 |
| OC3SNGn.1174-202a_x_at | DIS3L | 555 |
| OC3P.8771.C1_s_at | DLL1 | 556 |
| OC3P.14576.C1_s_at | DLX5 | 557 |
| OC3P.3528.C1_s_at | DTX3L | 558 |
| OCRS.1427_s_at | DTX3L | 559 |
| OCADNP.8516_s_at | EFNB3 | 560 |
| OC3P.9384.C1_s_at | EFNB3 | 561 |
| ADXBad27_at | EHF | N/A |
| OCHPRC.60_s_at | EHF | 562 |
| OC3P.3119.C1-342a_s_at | EHF | 563 |
| OCADNP.10217_s_at | EHF | 564 |
| OC3SNGn.2971-1016a_s_at | EHF | 565 |
| OCHP.22_s_at | EHF | 566 |
| OCMX.12473.C1_s_at | EHF | 567 |
| OCRS.1860_s_at | EHF | 568 |
| OC3P.6113.C1_s_at | EHF | 569 |
| OC3SNGnh.4034_s_at | EHF | 570 |
| ADXStrongB91_at | EHF | N/A |
| ADXBad43_at | EHF | N/A |
| OCADA.6511_s_at | EHF | 571 |
| OCMX5NG.5461_s_at | EIF2AK1 | 572 |
| OC3SNGnh.14331_x_at | EIF2AK1 | 573 |
| OC3P.301.C1_s_at | EIF2AK1 | 574 |
| OC3P.2826.C1_s_at | EIF2AK1 | 575 |
| OC3P.2826.C1-632a_s_at | EIF2AK1 | 576 |
| OCADNP.2363_s_at | ENPP1 | 577 |
| OCADA.8789_s_at | ENPP1 | 578 |
| OCHP.1084_s_at | ENPP1 | 579 |
| OCADA.3370_s_at | ENPP1 | 580 |
| OCADA.6389_s_at | ETV1 | 581 |
| OCADNP.4628_s_at | ETV1 | 582 |
| OC3SNG.2163-2941a_s_at | ETV1 | 583 |
| OCADNP.7847_s_at | ETV1 | 584 |
| OCRS.1862_s_at | ETV1 | 585 |
| OC3SNGn.480-2043a_s_at | ETV1 | 586 |
| OCADNP.5347_s_at | ETV1 | 587 |
| OC3SNGnh.18545_at | FAAH2 | 588 |
| OC3SNGnh.18545_x_at | FAAH2 | 589 |
| OCMXSNG.4800_x_at | FAAH2 | 590 |
| OC3SNGnh.14393_x_at | FAAH2 | 591 |
| OC3SNGnh.13606_x_at | FAAH2 | 592 |
| OC3SNGnh.14393_at | FAAH2 | 593 |
| OC3SNG.6004-30a_s_at | FAAH2 | 594 |
| OC3P.4839.C1_s_at | FAM101A | 595 |
| ADXUglyB43_at | FAM101A | N/A |
| OC3P.8169.C1_s_at | FAM101B | 596 |
| OCRS2.566_s_at | FAM101B | 597 |
| OC3P.9099.C1_s_at | FAM101B | 598 |
| OC3SNGn.7559-1580a_at | FAM198B | 599 |
| OC3P.6417.C1_s_at | FAM198B | 600 |
| OCRS2.4931_s_at | FAM198B | 601 |
| OCADA.10843_s_at | FAM198B | 602 |
| OCADA.5341_s_at | FAM19A5 | 603 |
| OC3P.13915.C1_s_at | FAM19A5 | 604 |
| OC3P.14112.C1_s_at | FAM19A5 | 605 |
| OC3SNGnh.2090_x_at | FAM86A | 607 |
| OC3P.2572.C4_s_at | FAM86A | 608 |
| OCRS2.951_x_at | FAM86A | 606 |
| OC3SNGnh.2090_x_at | FAM86C | 607 |
| OC3P.2572.C4_s_at | FAM86C | 608 |
| OC3SNG.4266-25a_s_at | FAT4 | 609 |
| OC3SNG.1815-80a_s_at | FBLN2 | 610 |
| OCHP.1078_s_at | FBLN2 | 611 |
| OCADA.6796_s_at | FIGN | 612 |
| OC3P.15318.C1_at | FIGN | 613 |
| OCADA.6194_s_at | FIGN | 614 |
| OCADA.2860_s_at | FIGN | 615 |
| OCADNP.12019_s_at | FIGN | 616 |
| OC3P.15266.C1_x_at | FIGN | 617 |
| OC3P.7321.C1_s_at | FZD2 | 618 |
| ADXBad26_at | FZD2 | N/A |
| OC3P.7321.C1_x_at | FZD2 | 619 |
| OC3P.7321.C1_at | FZD2 | 620 |
| OC3P.6165.C1_s_at | GABRE | 621 |
| OC3SNGn.6359-34a_s_at | GABRE | 622 |
| OC3SNGn.6583-10627a_at | GABRE | 623 |
| OC3SNGn.6583-10627a_x_at | GABRE | 624 |
| OCMX.833.C13_s_at | GABRE | 625 |
| OCADA.11121_s_at | GATA2 | 626 |
| OCADA.3908_s_at | GATA2 | 627 |
| OCADNP.1974_s_at | GBP1 | 628 |
| OCADNP.2962_s_at | GBP1 | 629 |
| OCHP.1438_x_at | GBP1 | 630 |
| OCRS2.4406_x_at | GBP1 | 631 |
| OCADA.10565_s_at | GBP1 | 632 |
| OC3P.1927.C1_x_at | GBP1 | 633 |
| OC3SNGnh.19643_x_at | GBP3 | 634 |
| OC3SNGnh.19644_x_at | GBP3 | 635 |
| OC3P.1927.C2_s_at | GBP3 | 636 |
| OCMX.605.C1_at | GLRX | 637 |
| OCHP.1436_s_at | GLRX | 638 |
| OCMX.605.C1_x_at | GLRX | 639 |
| OC3SNGnh.7530_at | GLRX | 640 |
| OCMX.606.C1_s_at | GLRX | 641 |
| OC3SNGnh.7530_x_at | GLRX | 642 |
| OCADNP.8335_s_at | GLRX | 643 |
| OCMX.606.C1_at | GLRX | 644 |
| OCRS2.6438_s_at | GNAI1 | 645 |
| OC3P.1142.C1_s_at | GNAI1 | 646 |
| ADXGood98_at | GNAI1 | N/A |
| OC3SNG.3351-135a_s_at | GPC2 | 647 |
| OC3SNG.5195-46a_s_at | GPT | 648 |
| OC3SNG.5195-46a_x_at | GPT | 649 |
| OC3P.9125.C1_s_at | GSDMC | 650 |
| OCADA.4167_s_at | HDHD1 | 651 |
| OC3SNGnh.18826_at | HDHD1 | 652 |
| OC3P.7901.C1_s_at | HDHD1 | 653 |
| OC3P.2028.C1_s_at | HLA-DPA1 | 654 |
| ADXUglyB19_at | HLA-DPA1 | N/A |
| OC3SNGn.2735-12a_s_at | HLA-DPA1 | 655 |
| OCHP.902_s_at | HTRA1 | 656 |
| OC3SNGn.4796-28001a_s_at | IFI16 | 657 |
| OC3SNG.2113-18a_s_at | IFI16 | 658 |
| OC3SNGn.6068-1286a_s_at | IFI16 | 659 |
| OC3SNGn.4797-39932a_s_at | IFI16 | 660 |
| OCADNP.5197_s_at | IGFBP4 | 661 |
| OC3SNG.5134-22a_s_at | IGFBP4 | 662 |
| OC3SNGnh.6036_s_at | IGFBP4 | 663 |
| ADXStrongB37_at | IGFBP4 | N/A |
| OCADNP.7979_s_at | KCND2 | 664 |
| OCEM.617_s_at | KCND2 | 665 |
| OCMX.2694.C1_s_at | KDM5B | 666 |
| OC3P.7187.C1_s_at | KDM5B | 667 |
| OCADA.11372_s_at | KDM5B | 668 |
| OCEM.1229_at | KDM5B | 669 |
| OC3P.13885.C1_s_at | KIF26A | 670 |
| OCADNP.7032_s_at | LATS2 | 671 |
| OCADA.9355_s_at | LATS2 | 672 |
| OC3P.13211.C1_s_at | LATS2 | 673 |
| OCADA.7506_s_at | LATS2 | 674 |
| OCEM.59_x_at | LGALS9 | 675 |
| OC3P.1033.C1_s_at | LGALS9 | 676 |
| OC3SNGnh.10517_at | LRP8 | 677 |
| OCADA.11886_s_at | LRP8 | 678 |
| OCADA.11978_s_at | LRP8 | 679 |
| OC3P.8630.C1_s_at | LRP8 | 680 |
| OC3SNGnh.10517_at | LRP8 | 681 |
| OCADNP.9495_s_at | LRP8 | 682 |
| OCADNP.5625_s_at | LRRN1 | 683 |
| OCRS2.6196_at | LRRN1 | 684 |
| OC3SNGn.971-6a_at | LRRN1 | 685 |
| OC3SNG.5795-17a_s_at | LRRN4CL | 686 |
| OCADA.663_s_at | LRRN4CL | 687 |
| OCHP.1105_s_at | LTBP2 | 688 |
| OC3P.5700.C1_s_at | LTBP2 | 689 |
| OCMX.3091.C3_s_at | LY6E | 690 |
| OC3SNG.1862-17a_s_at | LY6E | 691 |
| OC3P.177.C1_s_at | LY6E | 692 |
| OC3SNGn.300-11a_s_at | LYRM7 | 693 |
| OC3SNG.5278-785a_x_at | LYRM7 | 694 |
| ADXGood103_at | LYRM7 | N/A |
| OC3SNGnh.8177_x_at | LYRM7 | 695 |
| OC3SNG.2044-750a_s_at | LYRM7 | 696 |
| OC3P.5073.C1_s_at | MAT2B | 697 |
| OC3P.5073.C1_x_at | MAT2B | 698 |
| OC3P.13642.C1_s_at | MEG3 | 699 |
| OCADNP.10552_s_at | MEG3 | 700 |
| OCADA.3017_s_at | MEG3 | 701 |
| OC3P.9532.C1_s_at | MEG3 | 702 |
| OC3SNGn.3096-5a_s_at | MEG3 | 703 |
| OCADNP.14835_s_at | MEG3 | 704 |
| OC3SNGn.3208-51a_s_at | MEG3 | 705 |
| OC3SNGnh.10745_x_at | MEG3 | 706 |
| OCADNP.12059_s_at | MEG3 | 707 |
| OC3P.3104.C1_s_at | MEIS3P1 | 709 |
| OC3P.12137.C1_x_at | MEIS3P1 | 708 |
| OC3P.3104.C1_s_at | MEIS3P2 | 709 |
| OCADNP.11373_x_at | MEIS3P2 | 710 |
| OC3P.4714.C1_at | MFAP4 | 711 |
| OC3SNG.2440-25a_s_at | MFAP4 | 712 |
| OCMX.8836.C3_x_at | MFAP4 | 713 |
| OC3SNGnh.3422_s_at | MIR1245 | 714 |
| OC3P.1163.C3_s_at | MMP2 | 715 |
| OCHP.374_s_at | MMP2 | 716 |
| OCADNP.7251_s_at | MMP2 | 717 |
| OCADA.2310_s_at | MMP2 | 718 |
| OC35NGnh.2965_x_at | MN1 | 719 |
| OCRS2.6707_x_at | MN1 | 720 |
| OC3P.8382.C1_x_at | MN1 | 721 |
| OC3SNGnh.7844_at | MN1 | 722 |
| OCADA.3580_s_at | MRVI1 | 723 |
| OC3P.1058.C1_s_at | MRVI1 | 724 |
| OC3P.13126.C1_s_at | MRVI1 | 725 |
| OCADNP.10237_s_at | MRVI1 | 726 |
| OC3P.12965.C1_x_at | MSRB3 | 727 |
| OCADA.2263_s_at | MSRB3 | 728 |
| OC3SNGn.2476-2808a_s_at | MSRB3 | 729 |
| OC3P.12245.C1_s_at | MSRB3 | 730 |
| OC3SNGn.2475-1707a_s_at | MSRB3 | 731 |
| OCADA.215_s_at | MSRB3 | 732 |
| OCEM.2176_at | MTM1 | 733 |
| OC3P.7705.C1_s_at | MTM1 | 734 |
| OCADA.7806_x_at | MTM1 | 735 |
| OC3SNGnh.16755_at | MYO1B | 736 |
| OC3SNGn.2539-1215a_s_at | MYO1B | 737 |
| OC3P.4399.C1_x_at | MYO1B | 738 |
| OC3SNGn.8543-1096a_s_at | MYO1B | 739 |
| OCADNP.12332_x_at | MYO1B | 740 |
| OCADNP.5849_s_at | NKD2 | 741 |
| OCRS.1038_x_at | NUDT10 | 742 |
| OCMX.1935.C2_x_at | NUDT10 | 743 |
| OCADNP.5059_s_at | NUDT10 | 744 |
| OCRS.1038_at | NUDT10 | 745 |
| OCADA.81_x_at | NXNL2 | 746 |
| OC3SNGnh.3578_s_at | NXNL2 | 747 |
| OC3P.6323.C1-387a_s_at | P4HA1 | 748 |
| OC3SNG.2842-16a_s_at | P4HA1 | 749 |
| OC3SNGnh.5686_x_at | P4HA1 | 750 |
| OC3P.577.C3_x_at | P4HA1 | 751 |
| OC3SNGnh.14212_at | P4HA1 | 752 |
| OC3SNGnh.2575_s_at | PARP14 | 753 |
| OC3P.3721.C1_s_at | PARP14 | 754 |
| OCEM.1594_s_at | PARP14 | 755 |
| OC3P.11978.C1_s_at | PARP14 | 756 |
| ADXUglyB44_at | PARP14 | N/A |
| OC3SNGnh.4719_x_at | PARP14 | 757 |
| OCRS2.3088_s_at | PCOLCE | 758 |
| OC3P.5048.C1_s_at | PCOLCE | 759 |
| OCMXSNG.2345_s_at | PCOLCE | 760 |
| ADXStrong15_at | PIGR | N/A |
| OCHPRC.55_s_at | PIGR | 761 |
| OCADNP.7555_s_at | PIGR | 762 |
| ADXBad46_at | PIGR | N/A |
| OC3P.5246.C1_s_at | PKHD1L1 | 763 |
| OCRS2.2200_s_at | PKHD1L1 | 764 |
| OC3SNGnh.1242_x_at | PKHD1L1 | 765 |
| OCHP.105_s_at | PKHD1L1 | 766 |
| OCADNP.15163_s_at | PKHD1L1 | 767 |
| OCADNP.5491_s_at | PLSCR1 | 768 |
| OCHP.484_s_at | PLSCR1 | 769 |
| OC3P.343.C1-620a_s_at | PLSCR1 | 770 |
| OCADA.9243_s_at | PLSCR1 | 771 |
| OC3P.12249.C1_s_at | POC1B | 772 |
| OCADNP.8935_s_at | POC1B | 773 |
| OC3SNGn.2327-2492a_s_at | POC1B | 774 |
| OC3P.324.C1_x_at | POC1B | 775 |
| ADXUglyB39_at | POU2F3 | N/A |
| OCADA.9784_s_at | POU2F3 | 776 |
| OCADA.8436_s_at | POU2F3 | 777 |
| OCADNP.16713_x_at | POU2F3 | 778 |
| OC3SNGn.207-610a_s_at | POU2F3 | 779 |
| OC3SNGnh.9534_at | PRICKLE2 | 780 |
| OC3P.5913.C1_s_at | PRICKLE2 | 781 |
| OC3SNGnh.9534_x_at | PRICKLE2 | 782 |
| ADXStrong33_at | PRICKLE2 | N/A |
| OC3SNGnh.5282_x_at | PRKG1 | 783 |
| OCMX.3589.C1_at | PRKG1 | 784 |
| OCADNP.7986_s_at | PRKG1 | 785 |
| OC3SNGnh.5282_at | PRKG1 | 786 |
| OC3SNGnh.17864_x_at | PRKG1 | 787 |
| OCADNP.14238_s_at | PRKG1 | 788 |
| OC3SNGnh.17059_s_at | PRKG1 | 789 |
| OCMXSNG.413_x_at | PRKG1 | 790 |
| OCADNP.8589_s_at | PRKG1 | 791 |
| OCEM.2215_at | PRKG1 | 792 |
| OCADNP.11971_s_at | PRKG1 | 793 |
| OCMXSNG.413_at | PRKG1 | 794 |
| OCADA.3268_s_at | PRKG1 | 795 |
| OC3P.943.C2_s_at | PSME1 | 796 |
| OC3P.943.C1_x_at | PSME1 | 797 |
| OC3P.943.C1_s_at | PSME1 | 798 |
| OC3P.11270.C1_s_at | RCAN2 | 799 |
| OC3P.9155.C1_s_at | RDBP | 800 |
| OCMXSNG.5467_x_at | RDBP | 801 |
| OCMXSNG.5045_s_at | RPL23AP1 | 802 |
| OC3SNGnh.19359_x_at | RPL23AP1 | 803 |
| OCHPRC.408_s_at | S100A13 | 804 |
| OC3SNGnh.19423_x_at | S100A13 | 805 |
| OC3SNGnh.4426_at | S100A13 | 806 |
| OCHPRC.408_x_at | S100A13 | 807 |
| OC3SNG.1837-24a_s_at | S100A13 | 808 |
| ADXGoodB16_at | S100A13 | N/A |
| OC3P.1647.C1_s_at | S100A13 | 809 |
| OC3SNGnh.8672_x_at | S100A13 | 810 |
| OC3SNG.5968-144a_x_at | S100A13 | 811 |
| OC3SNGnh.19423_at | S100A13 | 812 |
| OCADNP.3600_s_at | S100A13 | 813 |
| OCADNP.3717_s_at | SAMD9L | 814 |
| OC3P.5848.C1_s_at | SAMD9L | 815 |
| OC3P.9264.C1_s_at | SAMD9L | 816 |
| ADXUgly26_at | SAMD9L | N/A |
| OC3P.10487.C1_s_at | SAMD9L | 817 |
| OC3P.6715.C1_s_at | SECTM1 | 818 |
| OCRS.984_s_at | SECTM1 | 819 |
| OC3SNGnh.7173_x_at | SEMA3C | 820 |
| OC3SNGnh.1972_s_at | SEMA3C | 821 |
| OCADNP.13163_s_at | SEMA3C | 822 |
| OC3P.12081.C1_s_at | SEMA3C | 823 |
| OC3SNGn.4029-2824a_x_at | SERPINB1 | 824 |
| OCHP.1509_s_at | SERPINB1 | 825 |
| OC3P.1480.C1_s_at | SERPINB1 | 826 |
| OCADNP.4790_s_at | SERPINB1 | 827 |
| OC3P.2388.C1_s_at | SERPINB1 | 828 |
| OC3SNGn.4029-2824a_at | SERPINB1 | 829 |
| OC3P.6843.C1-308a_s_at | SLC22A17 | 830 |
| OC3P.6843.C1_at | SLC22A17 | 831 |
| OCADA.8596_s_at | SLC26A10 | 832 |
| OCRS2.621_at | SLC26A10 | 833 |
| OCRS2.621_s_at | SLC26A10 | 834 |
| OCRS2.621_x_at | SLC26A10 | 835 |
| OCADNP.652_s_at | SLC44A4 | 836 |
| OCHP.204_x_at | SLC44A4 | 837 |
| OCADNP.9262_s_at | SLC44A4 | 838 |
| OC3P.11858.C1_x_at | SLC44A4 | 839 |
| OCRS2.12370_x_at | SNORD114-7 | 840 |
| OCRS2.12370_at | SNORD114-7 | 841 |
| OC3P.8666.C1_s_at | SP140L | 842 |
| OCADA.2122_at | SP140L | 843 |
| OCADA.2122_s_at | SP140L | 844 |
| OCADA.2122_x_at | SP140L | 845 |
| OC3SNGnh.1744_at | ST6GAL1 | 846 |
| OC3SNGnh.155_x_at | ST6GAL1 | 847 |
| OCADNP.4027_s_at | ST6GAL1 | 848 |
| OC3P.167.C1_s_at | ST6GAL1 | 849 |
| OC3SNGnh.155_at | ST6GAL1 | 850 |
| OCADA.411_s_at | ST6GAL2 | 851 |
| OCRS.467_at | ST6GAL2 | 852 |
| OCADA.7427_s_at | ST6GAL2 | 853 |
| OCADNP.2470_s_at | SYTL4 | 854 |
| OC3SNGnh.16147_x_at | SYTL4 | 855 |
| OCADA.1925_x_at | SYTL4 | 856 |
| OC3P.12165.C1_s_at | SYTL4 | 857 |
| OC3SNGnh.20531_x_at | TC2N | 858 |
| OC3SNGn.1702-2648a_s_at | TC2N | 859 |
| OC3P.11326.C1_x_at | TC2N | 860 |
| OCADA.4683_s_at | TC2N | 861 |
| ADXUglyB22_at | TC2N | N/A |
| OC3SNGnh.16817_x_at | TC2N | 862 |
| OC3SNGnh.20530_x_at | TC2N | 863 |
| OCHP.1870_s_at | TC2N | 864 |
| OCADNP.230_s_at | TC2N | 865 |
| ADXUglyB50_at | TC2N | N/A |
| OCADA.4438_s_at | TCF4 | 866 |
| OC3P.4112.C1_s_at | TCF4 | 867 |
| OCHP.1876_s_at | TCF4 | 868 |
| OCADA.7185_s_at | TCF4 | 869 |
| OC3SNGnh.10608_s_at | TCF4 | 870 |
| OC3SNGnh.4569_x_at | TCF4 | 871 |
| OCADA.8009_s_at | TCF4 | 872 |
| OCADNP.14530_s_at | TCF4 | 873 |
| OC3SNG.2691-3954a_s_at | TCF4 | 874 |
| OC3SNGnh.10608_x_at | TCF4 | 875 |
| OC3P.3507.C1_s_at | TCF4 | 876 |
| OC3SNG.129-32a_s_at | TCIRG1 | 877 |
| OCRS2.3202_s_at | TCIRG1 | 878 |
| OCEM.457_x_at | TCIRG1 | 879 |
| OCEM.457_at | TCIRG1 | 880 |
| OCADNP.2642_s_at | TMEM169 | 881 |
| OC3P.6478.C1_s_at | TMEM200A | 882 |
| OC3P.6478.C1-363a_s_at | TM EM200A | 883 |
| OC3P.12427.C1_s_at | TMEM62 | 884 |
| OC3SNGn.2801-166a_s_at | TWIST1 | 885 |
| OCRS2.11542_s_at | TWIST1 | 886 |
| OC3SNGnh.13363_s_at | TXK | 887 |
| OC3SNGnh.17188_at | TXK | 888 |
| OC3SNGnh.17188_x_at | TXK | 889 |
| OCEM.1963_at | TXK | 890 |
| OCADNP.7909_s_at | TXK | 891 |
| OC3P.72.C6_x_at | TXK | 892 |
| OC3SNGnh.9832_x_at | TXK | 893 |
| OCADA.11004_s_at | UPK2 | 894 |
| OC3SNGnh.17460_at | USP53 | 895 |
| OCADNP.6200_s_at | USP53 | 896 |
| OC3SNG.3711-13a_s_at | USP53 | 897 |
| OCADA.7608_s_at | USP53 | 898 |
| ADXBad22_at | USP53 | N/A |
| OC3SNGnh.3076_s_at | USP53 | 899 |
| OC3SNGnh.20367_s_at | USP53 | 900 |
| OC3P.11072.C1_s_at | UTP14A | 901 |
| OC3SNGnh.14019_x_at | UTP14A | 902 |
| OC3P.15028.C1_s_at | VCAN | 903 |
| OCADNP.9657_s_at | VCAN | 904 |
| OCMX.15173.C1_s_at | VCAN | 905 |
| OCADNP.6197_s_at | VCAN | 906 |
| OCRS2.1143_s_at | VCAN | 907 |
| OC3SNGnh.16280_x_at | VCAN | 908 |
| OC3P.1200.C1_s_at | VCAN | 909 |
| OCADNP.7898_s_at | WNT11 | 910 |
| OC3P.12878.C1_s_at | WNT11 | 911 |
| OC3P.14348.C1_s_at | ZNF469 | 912 |

Accordingly, the method may comprise measuring the expression levels of at least one of GABRE, HLA-DPA1, CHI3L1, KCND2, GBP3, UPK2, SYTL4, LRRN1, USP53 and POU2F3. In specific embodiments the method comprises measuring the expression levels of each of GABRE, HLA-DPA1, CHI3L1, KCND2, GBP3, UPK2, SYTL4, LRRN1, USP53 and POU2F3. In further embodiments the method comprises measuring the expression levels of each of the biomarkers from Table F.

The method may comprise measuring the expression levels of at least 10, 11, 12, 13, 14, 15, 16, 17, 18, 19, 20, 25, 30, 40, 50, 60, 70, 80, 90, 100, 110, 120, 130, 140, 150, 160, 170, 180, 190, 200, 210, 220, 230 or each of the biomarkers from Table I. In certain embodiments the method may comprise measuring the expression levels of 10-25 biomarkers from Table I. The inventors have shown that measuring the expression levels of at least 10 of the markers in Table I enables the subtype to be reliably detected.

TABLE I

| GeneSymbol | GeneWeights | GeneBias |
|---|---|---|
| CRISP3 | 0.009244671 | 4.25279 |
| C10orf81 | 0.007440862 | 4.16685 |
| FBN3 | −0.007135587 | 6.564573 |
| C10orf114 | −0.006683214 | 5.254974 |
| UBD | 0.006650945 | 7.58811 |
| SFRP4 | −0.006511453 | 5.633072 |
| SCGB1D2 | 0.006029484 | 6.09871 |
| CXCL10 | 0.00600034 | 4.105151 |
| DEFB1 | 0.005933262 | 8.354037 |
| CKMT1B | 0.00588501 | 5.604975 |
| PKIA | −0.005796545 | 5.482019 |
| SNORD114-1 | −0.005771097 | 5.340838 |
| HOXA2 | −0.005764275 | 4.239838 |
| UNC5A | 0.005745289 | 5.960387 |
| GBP5 | 0.00567945 | 6.057223 |
| CYP4B1 | 0.005672014 | 5.585854 |
| CTSK | −0.005598646 | 5.849366 |
| BIRC3 | 0.005283614 | 8.531431 |
| LUM | −0.005266949 | 8.364716 |
| NCCRP1 | −0.005084004 | 5.756969 |
| MLLT11 | −0.004961885 | 6.827706 |
| FAM3B | 0.004958968 | 5.101375 |
| RPL9P16 | −0.004910088 | 6.453952 |
| ODZ3 | −0.004851049 | 4.104763 |
| RASL11B | −0.004842413 | 5.802979 |
| MT1G | 0.004809275 | 10.41099 |
| LRP4 | −0.004771008 | 4.664925 |
| PTPN7 | 0.004756756 | 7.284986 |
| COL11A1 | −0.004689519 | 3.541486 |
| TUBB4 | −0.004672931 | 6.359269 |
| SFRP5 | −0.00466637 | 4.142028 |
| CXCL12 | −0.004629236 | 5.117755 |
| TMEM98 | −0.004582999 | 6.070847 |
| TMEM47 | −0.004543117 | 3.355884 |
| SFRP2 | −0.004534184 | 5.576766 |
| KCNJ4 | −0.004467993 | 7.086926 |
| ADAMTS14 | −0.004465207 | 7.399778 |
| EPYC | −0.004441812 | 2.12021 |
| SMAD9 | −0.004437793 | 4.524905 |
| MIR142 | 0.004432341 | 9.492219 |
| MT1L | 0.004420979 | 8.917118 |
| HSPA2 | −0.004393242 | 6.05552 |
| EFS | −0.004375145 | 6.606757 |
| SALL2 | −0.004372373 | 9.157514 |
| CXCL11 | 0.004349799 | 3.526785 |
| ZNF711 | −0.00432014 | 6.528174 |
| IFI44L | 0.004316914 | 5.521583 |
| FAM111B | 0.00430404 | 7.339351 |
| SNORD114-19 | −0.004253407 | 3.757937 |
| ARHGAP28 | −0.004181503 | 4.26543 |
| MSI1 | −0.004167701 | 9.326208 |
| IFI27 | 0.004158526 | 11.45663 |
| NPBWR2 | −0.004145141 | 3.83414 |
| APOL6 | 0.004144974 | 6.173161 |
| THSD4 | 0.004126649 | 5.690818 |
| SLC40A1 | 0.004120522 | 5.142685 |
| CTGF | −0.004106249 | 8.871794 |
| C1orf130 | 0.004067685 | 4.223416 |
| SERPINA1 | 0.004021107 | 8.004173 |
| GPR126 | 0.00400077 | 4.54778 |
| APOL3 | 0.003991698 | 4.01636 |
| SRPX2 | −0.003974194 | 5.049348 |
| COL5A2 | −0.003955444 | 3.591515 |
| MICB | 0.003953138 | 6.388161 |
| CREB3L1 | −0.003911838 | 5.925211 |
| CDKN2C | 0.003889232 | 4.130717 |
| MIR143 | −0.003887926 | 4.429746 |
| CP | 0.003859011 | 5.769209 |
| F2R | −0.003856683 | 4.222794 |
| HLA-DMB | 0.003854578 | 7.489806 |
| FZD4 | 0.003835921 | 6.543752 |
| BTLA | 0.003811543 | 2.668735 |
| ETV7 | 0.00380241 | 4.308987 |
| FAT2 | 0.003791829 | 8.278542 |
| SNCAIP | −0.003787534 | 4.872882 |
| LPAR4 | −0.003781515 | 3.390116 |
| KIAA1324L | −0.003767177 | 4.149923 |
| PTGIS | −0.00372008 | 3.440601 |
| OAS2 | 0.003714546 | 5.35268 |
| AMYP1 | 0.003642358 | 4.651577 |
| PDGFD | −0.003617694 | 4.859654 |
| SERPINE1 | −0.003611522 | 5.967665 |
| THY1 | −0.003600739 | 8.04439 |
| TLR3 | 0.003559666 | 3.031327 |
| GPC6 | −0.00352027 | 3.099243 |
| TMC5 | 0.003486432 | 4.595376 |
| VIM | −0.003473684 | 6.670068 |
| CXCL14 | −0.003442516 | 4.866348 |
| IL15 | 0.003423676 | 3.804955 |
| SORL1 | 0.003413305 | 4.86007 |
| DTX1 | −0.003411875 | 5.52703 |
| PHACTR3 | −0.003369515 | 2.389338 |
| TERC | 0.003345052 | 6.451543 |
| TCF19 | 0.003339104 | 6.786973 |
| TMEM173 | 0.00333562 | 7.37983 |
| GOLGA2B | 0.003305893 | 3.913176 |
| METTL7B | 0.003292198 | 4.251683 |
| KLRK1 | 0.003277955 | 3.255008 |
| LRFN5 | −0.003255765 | 3.659329 |
| OLFML1 | −0.003250239 | 4.37426 |
| PVT1 | 0.00323521 | 6.364487 |
| CEACAM1 | 0.003213045 | 4.457571 |
| SRSF12 | −0.003178071 | 4.193823 |
| ADAMTSL2 | −0.003166265 | 5.4852 |
| SDC1 | −0.003141406 | 7.111513 |
| NXF2B | −0.003111687 | 4.226044 |
| NXF2 | −0.003110081 | 4.225574 |
| APOL1 | 0.003107861 | 7.133371 |
| ALOX5AP | 0.003107153 | 3.680016 |
| SNCG | 0.003097788 | 6.15653 |
| MYC | 0.003079695 | 5.950406 |
| PTRF | −0.003065554 | 7.328583 |
| SNORD114-18 | −0.003064175 | 3.111597 |
| C8orf55 | 0.003049858 | 8.256593 |
| C5orf4 | 0.003023007 | 5.041276 |
| MPDZ | −0.003020738 | 5.691978 |
| SIPA1L2 | −0.003012915 | 5.536502 |
| IFIH1 | 0.003011551 | 3.766603 |
| GALNT1 | −0.003009285 | 6.214229 |
| ROM1 | 0.003003676 | 8.371344 |
| GNG11 | −0.002978147 | 6.079215 |
| COL16A1 | −0.002969937 | 5.391862 |
| RNF113A | 0.002934491 | 7.947432 |
| FZD1 | −0.002929204 | 4.21814 |
| BICC1 | −0.0029214 | 3.748219 |
| NKD1 | −0.002904233 | 4.251593 |
| NRBP2 | 0.00290069 | 8.015463 |
| PARP9 | 0.002890116 | 5.683993 |
| RBMS3 | −0.002877296 | 4.643674 |
| GAS7 | −0.00287466 | 5.679247 |
| TNNI2 | −0.002872443 | 6.833335 |
| HSD17B8 | 0.002860611 | 6.586169 |
| NOTCH3 | −0.002855475 | 8.454157 |
| MEX3B | −0.002855225 | 3.211679 |
| EYA4 | −0.002849764 | 4.787113 |
| PPP1R16A | 0.002828479 | 6.876051 |
| CSRP2 | −0.002826031 | 7.12461 |
| HIF3A | −0.00280492 | 5.061668 |
| CHODL | 0.00279322 | 3.544441 |
| GPR176 | −0.002786706 | 4.252543 |
| VTCN1 | 0.002784647 | 6.131865 |
| PPP1R3B | −0.002779249 | 3.805854 |
| TMEM87B | 0.002771082 | 4.031005 |
| MOBKL2C | 0.002762945 | 7.424328 |
| MBNL3 | 0.002755567 | 3.432856 |
| TGFB3 | −0.002719409 | 5.332476 |
| ATP5J2P3 | 0.002716142 | 4.4555 |
| GPR124 | −0.002697971 | 5.165409 |
| PLXDC1 | −0.002697398 | 5.409047 |
| KIAA1486 | −0.002691441 | 7.697995 |
| KIAA1324 | 0.002688194 | 4.282685 |
| RNPC3 | 0.00267959 | 5.760009 |
| SYPL1 | 0.002648552 | 6.563364 |
| FAM96A | 0.002639649 | 6.181063 |
| TMOD4 | 0.002636074 | 4.746564 |
| SOX4 | −0.002592547 | 9.822965 |
| TIGD5 | 0.002586689 | 6.75499 |
| HLA-B | 0.002577418 | 7.629468 |
| PMP22 | −0.002571323 | 5.568301 |
| PPA1 | 0.00256965 | 9.239775 |
| BMP4 | −0.002542171 | 5.068577 |
| SRPK1 | 0.002541721 | 4.318048 |
| APOBEC3F | 0.00253947 | 5.728234 |
| HSD17614 | −0.00253867 | 7.55482 |
| PLCG1 | −0.00253365 | 7.434086 |
| PTGFRN | −0.002528775 | 5.927735 |
| COPZ2 | −0.002526837 | 5.134159 |
| PRPS2 | 0.002521435 | 6.943428 |
| PHC1 | −0.002519973 | 6.403549 |
| ILDR1 | 0.002519955 | 5.397283 |
| HCCS | 0.002519578 | 6.968027 |
| FJX1 | 0.002512224 | 6.501211 |
| VIPR1 | 0.00248841 | 3.390426 |
| TBC1D26 | −0.002480205 | 4.517079 |
| SDK1 | −0.002464848 | 3.992404 |
| RAB31 | −0.002455378 | 5.320999 |
| MAP3K13 | 0.002451542 | 4.170586 |
| IGFBP7 | −0.002443125 | 5.7624 |
| MX1 | 0.002435356 | 5.723388 |
| HTRA3 | −0.00242504 | 6.086372 |
| PMEPA1 | −0.002423218 | 6.316297 |
| NMNAT2 | 0.002411854 | 4.493685 |
| MYLIP | 0.002396765 | 6.467381 |
| BMF | −0.00239054 | 6.066753 |
| UNC5C | −0.002372973 | 4.261761 |
| B2M | 0.002368988 | 6.658859 |
| UBA7 | 0.002361512 | 8.518656 |
| SPDEF | 0.002357685 | 6.619913 |
| MTCP1 | 0.002341771 | 6.81278 |
| SNORD114-31 | −0.002338204 | 5.484037 |
| HERC6 | 0.002335968 | 5.857723 |
| BRF2 | −0.002323538 | 5.680577 |
| CHSY1 | −0.00228656 | 7.501669 |
| HSPBL3 | 0.002280481 | 8.578614 |
| C20orf3 | −0.002260827 | 8.781748 |
| DNMT3A | −0.002228757 | 7.020806 |
| OLFML3 | −0.002201975 | 6.717051 |
| DCAF5 | −0.002193965 | 6.117841 |
| SSH3 | 0.002182142 | 8.29951 |
| NPR1 | 0.002162441 | 7.269251 |
| DAAM1 | −0.0021509 | 5.886589 |
| HCG27 | 0.002145793 | 5.637696 |
| GRB10 | −0.002122228 | 6.372689 |
| HLA-DRB6 | 0.002075768 | 5.388441 |
| FAAH | 0.002072052 | 6.193823 |
| PUF60 | 0.002069218 | 8.621513 |
| ADAMTS10 | −0.002063412 | 5.207659 |
| ITGB1 | −0.002050701 | 5.441381 |
| ATXN7L3 | −0.002033507 | 8.759396 |
| CC2D1B | 0.002033207 | 5.173507 |
| SNORD46 | 0.001985667 | 10.44473 |
| ZBTB42 | 0.001963734 | 6.248473 |
| C6orf203 | 0.00194317 | 8.555232 |
| DBN1 | −0.001938651 | 9.151773 |
| NDUFS3 | 0.001932125 | 10.30757 |
| PCYOX1 | −0.001928012 | 6.843865 |
| ACTR1A | −0.001923873 | 6.222051 |
| PLEKHG2 | −0.001878479 | 6.339362 |
| PSMA5 | 0.001877248 | 7.908692 |
| MAL | −0.001866829 | 6.959474 |
| SQRDL | 0.001812312 | 6.762735 |
| DDR1 | 0.001781903 | 9.872079 |
| SERPINF1 | −0.00175887 | 10.81461 |
| SEC23A | −0.001701844 | 6.294431 |
| KDM5A | −0.001686649 | 6.389162 |
| RGPD2 | −0.001626152 | 6.125918 |
| LRRC14 | 0.001603355 | 6.772038 |
| RANBP2 | −0.001596694 | 6.338053 |
| MICA | 0.001512553 | 5.489141 |
| FBLN1 | −0.001484613 | 5.872453 |
| OGT | 0.001415954 | 7.57769 |
| EIF4EBP3 | 0.001335629 | 6.514681 |

The biomarkers from Table I are ranked in Table J from most important to least important based upon hazard ratio reduction when the genes are included versus when they are excluded. The genes/biomarkers may be selected for inclusion in a panel of biomarkers/a signature based on their ranking. Table K illustrates probesets that can be used to detect expression of the biomarkers.

TABLE J

| Gene | Total Delta HR | Rank |
|---|---|---|
| MT1L | 0.615118068 | 1 |
| MT1G | 0.472180746 | 2 |
| LRP4 | 0.428241646 | 3 |
| RASL11B | 0.424158825 | 4 |
| IFI27 | 0.32213756 | 5 |
| PKIA | 0.291930312 | 6 |
| ALOX5AP | 0.272480316 | 7 |
| UBD | 0.242546709 | 8 |
| MEX3B | 0.230392762 | 9 |
| TMEM98 | 0.229231657 | 10 |
| FBN3 | 0.227026061 | 11 |
| CXCL10 | 0.21976009 | 12 |
| ZNF711 | 0.214223021 | 13 |
| MSI1 | 0.192206467 | 14 |
| FAM3B | 0.18592276 | 15 |
| DTX1 | 0.183405107 | 16 |
| CP | 0.183009243 | 17 |
| DEFB1 | 0.173812067 | 18 |
| NRBP2 | 0.168297955 | 19 |
| METTL7B | 0.165287654 | 20 |
| TLR3 | 0.163657588 | 21 |
| CXCL11 | 0.155146275 | 22 |
| NXF2 | 0.152354088 | 23 |
| SNCG | 0.151636955 | 24 |
| IFI44L | 0.15043688 | 25 |
| MOBKL2C | 0.148007901 | 26 |
| NPR1 | 0.144504148 | 27 |
| NXF2B | 0.143829433 | 28 |
| TMEM87B | 0.143514747 | 29 |
| SRSF12 | 0.14192475 | 30 |
| SLC40A1 | 0.14006344 | 31 |
| C10orf114 | 0.138709815 | 32 |
| SOX4 | 0.137379065 | 33 |
| APOL6 | 0.132619361 | 34 |
| APOL3 | 0.131804118 | 35 |
| TMEM173 | 0.127263861 | 36 |
| UNC5A | 0.11842845 | 37 |
| HLA-DMB | 0.118263574 | 38 |
| GPC6 | 0.113746774 | 39 |
| BIRC3 | 0.1130983 | 40 |
| KIAA1486 | 0.110209853 | 41 |
| GPR126 | 0.109454197 | 42 |
| MIR142 | 0.108675197 | 43 |
| HSPBL3 | 0.107843483 | 44 |
| GBP5 | 0.10446511 | 45 |
| VTCN1 | 0.102993036 | 46 |
| EFS | 0.102594908 | 47 |
| IFIH1 | 0.10045923 | 48 |
| APOL1 | 0.100123166 | 49 |
| ILDR1 | 0.100043711 | 50 |
| MX1 | 0.099707498 | 51 |
| PUF60 | 0.098560494 | 52 |
| MICB | 0.097058318 | 53 |
| MICA | 0.095790241 | 54 |
| HERC6 | 0.091124393 | 55 |
| PPP1R16A | 0.090566038 | 56 |
| PHACTR3 | 0.088649365 | 57 |
| BTLA | 0.088347137 | 58 |
| PLCG1 | 0.087624812 | 59 |
| SALL2 | 0.086781935 | 60 |
| C1orf130 | 0.086312394 | 61 |
| VIM | 0.083062394 | 62 |
| IL15 | 0.082662071 | 63 |
| SERPINA1 | 0.080336497 | 64 |
| ROM1 | 0.07576285 | 65 |
| FAT2 | 0.07540916 | 66 |
| KLRK1 | 0.075409095 | 67 |
| PTPN7 | 0.072950165 | 68 |
| PARP9 | 0.071381591 | 69 |
| ATP5J2P3 | 0.068319455 | 70 |
| C8orf55 | 0.067706631 | 71 |
| HLA-DRB6 | 0.065799796 | 72 |
| UBA7 | 0.064343371 | 73 |
| AMYP1 | 0.062359242 | 74 |
| PPP1R3B | 0.061652663 | 75 |
| OAS2 | 0.061174581 | 76 |
| RGPD2 | 0.06018489 | 77 |
| CHSY1 | 0.056973948 | 78 |
| SDK1 | 0.054082406 | 79 |
| MIR143 | 0.053547598 | 80 |
| B2M | 0.053469453 | 81 |
| NPBWR2 | 0.053118153 | 82 |
| SSH3 | 0.05155016 | 83 |
| NDUFS3 | 0.050357674 | 84 |
| SNORD46 | 0.049505727 | 85 |
| LRRC14 | 0.04834913 | 86 |
| SYPL1 | 0.048048239 | 87 |
| GRB10 | 0.042893881 | 88 |
| RANBP2 | 0.042771834 | 89 |
| LRFN5 | 0.04189327 | 90 |
| NKD1 | 0.041594518 | 91 |
| DNMT3A | 0.040633094 | 92 |
| PCYOX1 | 0.040460762 | 93 |
| APOBEC3F | 0.037846365 | 94 |
| BRF2 | 0.03775925 | 95 |
| MYC | 0.037625087 | 96 |
| HCG27 | 0.03651511 | 97 |
| RNPC3 | 0.036449685 | 98 |
| FAM96A | 0.036099171 | 99 |
| ZBTB42 | 0.035762757 | 100 |
| IGFBP7 | 0.035704168 | 101 |
| MAP3K13 | 0.035039881 | 102 |
| GALNT1 | 0.034633608 | 103 |
| MYLIP | 0.034121783 | 104 |
| PHC1 | 0.031292623 | 105 |
| FJX1 | 0.030921305 | 106 |
| CSRP2 | 0.029128198 | 107 |
| HLA-B | 0.028631601 | 108 |
| HSD17B8 | 0.027873252 | 109 |
| PTGFRN | 0.027233148 | 110 |
| DCAF5 | 0.026405405 | 111 |
| TMEM47 | 0.021956786 | 112 |
| SQRDL | 0.021004945 | 113 |
| ETV7 | 0.019282689 | 114 |
| C5orf4 | 0.018300269 | 115 |
| KDM5A | 0.017375372 | 116 |
| NMNAT2 | 0.016695136 | 117 |
| CYP4B1 | 0.014669028 | 118 |
| CC2D1B | 0.014147408 | 119 |
| EIF4EBP3 | 0.013653958 | 120 |
| LPAR4 | 0.013583634 | 121 |
| SNORD114-31 | 0.011357203 | 122 |
| SIPA1L2 | 0.010649087 | 123 |
| ITGB1 | 0.010477821 | 124 |
| ADAMTS10 | 0.010139752 | 125 |
| MLLT11 | 0.010014206 | 126 |
| OGT | 0.009114642 | 127 |
| EYA4 | 0.007618687 | 128 |
| TMC5 | 0.006544943 | 129 |
| ATXN7L3 | 0.005848973 | 130 |
| VIPR1 | 0.005324997 | 131 |
| MTCP1 | 0.00297225 | 132 |
| C20orf3 | 0.002054112 | 133 |
| NOTCH3 | 0.001374142 | 134 |
| PLEKHG2 | 0.000697928 | 135 |
| SNCAIP | −0.000937809 | 136 |
| DAAM1 | −0.001532018 | 137 |
| BMF | −0.002501529 | 138 |
| TIGD5 | −0.004913775 | 139 |
| PSMA5 | −0.004951732 | 140 |
| SNORD114-18 | −0.007256399 | 141 |
| TBC1D26 | −0.00805853 | 142 |
| SEC23A | −0.008366824 | 143 |
| RNF113A | −0.008502226 | 144 |
| FAAH | −0.009699661 | 145 |
| TMOD4 | −0.009707802 | 146 |
| GNG11 | −0.00986732 | 147 |
| RPL9P16 | −0.011949323 | 148 |
| ARHGAP28 | −0.012754103 | 149 |
| UNC5C | −0.013324554 | 150 |
| RBMS3 | −0.014284394 | 151 |
| BMP4 | −0.016512281 | 152 |
| CHODL | −0.019546582 | 153 |
| TERC | −0.020201664 | 154 |
| GPR176 | −0.021146329 | 155 |
| PPA1 | −0.021176568 | 156 |
| DDR1 | −0.021757339 | 157 |
| ACTR1A | −0.023596243 | 158 |
| GPR124 | −0.02574171 | 159 |
| SMAD9 | −0.026817767 | 160 |
| C6orf203 | −0.029106466 | 161 |
| DBN1 | −0.030827615 | 162 |
| SDC1 | −0.032523027 | 163 |
| SPDEF | −0.033787647 | 164 |
| TNNI2 | −0.035527955 | 165 |
| MPDZ | −0.037447958 | 166 |
| PRPS2 | −0.039602179 | 167 |
| PVT1 | −0.04027777 | 168 |
| KIAA1324 | −0.041499097 | 169 |
| SCGB1D2 | −0.043682554 | 170 |
| MBNL3 | −0.045374866 | 171 |
| SORL1 | −0.049145596 | 172 |
| FBLN1 | −0.049870444 | 173 |
| SRPX2 | −0.051419372 | 174 |
| HCCS | −0.053517069 | 175 |
| HTRA3 | −0.05393539 | 176 |
| PMP22 | −0.056596896 | 177 |
| HIF3A | −0.058792401 | 178 |
| ADAMTSL2 | −0.059012281 | 179 |
| CDKN2C | −0.059303226 | 180 |
| F2R | −0.064443812 | 181 |
| GOLGA2B | −0.075799765 | 182 |
| CEACAM1 | −0.080861206 | 183 |
| BICC1 | −0.081748924 | 184 |
| OLFML1 | −0.089688046 | 185 |
| GAS7 | −0.091550492 | 186 |
| TUBB4 | −0.094233082 | 187 |
| SFRP5 | −0.095268495 | 188 |
| PMEPA1 | −0.098648425 | 189 |
| SNORD114-19 | −0.099307115 | 190 |
| SRPK1 | −0.103289867 | 191 |
| MAL | −0.106958728 | 192 |
| HSPA2 | −0.109505993 | 193 |
| NCCRP1 | −0.111600258 | 194 |
| PTGIS | −0.113102299 | 195 |
| KIAA1324L | −0.115645454 | 196 |
| FZD4 | −0.117484004 | 197 |
| TCF19 | −0.125969306 | 198 |
| SERPINF1 | −0.129991571 | 199 |
| PTRF | −0.132998458 | 200 |
| PLXDC1 | −0.133187724 | 201 |
| TGFB3 | −0.149423417 | 202 |
| COPZ2 | −0.150978011 | 203 |
| COL16A1 | −0.152779464 | 204 |
| THSD4 | −0.153534385 | 205 |
| HSD17614 | −0.157660215 | 206 |
| RAB31 | −0.162011114 | 207 |
| OLFML3 | −0.165389996 | 208 |
| KCNJ4 | −0.16970666 | 209 |
| PDGFD | −0.181432458 | 210 |
| FZD1 | −0.183922929 | 211 |
| C10orf81 | −0.189396338 | 212 |
| THY1 | −0.203086307 | 213 |
| SERPINE1 | −0.203441585 | 214 |
| ADAMTS14 | −0.221523101 | 215 |
| CREB3L1 | −0.248768165 | 216 |
| CTGF | −0.250513415 | 217 |
| CRISP3 | −0.257890987 | 218 |
| SNORD114-1 | −0.261554435 | 219 |
| FAM111B | −0.31605279 | 220 |
| CXCL14 | −0.356310031 | 221 |
| COL5A2 | −0.373994826 | 222 |
| SFRP4 | −0.443299754 | 223 |
| ODZ3 | −0.452582522 | 224 |
| CKMT1B | −0.464675681 | 225 |
| HOXA2 | −0.466941259 | 226 |
| CXCL12 | −0.500158659 | 227 |
| SFRP2 | −0.511192219 | 228 |
| EPYC | −0.53044603 | 229 |
| CTSK | −0.548238141 | 230 |
| COL11A1 | −0.548627827 | 231 |
| LUM | −0.666936872 | 232 |

TABLE K

| Probeset | Gene | SEQ ID No. |
|---|---|---|
| OC3SNGnh.12195_x_at | ACTR1A | 913 |
| ADXStrong36_at | ACTR1A | N/A |
| OC3P.4237.C1_s_at | ACTR1A | 914 |
| OC3P.2875.C1_s_at | ACTR1A | 915 |
| OC3SNGn.2781-864a_s_at | ACTR1A | 916 |
| OCADA.3613_s_at | ADAMTS10 | 917 |
| OC3SNGnh.16809_s_at | ADAMTS10 | 918 |
| OCADA.3613_x_at | ADAMTS10 | 919 |
| OC3SNGnh.15138_x_at | ADAMTS10 | 920 |
| OCADNP.16013_s_at | ADAMTS10 | 921 |
| OC3SNGnh.16809_at | ADAMTS10 | 922 |
| OC3P.10843.C1_s_at | ADAMTS14 | 923 |
| OC3P.10512.C1_s_at | ADAMTSL2 | 924 |
| OCRS2.7089_s_at | ADAMTSL2 | 925 |
| OCADNP.9212_s_at | ALOX5AP | 926 |
| OC3SNGn.6061-323a_s_at | ALOX5AP | 927 |
| OCHP.1634_x_at | AMYP1 | 928 |
| OCRS2.10811_s_at | AMYP1 | 929 |
| OCRS2.2503_s_at | AMYP1 | 930 |
| OCUTR.200_s_at | APOBEC3F | 931 |
| OCADNP.5415_x_at | APOBEC3F | 932 |
| OC3SNGn.8424-313a_x_at | APOBEC3F | 933 |
| OC3P.8406.C1_x_at | APOBEC3F | 934 |
| OCADA.5213_s_at | APOBEC3F | 935 |
| OC3P.8406.C1_s_at | APOBEC3F | 936 |
| OC3SNGn.2950-782a_x_at | APOL1 | 937 |
| OC3SNGnh.16528_x_at | APOL1 | 938 |
| OC3SNGnh.16528_at | APOL1 | 939 |
| OC3P.1177.C1_x_at | APOL1 | 940 |
| OC3P.1177.C2_s_at | APOL3 | 941 |
| OC3SNGnh.7607_x_at | APOL3 | 942 |
| OC3P.5638.C1_x_at | APOL6 | 943 |
| OC3SNG.3005-7069a_s_at | APOL6 | 944 |
| OCADA.7386_s_at | ARHGAP28 | 945 |
| OCADNP.8921_s_at | ARHGAP28 | 946 |
| OCRS2.820_s_at | ATP5J2P3 | 947 |
| OCRS2.5034_s_at | ATXN7L3 | 948 |
| OC3SNG.2893-43a_s_at | ATXN7L3 | 949 |
| OCMXSNG.5067_s_at | B2M | 950 |
| OC3P.405.CB2_x_at | B2M | 951 |
| ADXGoodB50_at | B2M | N/A |
| OC3P.405.CB1_x_at | B2M | 952 |
| OCADNP.3105_s_at | B2M | 953 |
| OCADNP.4353_s_at | B2M | 954 |
| OCEM.1629_x_at | B2M | 955 |
| OCADNP.1950_s_at | BICC1 | 956 |
| OCADA.10388_s_at | BICC1 | 957 |
| OCMXSNG.4199_x_at | BICC1 | 958 |
| OC3SNGnh.7031_s_at | BICC1 | 959 |
| OCRS2.4990_s_at | BICC1 | 960 |
| OC3SNGnh.6778_s_at | BICC1 | 961 |
| OC3SNGnh.11887_x_at | BICC1 | 962 |
| OC3SNG.710-16934a_s_at | BIRC3 | 963 |
| OC3SNG.1178-15a_s_at | BIRC3 | 964 |
| OC3P.6452.C1_s_at | BMF | 965 |
| OC3SNGn.2995-3680a_s_at | BMF | 966 |
| OC3SNG.1690-1116a_s_at | BMP4 | 967 |
| OC3SNG.6227-154a_s_at | BMP4 | 968 |
| OCHP.1932_s_at | BMP4 | 969 |
| OCMX.1053.C1_x_at | BRF2 | 970 |
| OCMXSNG.2477_at | BRF2 | 971 |
| ADXStrong39_at | BRF2 | N/A |
| OCMX.1053.C1_at | BRF2 | 972 |
| OCADNP.8779_s_at | BRF2 | 973 |
| OCADNP.8778_s_at | BRF2 | 974 |
| ADXGood98_at | BRF2 | N/A |
| OC3SNGnh.11044_s_at | BTLA | 975 |
| OCRS.1136_s_at | BTLA | 976 |
| OC3SNGn.174-1a_s_at | C10orf114 | 977 |
| OC3SNG.1180-19a_s_at | C10orf81 | 978 |
| OC3SNGn.301-8a_s_at | C10orf81 | 979 |
| OC3P.5692.C1_s_at | C10orf81 | 980 |
| OC3SNGn.7786-6a_s_at | C10orf81 | 981 |
| OC3SNG.1287-14a_s_at | C1orf130 | 982 |
| OC3P.2845.C1_s_at | C20orf3 | 983 |
| OC3P.2845.C1_at | C20orf3 | 984 |
| OC3SNGnh.9851_x_at | C5orf4 | 985 |
| OC3P.5410.C1_s_at | C5orf4 | 986 |
| OC3SNGnh.9851_at | C5orf4 | 987 |
| OC3SNG.887-30a_x_at | C6orf203 | 988 |
| ADXGood87_at | C6orf203 | N/A |
| OC3SNG.4961-30a_x_at | C6orf203 | 989 |
| OC3SNG.2275-28a_x_at | C6orf203 | 990 |
| OC3P.7754.C1_x_at | C8orf55 | 991 |
| OCRS.1072_s_at | CC2D1B | 992 |
| OC3P.8147.C1_s_at | CC2D1B | 993 |
| OCADNP.6491_s_at | CC2D1B | 994 |
| OCADA.5455_s_at | CC2D1B | 995 |
| OCADNP.9668_s_at | CDKN2C | 996 |
| OC3P.12264.C1_x_at | CDKN2C | 997 |
| OC3SNGn.8263-35a_x_at | CEACAM1 | 998 |
| OC3SNGn.2117-1801a_s_at | CEACAM1 | 999 |
| OCHP.710_s_at | CEACAM1 | 1000 |
| OC3P.13249.C1_x_at | CHODL | 1001 |
| OCMX.7042.C1_s_at | CHODL | 1002 |
| OCMX.15594.C1_s_at | CHODL | 1003 |
| OCMXSNG.1530_s_at | CHODL | 1004 |
| OC3SNG.3556-78a_s_at | CHODL | 1005 |
| OCMX.7042.C1_x_at | CHODL | 1006 |
| OC3SNGn.4742-71060a_s_at | CHODL | 1007 |
| OC3SNG.549-201852a_s_at | CHODL | 1008 |
| OC3SNGn.4741-34831a_s_at | CHODL | 1009 |
| OCEM.1035_s_at | CHODL | 1010 |
| OC3P.5287.C1_at | CHSY1 | 1011 |
| OC3P.5894.C1_s_at | CHSY1 | 1012 |
| OC3P.4600.C1_s_at | CKMT1B | 1013 |
| OC3P.1561.C1_s_at | COL11A1 | 1014 |
| OC3P.6907.C1_s_at | COL11A1 | 1015 |
| OC3P.1561.C1_x_at | COL11A1 | 1016 |
| OCADA.4133_s_at | COL11A1 | 1017 |
| OC3SNGnh.16343_x_at | COL11A1 | 1018 |
| OC3P.3047.C1_x_at | COL16A1 | 1019 |
| OC3P.3047.C1-304a_s_at | COL16A1 | 1020 |
| OC3SNGnh.6481_s_at | COL16A1 | 1021 |
| OCMX.338.C1_at | COL5A2 | 1022 |
| OC3P.6029.C1_s_at | COL5A2 | 1023 |
| OCRS2.8960_s_at | COL5A2 | 1024 |
| OCMX.338.C1_x_at | COL5A2 | 1025 |
| OC3P.2713.C1_s_at | COL5A2 | 1026 |
| OC3P.12307.C1_x_at | COL5A2 | 1027 |
| OC3SNGnh.20566_s_at | COPZ2 | 1028 |
| OCADA.4902_s_at | COPZ2 | 1029 |
| OC3SNGnh.4100_at | CP | 1030 |
| OCMX.4331.C3_s_at | CP | 1031 |
| OCADA.4957_s_at | CP | 1032 |
| OCADNP.7608_s_at | CP | 1033 |
| OC3SNG.1600-2703a_s_at | CP | 1034 |
| OC3SNGn.5770-13089a_at | CP | 1035 |
| OCHP.124_s_at | CP | 1036 |
| OC3P.2585.C1_x_at | CP | 1037 |
| OCHPRC.52_s_at | CP | 1038 |
| OCHP.193_s_at | CP | 1039 |
| OC3P.2361.C1_s_at | CP | 1040 |
| OC3SNG.67-21a_s_at | CREB3L1 | 1041 |
| OC3SNG.1826-29a_x_at | CRISP3 | 1042 |
| OC3SNGnh.3590_at | CSRP2 | 1043 |
| OCHP.1027_s_at | CSRP2 | 1044 |
| OCADNP.9526_s_at | CTGF | 1045 |
| OC3P.1178.C1_at | CTGF | 1046 |
| OC3P.1178.C1_x_at | CTGF | 1047 |
| OC3P.4572.C1_s_at | CTSK | 1048 |
| OC3P.3318.C1_s_at | CXCL10 | 1049 |
| OCADA.10769_s_at | CXCL11 | 1050 |
| OCADA.9983_s_at | CXCL11 | 1051 |
| OCHP.873_s_at | CXCL12 | 1052 |
| OCHP.852_s_at | CXCL12 | 1053 |
| OCHP.913_s_at | CXCL12 | 1054 |
| OCADA.8979_s_at | CXCL14 | 1055 |
| OCHP.1072_s_at | CXCL14 | 1056 |
| OC3SNG.240-1128a_s_at | CXCL14 | 1057 |
| OCHP.1896_s_at | CYP4B1 | 1058 |
| OCADNP.709_s_at | CYP4B1 | 1059 |
| OCADNP.2336_s_at | DAAM1 | 1060 |
| OCADNP.4315_s_at | DAAM1 | 1061 |
| OC3P.15553.C1_s_at | DAAM1 | 1062 |
| OC3SNGn.2635-651a_s_at | DAAM1 | 1063 |
| OC3SNGnh.12060_s_at | DAAM1 | 1064 |
| OCADA.7103_s_at | DAAM1 | 1065 |
| OCRS.1398_at | DBN1 | 1066 |
| OC3P.298.C1_s_at | DBN1 | 1067 |
| OCRS.1398_x_at | DBN1 | 1068 |
| OCADA.8592_s_at | DBN1 | 1069 |
| OC3SNG.5293-38a_s_at | DCAF5 | 1070 |
| OCADA.3135_s_at | DCAF5 | 1071 |
| OC3P.12587.C1_s_at | DCAF5 | 1072 |
| OC3P.9318.C1_s_at | DCAF5 | 1073 |
| OC3P.9525.C1_x_at | DDR1 | 1074 |
| OC3SNG.1859-16a_s_at | DDR1 | 1075 |
| OC3SNGn.6552-124a_s_at | DEFB1 | 1076 |
| OCRS2.12509_s_at | DEFB1 | 1077 |
| ADXStrongB6_at | DNMT3A | N/A |
| OC3P.9719.C1_at | DNMT3A | 1078 |
| OCRS2.1573_s_at | DNMT3A | 1079 |
| OC3SNGnh.5575_x_at | DNMT3A | 1080 |
| OCADNP.9700_s_at | DNMT3A | 1081 |
| OC3SNGnh.16027_x_at | DNMT3A | 1082 |
| OCMXSNG.4423_x_at | DNMT3A | 1083 |
| OC3P.9719.C1_s_at | DNMT3A | 1084 |
| OC3P.9719.C1-476a_s_at | DNMT3A | 1085 |
| OCMXSNG.4423_at | DNMT3A | 1086 |
| OC3SNGnh.7008_x_at | DNMT3A | 1087 |
| OC3SNG.804-53a_s_at | DTX1 | 1088 |
| OC3SNGnh.3248_x_at | DTX1 | 1089 |
| OCADA.1205_s_at | DTX1 | 1090 |
| OC3P.2375.C1_s_at | EFS | 1091 |
| OCADNP.10111_s_at | EFS | 1092 |
| OC3P.2318.C1_s_at | EIF4EBP3 | 1093 |
| OC3SNGnh.19542_s_at | EIF4EBP3 | 1094 |
| OCADA.9737_s_at | EPYC | 1095 |
| OC3SNG.3070-45a_s_at | ETV7 | 1096 |
| OCRS2.11702_x_at | ETV7 | 1097 |
| OCEM.668_s_at | ETV7 | 1098 |
| OC3P.6561.C1_s_at | EYA4 | 1099 |
| ADXUglyB80_at | EYA4 | N/A |
| OCRS.391_s_at | EYA4 | 1100 |
| OC3SNGnh.2970_x_at | EYA4 | 1101 |
| OC3SNGnh.15042_x_at | EYA4 | 1102 |
| OCADNP.15820_s_at | F2R | 1103 |
| OCHP.779_x_at | F2R | 1104 |
| OC3SNG.712-38a_s_at | F2R | 1105 |
| OC3P.6713.C1_s_at | FAAH | 1106 |
| OCADA.835_s_at | FAM111B | 1107 |
| OCRS2.11211_x_at | FAM111B | 1108 |
| OCRS2.11211_at | FAM111B | 1109 |
| OCHP.614_s_at | FAM3B | 1110 |
| OC3P.10042.C1_s_at | FAM3B | 1111 |
| OC3SNG.854-20a_s_at | FAM96A | 1112 |
| OC3P.11005.C1_s_at | FAT2 | 1113 |
| OC3P.2096.C1_x_at | FBLN1 | 1114 |
| OC3P.2147.C1-478a_s_at | FBLN1 | 1115 |
| OCHP.904_x_at | FBLN1 | 1116 |
| OCHP.212_s_at | FBLN1 | 1117 |
| OCADNP.9451_s_at | FBLN1 | 1118 |
| OC3P.1250.C1_s_at | FBLN1 | 1119 |
| OCMX.2648.C1_s_at | FBLN1 | 1120 |
| OCHP.899_s_at | FBLN1 | 1121 |
| OC3P.11075.C1_s_at | FBN3 | 1122 |
| OCRS2.5152_s_at | FJX1 | 1123 |
| OC3P.6045.C1_s_at | FJX1 | 1124 |
| OC3P.4921.C1_at | FZD1 | 1125 |
| OC3P.4921.C1-347a_s_at | FZD1 | 1126 |
| OCADNP.7579_s_at | FZD1 | 1127 |
| OC3P.4921.C1_x_at | FZD1 | 1128 |
| OC3SNGn.1967-29a_s_at | FZD4 | 1129 |
| OCADNP.7425_s_at | FZD4 | 1130 |
| OC3P.2042.C1_s_at | FZD4 | 1131 |
| OC3P.13199.C1_s_at | GALNT1 | 1132 |
| OC3SNGnh.8607_x_at | GALNT1 | 1133 |
| OC3P.6817.C1_s_at | GALNT1 | 1134 |
| OCADNP.10124_s_at | GALNT1 | 1135 |
| OCADNP.12320_s_at | GALNT1 | 1136 |
| OCADA.4308_s_at | GALNT1 | 1137 |
| OC3SNG.1687-462a_s_at | GALNT1 | 1138 |
| OC3P.8087.C1_s_at | GAS7 | 1139 |
| OC3SNGn.2341-4940a_s_at | GAS7 | 1140 |
| OC3SNGn.2340-3426a_s_at | GAS7 | 1141 |
| OCADNP.9441_s_at | GAS7 | 1142 |
| OCADA.10080_s_at | GAS7 | 1143 |
| ADXStrongB54_at | GAS7 | N/A |
| OCADA.10109_s_at | GAS7 | 1144 |
| OCADA.1734_s_at | GAS7 | 1145 |
| OC3P.1629.C1_s_at | GBP5 | 1146 |
| OC3SNGn.3058-31a_s_at | GBP5 | 1147 |
| OC3SNGn.8331-31a_s_at | GBP5 | 1148 |
| OC3P.12320.C1_s_at | GNG11 | 1149 |
| OC3P.9220.C1_s_at | GOLGA2B | 1150 |
| OCADNP.11902_s_at | GPC6 | 1151 |
| OC3SNGnh.342_x_at | GPC6 | 1152 |
| OCADA.7642_s_at | GPC6 | 1153 |
| OCADA.4306_s_at | GPC6 | 1154 |
| OCADA.12782_s_at | GPC6 | 1155 |
| OCRS.951_s_at | GPC6 | 1156 |
| OCADNP.14363_s_at | GPC6 | 1157 |
| OCADNP.13892_s_at | GPC6 | 1158 |
| OC3SNGnh.10610_x_at | GPC6 | 1159 |
| OCADA.4214_s_at | GPC6 | 1160 |
| OCRS2.8554_s_at | GPR124 | 1161 |
| OC3P.7680.C1-589a_s_at | GPR124 | 1162 |
| OC3P.7680.C1_at | GPR124 | 1163 |
| OC3SNGn.3383-29a_s_at | GPR126 | 1164 |
| OCADNP.12006_s_at | GPR126 | 1165 |
| OC3P.11725.C1_at | GPR176 | 1166 |
| OCADNP.7882_s_at | GPR176 | 1167 |
| OCADNP.15707_s_at | GPR176 | 1168 |
| OC3P.11725.C1_s_at | GPR176 | 1169 |
| OC3P.13228.C1_s_at | GRB10 | 1170 |
| ADXGoodB21_at | GRB10 | N/A |
| OCADNP.8343_s_at | GRB10 | 1171 |
| OCADA.8023_s_at | GRB10 | 1172 |
| OC3P.9535.C1_s_at | GRB10 | 1173 |
| ADXGood101_at | HCCS | N/A |
| OC3P.3092.C1_s_at | HCCS | 1174 |
| OC3SNG.6061-26a_s_at | HCCS | 1175 |
| OCRS2.11321_s_at | HCG27 | 1176 |
| OC3P.3875.C1_s_at | HERC6 | 1177 |
| OCADA.1952_s_at | HERC6 | 1178 |
| OC3SNGn.7249-10a_x_at | HIF3A | 1179 |
| OCADA.572_s_at | HIF3A | 1180 |
| OCADNP.8797_s_at | HIF3A | 1181 |
| OCADA.452_s_at | HIF3A | 1182 |
| OCADNP.5407_s_at | HIF3A | 1183 |
| OCADNP.5866_s_at | HIF3A | 1184 |
| OCEM.1965_x_at | HLA-B | 1185 |
| OCADNP.9529_x_at | HLA-B | 1186 |
| OCADNP.9519_x_at | HLA-B | 1187 |
| OCADNP.8709_x_at | HLA-B | 1188 |
| OCRS2.731_x_at | HLA-B | 1189 |
| OC3P.141.C12_x_at | HLA-B | 1190 |
| OC3P.141.C17_x_at | HLA-B | 1191 |
| OC3P.4729.C1_s_at | HLA-DMB | 1192 |
| OCMX.15188.C1_s_at | HLA-DMB | 1193 |
| OCRS2.11859_s_at | HLA-DRB6 | 1194 |
| OC3SNGn.5065-56a_x_at | HLA-DRB6 | 1195 |
| OCADNP.4750_x_at | HLA-DRB6 | 1196 |
| OCADNP.6175_x_at | HLA-DRB6 | 1197 |
| OCADA.5023_s_at | HOXA2 | 1198 |
| OC3SNG.4039-40a_s_at | HSD17B14 | 1199 |
| OC3SNG.813-28a_s_at | HSD17B14 | 1200 |
| OC3P.15241.C1_s_at | HSD17B8 | 1201 |
| OC3P.4924.C1_s_at | HSPA2 | 1202 |
| OC3P.4924.C1-306a_s_at | HSPA2 | 1203 |
| OCRS2.3397_s_at | HSPBL3 | 1204 |
| OCHP.611_s_at | HSPBL3 | 1205 |
| OC3P.12955.C1_s_at | HTRA3 | 1206 |
| OC3SNG.638-18a_s_at | HTRA3 | 1207 |
| OC3SNGn.8155-20a_x_at | IFI27 | 1208 |
| OC3P.2271.C3_s_at | IFI27 | 1209 |
| OC3P.12110.C1_s_at | IFI44L | 1210 |
| OC3P.9547.C1_x_at | IFI44L | 1211 |
| OC3P.9547.C1_at | IFI44L | 1212 |
| ADXBad32_at | IFI44L | N/A |
| OC3P.9280.C1_x_at | IFI44L | 1213 |
| OCADA.488_s_at | IFIH1 | 1214 |
| ADXUglyB47_at | IFIH1 | N/A |
| OC3SNGnh.3305_s_at | IFIH1 | 1215 |
| OC3P.10280.C1_s_at | IFIH1 | 1216 |
| OCADA.5602_s_at | IFIH1 | 1217 |
| OCADNP.3740_s_at | IGFBP7 | 1218 |
| OCMX.11971.C1_s_at | IGFBP7 | 1219 |
| OC3SNGn.4133-3670a_x_at | IGFBP7 | 1220 |
| OC3SNGnh.5634_s_at | IGFBP7 | 1221 |
| OC3SNGn.5009-5456a_x_at | IGFBP7 | 1222 |
| ADXGoodB24_at | IGFBP7 | N/A |
| OCADNP.3131_x_at | IGFBP7 | 1223 |
| OC3SNG.1653-16a_s_at | IGFBP7 | 1224 |
| OCADNP.4032_s_at | IGFBP7 | 1225 |
| OCADNP.4758_s_at | IL15 | 1226 |
| OC3SNG.2608-26a_s_at | IL15 | 1227 |
| OC3SNGnh.17571_x_at | IL15 | 1228 |
| OCADNP.7752_s_at | IL15 | 1229 |
| OC3SNGnh.17571_at | IL15 | 1230 |
| OCRS2.6584_s_at | ILDR1 | 1231 |
| OC3SNG.1239-107a_s_at | ILDR1 | 1232 |
| OCADNP.370_s_at | ILDR1 | 1233 |
| OCADNP.4263_s_at | ITGB1 | 1234 |
| OCHP.774_x_at | ITGB1 | 1235 |
| OCHP.334_s_at | ITGB1 | 1236 |
| OCHP.798_x_at | ITGB1 | 1237 |
| OCHP.744_s_at | ITGB1 | 1238 |
| OCADNP.408_s_at | ITGB1 | 1239 |
| OCHP.761_x_at | ITGB1 | 1240 |
| OCADNP.17259_s_at | KCNJ4 | 1241 |
| OCADA.9900_s_at | KCNJ4 | 1242 |
| OCADA.9429_s_at | KDM5A | 1243 |
| OC3SNGnh.17035_at | KDM5A | 1244 |
| OCMX.12398.C1_x_at | KDM5A | 1245 |
| OC3P.6882.C1_s_at | KDM5A | 1246 |
| OC3SNGnh.17668_x_at | KDM5A | 1247 |
| OCHP.1380_s_at | KDM5A | 1248 |
| OC3P.12897.C1_s_at | KDM5A | 1249 |
| OCADNP.2795_s_at | KDM5A | 1250 |
| OC3SNGnh.17035_x_at | KDM5A | 1251 |
| OCADA.4719_s_at | KDM5A | 1252 |
| OC3SNGnh.12409_x_at | KIAA1324 | 1253 |
| ADXBad44_at | KIAA1324 | N/A |
| OC3SNG.4404-2900a_x_at | KIAA1324 | 1254 |
| ADXStrongB45_at | KIAA1324 | N/A |
| OCADNP.5286_s_at | KIAA1324 | 1255 |
| OCMX.11681.C1_at | KIAA1324 | 1256 |
| OCMX.11681.C1_x_at | KIAA1324 | 1257 |
| OC3SNGnh.4924_x_at | KIAA1324 | 1258 |
| OC3SNG.3368-36a_s_at | KIAA1324 | 1259 |
| ADXBad2_at | KIAA1324 | N/A |
| OC3SNG.35-2898a_x_at | KIAA1324 | 1260 |
| OC3P.10299.C1_s_at | KIAA1324 | 1261 |
| OC3SNGn.244-94a_s_at | KIAA1324L | 1262 |
| OCADNP.6595_s_at | KIAA1324L | 1263 |
| OCMX.12418.C1_at | KIAA1486 | 1264 |
| OCADNP.745_s_at | KLRK1 | 1265 |
| OCEM.419_s_at | KLRK1 | 1266 |
| OCADA.9684_s_at | KLRK1 | 1267 |
| ADXUglyB24_at | LPAR4 | N/A |
| OCADA.9771_s_at | LPAR4 | 1268 |
| OCADA.7662_s_at | LRFN5 | 1269 |
| OCADNP.2843_s_at | LRFN5 | 1270 |
| OC3P.7872.C1_s_at | LRP4 | 1271 |
| OCADA.8975_s_at | LRP4 | 1272 |
| ADXUgly12_at | LRRC14 | N/A |
| OC3P.10946.C1_s_at | LRRC14 | 1273 |
| OCHP.1534_x_at | LUM | 1274 |
| OCHP.1534_s_at | LUM | 1275 |
| OCEM.2131_at | MAL | 1276 |
| OCHP.146_s_at | MAL | 1277 |
| OCEM.2131_s_at | MAL | 1278 |
| ADXGoodB51_at | MAL | N/A |
| OCEM.1462_s_at | MAP3K13 | 1279 |
| OC3P.9313.C1_s_at | MAP3K13 | 1280 |
| OCEM.1462_at | MAP3K13 | 1281 |
| OCADNP.11967_s_at | MAP3K13 | 1282 |
| OC3P.12558.C1_s_at | MAP3K13 | 1283 |
| OCADNP.8546_s_at | MAP3K13 | 1284 |
| OC3SNGnh.670_s_at | MAP3K13 | 1285 |
| OCADA.1770_s_at | MAP3K13 | 1286 |
| OCADA.10625_s_at | MAP3K13 | 1287 |
| OCMX.11265.C1_x_at | MBNL3 | 1288 |
| OC3SNGn.7601-3a_s_at | MBNL3 | 1289 |
| OCADNP.12040_s_at | MBNL3 | 1290 |
| OC3P.15006.C1_s_at | MBNL3 | 1291 |
| OCADNP.9948_s_at | MBNL3 | 1292 |
| OCMX.11265.C1_at | MBNL3 | 1293 |
| OCRS.637_s_at | MBNL3 | 1294 |
| OC3P.10771.C1_s_at | METTL7B | 1295 |
| OCADA.11193_s_at | MEX3B | 1296 |
| OC3SNGn.1875-54a_s_at | MEX3B | 1297 |
| OCADNP.936_at | MICA | 1298 |
| OCADNP.936_x_at | MICA | 1299 |
| OC3P.10120.C1_s_at | MICA | 1306 |
| OCRS2.6328_x_at | MICA | 1300 |
| OCEM.1828_at | MICA | 1301 |
| OC3P.10120.C1_x_at | MICA | 1302 |
| OC3SNGnh.18192_x_at | MICA | 1303 |
| OCEM.1828_x_at | MICA | 1304 |
| OC3P.3683.C1_s_at | MICB | 1305 |
| OC3P.10120.C1_s_at | MICB | 1306 |
| OCADA.3772_s_at | MIR142 | 1307 |
| OCADA.3728_s_at | MIR142 | 1308 |
| OC3SNGnh.5895_s_at | MIR143 | 1309 |
| OC3P.12440.C1_s_at | MLLT11 | 1310 |
| OCADNP.5252_s_at | MOBKL2C | 1311 |
| OC3P.8598.C1_x_at | MOBKL2C | 1312 |
| OC3P.11340.C1_s_at | MPDZ | 1313 |
| OCADA.11052_s_at | MPDZ | 1314 |
| OCADNP.9320_s_at | MSI1 | 1315 |
| OCRS.626_at | MSI1 | 1316 |
| OCRS.626_x_at | MSI1 | 1317 |
| OC3SNG.5240-30a_s_at | MT1G | 1318 |
| OC3P.355.C6_x_at | MT1L | 1319 |
| OC3SNG.429-358a_x_at | MT1L | 1320 |
| OC3SNGn.7152-2a_s_at | MT1L | 1321 |
| OCMXSNG.3748_s_at | MTCP1 | 1322 |
| OC3SNG.2207-16a_s_at | MTCP1 | 1323 |
| OCADNP.13496_s_at | MTCP1 | 1324 |
| ADXGood103_at | MTCP1 | N/A |
| OCADA.8530_s_at | MTCP1 | 1325 |
| OC3P.3173.C1_s_at | MX1 | 1326 |
| OC3SNGnh.18345_s_at | MX1 | 1327 |
| OCMXSNG.4976_s_at | MX1 | 1328 |
| OC3SNGn.3343-1542a_s_at | MX1 | 1329 |
| OCMXSNG.5222_s_at | MX1 | 1330 |
| OC3SNGnh.19645_s_at | MX1 | 1331 |
| OC3SNGnh.18497_s_at | MX1 | 1332 |
| ADXStrong8_at | MX1 | N/A |
| OC3SNG.1890-21a_x_at | MYC | 1333 |
| OCRS2.1860_s_at | MYC | 1334 |
| OCADNP.7405_s_at | MYC | 1335 |
| OCADNP.16462_s_at | MYC | 1336 |
| OCHP.226_x_at | MYC | 1337 |
| OC3P.4871.C1_x_at | MYC | 1338 |
| ADXGoodB73_at | MYLIP | N/A |
| OC3P.7441.C2_s_at | MYLIP | 1339 |
| OC3P.2046.C1_x_at | MYLIP | 1340 |
| OC3P.12894.C1_s_at | NCCRP1 | 1341 |
| OC3SNG.4346-38a_s_at | NDUFS3 | 1342 |
| OC3P.5365.C2_s_at | NDUFS3 | 1343 |
| OCADNP.2704_s_at | NKD1 | 1344 |
| OCADA.113_s_at | NKD1 | 1345 |
| OCMX.15105.C1_x_at | NKD1 | 1346 |
| OCMX.15105.C1_at | NKD1 | 1347 |
| OC3P.10474.C1_s_at | NKD1 | 1348 |
| OC3P.10474.C1-853a_s_at | NKD1 | 1349 |
| OCEM.1474_s_at | NMNAT2 | 1350 |
| OC3P.1757.C1_s_at | NMNAT2 | 1351 |
| OCADNP.104_s_at | NMNAT2 | 1352 |
| OCMXSNG.1881_x_at | NMNAT2 | 1353 |
| OC3P.289.C1-454a_s_at | NMNAT2 | 1354 |
| OCMXSNG.1881_at | NMNAT2 | 1355 |
| OC3P.289.C1_at | NMNAT2 | 1356 |
| ADXStrong55_at | NOTCH3 | N/A |
| OCMX.1198.C1_s_at | NOTCH3 | 1357 |
| OCHP.199_s_at | NOTCH3 | 1358 |
| OCADNP.5270_s_at | NOTCH3 | 1359 |
| OC3P.3532.C1_s_at | NOTCH3 | 1360 |
| OCADNP.17585_s_at | NPBWR2 | 1361 |
| OC3SNG.2752-12a_s_at | NPR1 | 1362 |
| OC3P.11825.C1_x_at | NPR1 | 1363 |
| OCRS2.4332_s_at | NRBP2 | 1364 |
| OC3P.5923.C1-395a_s_at | NRBP2 | 1365 |
| OC3SNG.387-9a_s_at | NXF2 | 1366 |
| OC3SNG.387-9a_s_at | NXF2B | 1366 |
| OC3P.1918.C1_at | OAS2 | 1367 |
| OC3P.1918.C1_x_at | OAS2 | 1368 |
| OC3P.9078.C1_s_at | OAS2 | 1369 |
| OC3SNGnh.19480_x_at | OAS2 | 1370 |
| OC3P.14637.C1_s_at | OAS2 | 1371 |
| ADXBad43_at | OAS2 | N/A |
| OC3P.1918.C1-567a_s_at | OAS2 | 1372 |
| OC3SNGnh.13341_x_at | ODZ3 | 1373 |
| OCADA.1894_s_at | ODZ3 | 1374 |
| OCADA.10233_s_at | ODZ3 | 1375 |
| OCADNP.15544_s_at | ODZ3 | 1376 |
| OCRS.2100_at | ODZ3 | 1377 |
| OCRS.2100_x_at | ODZ3 | 1378 |
| OC3P.6938.C1_s_at | OGT | 1379 |
| OC3P.1091.C2_s_at | OGT | 1380 |
| OC3SNGn.4615-28062a_s_at | OGT | 1381 |
| ADXGoodB20_at | OGT | N/A |
| OC3P.1091.C1-398a_s_at | OGT | 1382 |
| ADXGoodB90_at | OGT | N/A |
| OCADA.13060_s_at | OGT | 1383 |
| OC3SNGnh.17759_x_at | OGT | 1384 |
| OC3P.1091.C1_s_at | OGT | 1385 |
| ADXStrong32_at | OGT | N/A |
| ADXGoodB59_at | OGT | N/A |
| OC3P.3843.C1-466a_s_at | OLFML1 | 1386 |
| ADXBad25_at | OLFML1 | N/A |
| OCHPRC.93_s_at | OLFML1 | 1387 |
| OC3P.11342.C1_s_at | OLFML3 | 1388 |
| OC3P.14601.C1_s_at | PARP9 | 1389 |
| OC3SNGnh.18057_at | PARP9 | 1390 |
| OC3SNGnh.17896_x_at | PARP9 | 1391 |
| OC3P.1893.C1_s_at | PARP9 | 1392 |
| OC3SNGn.261-2564a_s_at | PCYOX1 | 1393 |
| OC3P.5613.C1_s_at | PCYOX1 | 1394 |
| OC3SNG.18-15a_x_at | PCYOX1 | 1395 |
| OC3SNGn.8530-2270a_s_at | PCYOX1 | 1396 |
| OCADNP.7249_s_at | PDGFD | 1397 |
| OC3P.9761.C1_s_at | PDGFD | 1398 |
| OC3SNGn.713-1810a_s_at | PDGFD | 1399 |
| OC3SNGnh.16119_at | PDGFD | 1400 |
| OC3SNGnh.10361_x_at | PDGFD | 1401 |
| OC3SNGnh.16119_x_at | PDGFD | 1402 |
| OC3P.5664.C1_s_at | PHACTR3 | 1403 |
| OCADA.2200_x_at | PHACTR3 | 1404 |
| OCADA.2200_s_at | PHACTR3 | 1405 |
| OC3SNGn.2640-38a_s_at | PHC1 | 1406 |
| OCRS2.10640_s_at | PHC1 | 1407 |
| OC3P.8943.C1_s_at | PHC1 | 1408 |
| OCADA.1865_s_at | PKIA | 1409 |
| OCADA.8754_s_at | PKIA | 1410 |
| ADXStrong5_at | PKIA | N/A |
| OCADA.9633_s_at | PKIA | 1411 |
| ADXGoodB7_at | PLCG1 | N/A |
| OC3P.8718.C1_s_at | PLCG1 | 1412 |
| OCADA.5765_s_at | PLCG1 | 1413 |
| OC3P.9725.C1_s_at | PLEKHG2 | 1414 |
| OCADA.4384_s_at | PLEKHG2 | 1415 |
| OC3SNGnh.18488_x_at | PLEKHG2 | 1416 |
| OC3P.9725.C1_at | PLEKHG2 | 1417 |
| OC3SNGnh.18488_at | PLEKHG2 | 1418 |
| OCADA.2995_s_at | PLEKHG2 | 1419 |
| OCMX.11286.C1_s_at | PLXDC1 | 1420 |
| OC3P.13016.C1_s_at | PLXDC1 | 1421 |
| OC3P.11901.C1_s_at | PLXDC1 | 1422 |
| OC3P.3077.C1_s_at | PMEPA1 | 1423 |
| OCHP.1061_s_at | PMEPA1 | 1424 |
| ADXGood72_at | PMP22 | N/A |
| OCADA.9170_s_at | PMP22 | 1425 |
| OC3P.10622.C1_s_at | PMP22 | 1426 |
| OC3SNGnh.8944_s_at | PMP22 | 1427 |
| OCUTR.101_x_at | PPA1 | 1428 |
| OC3P.655.C1_s_at | PPA1 | 1429 |
| OCRS2.12824_x_at | PPP1R16A | 1430 |
| OC3P.59.C1_x_at | PPP1R16A | 1431 |
| OCMXSNG.1294_at | PPP1R16A | 1432 |
| OCMXSNG.1294_x_at | PPP1R16A | 1433 |
| OC3P.1874.C1_s_at | PPP1R3B | 1434 |
| OC3P.12058.C1_s_at | PPP1R3B | 1435 |
| OC3SNGn.3329-2837a_s_at | PPP1R3B | 1436 |
| OC3P.13688.C1_s_at | PRPS2 | 1437 |
| OC3SNGnh.18818_x_at | PRPS2 | 1438 |
| OC3SNG.1788-52a_s_at | PSMA5 | 1439 |
| OC3SNG.6266-52a_x_at | PSMA5 | 1440 |
| OCADA.1277_x_at | PSMA5 | 1441 |
| OCADA.2865_x_at | PSMA5 | 1442 |
| OC3P.5663.C1_s_at | PTGFRN | 1443 |
| OC3P.6990.C1_s_at | PTGFRN | 1444 |
| OCADNP.8703_s_at | PTGIS | 1445 |
| OC3SNGnh.8373_x_at | PTGIS | 1446 |
| OC3SNGnh.8373_at | PTGIS | 1447 |
| OCADNP.9600_s_at | PTGIS | 1448 |
| OC3P.10183.C1_s_at | PTPN7 | 1449 |
| OCADNP.998_x_at | PTRF | 1450 |
| OC3SNG.1416-18a_s_at | PTRF | 1451 |
| OC3P.12255.C1_x_at | PTRF | 1452 |
| OC3SNG.4882-18a_x_at | PTRF | 1453 |
| OC3SNGnh.10165_x_at | PTRF | 1454 |
| OCADNP.8300_s_at | PTRF | 1455 |
| OCHP.964_s_at | PUF60 | 1456 |
| OCHP.1513_s_at | PUF60 | 1457 |
| OCADNP.6711_s_at | PVT1 | 1458 |
| OC3SNGnh.19746_s_at | PVT1 | 1459 |
| OC3P.12914.C1_x_at | PVT1 | 1460 |
| OC3SNGnh.7033_x_at | PVT1 | 1461 |
| OCADA.7024_s_at | PVT1 | 1462 |
| OC3P.12590.C1_s_at | PVT1 | 1463 |
| OC3SNGnh.18875_at | PVT1 | 1464 |
| OC3SNGnh.8972_x_at | PVT1 | 1465 |
| OCADNP.15592_s_at | PVT1 | 1466 |
| OCADA.9299_s_at | PVT1 | 1467 |
| OCADA.2476_s_at | PVT1 | 1468 |
| OC3P.12914.C1_at | PVT1 | 1469 |
| OCADNP.14125_s_at | PVT1 | 1470 |
| OC3SNGnh.18875_x_at | PVT1 | 1471 |
| OC3SNGnh.2328_s_at | PVT1 | 1472 |
| OC3SNGnh.2478_at | PVT1 | 1473 |
| OC3P.8262.C1_s_at | RAB31 | 1474 |
| OC3SNGnh.17870_s_at | RAB31 | 1475 |
| OC3P.11285.C1_s_at | RAB31 | 1476 |
| OCHP.1160_s_at | RAB31 | 1477 |
| OCMX.11222.C1_at | RAB31 | 1478 |
| OCMX.268.C1_s_at | RANBP2 | 1497 |
| OCRS.1769_x_at | RANBP2 | 1479 |
| OC3SNGnh.6542_at | RANBP2 | 1480 |
| OCADA.3091_s_at | RANBP2 | 1481 |
| OCMX.111.C1_s_at | RANBP2 | 1499 |
| OC3P.1162.C1_s_at | RANBP2 | 1482 |
| OCADA.6773_s_at | RANBP2 | 1503 |
| OC3P.11562.C1_s_at | RANBP2 | 1483 |
| OC3P.12656.C1_s_at | RASL11B | 1484 |
| OCRS.1829_at | RBMS3 | 1485 |
| OC3SNGnh.7044_at | RBMS3 | 1486 |
| OCRS.1829_s_at | RBMS3 | 1487 |
| OC3SNGnh.5586_x_at | RBMS3 | 1488 |
| OCADNP.13042_s_at | RBMS3 | 1489 |
| OCADA.2087_s_at | RBMS3 | 1490 |
| OC3SNGnh.7618_at | RBMS3 | 1491 |
| OCMX.1364.C1_x_at | RBMS3 | 1492 |
| OCADA.5823_s_at | RBMS3 | 1493 |
| OC3SNGnh.7224_x_at | RBMS3 | 1494 |
| OC3SNGnh.7224_at | RBMS3 | 1495 |
| OCADA.6168_s_at | RBMS3 | 1496 |
| OCMX.268.C1_s_at | RGPD2 | 1497 |
| OCRS2.11784_s_at | RGPD2 | 1498 |
| OCMX.111.C1_s_at | RGPD2 | 1499 |
| OC3SNGnh.20500_s_at | RGPD2 | 1500 |
| OC3SNGnh.18250_x_at | RGPD2 | 1501 |
| OCRS2.10139_s_at | RGPD2 | 1502 |
| OCADA.6773_s_at | RGPD2 | 1503 |
| OC3P.10240.C1_s_at | RNF113A | 1504 |
| OC3SNG.4959-20a_x_at | RNPC3 | 1505 |
| OC3SNG.885-20a_s_at | RNPC3 | 1506 |
| OCADA.3100_x_at | RNPC3 | 1507 |
| OC3SNG.3327-15a_s_at | ROM1 | 1508 |
| OCRS2.6255_s_at | RPL9P16 | 1509 |
| OC3P.5036.C1_s_at | SALL2 | 1510 |
| OC35NGnh.19445_s_at | SCGB1D2 | 1511 |
| OCHP.701_s_at | SDC1 | 1512 |
| OC3SNGn.2091-716a_s_at | SDC1 | 1513 |
| OC3SNGnh.11631_s_at | SDK1 | 1514 |
| OC3P.15017.C1_x_at | SDK1 | 1515 |
| OC3SNGnh.18247_x_at | SDK1 | 1516 |
| OC3SNGnh.10694_x_at | SDK1 | 1517 |
| OC3SNGnh.2027_at | SDK1 | 1518 |
| OC3SNGnh.11631_at | SDK1 | 1519 |
| OC3SNGnh.13374_x_at | SDK1 | 1520 |
| OC3P.4796.C1_s_at | SDK1 | 1521 |
| OC3SNGnh.12868_at | SDK1 | 1522 |
| OC3SNGnh.15230_s_at | SDK1 | 1523 |
| OCRS2.2187_s_at | SDK1 | 1524 |
| OCRS2.5977_s_at | SDK1 | 1525 |
| OC3SNGnh.14168_x_at | SDK1 | 1526 |
| OC3SNGnh.14168_at | SDK1 | 1527 |
| OC3SNGnh.5808_s_at | SEC23A | 1528 |
| OC3P.2059.C1_s_at | SEC23A | 1529 |
| OCADNP.7566_s_at | SEC23A | 1530 |
| OC3SNGn.2856-15a_s_at | SERPINA1 | 1531 |
| OCADA.3610_s_at | SERPINA1 | 1532 |
| OC3SNGn.5875-4740a_s_at | SERPINE1 | 1533 |
| OC3P.2161.C1_s_at | SERPINE1 | 1534 |
| OCADNP.1839_x_at | SERPINE1 | 1535 |
| OC3SNGn.5874-2592a_s_at | SERPINE1 | 1536 |
| OC3SNGn.5873-1900a_s_at | SERPINE1 | 1537 |
| OCHP.456_s_at | SERPINE1 | 1538 |
| OCMX.148.C44_x_at | SERPINE1 | 1539 |
| OC3SNGn.5872-1154a_x_at | SERPINE1 | 1540 |
| ADXGoodB78_at | SERPINE1 | N/A |
| OC3P.12796.C1_s_at | SERPINE1 | 1541 |
| OC3SNGn.4423-537a_x_at | SERPINE1 | 1542 |
| OCHP.781_s_at | SERPINF1 | 1543 |
| ADXStrong15_at | SERPINF1 | N/A |
| OCEM.1960_at | SERPINF1 | 1544 |
| ADXStrong8_at | SERPINF1 | N/A |
| OC3SNGn.251-21a_s_at | SFRP2 | 1545 |
| OC3P.13621.C1_s_at | SFRP2 | 1546 |
| OC3P.10602.C1_s_at | SFRP4 | 1547 |
| OC3P.10602.C1-303a_s_at | SFRP4 | 1548 |
| OCHP.1367_s_at | SFRP4 | 1549 |
| OCADNP.8054_s_at | SFRP5 | 1550 |
| OC3SNG.617-604a_s_at | SIPA1L2 | 1551 |
| OCADNP.1208_s_at | SIPA1L2 | 1552 |
| ADXGoodB32_at | SIPA1L2 | N/A |
| OCADNP.12385_s_at | SIPA1L2 | 1553 |
| OC3P.2917.C1_s_at | SIPA1L2 | 1554 |
| OC3SNGnh.7545_s_at | SLC40A1 | 1555 |
| OC3SNG.305-10a_s_at | SLC40A1 | 1556 |
| OC3P.10870.C1-466a_s_at | SLC40A1 | 1557 |
| OC3P.10870.C1_s_at | SLC40A1 | 1558 |
| OC3SNGnh.12974_s_at | SLC40A1 | 1559 |
| OCRS.1977_at | SMAD9 | 1560 |
| OCADNP.7805_s_at | SMAD9 | 1561 |
| OCADA.8714_s_at | SMAD9 | 1562 |
| OC3SNGnh.5026_at | SNCAIP | 1563 |
| OC3P.12279.C1_s_at | SNCAIP | 1564 |
| OC3SNGnh.7087_x_at | SNCAIP | 1565 |
| OCHP.747_s_at | SNCG | 1566 |
| OCRS2.1421_x_at | SNORD114-1 | 1567 |
| OCRS2.1421_at | SNORD114-1 | 1568 |
| OCRS2.12766_at | SNORD114-18 | 1571 |
| OCRS2.8346_at | SNORD114-18 | 1569 |
| OCRS2.8346_x_at | SNORD114-18 | 1570 |
| OCRS2.12766_x_at | SNORD114-18 | 1572 |
| OCRS2.12766_at | SNORD114-19 | 1571 |
| OCRS2.12766_x_at | SNORD114-19 | 1572 |
| OCRS2.3148_at | SNORD114-31 | 1573 |
| OCRS2.3148_x_at | SNORD114-31 | 1574 |
| OCRS2.4372_at | SNORD46 | 1575 |
| OCRS2.4372_x_at | SNORD46 | 1576 |
| OC3P.855.C1_x_at | SORL1 | 1577 |
| OC3P.4739.C1-665a_s_at | SORL1 | 1578 |
| OC3SNGnh.3558_x_at | SORL1 | 1579 |
| OC3P.4739.C1_s_at | SORL1 | 1580 |
| OC3P.855.C1-303a_s_at | SORL1 | 1581 |
| OC3SNGnh.3558_at | SORL1 | 1582 |
| OC3P.855.C1_at | SORL1 | 1583 |
| OCRS2.7312_s_at | SORL1 | 1584 |
| OCMX.4125.C1_at | SORL1 | 1585 |
| OCADNP.11708_s_at | SORL1 | 1586 |
| OCADA.2870_s_at | SOX4 | 1587 |
| OCADA.9338_s_at | SOX4 | 1588 |
| OC3SNG.1802-713a_s_at | SOX4 | 1589 |
| OC3P.9406.C1_s_at | SOX4 | 1590 |
| OC3P.10314.C1_s_at | SPDEF | 1591 |
| OC3SNGnh.18260_x_at | SQRDL | 1592 |
| OC3SNGnh.9160_x_at | SQRDL | 1593 |
| OC3P.2220.C1_s_at | SQRDL | 1594 |
| OC3SNGnh.16216_x_at | SRPK1 | 1595 |
| OCHP.676_s_at | SRPK1 | 1596 |
| OC3SNGnh.9486_x_at | SRPK1 | 1597 |
| OC3SNGnh.2729_x_at | SRPX2 | 1598 |
| OC3P.12547.C1_s_at | SRPX2 | 1599 |
| OCADA.5796_s_at | SRPX2 | 1600 |
| OC3SNG.2635-30a_s_at | SRSF12 | 1601 |
| OCADNP.22_s_at | SRSF12 | 1602 |
| OCRS2.6419_s_at | SRSF12 | 1603 |
| OC3P.7155.C1_s_at | SSH3 | 1604 |
| OC3P.13645.C1_s_at | SYPL1 | 1605 |
| OC3P.2792.C1_x_at | SYPL1 | 1606 |
| OCRS2.1456_at | TBC1D26 | 1607 |
| OCRS2.1456_s_at | TBC1D26 | 1608 |
| OC3SNG.5377-16a_s_at | TBC1D26 | 1609 |
| OC3P.7002.C1-421a_s_at | TCF19 | 1610 |
| ADXGood6_at | TCF19 | N/A |
| OCRS2.7197_s_at | TCF19 | 1611 |
| OCHP.901_s_at | TERC | 1612 |
| OC3P.10233.C1_x_at | TGFB3 | 1613 |
| OCADA.11350_at | TGFB3 | 1614 |
| OC3P.10233.C1_s_at | TGFB3 | 1615 |
| OCUTR.173_s_at | THSD4 | 1616 |
| OC3SNGn.8831-5086a_s_at | THSD4 | 1617 |
| OCADA.4455_s_at | THSD4 | 1618 |
| OC3SNGnh.772_at | THSD4 | 1619 |
| OC3SNGnh.15786_x_at | THSD4 | 1620 |
| OC3SNGnh.2176_x_at | THSD4 | 1621 |
| OC3SNGnh.17621_x_at | THSD4 | 1622 |
| OC3SNGnh.12000_x_at | THSD4 | 1623 |
| OC3SNGnh.18146_x_at | THSD4 | 1624 |
| OC3P.15051.C1_x_at | THSD4 | 1625 |
| OC3P.15419.C1_at | THSD4 | 1626 |
| OC3SNGnh.13191_s_at | THSD4 | 1627 |
| OC3SNGnh.18810_x_at | THSD4 | 1628 |
| OC3SNGnh.17600_x_at | THSD4 | 1629 |
| OC3SNGnh.772_x_at | THSD4 | 1630 |
| OC3P.14917.C1_s_at | THSD4 | 1631 |
| OC3SNGnh.2426_x_at | THSD4 | 1632 |
| OC3SNGnh.18810_at | THSD4 | 1633 |
| OC3P.4324.C1_s_at | THSD4 | 1634 |
| OCADA.5329_s_at | THSD4 | 1635 |
| OCUTR.228_x_at | THSD4 | 1636 |
| OCMX.13245.C1_x_at | THSD4 | 1637 |
| OC3P.4993.C1_at | THSD4 | 1638 |
| OC3P.12061.C1_s_at | THSD4 | 1639 |
| OC3SNGnh.17191_s_at | THSD4 | 1640 |
| OCMX.13245.C1_at | THSD4 | 1641 |
| OC3SNGnh.11620_at | THSD4 | 1642 |
| OCMX.14285.C1_x_at | THSD4 | 1643 |
| OC3P.5043.C1_at | THSD4 | 1644 |
| OC3SNGnh.18146_at | THSD4 | 1645 |
| OC3P.4993.C1_s_at | THSD4 | 1646 |
| OC3SNGnh.17441_at | THSD4 | 1647 |
| OC3SNGnh.18103_at | THSD4 | 1648 |
| OC3SNGnh.2426_at | THSD4 | 1649 |
| OC3P.15419.C1_x_at | THSD4 | 1650 |
| OC3SNG.359-662a_s_at | THY1 | 1651 |
| OC3P.2790.C1_s_at | THY1 | 1652 |
| OCHP.607_s_at | THY1 | 1653 |
| OC3P.9682.C1_s_at | TIGD5 | 1654 |
| OCADA.9719_s_at | TLR3 | 1655 |
| OCADA.6345_s_at | TMC5 | 1656 |
| OCADNP.5555_s_at | TMC5 | 1657 |
| OC3P.6033.C1_x_at | TMC5 | 1658 |
| OC3P.1529.C1_s_at | TMC5 | 1659 |
| OC3SNGnh.17082_x_at | TMC5 | 1660 |
| OC3P.3724.C2-437a_s_at | TMEM173 | 1661 |
| OC3P.3724.C2_s_at | TMEM173 | 1662 |
| OC3SNGn.1012-2074a_s_at | TMEM47 | 1663 |
| OC3P.2151.C1_s_at | TMEM47 | 1664 |
| OC3P.13714.C1_s_at | TMEM87B | 1665 |
| OC3SNGnh.4981_at | TMEM87B | 1666 |
| OC3P.2037.C1-520a_s_at | TMEM87B | 1667 |
| OC3SNGnh.4981_x_at | TMEM87B | 1668 |
| OCRS.923_s_at | TMEM87B | 1669 |
| OCADA.6525_s_at | TMEM87B | 1670 |
| OC3P.2037.C1_s_at | TMEM87B | 1671 |
| OC3P.715.C1_x_at | TMEM98 | 1672 |
| OC3P.715.C1_s_at | TMEM98 | 1673 |
| OCMX.14198.C1_x_at | TMEM98 | 1674 |
| OC3P.715.C1_at | TMEM98 | 1675 |
| OCMX.14198.C1_at | TMEM98 | 1676 |
| OC3SNGn.4429-110a_x_at | TMOD4 | 1677 |
| OC3SNGn.395-1a_s_at | TMOD4 | 1678 |
| OC3SNGn.4429-110a_at | TMOD4 | 1679 |
| OC3SNGn.7784-157a_x_at | TMOD4 | 1680 |
| OC3SNGn.1587-1a_s_at | TNNI2 | 1681 |
| OC3SNG.5440-21a_s_at | TNNI2 | 1682 |
| OC3P.10278.C1_x_at | TUBB4 | 1683 |
| OC3P.9430.C1_s_at | UBA7 | 1684 |
| OC3P.1506.C1_s_at | UBD | 1685 |
| OC3P.14896.C1_s_at | UNC5A | 1686 |
| OCADA.3211_s_at | UNC5C | 1687 |
| OCADNP.13201_s_at | UNC5C | 1688 |
| OCADNP.684_s_at | UNC5C | 1689 |
| OC3SNGnh.14349_x_at | UNC5C | 1690 |
| OCMX.12995.C1_at | UNC5C | 1691 |
| OCHP.603_s_at | UNC5C | 1692 |
| OCMX.12995.C1_x_at | UNC5C | 1693 |
| OC3P.1185.C2_x_at | VIM | 1694 |
| OC3SNG.420-22a_x_at | VIM | 1695 |
| OC3SNGn.6624-5a_x_at | VIM | 1696 |
| ADXUglyB15_at | VIPR1 | N/A |
| OC3P.12378.C1_s_at | VIPR1 | 1697 |
| ADXStrongB45_at | VTCN1 | N/A |
| OC3SNGnh.12766_x_at | VTCN1 | 1698 |
| OC3SNGnh.17514_at | VTCN1 | 1699 |
| OCHP.189_s_at | VTCN1 | 1700 |
| OC3SNGnh.18452_x_at | VTCN1 | 1701 |
| OC3SNGnh.17514_x_at | VTCN1 | 1702 |
| OCRS2.2500_s_at | VTCN1 | 1703 |
| OCRS2.7154_s_at | ZBTB42 | 1704 |
| OC3P.10867.C1_s_at | ZBTB42 | 1705 |
| OCADNP.8116_s_at | ZNF711 | 1706 |
| OCRS.1792_s_at | ZNF711 | 1707 |

Accordingly, the method may comprise measuring the expression levels of at least one of MT1L, MT1G, LRP4, RASL11B, IFI27, PKIA, ALOX5AP, UBD, MEX3B, and TMEM98. In specific embodiments the method comprises measuring the expression levels of each of MT1L, MT1G, LRP4, RASL11B, IFI27, PKIA, ALOX5AP, UBD, MEX3B, and TMEM98. In further embodiments the method comprises measuring the expression levels of each of the biomarkers listed in Table I.

The method may comprise measuring the expression levels of at least 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 185 or each of the biomarkers from Table L. In certain embodiments the method may comprise measuring the expression levels of 15-26 biomarkers from Table L. The inventors have shown that measuring the expression levels of at least 15 of the biomarkers in Table L enables the subtype to be reliably detected.

TABLE L

| GeneSymbol | weights | bias |
|---|---|---|
| AARS | −0.65639 | 7.401032 |
| ABCA17P | 0.922294 | 3.334055 |
| ABCA9 | −0.58363 | 4.493582 |
| ADAMTSL2 | −0.38509 | 5.279609 |
| ADRM1 | 1.026765 | 7.596166 |
| AEBP1 | −0.60204 | 7.326171 |
| ANO7 | −0.71308 | 4.327116 |
| APOBEC3F | 1.033609 | 5.893126 |
| APOBEC3G | 0.367923 | 6.401795 |
| ATP5J2P3 | 0.485092 | 4.631863 |
| ATP6V1B1 | 0.453586 | 9.658557 |
| BTLA | 0.584921 | 2.916224 |
| C10orf114 | −0.27237 | 4.821022 |
| C11orf9 | −0.93444 | 5.988653 |
| C1orf130 | 0.618969 | 4.487537 |
| C20orf103 | −0.37885 | 4.647136 |
| C6orf124 | −1.16213 | 5.077056 |
| C7orf27 | −0.67931 | 7.460152 |
| C9orf125 | 0.652801 | 4.915404 |
| CACHD1 | 0.487651 | 5.033627 |
| CALU | −1.02742 | 7.520193 |
| CAMTA1 | −0.89487 | 5.421286 |
| CC2D1B | 0.948738 | 5.305526 |
| CDKN2C | 0.464217 | 4.383252 |
| CHGA | 0.367516 | 4.820369 |
| CHODL | 0.563157 | 3.725809 |
| CLDN6 | −0.22546 | 5.014718 |
| CNOT10 | 0.74618 | 4.275432 |
| COL10A1 | −0.27582 | 6.050837 |
| COL16A1 | −0.72725 | 5.199019 |
| CPD | 0.784465 | 5.488085 |
| CTNNBL1 | −0.8148 | 5.385506 |
| DAAM1 | −0.88057 | 5.746927 |
| DCAF5 | −0.98274 | 5.975384 |
| DDR2 | −1.02071 | 5.653505 |
| DEF8 | 1.11319 | 5.55285 |
| DIS3L | 0.419915 | 5.918805 |
| DLL1 | −0.48834 | 3.669433 |
| DSG2 | −0.72621 | 5.919129 |
| EFNB3 | −0.74559 | 4.919776 |
| EGFLAM | −0.52165 | 4.71277 |
| EID2 | −0.44237 | 5.773564 |
| EIF2AK1 | −0.3068 | 5.658335 |
| EIF4EBP1 | −0.71459 | 7.109601 |
| ENDOU | 0.293396 | 5.348262 |
| ERAP2 | 0.584489 | 5.095089 |
| FAAH2 | 0.173995 | 4.956479 |
| FAM117B | 1.128773 | 4.655475 |
| FAM131B | −0.52377 | 6.175618 |
| FAM134A | 0.849807 | 6.590952 |
| FAM198B | −0.4639 | 2.707193 |
| FAM19A5 | −0.25257 | 3.907392 |
| FAM201A | 0.250566 | 3.910334 |
| FAM86A | 0.634045 | 6.60684 |
| FAT2 | 0.319655 | 8.524751 |
| FAT4 | −0.23655 | 2.889227 |
| FHL2 | 0.537704 | 4.2723 |
| FIGN | −0.34634 | 4.423745 |
| FJX1 | 1.051816 | 6.664334 |
| FRMD8 | 0.532185 | 9.590093 |
| GABRE | 0.239505 | 5.30313 |
| GALNT1 | −0.45313 | 6.018831 |
| GBAP1 | 0.911469 | 4.886108 |
| GBP1 | 0.312193 | 5.58982 |
| GLRX | −0.49583 | 2.318808 |
| GNAI1 | −0.55211 | 6.922587 |
| GNG11 | −0.56131 | 5.885839 |
| GOLGA2B | 0.523479 | 4.127833 |
| GOLGA7 | −0.89626 | 7.894601 |
| GPR124 | −0.61873 | 4.990225 |
| GPR87 | −0.55846 | 2.384806 |
| HCG27 | 0.681432 | 5.777026 |
| HDHD1 | 0.852639 | 5.762428 |
| HECTD3 | 1.031804 | 7.320371 |
| HGSNAT | −0.95292 | 7.324317 |
| HLA-DMB | 0.342698 | 7.74009 |
| HLA-DPA1 | 0.424987 | 6.141466 |
| HOXB3 | 0.769857 | 5.110344 |
| HRASLS | 0.593993 | 5.07244 |
| HSD17B14 | −0.72006 | 7.38998 |
| HSPBP1 | −1.26293 | 7.536136 |
| HTRA1 | −0.53755 | 8.855317 |
| IGFBP7 | −0.63907 | 5.603764 |
| IPO8 | −1.10956 | 7.762268 |
| ITGA11 | −0.54575 | 4.085074 |
| IVNS1ABP | −1.29404 | 7.327752 |
| KCND2 | 0.152994 | 6.978517 |
| KDM5A | −0.77944 | 6.279645 |
| KHDRBS3 | 0.744668 | 3.720225 |
| KIAA1324 | 0.423355 | 4.457234 |
| KIF26A | −0.49085 | 5.151089 |
| LATS2 | −0.84391 | 4.366105 |
| LILRB1 | 0.547184 | 6.286473 |
| LONRF3 | −0.69342 | 3.550519 |
| LRRC47 | 1.147953 | 7.164294 |
| LYRM7 | 1.507855 | 6.993756 |
| MALL | −0.67656 | 6.270219 |
| MAPK1IP1L | −0.76504 | 4.371223 |
| 09-Mar | 0.790383 | 4.372016 |
| MAT2B | 0.508078 | 9.368301 |
| MDH1B | 0.707623 | 4.723468 |
| MED29 | −0.59144 | 7.58716 |
| MIR1245 | −0.21849 | 4.967581 |
| MIR1825 | 0.735714 | 8.113055 |
| MMP13 | −0.2623 | 3.383705 |
| MRVI1 | −0.46315 | 4.85013 |
| MS4A8B | −0.75325 | 2.629675 |
| MT1L | 0.449177 | 9.204179 |
| MTM1 | 0.661607 | 5.61342 |
| MYLIP | 0.478751 | 6.623007 |
| MZT1 | −0.3857 | 6.393013 |
| NCCRP1 | −0.26899 | 5.426857 |
| NDUFAF4 | 0.701993 | 5.308435 |
| NEU1 | −0.77738 | 6.786668 |
| NKD1 | −0.53162 | 4.063017 |
| NMNAT2 | 0.698227 | 4.65029 |
| NOX4 | −0.26589 | 4.562509 |
| NTN4 | 0.338464 | 3.756298 |
| OGFOD2 | 0.919712 | 6.370094 |
| OXNAD1 | −1.1043 | 4.910198 |
| PARP9 | 0.627251 | 5.871653 |
| PCOLCE | −0.74142 | 6.433086 |
| PKHD1L1 | 0.279131 | 3.674874 |
| POLH | 1.022503 | 5.778668 |
| PPA1 | 0.606982 | 9.406626 |
| PPP1R14A | −1.10798 | 5.575699 |
| PPP1R3B | −0.40058 | 3.625393 |
| PPTC7 | −0.92157 | 4.074024 |
| PQLC3 | 0.602949 | 8.622679 |
| PROSC | −1.08894 | 4.917455 |
| PRPS2 | 0.612148 | 7.107149 |
| PRR5L | 0.516817 | 5.137202 |
| PRRT1 | −0.85902 | 4.475276 |
| PTPN7 | 0.23212 | 7.59385 |
| RAB25 | 0.422749 | 8.078456 |
| RANBP3 | 1.272601 | 5.744696 |
| RASAL3 | 0.497973 | 7.040146 |
| RASSF2 | −0.58881 | 3.897682 |
| RIOK3 | −1.16178 | 7.767987 |
| RORA | 0.871987 | 5.720607 |
| SCEL | −0.31467 | 2.339399 |
| SCN3B | 0.498042 | 5.406948 |
| SERPINA5 | −0.37896 | 4.633783 |
| SIPA1L2 | −0.53172 | 5.340869 |
| SLC25A20 | −0.53431 | 3.506854 |
| SLC25A45 | 0.673913 | 7.136345 |
| SLC26A10 | −0.93989 | 5.545123 |
| SLC35A1 | 0.707593 | 7.117949 |
| SLC44A4 | 0.411808 | 6.293524 |
| SNORD119 | 0.586489 | 5.795974 |
| SP100 | 0.667182 | 5.892435 |
| SP140L | 0.640598 | 5.472334 |
| SPG20 | −0.43428 | 4.996652 |
| SRPK1 | 0.622519 | 4.483086 |
| ST6GAL1 | 0.313862 | 4.541053 |
| SYN1 | 1.579045 | 5.950278 |
| SYT13 | 0.47853 | 4.679104 |
| SYTL4 | −0.61486 | 3.764143 |
| TATDN2 | 1.033457 | 7.150942 |
| TBC1D26 | −0.64087 | 4.356035 |
| TBX3 | −0.65687 | 4.556336 |
| TCF4 | −0.47897 | 4.755404 |
| THY1 | −0.35956 | 7.810588 |
| TLR3 | 0.59882 | 3.262462 |
| TMEM169 | −0.36994 | 4.129678 |
| TMEM173 | 0.464915 | 7.596418 |
| TMEM200A | −0.1415 | 3.309473 |
| TMEM200B | −0.43728 | 5.149805 |
| TMEM222 | 0.735448 | 6.376735 |
| TMEM30B | −0.73339 | 4.590222 |
| TMEM55B | −0.87579 | 6.509398 |
| TMEM56 | 0.670554 | 3.237535 |
| TMEM62 | 0.58294 | 5.776118 |
| TMEM87B | 0.918136 | 4.210936 |
| TMOD4 | 0.80627 | 4.917728 |
| TNKS2 | −0.61379 | 5.36376 |
| TNNI2 | −0.41583 | 6.646823 |
| TRRAP | −0.54824 | 5.276388 |
| TSPAN8 | −0.76554 | 5.705074 |
| TWIST1 | −0.18936 | 7.048776 |
| TXK | 0.806144 | 3.558338 |
| UPK2 | −0.33133 | 2.785719 |
| UST | −0.42458 | 6.774158 |
| WBSCR17 | −0.61591 | 4.189211 |
| ZNF426 | 0.643991 | 3.717797 |
| ZNF532 | −0.65961 | 4.723125 |
| ZNF720 | −0.88277 | 5.366577 |
| ZNF818P | 0.484876 | 4.027402 |

The biomarkers from Table L are ranked in Table M from most important to least important based upon hazard ratio reduction when the genes are included versus when they are excluded. The genes/biomarkers may be selected for inclusion in a panel of biomarkers/a signature based on their ranking. Table N illustrates probesets that can be used to detect expression of the biomarkers.

TABLE M

| GENE | DELTA HR | RANK |
|---|---|---|
| MT1L | 0.908866717 | 1 |
| GABRE | 0.667217276 | 2 |
| KCND2 | 0.591077431 | 3 |
| UPK2 | 0.55842258 | 4 |
| HLA-DPA1 | 0.534607997 | 5 |
| SYTL4 | 0.505566469 | 6 |
| SCEL | 0.30431854 | 7 |
| MZT1 | 0.250806306 | 8 |
| EFNB3 | 0.237987091 | 9 |
| DLL1 | 0.233789098 | 10 |
| TLR3 | 0.205307637 | 11 |
| TMEM173 | 0.194369459 | 12 |
| TMEM87B | 0.193175461 | 13 |
| SCN3B | 0.192271191 | 14 |
| PRRT1 | 0.179933038 | 15 |
| GBP1 | 0.179466776 | 16 |
| TMEM200B | 0.17777205 | 17 |
| SLC25A45 | 0.161031544 | 18 |
| HLA-DMB | 0.160341067 | 19 |
| RASAL3 | 0.157414323 | 20 |
| APOBEC3G | 0.149623496 | 21 |
| MAPK1IP1L | 0.144838522 | 22 |
| TMEM30B | 0.1347231 | 23 |
| SLC25A20 | 0.134309271 | 24 |
| LILRB1 | 0.12938888 | 25 |
| ABCA9 | 0.128671 | 26 |
| C1orf130 | 0.125179667 | 27 |
| MAT2B | 0.118737998 | 28 |
| BTLA | 0.108872863 | 29 |
| FAT2 | 0.10593471 | 30 |
| SP140L | 0.105840398 | 31 |
| POLC3 | 0.105644375 | 32 |
| GNAI1 | 0.105622924 | 33 |
| ERAP2 | 0.102461512 | 34 |
| ABCA17P | 0.098727035 | 35 |
| KHDRBS3 | 0.097222352 | 36 |
| ENDOU | 0.094403985 | 37 |
| EIF4EBP1 | 0.092989305 | 38 |
| PRR5L | 0.092468206 | 39 |
| IVNS1ABP | 0.092283009 | 40 |
| C10orf114 | 0.085519515 | 41 |
| ATP6V1B1 | 0.083089486 | 42 |
| GBAP1 | 0.080820611 | 43 |
| PTPN7 | 0.079381537 | 44 |
| PARP9 | 0.076485924 | 45 |
| CLDN6 | 0.076372844 | 46 |
| LONRF3 | 0.075339299 | 47 |
| ATP5J2P3 | 0.074918776 | 48 |
| ADRM1 | 0.072902153 | 49 |
| MIR1825 | 0.071481869 | 50 |
| FRMD8 | 0.071050122 | 51 |
| SLC26A10 | 0.070430629 | 52 |
| TSPAN8 | 0.069471845 | 53 |
| PROSC | 0.068444648 | 54 |
| SLC44A4 | 0.064557733 | 55 |
| RAB25 | 0.06242119 | 56 |
| RIOK3 | 0.059023943 | 57 |
| PPP1R3B | 0.058984119 | 58 |
| SYT13 | 0.049666341 | 59 |
| SP100 | 0.048903812 | 60 |
| MS4A8B | 0.047361692 | 61 |
| HGSNAT | 0.04711386 | 62 |
| DSG2 | 0.04608177 | 63 |
| SNORD119 | 0.045892653 | 64 |
| C9orf125 | 0.045268656 | 65 |
| EIF2AK1 | 0.043910334 | 66 |
| ZNF720 | 0.039607146 | 67 |
| MTM1 | 0.039550106 | 68 |
| HSPBP1 | 0.038969628 | 69 |
| TBX3 | 0.038421349 | 70 |
| HCG27 | 0.037923398 | 71 |
| DEF8 | 0.037872255 | 72 |
| OGFOD2 | 0.037771874 | 73 |
| ANO7 | 0.036694304 | 74 |
| HECTD3 | 0.03521687 | 75 |
| DCAF5 | 0.03519632 | 76 |
| TRRAP | 0.035103978 | 77 |
| FAM117B | 0.034274233 | 78 |
| RORA | 0.033127429 | 79 |
| MYLIP | 0.031501136 | 80 |
| APOBEC3F | 0.029945075 | 81 |
| IPO8 | 0.029292849 | 82 |
| C7orf27 | 0.027840666 | 83 |
| GALNT1 | 0.027742171 | 84 |
| TMEM55B | 0.026757321 | 85 |
| SYN1 | 0.026561904 | 86 |
| GOLGA7 | 0.026164524 | 87 |
| OXNAD1 | 0.025075483 | 88 |
| FAT4 | 0.024030579 | 89 |
| LYRM7 | 0.022365957 | 90 |
| NKD1 | 0.02217 | 91 |
| IGFBP7 | 0.022093298 | 92 |
| FJX1 | 0.021930692 | 93 |
| FAM134A | 0.020052167 | 94 |
| CAMTA1 | 0.019759097 | 95 |
| FAM198B | 0.018378557 | 96 |
| TNKS2 | 0.017848434 | 97 |
| RANBP3 | 0.017015191 | 98 |
| TMEM222 | 0.016515538 | 99 |
| CTNNBL1 | 0.015872357 | 100 |
| C6orf124 | 0.014534662 | 101 |
| KDM5A | 0.013576727 | 102 |
| ZNF532 | 0.012421816 | 103 |
| AARS | 0.012306547 | 104 |
| MARCH9 | 0.011614808 | 105 |
| CALU | 0.010527118 | 106 |
| NMNAT2 | 0.006468214 | 107 |
| FAM131B | 0.006429583 | 108 |
| TATDN2 | 0.005833596 | 109 |
| CC2D1B | 0.00450517 | 110 |
| PPP1R14A | 0.003255542 | 111 |
| PPTC7 | 0.002737645 | 112 |
| EID2 | 0.002372556 | 113 |
| SERPINA5 | −0.000503962 | 114 |
| CPD | −0.003015939 | 115 |
| GPR87 | −0.005891465 | 116 |
| HOXB3 | −0.006448662 | 117 |
| SIPA1L2 | −0.009142482 | 118 |
| FAM19A5 | −0.016750461 | 119 |
| ZNF426 | −0.017744701 | 120 |
| TMOD4 | −0.021005842 | 121 |
| DAAM1 | −0.028613335 | 122 |
| TBC1D26 | −0.028805165 | 123 |
| POLH | −0.029750395 | 124 |
| C20orf103 | −0.033242781 | 125 |
| WBSCR17 | −0.037692836 | 126 |
| NDUFAF4 | −0.040356361 | 127 |
| CNOT10 | −0.041114163 | 128 |
| MDH1B | −0.043254001 | 129 |
| LRRC47 | −0.043956122 | 130 |
| MED29 | −0.045907542 | 131 |
| ST6GAL1 | −0.046074486 | 132 |
| NEU1 | −0.052972048 | 133 |
| GPR124 | −0.052992737 | 134 |
| PPA1 | −0.0591455 | 135 |
| FHL2 | −0.06017306 | 136 |
| TNNI2 | −0.063216964 | 137 |
| GNG11 | −0.063915596 | 138 |
| TXK | −0.066621406 | 139 |
| FAM86A | −0.066886683 | 140 |
| SLC35A1 | −0.06777196 | 141 |
| UST | −0.074326855 | 142 |
| CHODL | −0.076775005 | 143 |
| PRPS2 | −0.079107843 | 144 |
| C11orf9 | −0.090905443 | 145 |
| SPG20 | −0.094902921 | 146 |
| LATS2 | −0.096137531 | 147 |
| KIAA1324 | −0.097600443 | 148 |
| PKHD1L1 | −0.097977563 | 149 |
| ADAMTSL2 | −0.104445295 | 150 |
| ZNF818P | −0.106667387 | 151 |
| TMEM62 | −0.113695553 | 152 |
| NTN4 | −0.11394366 | 153 |
| CDKN2C | −0.115202927 | 154 |
| FIGN | −0.118426675 | 155 |
| DDR2 | −0.122492204 | 156 |
| MALL | −0.124483421 | 157 |
| TCF4 | −0.13040915 | 158 |
| FAM201A | −0.148492922 | 159 |
| CACHD1 | −0.158203051 | 160 |
| PCOLCE | −0.163832567 | 161 |
| EGFLAM | −0.173262928 | 162 |
| SRPK1 | −0.176833669 | 163 |
| TMEM169 | −0.177073006 | 164 |
| GOLGA2B | −0.179753363 | 165 |
| DIS3L | −0.185618926 | 166 |
| HTRA1 | −0.187842746 | 167 |
| HRASLS | −0.196261694 | 168 |
| NCCRP1 | −0.20711311 | 169 |
| HDHD1 | −0.213988023 | 170 |
| GLRX | −0.222216581 | 171 |
| COL16A1 | −0.229012506 | 172 |
| ITGA11 | −0.235998942 | 173 |
| RASSF2 | −0.238807477 | 174 |
| AEBP1 | −0.24863769 | 175 |
| NOX4 | −0.252796981 | 176 |
| TMEM56 | −0.255940603 | 177 |
| KIF26A | −0.268124669 | 178 |
| HSD17B14 | −0.278110087 | 179 |
| MRVI1 | −0.295208886 | 180 |
| TWIST1 | −0.302130162 | 181 |
| THY1 | −0.3135314 | 182 |
| FAAH2 | −0.344580603 | 183 |
| TMEM200A | −0.385470923 | 184 |
| CHGA | −0.479861362 | 185 |
| COL10A1 | −0.654186132 | 186 |
| MIR1245 | −0.741380447 | 187 |
| MMP13 | −0.896991441 | 188 |

TABLE N

| Probeset | Gene | SEQ ID No. |
|---|---|---|
| OC3P.1619.C1_s_at | AARS | 1708 |
| OC3P.1619.C1_at | AARS | 1709 |
| OC3P.1619.C1_x_at | AARS | 1710 |
| OCADA.3819_s_at | ABCA17P | 1711 |
| OCRS2.4361_s_at | ABCA17P | 1712 |
| OCRS2.11473_s_at | ABCA17P | 1713 |
| OCADNP.4777_s_at | ABCA9 | 1714 |
| OC3P.9255.C1_s_at | ABCA9 | 1715 |
| OC3SNGn.2213-221a_x_at | ABCA9 | 1716 |
| OCADNP.12230_s_at | ABCA9 | 1717 |
| OC3SNGnh.2310_x_at | ABCA9 | 1718 |
| OCADNP.5182_s_at | ABCA9 | 1719 |
| OC3P.10512.C1_s_at | ADAMTSL2 | 1720 |
| OCRS2.7089_s_at | ADAMTSL2 | 1721 |
| OC3P.3283.C2_at | ADRM1 | 1722 |
| OC3SNG.5165-18a_s_at | ADRM1 | 1723 |
| OC3SNGn.2266-7a_s_at | ADRM1 | 1724 |
| OCMXSNG.5475_at | AEBP1 | 1725 |
| OCMXSNG.2603_at | AEBP1 | 1726 |
| ADXStrongB47_at | AEBP1 | N/A |
| OCHP.1649_s_at | AEBP1 | 1727 |
| OC3P.3458.C1_s_at | AEBP1 | 1728 |
| ADXStrongB42_at | AEBP1 | N/A |
| OCMXSNG.5474_at | AEBP1 | 1729 |
| OCMXSNG.5474_x_at | AEBP1 | 1730 |
| OC3P.6301.C1_s_at | ANO7 | 1731 |
| OC3P.6301.C1_at | ANO7 | 1732 |
| OCRS2.2777_s_at | ANO7 | 1733 |
| OCUTR.200_s_at | APOBEC3F | 1734 |
| OCADNP.5415_x_at | APOBEC3F | 1735 |
| OC3SNGn.8424-313a_x_at | APOBEC3F | 1736 |
| OC3P.8406.C1_x_at | APOBEC3F | 1737 |
| OCADA.5213_s_at | APOBEC3F | 1738 |
| OC3P.8406.C1_s_at | APOBEC3F | 1739 |
| OC3SNG.5308-20a_s_at | APOBEC3G | 1740 |
| OCADNP.16260_s_at | APOBEC3G | 1741 |
| OCRS2.820_s_at | ATP5J2P3 | 1742 |
| OC3SNG.5860-81a_s_at | ATP6V1B1 | 1743 |
| OCHP.1217_x_at | ATP6V1B1 | 1744 |
| OC3SNGnh.11044_s_at | BTLA | 1745 |
| OCRS.1136_s_at | BTLA | 1746 |
| OC3SNGn.174-1a_s_at | C10orf114 | 1747 |
| OC3P.860.C1_s_at | C11orf9 | 1748 |
| OCADNP.4793_s_at | C11orf9 | 1749 |
| OC3SNG.1287-14a_s_at | C1orf130 | 1750 |
| OC3P.7546.C1_s_at | C20orf103 | 1751 |
| OCRS2.8279_s_at | C6orf124 | 1752 |
| OCRS2.4080_s_at | C6orf124 | 1753 |
| OC3P.9696.C1_s_at | C7orf27 | 1754 |
| OC3P.5130.C1_at | C9orf125 | 1755 |
| OC3P.15373.C1_s_at | C9orf125 | 1756 |
| OC3P.5130.C1-322a_s_at | C9orf125 | 1757 |
| OCADA.6915_s_at | CACHD1 | 1758 |
| OC3SNGnh.6598_at | CACHD1 | 1759 |
| OC3SNGnh.5252_at | CACHD1 | 1760 |
| OC3SNGnh.5308_x_at | CACHD1 | 1761 |
| OC3P.5821.C1_s_at | CACHD1 | 1762 |
| OC3SNGnh.6598_x_at | CACHD1 | 1763 |
| OC3SNGnh.5252_s_at | CACHD1 | 1764 |
| OC3SNGnh.5955_at | CACHD1 | 1765 |
| OC3SNGnh.4213_x_at | CACHD1 | 1766 |
| ADXGood25_at | CALU | N/A |
| OC3SNGnh.9873_s_at | CALU | 1767 |
| OC3SNG.123-901a_s_at | CALU | 1768 |
| OCADNP.14456_x_at | CALU | 1769 |
| OC3P.2001.C2-449a_s_at | CALU | 1770 |
| OCADNP.7231_s_at | CALU | 1771 |
| OC3SNGnh.11073_x_at | CALU | 1772 |
| OC3P.13898.C1_s_at | CALU | 1773 |
| OCHP.1141_s_at | CALU | 1774 |
| OCADNP.3994_s_at | CALU | 1775 |
| OC3SNG.1183-1605a_s_at | CAMTA1 | 1776 |
| OC3SNGnh.10266_at | CAMTA1 | 1777 |
| OC3SNG.1182-16a_s_at | CAMTA1 | 1778 |
| OC3SNGnh.16971_at | CAMTA1 | 1779 |
| OCADA.12240_s_at | CAMTA1 | 1780 |
| OC3SNGnh.12316_x_at | CAMTA1 | 1781 |
| OC3P.13685.C1_s_at | CAMTA1 | 1782 |
| OC3P.9592.C1_s_at | CAMTA1 | 1783 |
| OC3SNGnh.10266_x_at | CAMTA1 | 1784 |
| OCADA.467_s_at | CAMTA1 | 1785 |
| OCADNP.13448_s_at | CAMTA1 | 1786 |
| OCRS.1072_s_at | CC2D1B | 1787 |
| OC3P.8147.C1_s_at | CC2D1B | 1788 |
| OCADNP.6491_s_at | CC2D1B | 1789 |
| OCADA.5455_s_at | CC2D1B | 1790 |
| OCADNP.9668_s_at | CDKN2C | 1791 |
| OC3P.12264.C1_x_at | CDKN2C | 1792 |
| OC3SNGn.3112-55a_s_at | CHGA | 1793 |
| ADXBad17_at | CHGA | N/A |
| OC3P.13249.C1_x_at | CHODL | 1794 |
| OCMX.7042.C1_s_at | CHODL | 1795 |
| OCMX.15594.C1_s_at | CHODL | 1796 |
| OCMXSNG.1530_s_at | CHODL | 1797 |
| OC3SNG.3556-78a_s_at | CHODL | 1798 |
| OCMX.7042.C1_x_at | CHODL | 1799 |
| OC3SNGn.4742-71060a_s_at | CHODL | 1800 |
| OC3SNG.549-201852a_s_at | CHODL | 1801 |
| OC3SNGn.4741-34831a_s_at | CHODL | 1802 |
| OCEM.1035_s_at | CHODL | 1803 |
| OCHPRC.81_x_at | CLDN6 | 1804 |
| OCRS2.7326_x_at | CLDN6 | 1805 |
| OC3SNG.2953-20a_x_at | CLDN6 | 1806 |
| OCADNP.9501_s_at | CLDN6 | 1807 |
| OC3P.9796.C1_x_at | CNOT10 | 1808 |
| OC3P.9796.C1_at | CNOT10 | 1809 |
| OCADNP.7022_s_at | CNOT10 | 1810 |
| OCRS.383_s_at | COL10A1 | 1811 |
| OC3SNG.1834-947a_s_at | COL10A1 | 1812 |
| OC3P.3047.C1_x_at | COL16A1 | 1813 |
| OC3P.3047.C1-304a_s_at | COL16A1 | 1814 |
| OC3SNGnh.6481_s_at | COL16A1 | 1815 |
| OCADNP.7339_s_at | CPD | 1816 |
| OC3SNGnh.14957_x_at | CPD | 1817 |
| OC3P.6221.C1_x_at | CPD | 1818 |
| OC3P.6221.C1_at | CPD | 1819 |
| OC3P.13725.C1_s_at | CPD | 1820 |
| OC3SNGn.373-984a_s_at | CPD | 1821 |
| OC3SNG.1724-28a_s_at | CPD | 1822 |
| OC3SNGnh.18477_x_at | CTNNBL1 | 1823 |
| OCHP.1190_s_at | CTNNBL1 | 1824 |
| OCADNP.2336_s_at | DAAM1 | 1825 |
| OCADNP.4315_s_at | DAAM1 | 1826 |
| OC3P.15553.C1_s_at | DAAM1 | 1827 |
| OC3SNGn.2635-651a_s_at | DAAM1 | 1828 |
| OC3SNGnh.12060_s_at | DAAM1 | 1829 |
| OCADA.7103_s_at | DAAM1 | 1830 |
| OC3SNG.5293-38a_s_at | DCAF5 | 1831 |
| OCADA.3135_s_at | DCAF5 | 1832 |
| OC3P.12587.C1_s_at | DCAF5 | 1833 |
| OC3P.9318.C1_s_at | DCAF5 | 1834 |
| ADXUgly11_at | DDR2 | N/A |
| OC3SNG.1306-60a_s_at | DDR2 | 1835 |
| OC3P.10616.C1_s_at | DEF8 | 1836 |
| OC3P.14941.C1_s_at | DEF8 | 1837 |
| OC3P.7775.C1_s_at | DIS3L | 1838 |
| OC3SNGn.1174-202a_x_at | DIS3L | 1839 |
| OC3P.8771.C1_s_at | DLL1 | 1840 |
| OCADNP.14063_s_at | DSG2 | 1841 |
| OC3P.2533.C1_s_at | DSG2 | 1842 |
| OC3P.2533.C1_x_at | DSG2 | 1843 |
| OC3P.13694.C1_s_at | DSG2 | 1844 |
| OCADNP.8516_s_at | EFNB3 | 1845 |
| OC3P.9384.C1_s_at | EFNB3 | 1846 |
| OCRS.1751_s_at | EGFLAM | 1847 |
| OC3P.13255.C1_s_at | EGFLAM | 1848 |
| OC3P.9989.C1_s_at | EID2 | 1849 |
| OCMXSNG.5461_s_at | EIF2AK1 | 1850 |
| OC3SNGnh.14331_x_at | EIF2AK1 | 1851 |
| OC3P.301.C1_s_at | EIF2AK1 | 1852 |
| OC3P.2826.C1_s_at | EIF2AK1 | 1853 |
| OC3P.2826.C1-632a_s_at | EIF2AK1 | 1854 |
| OC3P.12951.C1_s_at | EIF4EBP1 | 1855 |
| OCADNP.9346_s_at | ENDOU | 1856 |
| OCADA.3164_x_at | ERAP2 | 1857 |
| OC3P.7237.C1_x_at | ERAP2 | 1858 |
| OC3SNGnh.2998_s_at | ERAP2 | 1859 |
| OCADNP.14937_s_at | ERAP2 | 1860 |
| OCADA.6354_s_at | ERAP2 | 1861 |
| OC3SNGnh.18545_at | FAAH2 | 1862 |
| OC3SNGnh.18545_x_at | FAAH2 | 1863 |
| OCMXSNG.4800_x_at | FAAH2 | 1864 |
| OC3SNGnh.14393_x_at | FAAH2 | 1865 |
| OC3SNGnh.13606_x_at | FAAH2 | 1866 |
| OC3SNGnh.14393_at | FAAH2 | 1867 |
| OC3SNG.6004-30a_s_at | FAAH2 | 1868 |
| OCADNP.15681_s_at | FAM117B | 1869 |
| OC3SNGn.6969-10a_s_at | FAM117B | 1870 |
| OC3SNGn.1670-24a_s_at | FAM117B | 1871 |
| OC3SNGnh.15718_x_at | FAM117B | 1872 |
| OCMX.2476.C1_s_at | FAM117B | 1873 |
| OC3SNG.3088-16a_s_at | FAM131B | 1874 |
| ADXGood101_at | FAM134A | N/A |
| OC3SNG.1366-70a_s_at | FAM134A | 1875 |
| OC3SNGnh.7940_s_at | FAM134A | 1876 |
| OCADA.10797_s_at | FAM134A | 1877 |
| OC3SNGnh.5052_s_at | FAM134A | 1878 |
| OC3SNGn.7559-1580a_at | FAM198B | 1879 |
| OC3P.6417.C1_s_at | FAM198B | 1880 |
| OCRS2.4931_s_at | FAM198B | 1881 |
| OCADA.10843_s_at | FAM198B | 1882 |
| OCADA.5341_s_at | FAM19A5 | 1883 |
| OC3P.13915.C1_s_at | FAM19A5 | 1884 |
| OC3P.14112.C1_s_at | FAM19A5 | 1885 |
| OCADNP.960_s_at | FAM201A | 1886 |
| OCADA.814_s_at | FAM201A | 1887 |
| OC3SNGnh.2090_x_at | FAM86A | 1888 |
| OC3P.2572.C4_s_at | FAM86A | 1889 |
| OCRS2.951_x_at | FAM86A | 1890 |
| OC3P.11005.C1_s_at | FAT2 | 1891 |
| OC3SNG.4266-25a_s_at | FAT4 | 1892 |
| OCHP.668_s_at | FHL2 | 1893 |
| OC3P.12166.C1_at | FHL2 | 1894 |
| OC3P.12762.C1_at | FHL2 | 1895 |
| OC3P.13087.C1 x_at | FHL2 | 1896 |
| OC3SNGnh.7102_at | FHL2 | 1897 |
| OC3P.6364.C1 x_at | FHL2 | 1898 |
| OC3P.13087.C1_at | FHL2 | 1899 |
| OC3SNGnh.9422_at | FHL2 | 1900 |
| OC3SNGnh.5485_s_at | FHL2 | 1901 |
| OC3SNGnh.5485_x_at | FHL2 | 1902 |
| OCADA.6796_s_at | FIGN | 1903 |
| OC3P.15318.C1_at | FIGN | 1904 |
| OCADA.6194_s_at | FIGN | 1905 |
| OCADA.2860_s_at | FIGN | 1906 |
| OCADNP.12019_s_at | FIGN | 1907 |
| OC3P.15266.C1_x_at | FIGN | 1908 |
| OCRS2.5152_s_at | FJX1 | 1909 |
| OC3P.6045.C1_s_at | FJX1 | 1910 |
| OC3P.553.C1_s_at | FRMD8 | 1911 |
| OC3P.6165.C1_s_at | GABRE | 1912 |
| OC3SNGn.6359-34a_s_at | GABRE | 1913 |
| OC3SNGn.6583-10627a_at | GABRE | 1914 |
| OC3SNGn.6583-10627a_x_at | GABRE | 1915 |
| OCMX.833.C13_s_at | GABRE | 1916 |
| OC3P.13199.C1_s_at | GALNT1 | 1917 |
| OC3SNGnh.8607_x_at | GALNT1 | 1918 |
| OC3P.6817.C1_s_at | GALNT1 | 1919 |
| OCADNP.10124_s_at | GALNT1 | 1920 |
| OCADNP.12320_s_at | GALNT1 | 1921 |
| OCADA.4308_s_at | GALNT1 | 1922 |
| OC3SNG.1687-462a_s_at | GALNT1 | 1923 |
| OC3P.3730.C1-349a_s_at | GBAP1 | 1924 |
| OCADNP.16743_s_at | GBAP1 | 1925 |
| OCHP.1292_s_at | GBAP1 | 1926 |
| OCADNP.1974_s_at | GBP1 | 1927 |
| OCADNP.2962_s_at | GBP1 | 1928 |
| OCHP.1438_x_at | GBP1 | 1929 |
| OCRS2.4406_x_at | GBP1 | 1930 |
| OCADA.10565_s_at | GBP1 | 1931 |
| OC3P.1927.C1_x_at | GBP1 | 1932 |
| OCMX.605.C1_at | GLRX | 1933 |
| OCHP.1436_s_at | GLRX | 1934 |
| OCMX.605.C1_x_at | GLRX | 1935 |
| OC3SNGnh.7530_at | GLRX | 1936 |
| OCMX.606.C1_s_at | GLRX | 1937 |
| OC3SNGnh.7530_x_at | GLRX | 1938 |
| OCADNP.8335_s_at | GLRX | 1939 |
| OCMX.606.C1_at | GLRX | 1940 |
| OCRS2.6438_s_at | GNAI1 | 1941 |
| OC3P.1142.C1_s_at | GNAI1 | 1942 |
| ADXGood98_at | GNAI1 | N/A |
| OC3P.12320.C1_s_at | GNG11 | 1943 |
| OC3P.9220.C1_s_at | GOLGA2B | 1944 |
| OCRS2.11208_s_at | GOLGA7 | 1945 |
| OCRS2.8554_s_at | GPR124 | 1946 |
| OC3P.7680.C1-589a_s_at | GPR124 | 1947 |
| OC3P.7680.C1_at | GPR124 | 1948 |
| OCADA.10290_s_at | GPR87 | 1949 |
| OCRS2.11321_s_at | HCG27 | 1950 |
| OCADA.4167_s_at | HDHD1 | 1951 |
| OC3SNGnh.18826_at | HDHD1 | 1952 |
| OC3P.7901.C1_s_at | HDHD1 | 1953 |
| OC3P.10741.C1_s_at | HECTD3 | 1954 |
| OC3P.12375.C1_s_at | HGSNAT | 1955 |
| OC3SNG.1222-16a_x_at | HGSNAT | 1956 |
| OC3SNG.914-13a_s_at | HGSNAT | 1957 |
| OC3SNGnh.10720_s_at | HGSNAT | 1958 |
| OC3P.7601.C1_s_at | HGSNAT | 1959 |
| OC3P.4729.C1_s_at | HLA-DMB | 1960 |
| OCMX.15188.C1_s_at | HLA-DMB | 1961 |
| OC3P.2028.C1_s_at | HLA-DPA1 | 1962 |
| ADXUglyB19_at | HLA-DPA1 | N/A |
| OC3SNGn.2735-12a_s_at | HLA-DPA1 | 1963 |
| OCADNP.5108_s_at | HOXB3 | 1964 |
| OCEM.730_x_at | HOXB3 | 1965 |
| OCADNP.8237_s_at | HOXB3 | 1966 |
| OCEM.730_at | HOXB3 | 1967 |
| OCADA.7670_s_at | HOXB3 | 1968 |
| OC3P.10261.C1_s_at | HOXB3 | 1969 |
| OC3P.2857.C1_s_at | HOXB3 | 1970 |
| OC3SNG.3101-14a_s_at | HRASLS | 1971 |
| OC3SNG.5718-34a_s_at | HRASLS | 1972 |
| OCADA.10152_s_at | HRASLS | 1973 |
| OC3SNG.4039-40a_s_at | HSD17B14 | 1974 |
| OC3SNG.813-28a_s_at | HSD17B14 | 1975 |
| OC3P.9612.C1_s_at | HSPBP1 | 1976 |
| OC3P.9612.C1_x_at | HSPBP1 | 1977 |
| OCHP.902_s_at | HTRA1 | 1978 |
| OCADNP.3740_s_at | IGFBP7 | 1979 |
| OCMX.11971.C1_s_at | IGFBP7 | 1980 |
| OC3SNGn.4133-3670a_x_at | IGFBP7 | 1981 |
| OC3SNGnh.5634_s_at | IGFBP7 | 1982 |
| OC3SNGn.5009-5456a_x_at | IGFBP7 | 1983 |
| ADXGoodB24_at | IGFBP7 | N/A |
| OCADNP.3131_x_at | IGFBP7 | 1984 |
| OC3SNG.1653-16a_s_at | IGFBP7 | 1985 |
| OCADNP.4032_s_at | IGFBP7 | 1986 |
| OC3P.8137.C1_s_at | IPO8 | 1987 |
| OCADNP.7714_s_at | IPO8 | 1988 |
| OC3SNGnh.19520_s_at | ITGA11 | 1989 |
| OCADNP.587_s_at | ITGA11 | 1990 |
| OCMX.7412.C2_at | IVNS1ABP | 1991 |
| OC3P.8210.C1-530a_s_at | IVNS1ABP | 1992 |
| OC3P.9366.C1_at | IVNS1ABP | 1993 |
| OC3P.8210.C1_s_at | IVNS1ABP | 1994 |
| OC3SNGn.2064-1384a_s_at | IVNS1ABP | 1995 |
| OCADNP.13995_s_at | IVNS1ABP | 1996 |
| OCADNP.12825_s_at | IVNS1ABP | 1997 |
| OC3P.1136.C1_s_at | IVNS1ABP | 1998 |
| OC3P.15477.C1_s_at | IVNS1ABP | 1999 |
| OCADNP.7979_s_at | KCND2 | 2000 |
| OCEM.617_s_at | KCND2 | 2001 |
| OCADA.9429_s_at | KDM5A | 2002 |
| OC3SNGnh.17035_at | KDM5A | 2003 |
| OCMX.12398.C1_x_at | KDM5A | 2004 |
| OC3P.6882.C1_s_at | KDM5A | 2005 |
| OC3SNGnh.17668_x_at | KDM5A | 2006 |
| OCHP.1380_s_at | KDM5A | 2007 |
| OC3P.12897.C1_s_at | KDM5A | 2008 |
| OCADNP.2795_s_at | KDM5A | 2009 |
| OC3SNGnh.17035_x_at | KDM5A | 2010 |
| OCADA.4719_s_at | KDM5A | 2011 |
| OC3SNG.5949-16a_s_at | KHDRBS3 | 2012 |
| OC3P.14132.C1_s_at | KHDRBS3 | 2013 |
| OC3SNGnh.13220_s_at | KHDRBS3 | 2014 |
| OCMX.4202.C1_at | KHDRBS3 | 2015 |
| OCMX.4202.C1_x_at | KHDRBS3 | 2016 |
| OC3SNGnh.12409_x_at | KIAA1324 | 2017 |
| ADXBad44_at | KIAA1324 | N/A |
| OC3SNG.4404-2900a_x_at | KIAA1324 | 2018 |
| ADXStrongB45_at | KIAA1324 | N/A |
| OCADNP.5286_s_at | KIAA1324 | 2019 |
| OCMX.11681.C1_at | KIAA1324 | 2020 |
| OCMX.11681.C1_x_at | KIAA1324 | 2021 |
| OC3SNGnh.4924_x_at | KIAA1324 | 2022 |
| OC3SNG.3368-36a_s_at | KIAA1324 | 2023 |
| ADXBad2_at | KIAA1324 | N/A |
| OC3SNG.35-2898a_x_at | KIAA1324 | 2024 |
| OC3P.10299.C1_s_at | KIAA1324 | 2025 |
| OC3P.13885.C1_s_at | KIF26A | 2026 |
| OCADNP.7032_s_at | LATS2 | 2027 |
| OCADA.9355_s_at | LATS2 | 2028 |
| OC3P.13211.C1_s_at | LATS2 | 2029 |
| OCADA.7506_s_at | LATS2 | 2030 |
| OCADA.3519_s_at | LILRB1 | 2031 |
| OCHP.1361_x_at | LILRB1 | 2032 |
| ADXBad33_at | LILRB1 | N/A |
| ADXBad17_at | LILRB1 | N/A |
| OCADA.10299_s_at | LONRF3 | 2033 |
| OC3P.11154.C1_s_at | LONRF3 | 2034 |
| OC3P.7629.C1_s_at | LRRC47 | 2035 |
| OC3SNGn.300-11a_s_at | LYRM7 | 2036 |
| OC3SNG.5278-785a_x_at | LYRM7 | 2037 |
| ADXGood103_at | LYRM7 | N/A |
| OC3SNGnh.8177_x_at | LYRM7 | 2038 |
| OC3SNG.2044-750a_s_at | LYRM7 | 2039 |
| OC3P.13673.C1-400a_s_at | MALL | 2040 |
| OC3P.13673.C1_x_at | MALL | 2041 |
| OC3P.13673.C1_at | MALL | 2042 |
| OCRS.1341_at | MAPK1IP1L | 2043 |
| OC3P.4445.C1_s_at | MAPK1IP1L | 2044 |
| OC3SNGnh.17002_x_at | MAPK1IP1L | 2045 |
| OC3SNGn.2080-4885a_s_at | MAPK1IP1L | 2046 |
| OCADA.2389_at | MAPK1IP1L | 2047 |
| OC3P.4841.C1_s_at | MAPK1IP1L | 2048 |
| OC3SNGnh.17002_at | MAPK1IP1L | 2049 |
| OCRS.1341_x_at | MAPK1IP1L | 2050 |
| OC3SNGnh.1561_s_at | MAPK1IP1L | 2051 |
| OC3P.12193.C1_x_at | MARCH9 | 2052 |
| OCADA.3534_s_at | MARCH9 | 2053 |
| OC3SNGnh.2686_x_at | MARCH9 | 2054 |
| OC3P.12193.C1-488a_s_at | MARCH9 | 2055 |
| OC3P.12193.C1_at | MARCH9 | 2056 |
| OC3P.5073.C1_s_at | MAT2B | 2057 |
| OC3P.5073.C1_x_at | MAT2B | 2058 |
| ADXUgly23 at | MDH1B | N/A |
| OCADA.5923_s_at | MDH1B | 2059 |
| OCADNP.1018_s_at | MDH1B | 2060 |
| OC3SNG.704-39a_x_at | MED29 | 2061 |
| OCEM.259_at | MED29 | 2062 |
| OC3P.3851.C1_x_at | MED29 | 2063 |
| OC3SNGnh.3422_s_at | MIR1245 | 2064 |
| OC3P.3938.C1_x_at | MIR1825 | 2065 |
| OCADA.4427_s_at | MIR1825 | 2066 |
| OCHP.983_s_at | MMP13 | 2067 |
| OCADA.3580_s_at | MRVI1 | 2068 |
| OC3P.1058.C1_s_at | MRVI1 | 2069 |
| OC3P.13126.C1_s_at | MRVI1 | 2070 |
| OCADNP.10237_s_at | MRVI1 | 2071 |
| OC3P.1608.C1_s_at | MS4A8B | 2072 |
| OC3P.355.C6_x_at | MT1L | 2073 |
| OC3SNG.429-358a_x_at | MT1L | 2074 |
| OC3SNGn.7152-2a_s_at | MT1L | 2075 |
| OCEM.2176_at | MTM1 | 2076 |
| OC3P.7705.C1_s_at | MTM1 | 2077 |
| OCADA.7806_x_at | MTM1 | 2078 |
| ADXGoodB73_at | MYLIP | N/A |
| OC3P.7441.C2_s_at | MYLIP | 2079 |
| OC3P.2046.C1_x_at | MYLIP | 2080 |
| OCADA.2961_s_at | MZT1 | 2081 |
| OC3SNGnh.18633_x_at | MZT1 | 2082 |
| OC3P.12894.C1_s_at | NCCRP1 | 2083 |
| OC3SNGnh.4878_at | NDUFAF4 | 2084 |
| OC3SNGnh.4878_x_at | NDUFAF4 | 2085 |
| OC3P.14796.C1_x_at | NDUFAF4 | 2086 |
| OC3SNGnh.18072_x_at | NDUFAF4 | 2087 |
| ADXStrongB6_at | NEU1 | N/A |
| OC3P.831.C1_x_at | NEU1 | 2088 |
| OCHP.1043_s_at | NEU1 | 2089 |
| OCADNP.2704_s_at | NKD1 | 2090 |
| OCADA.113_s_at | NKD1 | 2091 |
| OCMX.15105.C1_x_at | NKD1 | 2092 |
| OCMX.15105.C1_at | NKD1 | 2093 |
| OC3P.10474.C1_s_at | NKD1 | 2094 |
| OC3P.10474.C1-853a_s_at | NKD1 | 2095 |
| OCEM.1474_s_at | NMNAT2 | 2096 |
| OC3P.1757.C1_s_at | NMNAT2 | 2097 |
| OCADNP.104_s_at | NMNAT2 | 2098 |
| OCMXSNG.1881_x_at | NMNAT2 | 2099 |
| OC3P.289.C1-454a_s_at | NMNAT2 | 2100 |
| OCMXSNG.1881_at | NMNAT2 | 2101 |
| OC3P.289.C1_at | NMNAT2 | 2102 |
| OCRS.320_s_at | NOX4 | 2103 |
| OCADNP.14954_s_at | NOX4 | 2104 |
| OC3SNGnh.13560_at | NTN4 | 2105 |
| OC3SNGnh.6387_at | NTN4 | 2106 |
| OCADA.7765_s_at | NTN4 | 2107 |
| OC3SNGnh.16553_x_at | NTN4 | 2108 |
| OC3SNGnh.16553_at | NTN4 | 2109 |
| OC3SNGnh.6387_x_at | NTN4 | 2110 |
| OC3SNGnh.19123_x_at | NTN4 | 2111 |
| OC3P.6445.C1_s_at | NTN4 | 2112 |
| OC3P.8596.C1_s_at | OGFOD2 | 2113 |
| OC3P.14537.C1_s_at | OGFOD2 | 2114 |
| OC3SNG.846-19a_s_at | OXNAD1 | 2115 |
| OC3SNGnh.17867_s_at | OXNAD1 | 2116 |
| OCADNP.2469_s_at | OXNAD1 | 2117 |
| OC3P.14601.C1_s_at | PARP9 | 2118 |
| OC3SNGnh.18057_at | PARP9 | 2119 |
| OC3SNGnh.17896_x_at | PARP9 | 2120 |
| OC3P.1893.C1_s_at | PARP9 | 2121 |
| OCRS2.3088_s_at | PCOLCE | 2122 |
| OC3P.5048.C1_s_at | PCOLCE | 2123 |
| OCMXSNG.2345_s_at | PCOLCE | 2124 |
| OC3P.5246.C1_s_at | PKHD1L1 | 2125 |
| OCRS2.2200_s_at | PKHD1L1 | 2126 |
| OC3SNGnh.1242_x_at | PKHD1L1 | 2127 |
| OCHP.105_s_at | PKHD1L1 | 2128 |
| OCADNP.15163_s_at | PKHD1L1 | 2129 |
| OCADNP.10209_s_at | POLH | 2130 |
| OCADA.4349_s_at | POLH | 2131 |
| OCADNP.8799_x_at | POLH | 2132 |
| OC3SNGn.4978-918a_s_at | POLH | 2133 |
| OCEM.1235_x_at | POLH | 2134 |
| OCUTR.101_x_at | PPA1 | 2135 |
| OC3P.655.C1_s_at | PPA1 | 2136 |
| ADXUgly36_at | PPP1R14A | N/A |
| OCHPRC.13_s_at | PPP1R14A | 2137 |
| OC3P.1874.C1_s_at | PPP1R3B | 2138 |
| OC3P.12058.C1_s_at | PPP1R3B | 2139 |
| OC3SNGn.3329-2837a_s_at | PPP1R3B | 2140 |
| OCADNP.11516_s_at | PPTC7 | 2141 |
| OCADNP.6056_s_at | PPTC7 | 2142 |
| OCRS.827_s_at | PPTC7 | 2143 |
| OC3SNG.5357-16a_s_at | PQLC3 | 2144 |
| OCADA.5737_s_at | PQLC3 | 2145 |
| OCADNP.3913_s_at | PROSC | 2146 |
| OC3SNGnh.3612_x_at | PROSC | 2147 |
| OC3P.10833.C1_x_at | PROSC | 2148 |
| OC3P.4515.C1_s_at | PROSC | 2149 |
| OC3SNGnh.3612_at | PROSC | 2150 |
| OC3P.7265.C1_x_at | PROSC | 2151 |
| ADXGood74_at | PROSC | N/A |
| OC3P.13688.C1_s_at | PRPS2 | 2152 |
| OC3SNGnh.18818_x_at | PRPS2 | 2153 |
| OC3P.15485.C1_s_at | PRR5L | 2154 |
| OC3SNG.1870-16a_at | PRR5L | 2155 |
| OC3SNG.1870-16a_x_at | PRR5L | 2156 |
| OCADNP.14409_s_at | PRR5L | 2157 |
| OCADA.10221_s_at | PRR5L | 2158 |
| OC3SNG.1753-12635a_s_at | PRRT1 | 2159 |
| OC3P.13346.C1_s_at | PRRT1 | 2160 |
| ADXStrongB43_at | PRRT1 | N/A |
| OCADNP.3007_s_at | PRRT1 | 2161 |
| OC3P.10183.C1_s_at | PTPN7 | 2162 |
| OC3SNGn.7993-61a_s_at | RAB25 | 2163 |
| OC3P.9633.C1_s_at | RANBP3 | 2164 |
| OCMXSNG.2939_at | RANBP3 | 2165 |
| OCADA.9981_s_at | RANBP3 | 2166 |
| ADXUglyB26_at | RANBP3 | N/A |
| OCADA.9572_s_at | RANBP3 | 2167 |
| OCADA.13086_s_at | RANBP3 | 2168 |
| OCADA.3307_s_at | RASAL3 | 2169 |
| OC3P.7431.C1_s_at | RASSF2 | 2170 |
| OC3SNGnh.16076_x_at | RIOK3 | 2171 |
| OC3P.11216.C1_s_at | RIOK3 | 2172 |
| OC3SNGnh.11220_x_at | RIOK3 | 2173 |
| OC3SNGnh.7191_x_at | RIOK3 | 2174 |
| OCADNP.4969_s_at | RIOK3 | 2175 |
| OCADNP.11029_s_at | RORA | 2176 |
| OCADNP.14736_s_at | RORA | 2177 |
| OC3SNGnh.15902_at | RORA | 2178 |
| OC3SNGnh.5170_x_at | RORA | 2179 |
| OC3SNGnh.5170_at | RORA | 2180 |
| OCADA.4803_s_at | RORA | 2181 |
| OC3SNGn.5422-69a_s_at | RORA | 2182 |
| OC3SNGnh.7784_s_at | RORA | 2183 |
| OCEM.154_x_at | RORA | 2184 |
| OC3SNGnh.8046_x_at | RORA | 2185 |
| OC3SNGnh.14507_x_at | RORA | 2186 |
| ADXStrong3_at | RORA | N/A |
| OCADNP.10800_s_at | RORA | 2187 |
| OCADNP.12239_s_at | RORA | 2188 |
| ADXStrongB80_at | RORA | N/A |
| OC3SNGnh.15902_x_at | RORA | 2189 |
| OCADA.5291_s_at | RORA | 2190 |
| OC3SNG.1661-145a_s_at | RORA | 2191 |
| ADXStrong13_at | RORA | N/A |
| ADXStrong9_at | RORA | N/A |
| OC3SNGnh.14507_at | RORA | 2192 |
| OC3P.14007.C1_s_at | RORA | 2193 |
| OC3P.14007.C1_x_at | RORA | 2194 |
| OC3SNGnh.5392_at | RORA | 2195 |
| OCADNP.13199_s_at | RORA | 2196 |
| ADXStrong7_at | RORA | N/A |
| OC3SNGnh.13160_s_at | RORA | 2197 |
| OC3P.7464.C1_x_at | RORA | 2198 |
| ADXStrongB91_at | RORA | N/A |
| ADXStrongB78_at | RORA | N/A |
| OC3P.7464.C1_at | RORA | 2199 |
| OC3SNGnh.12483_s_at | RORA | 2200 |
| OC3SNGnh.5392_x_at | RORA | 2201 |
| OC3P.13801.C1_s_at | SCEL | 2202 |
| OC3P.13801.C1-478a_s_at | SCEL | 2203 |
| OCADA.9767_s_at | SCEL | 2204 |
| OCADNP.605_s_at | SCEL | 2205 |
| OC3P.8365.C1_s_at | SCN3B | 2206 |
| OCHP.963_s_at | SERPINA5 | 2207 |
| OC3SNG.617-604a_s_at | SIPA1L2 | 2208 |
| OCADNP.1208_s_at | SIPA1L2 | 2209 |
| ADXGoodB32_at | SIPA1L2 | N/A |
| OCADNP.12385_s_at | SIPA1L2 | 2210 |
| OC3P.2917.C1_s_at | SIPA1L2 | 2211 |
| OC3SNGnh.19852_s_at | SLC25A20 | 2212 |
| OCADNP.7055_at | SLC25A45 | 2213 |
| OCADA.8596_s_at | SLC26A10 | 2214 |
| OCRS2.621_at | SLC26A10 | 2215 |
| OCRS2.621_s_at | SLC26A10 | 2216 |
| OCRS2.621_x_at | SLC26A10 | 2217 |
| OC3P.1533.C1_at | SLC35A1 | 2218 |
| OCADNP.652_s_at | SLC44A4 | 2219 |
| OCHP.204_x_t | SLC44A4 | 2220 |
| OCADNP.9262_s_at | SLC44A4 | 2221 |
| OC3P.11858.C1_x_at | SLC44A4 | 2222 |
| OCRS2.7902_at | SNORD119 | 2223 |
| OC3SNGn.172-18a_s_at | SP100 | 2224 |
| OC3P.14515.C1_s_at | SP100 | 2225 |
| OC3SNGn.6055-155a_s_at | SP100 | 2226 |
| OC3SNGnh.14536_x_at | SP100 | 2227 |
| OCADA.5491_s_at | SP100 | 2228 |
| OC3SNGn.7002-818a_x_at | SP100 | 2229 |
| OCADA.10095_s_at | SP100 | 2230 |
| OC3P.8666.C1_s_at | SP140L | 2231 |
| OCADA.2122_at | SP140L | 2232 |
| OCADA.2122_s_at | SP140L | 2233 |
| OCADA.2122_x_at | SP140L | 2234 |
| OCADNP.5031_s_at | SPG20 | 2235 |
| OC3SNGn.3066-1400a_s_at | SPG20 | 2236 |
| OC3P.5330.C1_s_at | SPG20 | 2237 |
| OCEM.1114_s_at | SPG20 | 2238 |
| OCADA.5138_s_at | SPG20 | 2239 |
| OC3SNGnh.16216_x_at | SRPK1 | 2240 |
| OCHP.676_s_at | SRPK1 | 2241 |
| OC3SNGnh.9486_x_at | SRPK1 | 2242 |
| OC3SNGnh.1744_at | ST6GAL1 | 2243 |
| OC3SNGnh.155_x_at | ST6GAL1 | 2244 |
| OCADNP.4027_s_at | ST6GAL1 | 2245 |
| OC3P.167.C1_s_at | ST6GAL1 | 2246 |
| OC3SNGnh.155_at | ST6GAL1 | 2247 |
| OCADNP.277_s_at | SYN1 | 2248 |
| OC3SNGn.6047-5a_s_at | SYN1 | 2249 |
| OCMX.3057.C3_at | SYN1 | 2250 |
| OC3P.7484.C1_s_at | SYT13 | 2251 |
| OCADNP.2470_s_at | SYTL4 | 2252 |
| OC3SNGnh.16147_x_at | SYTL4 | 2253 |
| OCADA.1925_x_at | SYTL4 | 2254 |
| OC3P.12165.C1_s_at | SYTL4 | 2255 |
| OCADA.2118_s_at | TATDN2 | 2256 |
| ADXStrong16_at | TATDN2 | N/A |
| OC3SNGn.769-1666a_s_at | TATDN2 | 2257 |
| OCHP.1166_s_at | TATDN2 | 2258 |
| OCRS2.1456_at | TBC1D26 | 2259 |
| OCRS2.1456_s_at | TBC1D26 | 2260 |
| OC3SNG.5377-16a_s_at | TBC1D26 | 2261 |
| OCADA.3459_s_at | TBX3 | 2262 |
| OCADNP.14673_s_at | TBX3 | 2263 |
| OC3P.6538.C1_s_at | TBX3 | 2264 |
| OCADNP.8834_s_at | TBX3 | 2265 |
| OCHP.649_s_at | TBX3 | 2266 |
| OCADA.4438_s_at | TCF4 | 2267 |
| OC3P.4112.C1_s_at | TCF4 | 2268 |
| OCHP.1876_s_at | TCF4 | 2269 |
| OCADA.7185_s_at | TCF4 | 2270 |
| OC3SNGnh.10608_s_at | TCF4 | 2271 |
| OC3SNGnh.4569_x_at | TCF4 | 2272 |
| OCADA.8009_s_at | TCF4 | 2273 |
| OCADNP.14530_s_at | TCF4 | 2274 |
| OC3SNG.2691-3954a_s_at | TCF4 | 2275 |
| OC3SNGnh.10608_x_at | TCF4 | 2276 |
| OC3P.3507.C1_s_at | TCF4 | 2277 |
| OC3SNG.359-662a_s_at | THY1 | 2278 |
| OC3P.2790.C1_s_at | THY1 | 2279 |
| OCHP.607_s_at | THY1 | 2280 |
| OCADA.9719_s_at | TLR3 | 2281 |
| OCADNP.2642_s_at | TMEM169 | 2282 |
| OC3P.3724.C2-437a_s_at | TMEM173 | 2283 |
| OC3P.3724.C2_s_at | TMEM173 | 2284 |
| OC3P.6478.C1_s_at | TMEM200A | 2285 |
| OC3P.6478.C1-363a_s_at | TMEM200A | 2286 |
| OCRS2.11454_s_at | TMEM200B | 2287 |
| OCADA.3157_s_at | TMEM200B | 2288 |
| OC3SNGnh.913_s_at | TMEM222 | 2289 |
| ADXGood11_at | TMEM222 | N/A |
| OC3P.2550.C1_s_at | TMEM222 | 2290 |
| OC3P.14967.C1_x_at | TMEM222 | 2291 |
| OC3P.4586.C1_s_at | TMEM30B | 2292 |
| OCADNP.15931_s_at | TMEM30B | 2293 |
| OCRS.1335_s_at | TMEM30B | 2294 |
| OC3P.6263.C1_s_at | TMEM55B | 2295 |
| OC3SNGnh.7925_s_at | TMEM56 | 2296 |
| OCRS2.9192_s_at | TMEM56 | 2297 |
| OCADNP.12494_s_at | TMEM56 | 2298 |
| OC3P.12427.C1_s_at | TMEM62 | 2299 |
| OC3P.13714.C1_s_at | TMEM87B | 2300 |
| OC3SNGnh.4981_at | TMEM87B | 2301 |
| OC3P.2037.C1-520a_s_at | TMEM87B | 2302 |
| OC3SNGnh.4981_x_at | TMEM87B | 2303 |
| OCRS.923_s_at | TMEM87B | 2304 |
| OCADA.6525_s_at | TMEM87B | 2305 |
| OC3P.2037.C1_s_at | TMEM87B | 2306 |
| OC3SNGn.4429-110a_x_at | TMOD4 | 2307 |
| OC3SNGn.395-1a_s_at | TMOD4 | 2308 |
| OC3SNGn.4429-110a_at | TMOD4 | 2309 |
| OC3SNGn.7784-157a_x_at | TMOD4 | 2310 |
| OC3SNGn.682-1836a_s_at | TNKS2 | 2311 |
| OC3P.5143.C1_s_at | TNKS2 | 2312 |
| OCADA.8373_s_at | TNKS2 | 2313 |
| OC3SNGn.1587-1a_s_at | TNNI2 | 2314 |
| OC3SNG.5440-21a_s_at | TNNI2 | 2315 |
| OC3SNGnh.12737_x_at | TRRAP | 2316 |
| OC3SNGnh.334_s_at | TRRAP | 2317 |
| OC3SNGnh.12737_at | TRRAP | 2318 |
| OCADNP.4013_s_at | TRRAP | 2319 |
| OC3SNGnh.334_at | TRRAP | 2320 |
| OCHP.1454_s_at | TRRAP | 2321 |
| OC3SNG.6204-21a_s_at | TSPAN8 | 2322 |
| OCHPRC.1350_at | TSPAN8 | 2323 |
| OC3SNGn.2801-166a_s_at | TWIST1 | 2324 |
| OCRS2.11542_s_at | TWIST1 | 2325 |
| OC3SNGnh.13363_s_at | TXK | 2326 |
| OC3SNGnh.17188_at | TXK | 2327 |
| OC3SNGnh.17188_x_at | TXK | 2328 |
| OCEM.1963_at | TXK | 2329 |
| OCADNP.7909_s_at | TXK | 2330 |
| OC3P.72.C6_x_at | TXK | 2331 |
| OC3SNGnh.9832_x_at | TXK | 2332 |
| OCADA.11004_s_at | UPK2 | 2333 |
| OC3SNGnh.91_s_at | UST | 2334 |
| OC3SNGn.350-2795a_s_at | UST | 2335 |
| ADXStrongB3_at | UST | N/A |
| OC3SNGnh.6725_x_at | UST | 2336 |
| OC3P.12648.C1_s_at | UST | 2337 |
| OC3SNGnh.17987_at | WBSCR17 | 2338 |
| OC3P.9629.C1_at | WBSCR17 | 2339 |
| OC3P.9629.C1_x_at | WBSCR17 | 2340 |
| OC3SNGnh.17288_x_at | WBSCR17 | 2341 |
| OC3SNGnh.14607_x_at | WBSCR17 | 2342 |
| OC3SNGnh.16415_x_at | WBSCR17 | 2343 |
| OCADA.2335_s_at | WBSCR17 | 2344 |
| OCADNP.4201_s_at | WBSCR17 | 2345 |
| OC3SNG.441-49a_s_at | WBSCR17 | 2346 |
| OCADA.7193_s_at | WBSCR17 | 2347 |
| OCADA.12324_s_at | WBSCR17 | 2348 |
| OC3SNGnh.14607_at | WBSCR17 | 2349 |
| OCADA.1886_s_at | ZNF426 | 2350 |
| OCADA.10995_x_at | ZNF426 | 2351 |
| OC3SNGnh.10916_x_at | ZNF426 | 2352 |
| OC3SNGnh.16594_x_at | ZNF532 | 2353 |
| OC3SNGnh.16594_at | ZNF532 | 2354 |
| OC3SNGn.321-1659a_s_at | ZNF532 | 2355 |
| OC3SNGn.5828-8a_x_at | ZNF532 | 2356 |
| OC3SNGnh.13417_x_at | ZNF532 | 2357 |
| OC3P.6619.C1_s_at | ZNF532 | 2358 |
| OC3P.12402.C1_s_at | ZNF532 | 2359 |
| OC3SNGnh.2646_x_at | ZNF720 | 2360 |
| OC3SNGnh.17078_s_at | ZNF720 | 2361 |
| OCADA.6654_s_at | ZNF720 | 2362 |
| OC3SNGn.8203-1695a_s_at | ZNF720 | 2363 |
| OC3SNGn.8204-2035a_s_at | ZNF720 | 2364 |
| OC3SNGnh.14440_s_at | ZNF818P | 2365 |

The method may comprise measuring the expression levels of at least one of MTL1, GABRE, KCND2, UPK2, HLA-DPA1, SYTL4, SCEL, MZT1, EFNB3, and DLL1. In specific embodiments the method comprises measuring the expression levels of each of MTL1, GABRE, KCND2, UPK2, HLA-DPA1, SYTL4, SCEL, MZT1, EFNB3, and DLL1. In further embodiments the method comprises measuring the expression levels of each of the biomarkers listed in Table L.

Methods for determining the expression levels of the biomarkers are described in greater detail herein. Typically, the methods may involve contacting a sample obtained from a subject with a detection agent, such as primers/probes/antibodies (as discussed in detail herein) specific for the biomarker and detecting expression products.

According to all aspects of the invention the expression level of the gene or genes may be measured by any suitable method. Genes may also be referred to, interchangeably, as biomarkers. In certain embodiments the expression level is determined at the level of protein, RNA or epigenetic modification. The epigenetic modification may be DNA methylation.

The expression level may be determined by immunohistochemistry. By Immunohistochemistry is meant the detection of proteins in cells of a tissue sample by using a binding reagent such as an antibody or aptamer that binds specifically to the proteins.

Accordingly, in a further aspect, the present invention relates to an antibody or aptamer that binds specifically to a protein product of at least one of the biomarkers listed herein.

The antibody may be of monoclonal or polyclonal origin. Fragments and derivative antibodies may also be utilised, to include without limitation Fab fragments, ScFv, single domain antibodies, nanoantibodies, heavy chain antibodies, aptamers etc. which retain peptide-specific binding function and these are included in the definition of “antibody”. Such antibodies are useful in the methods of the invention. They may be used to measure the level of a particular protein, or in some instances one or more specific isoforms of a protein. The skilled person is well able to identify epitopes that permit specific isoforms to be discriminated from one another.

Methods for generating specific antibodies are known to those skilled in the art. Antibodies may be of human or non-human origin (e.g. rodent, such as rat or mouse) and be humanized etc. according to known techniques (Jones et al., Nature (1986) May 29-Jun. 4; 321(6069):522-5; Roguska et al., Protein Engineering, 1996, 9(10):895-904; and Studnicka et al., Humanizing Mouse Antibody Frameworks While Preserving 3-D Structure. Protein Engineering, 1994, Vol. 7, pg 805).

In certain embodiments the expression level is determined using an antibody or aptamer conjugated to a label. By label is meant a component that permits detection, directly or indirectly. For example, the label may be an enzyme, optionally a peroxidase, or a fluorophore.

Where the antibody is conjugated to an enzyme a chemical composition may be used such that the enzyme catalyses a chemical reaction to produce a detectable product. The products of reactions catalyzed by appropriate enzymes can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers. In certain embodiments a secondary antibody is used and the expression level is then determined using an unlabeled primary antibody that binds to the target protein and a secondary antibody conjugated to a label, wherein the secondary antibody binds to the primary antibody.

Additional techniques for determining expression level at the level of protein include, for example, Westem blot, immunoprecipitation, immunocytochemistry, mass spectrometry, ELISA and others (see ImmunoAssay: A Practical Guide, edited by Brian Law, published by Taylor & Francis, Ltd., 2005 edition). To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies.

Measuring mRNA in a biological sample may be used as a surrogate for detection of the level of the corresponding protein in the biological sample. Thus, the expression level of any of the genes described herein can also be detected by detecting the appropriate RNA.

Accordingly, in specific embodiments the expression level is determined by microarray, northern blotting, or nucleic acid amplification. Nucleic acid amplification includes PCR and all variants thereof such as real-time and end point methods and qPCR. Typically, PCR includes of a series of 20-40 repeated temperature changes (cycles) with each cycle generally including 2-3 discrete temperature steps for denaturation, annealing and elongation. The cycling is often preceded by a single temperature step (called hold) at a high temperature (>90° C.), and followed by one hold at the end for final product extension or brief storage. The temperatures used and the length of time they are applied in each cycle vary based on a variety of parameters, including the enzyme used for DNA synthesis, the concentration dNTPs in the reaction, and the melting temperature (Tm) of the primers. For DNA polymerases that require heat activation the first step is heating the reaction to a temperature of 94-98° C. for 1-9 minutes. Then the reaction is heated to 94-98° C. for 20-30 seconds, which produces single-stranded DNA molecules. Next the reaction temperature is lowered to 50-65° C. for 20-40 seconds allowing annealing of the primers to the single-stranded DNA template. Typically the annealing temperature is about 3-5° C. below the Tm of the primers used. The temperature of the elongation step depends on the DNA polymerase used e.g. Taq polymerase has its optimum activity temperature at 75-80° C. At this step the DNA polymerase synthesizes a new DNA strand complementary to the DNA template strand by adding dNTPs that are complementary to the template. The extension time depends both on the DNA polymerase used and on the length of the DNA fragment to be amplified—a thousand bases per minute is usual. A final elongation may be performed at a temperature of 70-74° C. for 5-15 minutes after the last PCR cycle to ensure that any remaining single-stranded DNA is fully extended. A final hold at 4-15° C. for an indefinite time may be employed for short-term storage of the reaction. Other nucleic acid amplification techniques are well known in the art, and include methods such as NASBA, 3SR and Transcription Mediated Amplification (TMA). Other suitable amplification methods include the ligase chain reaction (LCR), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995), invader technology, strand displacement technology, and nick displacement amplification (WO 2004/067726). This list is not intended to be exhaustive; any nucleic acid amplification technique may be used provided the appropriate nucleic acid product is specifically amplified. Design of suitable primers and/or probes is within the capability of one skilled in the art. Various primer design tools are freely available to assist in this process such as the NCBI Primer-BLAST tool. Primers and/or probes may be at least 15, 16, 17, 18, 19, 20, 21, 22, 23, 24 or 25 (or more) nucleotides in length. mRNA expression levels may be measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

RNA expression may be determined by hybridization of RNA to a set of probes. The probes may be arranged in an array. Microarray platforms include those manufactured by companies such as Affymetrix, Illumina and Agilent. Examples of microarray platforms manufactured by Affymetrix include the U133 Plus2 array, the Almac proprietary Xcel™ array and the Almac proprietary Cancer DSAs®, including the Ovarian Cancer DSA®. In specific embodiments a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

The methods described herein may further comprise extracting total nucleic acid or RNA from the sample. Suitable methods are known in the art and include use of commercially available kits such as RNeasy and GeneJET RNA purification kit.

The invention also relates to a system or device for performing a method as described herein.

In a further aspect, the present invention relates to a system or test kit for performing a method as described herein, comprising:
a) one or more testing devices for determining the expression level of at least 3 biomarkers in a sample from the subject, wherein at least two of the biomarkers are from Table A and at least one of the biomarkers is from Table B or at least two biomarkers in a sample from the subject
b) a processor; and
c) storage medium comprising a computer application that, when executed by the processor, is configured to:
   (i) access and/or calculate the determined expression levels of the at least 3 biomarkers in a sample from the subject, wherein at least two of the biomarkers are from Table A and at least one of the biomarkers is from Table B or the at least two biomarkers in the sample on the one or more testing devices
   (ii) calculate whether there is an increased or decreased level of the biomarkersin the sample; and
   (iii) output from the processor the selection of whether to administer an anti-angiogenic therapeutic agent to a subject having a cancer and/or a prediction of the responsiveness of a subject with cancer to an anti-angiogenic therapeutic agent and/or the clinical prognosis of a subject with cancer.

By testing device is meant a combination of components that allows the expression level of a gene to be determined. The components may include any of those described above with respect to the methods for determining expression level at the level of protein, RNA or epigenetic modification. For example the components may be antibodies, primers, detection agents and so on. Components may also include one or more of the following: microscopes, microscope slides, x-ray film, radioactivity counters, scintillation counters, spectrophotometers, colorimeters, fluorometers, luminometers, and densitometers.

In certain embodiments the system or test kit further comprises a display for the output from the processor.

The invention also relates to a computer application or storage medium comprising a computer application as defined above.

In certain example embodiments, provided is a computer-implemented method, system, and a computer program product for selection of whether to administer an anti-angiogenic therapeutic agent to a subject having a cancer and/or prediction of the responsiveness of a subject with cancer to an anti-angiogenic therapeutic agent and/or determining the clinical prognosis of a subject with cancer, in accordance with the methods described herein. For example, the computer program product may comprise a non-transitory computer-readable storage device having computer-readable program instructions embodied thereon that, when executed by a computer, cause the computer to select whether to administer an anti-angiogenic therapeutic agent to a subject having a cancer and/or a predict the responsiveness of a subject with cancer to an anti-angiogenic therapeutic agent and/or determine the clinical prognosis of a subject with cancer as described herein. For example, the computer executable instructions may cause the computer to:

(i) access and/or calculate the determined expression levels of the at least 3 biomarkers in a sample from the subject, wherein at least two of the biomarkers are from Table A and at least one of the biomarkers is from Table B or the at least two biomarkers in a sample on one or more testing devices;

(ii) calculate whether there is an increased or decreased level of the at least 3 biomarkers in a sample from the subject, wherein at least two of the biomarkers are from Table A and at least one of the biomarkers is from Table B or the at least two biomarkers in the sample; and,

(iii) provide an output regarding the selection of whether to administer an anti-angiogenic therapeutic agent to a subject having a cancer and/or a prediction of the responsiveness of a subject with cancer to an anti-angiogenic therapeutic agent and/or the clinical prognosis of a subject with cancer.

In certain example embodiments, the computer-implemented method, system, and computer program product may be embodied in a computer application, for example, that operates and executes on a computing machine and a module. When executed, the application may select whether to administer an anti-angiogenic therapeutic agent to a subject having a cancer and/or a predict the responsiveness of a subject with cancer to an anti-angiogenic therapeutic agent and/or determine the clinical prognosis of a subject with cancer, in accordance with the example embodiments described herein.

As used herein, the computing machine may correspond to any computers, servers, embedded systems, or computing systems. The module may comprise one or more hardware or software elements configured to facilitate the computing machine in performing the various methods and processing functions presented herein. The computing machine may include various internal or attached components such as a processor, system bus, system memory, storage media, input/output interface, and a network interface for communicating with a network, for example.

The computing machine may be implemented as a conventional computer system, an embedded controller, a laptop, a server, a customized machine, any other hardware platform, such as a laboratory computer or device, for example, or any combination thereof. The computing machine may be a distributed system configured to function using multiple computing machines interconnected via a data network or bus system, for example.

The processor may be configured to execute code or instructions to perform the operations and functionality described herein, manage request flow and address mappings, and to perform calculations and generate commands. The processor may be configured to monitor and control the operation of the components in the computing machine. The processor may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor may be a single processing unit, multiple processing units, a single processing core, multiple processing cores, special purpose processing cores, co-processors, or any combination thereof. According to certain example embodiments, the processor, along with other components of the computing machine, may be a virtualized computing machine executing within one or more other computing machines.

The system memory may include non-volatile memories such as read-only memory (“ROM”), programmable read-only memory (“PROM”), erasable programmable read-only memory (“EPROM”), flash memory, or any other device capable of storing program instructions or data with or without applied power. The system memory may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory. The system memory may be implemented using a single memory module or multiple memory modules. While the system memory may be part of the computing machine, one skilled in the art will recognize that the system memory may be separate from the computing machine without departing from the scope of the subject technology. It should also be appreciated that the system memory may include, or operate in conjunction with, a non-volatile storage device such as the storage media.

The storage media may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media may store one or more operating systems, application programs and program modules such as module, data, or any other information. The storage media may be part of, or connected to, the computing machine. The storage media may also be part of one or more other computing machines that are in communication with the computing machine, such as servers, database servers, cloud storage, network attached storage, and so forth.

The module may comprise one or more hardware or software elements configured to facilitate the computing machine with performing the various methods and processing functions presented herein. The module may include one or more sequences of instructions stored as software or firmware in association with the system memory, the storage media, or both. The storage media may therefore represent examples of machine or computer readable media on which instructions or code may be stored for execution by the processor. Machine or computer readable media may generally refer to any medium or media used to provide instructions to the processor. Such machine or computer readable media associated with the module may comprise a computer software product. It should be appreciated that a computer software product comprising the module may also be associated with one or more processes or methods for delivering the module to the computing machine via a network, any signal-bearing medium, or any other communication or delivery technology. The module may also comprise hardware circuits or information for configuring hardware circuits such as microcode or configuration information for an FPGA or other PLD.

The input/output (“I/O”) interface may be configured to couple to one or more external devices, to receive data from the one or more external devices, and to send data to the one or more external devices. Such external devices along with the various internal devices may also be known as peripheral devices. The I/O interface may include both electrical and physical connections for operably coupling the various peripheral devices to the computing machine or the processor. The I/O interface may be configured to communicate data, addresses, and control signals between the peripheral devices, the computing machine, or the processor. The I/O interface may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface may be configured to implement only one interface or bus technology.

Alternatively, the I/O interface may be configured to implement multiple interfaces or bus technologies. The I/O interface may be configured as part of, all of, or to operate in conjunction with, the system bus. The I/O interface may include one or more buffers for buffering transmissions between one or more external devices, internal devices, the computing machine, or the processor.

The I/O interface may couple the computing machine to various input devices including mice, touch-screens, scanners, electronic digitizers, sensors, receivers, touchpads, trackballs, cameras, microphones, keyboards, any other pointing devices, or any combinations thereof. The I/O interface may couple the computing machine to various output devices including video displays, speakers, printers, projectors, tactile feedback devices, automation control, robotic components, actuators, motors, fans, solenoids, valves, pumps, transmitters, signal emitters, lights, and so forth.

The computing machine may operate in a networked environment using logical connections through the network interface to one or more other systems or computing machines across the network. The network may include wide area networks (WAN), local area networks (LAN), intranets, the Internet, wireless access networks, wired networks, mobile networks, telephone networks, optical networks, or combinations thereof. The network may be packet switched, circuit switched, of any topology, and may use any communication protocol.

Communication links within the network may involve various digital or an analog communication media such as fiber optic cables, free-space optics, waveguides, electrical conductors, wireless links, antennas, radio-frequency communications, and so forth. The processor may be connected to the other elements of the computing machine or the various peripherals discussed herein through the system bus. It should be appreciated that the system bus may be within the processor, outside the processor, or both. According to some embodiments, any of the processor, the other elements of the computing machine, or the various peripherals discussed herein may be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

Reagents, tools, and/or instructions for performing the methods described herein can be provided in a kit. Such a kit can include reagents for collecting a tissue sample from a patient, such as by biopsy, and reagents for processing the tissue. The kit can also include one or more reagents for performing a expression level analysis, such as reagents for performing nucleic acid amplification, including RT-PCR and qPCR, NGS, northern blot, proteomic analysis, or immunohistochemistry to determine expression levels of biomarkers in a sample of a patient. For example, primers for performing RT-PCR, probes for performing northern blot analyses, and/or antibodies or aptamers, as discussed herein, for performing proteomic analysis such as Westem blot, immunohistochemistry and ELISA analyses can be included in such kits. Appropriate buffers for the assays can also be included. Detection reagents required for any of these assays can also be included. The kits may be array or PCR based kits for example and may include additional reagents, such as a polymerase and/or dNTPs for example. The kits featured herein can also include an instruction sheet describing how to perform the assays for measuring expression levels.

The kit may include one or more primer pairs complementary to at least one of the biomarkers described herein.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing a gene expression analysis and interpreting the results.

The inventors have found that a range of signatures can point to the sub-type and can be identified using the teaching herein.

Accordingly, the invention also relates to a method of deriving a panel of at least 2 biomarkers, wherein the expression level(s) of the at least 2 biomarkers in a sample from a subject with a cancer allows the cancer to be allocated to a sub-type

(optionally) wherein the cancer sub-type is defined by the expression levels of the genes in Tables A and B

said method comprising the steps of:

sorting samples from a sample set of known pathology and/or clinical outcome on the basis of allocation to the sub-type

obtaining the expression profiles of the samples

analysing the expression profiles from the sample set using a mathematical model identifying from the results of the mathematical model one or more biomarkers expressed in the sample set that are most predictive of the cancer sub-type.

In certain embodiments the mathematical model is a parametric, non-parametric or semi-parametric model. In specific embodiments the mathematical model is Partial Least Squares (PLS), Shrinkage Discriminate Analysis (SDA), or Diagonal SDA (DSDA). Identifying from the results of the mathematical model one or more biomarkers expressed in the sample set that are most predictive of the cancer sub-type may comprise identifying one or more biomarkers for which area under the receiver operator characteristic curve (AUC) and/or Concordance Index (C-Index) are significant.

In certain embodiments the panel is derived by obtaining the expression profiles of samples from a sample set of known pathology and/or clinical outcome. The samples may originate from the same sample tissue type or different tissue types. As used herein an “expression profile” comprises a set of values representing the expression level for each biomarker analyzed from a given sample.

The expression profiles from the sample set are then analyzed using a mathematical model. Different mathematical models may be applied and include, but are not limited to, models from the fields of pattern recognition (Duda et al. Pattern Classification, 2^{nd} ed., John Wiley, New York 2001), machine learning (Schölkopf et al. Learning with Kernels, MIT Press, Cambridge 2002, Bishop, Neural Networks for Pattern Recognition, Clarendon Press, Oxford 1995), statistics (Hastie et al. The Elements of Statistical Learning, Springer, New York 2001), bioinformatics (Dudoit et al., 2002, J. Am. Statist. Assoc. 97:77-87, Tibshirani et al., 2002, Proc. Natl. Acad. Sci. USA 99:6567-6572) or chemometrics (Vandeginste, et al., Handbook of Chemometrics and Qualimetrics, Part B, Elsevier, Amsterdam 1998). The mathematical model identifies one or more biomarkers expressed in the sample set that are most predictive of the cancer sub-type. These one or more biomarkers define a panel or an expression signature. In certain example embodiments, the mathematical model defines a variable, such as a weight, for each identified biomarker. In certain example embodiments, the mathematical model defines a decision function. The decision function may further define a threshold score which separates the sample set into two classes such as, but not limited to, samples where the cancer belongs to the cancer sub-type and samples where the cancer does not belong to the sub-type. In one example embodiment, the decision function and panel or expression signature are defined using a linear classifier.

The overall expression data for a given sample may be normalized using methods known to those skilled in the art in order to correct for differing amounts of starting material, varying efficiencies of the extraction and amplification reactions.

In certain example embodiments, biomarkers useful for distinguishing between cancer subtypes can be determined by identifying biomarkers exhibiting the highest degree of variability between samples in the patient data set as determined using the expression detection methods and patient sample sets discussed above. Standard statistical methods known in the art for identifying highly variable data points in expression data may be used to identify the highly variable biomarkers. For example, a combined background and variance filter to the patient data set. The background filter is based on the selection of probe sets with expression E and expression variance var_{E} above the thresholds defined by background standard deviation σBg (from the Expression Console software) and quantile of the standard normal distribution z_{α} at a specified significance a probe sets were kept if:

E>log₂((zₐσ_{Bg})); log₂((var_{E})>2[log₂(σ_{Bg})−E−log₂(log(2))]

where a defines a significance threshold. In certain example embodiment, the significance threshold is 6.3·10⁻⁵. In another example embodiment, the significance threshold may be between 1.0·10⁻⁷ to 1.0·10⁻³.

In certain example embodiments, the highly variable biomarkers may be further analyzed to group samples in the patient data set into subtypes or clusters based on similar gene expression profiles. For examples, biomarkers may be clustered based on how highly correlated the up-regulation or down-regulation of their expression is to one another. Different clustering analysis techniques may be applied to gene expression data and include, but are not limited to hierarchical clustering, inclusive of agglomerative and divisive methods (Eisen et al., 1998, PNAS 25:14863-14868), k-mean family clustering, inclusive of hard and fuzzy methods (Tavazoie et al., 1999, Nat Genet, 22281-285; Gasch and Eisen, 2002, Genome Biology 3: RESEARCH0059), self-organizing maps (SOM) (Tamayo et al., 1999, PNAS 96:2907-2912), methods based on graph theory (Sharan and Shamir, 2000, Proc Int Conf Intell Syst Mol Biol., 8:307-16), biclustering methods (Tanay et al., 2002, Bioinformatics 18: Suppl 1:S136-44), and ensemble methods (Dudoit et al. 2003, Bioinformatics, 19:1090-9). In one example embodiment, hierarchical agglomerative clustering is used to identify the cancer subtypes.

During clustering, determination of the similarity of features (sample, gene) requires the specification of a similarity matrix and methods used to calculate the similarity include, but are not limited to Euclidean distance, maximum distance, Manhattan distance, Minkowski distance, Canberra distance, binary distance, kendall's tau, Pearson correlation, Spearman correlation.

During hierarchical clustering, inter-cluster distances are defined by linkage functions. Several linkage functions can be used to calculate inter-cluster distances and include, but are not limited to single linkage (Sneath, 1957, Journal of General Microbiology, 17:201-226), complete linkage (McQuitty, 1960, Educational and Psychological Measurement, 20:55-67; Sokal and Sneath, 1963, Principles of Numerical Taxonomy, San Francisco:Freeman), UPGMA/group average (Sokal and Michener, 1958, University of Kansas Scientific Bulletin, 38:1409-1438), UPGMC/unweighted centroid (Lance and Williams, 1965, Computer Journal, 8246:249), WPGMC/weighted centroid (Gower, 1967, Biometrics, 30:623-637) and Ward's method of minimum variance (Ward, 1963, Journal of the American Statistical Association, 58:236-244).

To determine the biological relevance of each subtype, the biomarkers within each cluster may be further mapped to their corresponding genes and annotated by cross-reference to one or more databases referencing metabolic and signaling pathways, human gene functions and disease association, and/or ontological categories (e.g. biological processes, cellular components, molecular functions). In another example embodiment, biomarkers in clusters that are up regulated and enriched for immune response general functional terms are grouped into a putative non-angiongenesis sample group and used for expression signature generation. In another example embodiment, biomarkers in clusters that are down regulated and enriched for angiogenesis and vasculature development and are up regulated and enriched for immune response general functional terms are grouped into a putative non-angiongenesis sample group and used for expression signature generation. Further details for conducting functional analysis of biomarker clusters is provided in the Examples section below.

The following methods may be used to derive panels or expression signatures for distinguishing between cancers that belong to the sub-type or not or between subjects that are responsive or non-responsive to anti-angiogenic therapeutics, or as prognostic indicators of certain cancer types, including expression signatures derived from the biomarkers disclosed above. In certain other example embodiments, the panel or expression signature is derived using a decision tree (Hastie et al. The Elements of Statistical Learning, Springer, New York 2001), a random forest (Breiman, 2001 Random Forests, Machine Learning 45:5), a neural network (Bishop, Neural Networks for Pattern Recognition, Clarendon Press, Oxford 1995), discriminant analysis (Duda et al. Patter Classification, 2nd ed., John Wiley, New York 2001), including, but not limited to linear, diagonal linear, quadratic and logistic discriminant analysis, a Prediction Analysis for Microarrays (PAM, (Tibshirani et al., 2002, Proc. Natl. Acad. Sci. USA 99:6567-6572)) or a Soft Independent Modeling of Class Analogy analysis. (SIMCA, (Wold, 1976, Pattern Recogn. 8:127-139)). Classification trees (Breiman, Leo; Friedman, J. H.; Olshen, R. A.; Stone, C. J. (1984). Classification and regression trees. Monterey, Calif.: Wadsworth & Brooks/Cole Advanced Books & Software. ISBN 978-0-412-04841-8) provide a means of predicting outcomes based on logic and rules. A classification tree is built through a process called binary recursive partitioning, which is an iterative procedure of splitting the data into partitions/branches. The goal is to build a tree that distinguishes among pre-defined classes. Each node in the tree corresponds to a variable. To choose the best split at a node, each variable is considered in turn, where every possible split is tried and considered, and the best split is the one which produces the largest decrease in diversity of the classification label within each partition. This is repeated for all variables, and the winner is chosen as the best splitter for that node. The process is continued at the next node and in this manner, a full tree is generated. One of the advantages of classification trees over other supervised learning approaches such as discriminant analysis, is that the variables that are used to build the tree can be either categorical, or numeric, or a mix of both. In this way it is possible to generate a classification tree for predicting outcomes based on say the directionality of gene expression. Random forest algorithms (Breiman, Leo (2001). “Random Forests”. Machine Learning 45 (1): 5-32. doi:10.1023/A:1010933404324) provide a further extension to classification trees, whereby a collection of classification trees are randomly generated to form a “forest” and an average of the predicted outcomes from each tree is used to make inference with respect to the outcome.

Biomarker expression values may be defined in combination with corresponding scalar weights on the real scale with varying magnitude, which are further combined through linear or non-linear, algebraic, trigonometric or correlative means into a single scalar value via an algebraic, statistical leaming, Bayesian, regression, or similar algorithms which together with a mathematically derived decision function on the scalar value provide a predictive model by which expression profiles from samples may be resolved into discrete classes of responder or non-responder, resistant or non-resistant, to a specified drug, drug class, molecular subtype, or treatment regimen. Such predictive models, including biomarker membership, are developed by learning weights and the decision threshold, optimized for sensitivity, specificity, negative and positive predictive values, hazard ratio or any combination thereof, under cross-validation, bootstrapping or similar sampling techniques, from a set of representative expression profiles from historical patient samples with known drug response and/or resistance.

In one embodiment, the biomarkers are used to form a weighted sum of their signals, where individual weights can be positive or negative. The resulting sum (“expression score”) is compared with a pre-determined reference point or value. The comparison with the reference point or value may be used to diagnose, or predict a clinical condition or outcome.

In certain example embodiments, the panel or expression signature is defined by a decision function. A decision function is a set of weighted expression values derived using a linear classifier. All linear classifiers define the decision function using the following equation:

f(x)=w′·x+b=Σwᵢ·xᵢ+b (1)

All measurement values, such as the microarray gene expression intensities xᵢ, for a certain sample are collected in a vector x. Each intensity is then multiplied with a corresponding weight wᵢ to obtain the value of the decision function f(x) after adding an offset term b. In deriving the decision function, the linear classifier will further define a threshold value that splits the gene expression data space into two disjoint sections. Example linear classifiers include but are not limited to partial least squares (PLS), (Nguyen et al., Bioinformatics 18 (2002) 39-50), support vector machines (SVM) (Schölkopf et al., Learning with Kernels, MIT Press, Cambridge 2002), and shrinkage discriminant analysis (SDA) (Ahdesmäki et al., Annals of applied statistics 4, 503-519 (2010)). In one example embodiment, the linear classifier is a PLS linear classifier.

The decision function is empirically derived on a large set of training samples, for example from patients showing a good or poor clinical prognosis. The threshold separates a patient group based on different characteristics such as, but not limited to, clinical prognosis before or after a given therapeutic treatment. The interpretation of this quantity, i.e. the cut-off threshold, is derived in the development phase (“training”) from a set of patients with known outcome. The corresponding weights and the responsiveness/resistance cut-off threshold for the decision score are fixed a priori from training data by methods known to those skilled in the art. In one example embodiment, Partial Least Squares Discriminant Analysis (PLS-DA) is used for determining the weights. (L. Ståhle, S. Wold, J. Chemom. 1 (1987) 185-196; D. V. Nguyen, D. M. Rocke, Bioinformatics 18 (2002) 39-50).

Effectively, this means that the data space, i.e. the set of all possible combinations of biomarker expression values, is split into two mutually exclusive groups corresponding to different clinical classifications or predictions, for example, one corresponding to good clinical prognosis and poor clinical prognosis. In the context of the overall classifier, relative over-expression of a certain biomarker can either increase the decision score (positive weight) or reduce it (negative weight) and thus contribute to an overall decision of, for example, a good clinical prognosis.

In certain example embodiments of the invention, the data is transformed non-linearly before applying a weighted sum as described above. This non-linear transformation might include increasing the dimensionality of the data. The non-linear transformation and weighted summation might also be performed implicitly, for example, through the use of a kernel function. (Schölkopf et al. Learning with Kernels, MIT Press, Cambridge 2002).

In certain example embodiments, the patient training set data is derived by isolated RNA from a corresponding cancer tissue sample set and determining expression values by hybridizing the cDNA amplified from the isolated RNA to a microarray. In certain example embodiments, the microarray used in deriving the panel or expression signature is a transcriptome array. As used herein a “transcriptome array” refers to a microarray containing probe sets that are designed to hybridize to sequences that have been verified as expressed in the diseased tissue of interest. Given alternative splicing and variable poly-A tail processing between tissues and biological contexts, it is possible that probes designed against the same gene sequence derived from another tissue source or biological context will not effectively bind to transcripts expressed in the diseased tissue of interest, leading to a loss of potentially relevant biological information. Accordingly, it is beneficial to verify what sequences are expressed in the disease tissue of interest before deriving a microarray probe set. Verification of expressed sequences in a particular disease context may be done, for example, by isolating and sequencing total RNA from a diseased tissue sample set and cross-referencing the isolated sequences with known nucleic acid sequence databases to verify that the probe set on the transcriptome array is designed against the sequences actually expressed in the diseased tissue of interest. Methods for making transcriptome arrays are described in United States Patent Application Publication No. 2006/0134663, which is incorporated herein by reference. In certain example embodiments, the probe set of the transcriptome array is designed to bind within 300 nucleotides of the 3′ end of a transcript. Methods for designing transcriptome arrays with probe sets that bind within 300 nucleotides of the 3′ end of target transcripts are disclosed in United States Patent Application Publication No. 2009/0082218, which is incorporated by reference herein. In certain example embodiments, the microarray used in deriving the gene expression profiles of the present invention is the Almac Ovarian Cancer DSA™ microarray (Almac Group, Craigavon, United Kingdom).

An optimal (linear) classifier can be selected by evaluating a (linear) classifier's performance using such diagnostics as “area under the curve” (AUC). AUC refers to the area under the curve of a receiver operating characteristic (ROC) curve, both of which are well known in the art. AUC measures are useful for comparing the accuracy of a classifier across the complete data range. (Linear) classifiers with a higher AUC have a greater capacity to classify unknowns correctly between two groups of interest (e.g., ovarian cancer samples and normal or control samples). ROC curves are useful for plotting the performance of a particular feature (e.g., any of the biomarkers described herein and/or any item of additional biomedical information) in distinguishing between two populations (e.g., individuals responding and not responding to a therapeutic agent). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of positive cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

In certain embodiments deriving a panel of at least 2 biomarkers, wherein the expression level(s) of the at least 2 biomarkers in a sample from a subject with a cancer allows the cancer to be allocated to a sub-type (optionally) wherein the cancer sub-type is defined by the expression levels of the genes in Tables A and B comprises obtaining the expression profiles of a training set of samples known to belong to the sub-type or not using microarray probes

mapping probes to genes and measuring gene expression using the log₂ transformation of the median probeset expression for each gene

within nested CV, performing quantile normalization following a pre-filtering to remove 75% of genes with low variance, low intensity, and high correlation to cDNA yield

ranking genes/features based on correlation adjusted t-scores² and discarding 10% of the least important genes until 5 genes remain

identifying a panel of at least 2 biomarkers for which AUC and C-Index (Concordance Index) for the Progression free survival (PFS) endpoint under cross-validation are significant.

In further embodiments deriving a panel of at least 2 biomarkers, wherein the expression level(s) of the at least 2 biomarkers in a sample from a subject with a cancer allows the cancer to be allocated to a sub-type (optionally) wherein the cancer sub-type is defined by the expression levels of the genes in Tables A and B comprises

obtaining the expression profiles of a training set of samples known to belong to the sub-type or not using microarray probes

mapping probes to genes and measuring gene expression using the log₂ transformation of the median probeset expression for each gene

within nested CV, performing quantile normalization following a pre-filtering to remove 75% of genes with low variance, low intensity, and high correlation to cDNA yield

using Recursive Feature Elimination (RFE) for feature reduction to discard 10% of the least important genes (based upon their discriminatory ability) until 5 genes remain

identifying a panel of at least 2 biomarkers for which AUC and C-Index (Concordance Index) for the Progression free survival (PFS) endpoint under cross-validation are significant.

The signatures/panels described herein may result from the application of the methods for deriving panels of biomarkers described herein.

According to all aspects of the invention the method may comprise allocating the cancer to the sub-type based on the expression level of a panel of one or more, optionally two or more, biomarkers derived using the method outlined above in a sample from the subject.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the examples described herein.

Although specific embodiments have been described above in detail, the description is merely for purposes of illustration. It should be appreciated, therefore, that many aspects described above are not intended as required or essential elements unless explicitly stated otherwise.

Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

### DESCRIPTION OF THE FIGURES

FIG. 1: Heat map showing unsupervised hierarchical clustering of gene expression data using the 1040 most variable genes in the 265 Edinburgh high grade serous ovarian carcinomas. Gene expression across all samples is represented horizontally. Functional processes corresponding to each gene cluster are labeled along the right of the figure. Angio, Immune, and Angiolmmune subgroups are labeled for each of the sample clusters, and color coded along the top as described in the legend box.

FIG. 2: Kaplan-Meier analysis of subgroups with respect to overall survival as defined by unsupervised clustering analysis of 265 Edinburgh high grade serous ovarian carcinomas

FIG. 3: AUC performance for predicting the molecular subtype calculated at a range of feature lengths. The red circle depicts the mean AUC performance of the 1000 random sampling of genes and the green error bars represent −/+2 standard deviations from the mean.

FIG. 4: C-index performance measured using the signature scores within the control arm for predicting the overall survival at a range of feature lengths. The red circle depicts the mean C-index performance of the 1000 random sampling of genes and the green error bars represent −/+2 standard deviations from the mean.

FIG. 5: Hazard ratio (HR) performance within the samples predicted as “Immune” for predicting the overall survival at a range of feature lengths. The red circle depicts the mean HR performance of the 1000 random sampling of genes and the green error bars represent −/+2 standard deviations from the mean.

FIG. 6: Signature development: AUC of training set under CV.

FIG. 7: Signature development: C-Index of training set under CV.

FIG. 8: Signature development: HR of training set under CV.

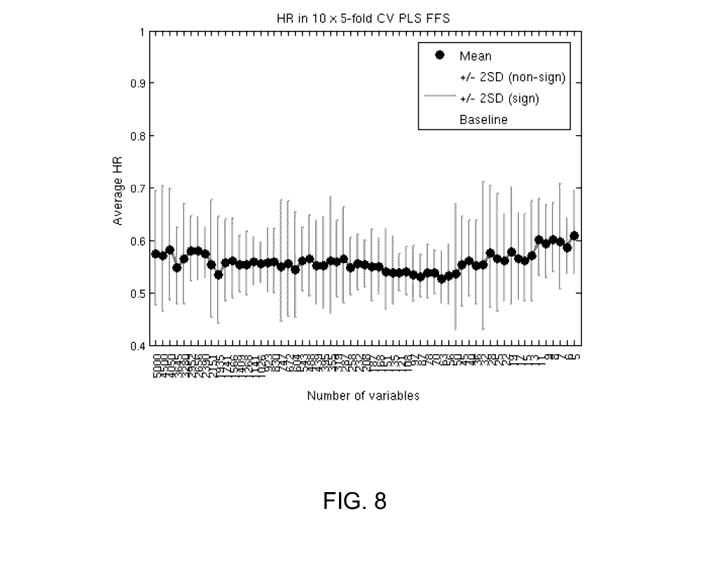

FIG. 9: Signature development: HR of ICON7 SOC samples under CV.

FIG. 10: Signature development: C-Index of ICON7 SOC samples under CV.

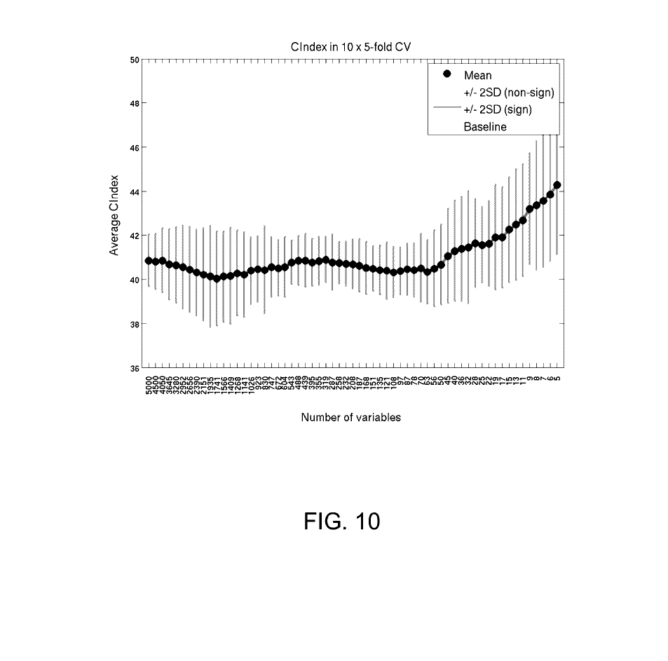

FIG. 11: Signature development: HR of ICON7 Immune samples under CV.

FIG. 12: Signature development: HR of ICON7 ProAngio samples under CV.

FIG. 13: Core set analysis: Immune63GeneSig_CoreGenes_lnternalVal.png.

FIG. 14: Core set analysis: Immune63GeneSig_CoreGenes_Tothill.png.

FIG. 15: Core set analysis: Immune63GeneSig_CoreGenes_ICON7_SOC.png.

FIG. 16: Minimum gene set analysis: Immune63GeneSig_MinGenes_Tothill.png.

FIG. 17: ICON7 SOC: Minimum gene set analysis: Immune63GeneSig_MinGenes_ICON7_SOC.png.

FIG. 18: ICON7 Immune: Minimum gene set analysis: Immune63GeneSig_MinGenes_ICON7_Immune.png.

FIG. 19: AUC (area under the receiver operator characteristic curve) performance of the training set measured under 10 repeats of five-fold cross validation using for predicting the Immune subtype. The performance for predicting the Immune subtype (AUC) was very strong at larger feature lengths and decreases as the number of features gets smaller. A feature length of 121 genes has been selected, which yields a significant AUC of 90.05 [87.80, 92.29].

FIG. 20: C-Index (concordance index) performance of the training set measured under 10 repeats of five-fold cross validation for predicting PFS (Progression Free Survival). A feature length of 121 genes yields a significant C-Index of 39.87 [38.31, 41.43].

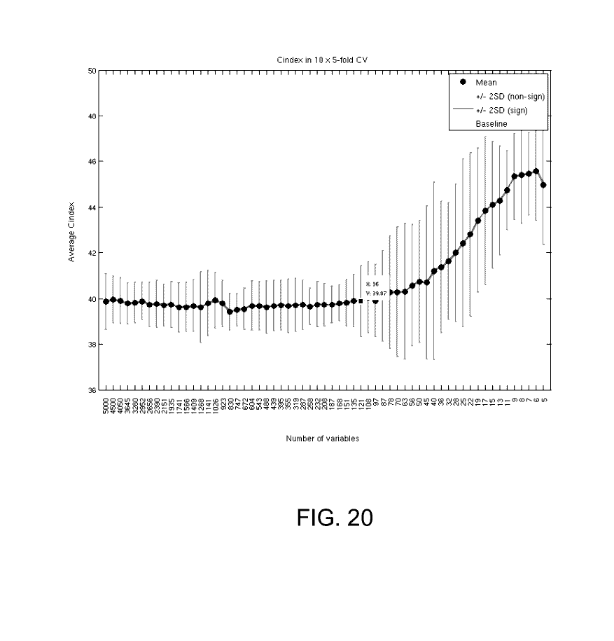

FIG. 21: Hazard Ratio (HR) performance of the ICON7 Standard of care (SOC) arm samples under 10 repeats of five-fold cross validation for the PFS endpoint. A feature length of 121 genes yields a significant HR of 0.55 [0.45, 0.67]. This demonstrates the prognostic utility of the signature in SOC samples.

FIG. 22: C-Index performance of the ICON7 Standard of care (SOC) arm samples under 10 repeats of five-fold cross validation for the PFS endpoint. A feature length of 121 genes yields a significant C-Index of 41.54 [39.94, 43.14]. This demonstrates the prognostic utility of the signature (independent of cut-off) in SOC samples.

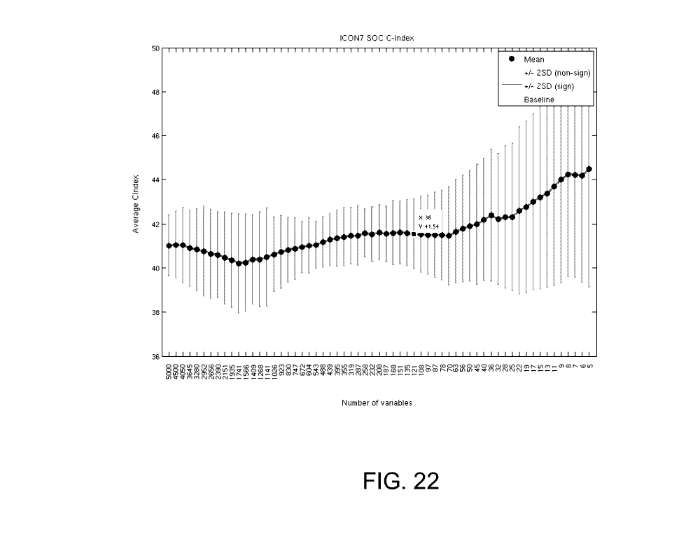

FIG. 23: HR performance of the ICON7 Immune group (as identified by the 63 gene signature) samples under 10 repeats of five-fold cross validation for the PFS endpoint. A feature length of 121 genes yields a significant HR of 1.80 [1.46, 2.22] showing lack of benefit of the addition of bevacuzimab in the Immune group.

FIG. 24: Core gene set analysis results for the 121 gene signature in the Internal validation sample set. Genes highlighted in red have the largest negative impact on the HR performance when removed from the signature.

FIG. 25: Core gene set analysis results for the 121 gene signature in the Tothill data set. Genes highlighted in red have the largest negative impact on the HR performance when removed from the signature.

FIG. 26: Core gene set analysis results for the 121 gene signature in the ICON7 SOC data set. Genes highlighted in red have the largest negative impact on the HR performance when removed from the signature.

FIG. 27: Minimum gene analysis results for the 121 gene signature in the Tothill data set. A significant HR can be achieved using at least 11 of the signature genes.

FIG. 28: Minimum gene analysis results for the 121 gene signature in the ICON7 SOC sample set. A significant HR can be achieved using at least 4 of the signature genes.

FIG. 29: Minimum gene analysis results for the 121 gene signature in the ICON7 Immune sample set. A significant HR can be achieved using at least 11 of the signature genes.

FIG. 30: AUC (area under the receiver operator characteristic curve) performance of the training set measured under 10 repeats of five-fold cross validation using for predicting the Immune subtype. The performance for predicting the Immune subtype (AUC) was very strong at larger feature lengths and decreases as the number of features gets smaller. A feature length of 232 genes has been selected, which yields a significant AUC of 94.29 [93.16, 95.42].

FIG. 31: C-Index (concordance index) performance of the training set measured under 10 repeats of five-fold cross validation for predicting PFS (Progression Free Survival). A feature length of 232 genes yields a significant C-Index of 39.35 [38.43, 40.27].

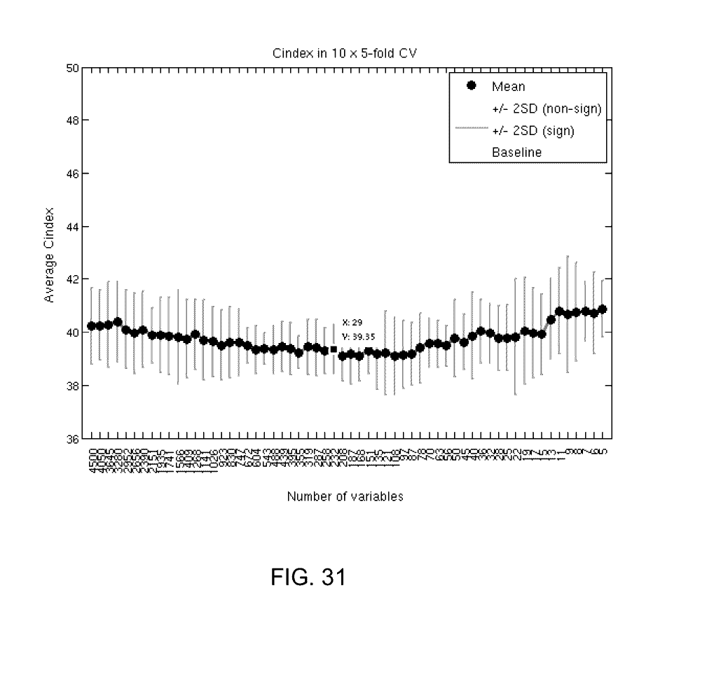

FIG. 32: Hazard Ratio (HR) performance of the ICON7 Standard of care (SOC) arm samples under 10 repeats of five-fold cross validation for the PFS endpoint. A feature length of 232 genes yields a significant HR of 0.57 [0.48, 0.67]. This demonstrates the prognostic utility of the signature in SOC samples.

FIG. 33: C-Index performance of the ICON7 Standard of care (SOC) arm samples under 10 repeats of five-fold cross validation for the PFS endpoint. A feature length of 232 genes yields a significant C-Index of 40.81 [39.52, 42.10]. This demonstrates the prognostic utility of the signature (independent of cut-off) in SOC samples.

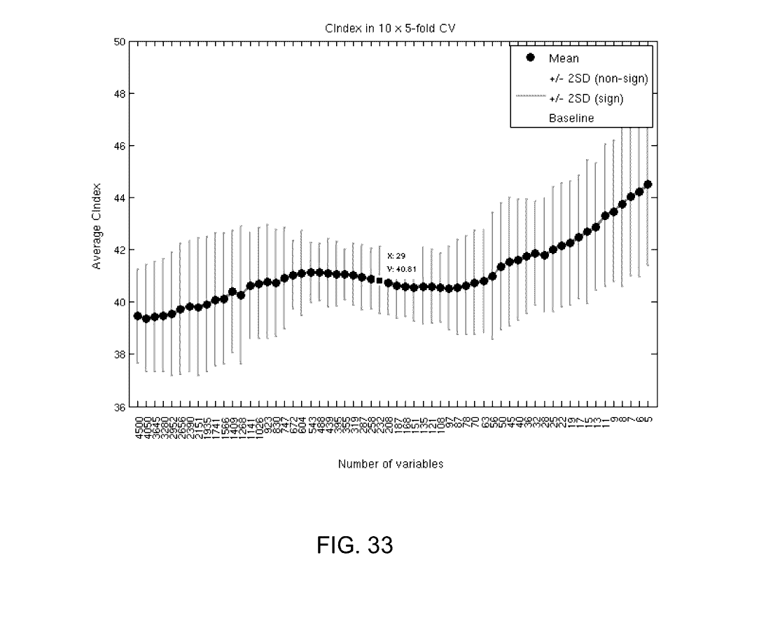

FIG. 34: HR performance of the ICON7 Immune group (as identified by the 63 gene signature) samples under 10 repeats of five-fold cross validation for the PFS endpoint. A feature length of 232 genes yields a significant HR of 1.63 [1.39, 1.99] showing lack of benefit of the addition of bevacuzimab in the Immune group.

FIG. 35: Core gene set analysis results for the 232 gene signature in the Internal validation sample set. Genes highlighted in red have the largest negative impact on the HR performance when removed from the signature.

FIG. 36: Core gene set analysis results for the 232 gene signature in the Tothill data set. Genes highlighted in red have the largest negative impact on the HR performance when removed from the signature.

FIG. 37: Core gene set analysis results for the 232 gene signature in the ICON7 SOC data set. Genes highlighted in red have the largest negative impact on the HR performance when removed from the signature.

FIG. 38: Minimum gene analysis results for the 232 gene signature in the Tothill data set. A significant HR can be achieved using at least 25 of the signature genes.

FIG. 39: Minimum gene analysis results for the 232 gene signature in the ICON7 SOC sample set. A significant HR can be achieved using at least 10 of the signature genes.

FIG. 40: Minimum gene analysis results for the 232 gene signature in the ICON7 Immune sample set. A significant HR can be achieved using at least 11 of the signature genes.

FIG. 41: Signature development: AUC of training set under CV.

FIG. 42: Signature development: C-Index of training set under CV.

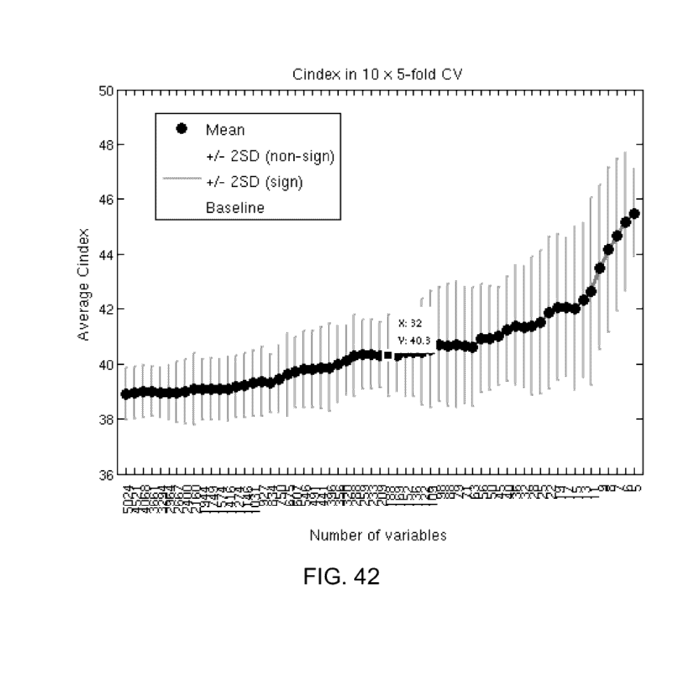

FIG. 43: Signature development: HR of ICON7 SOC samples under CV.

FIG. 44: Signature development: C-Index of ICON7 SOC samples under CV.

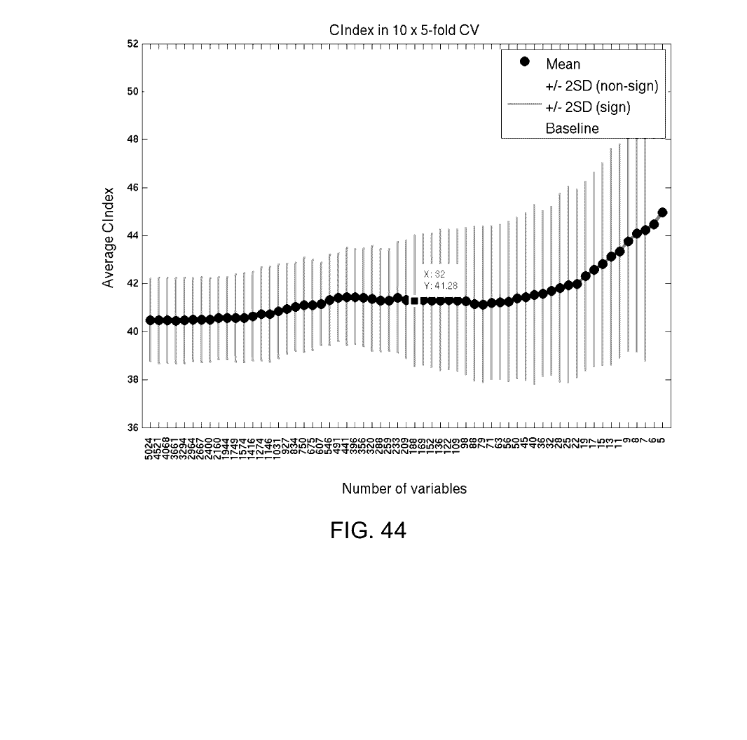

FIG. 45: Signature development: HR of ICON7 Immune samples under CV.

FIG. 46: Signature development: HR of ICON7 ProAngio samples under CV.

FIG. 47: Core set analysis: Immune_188GeneSig_CoreGenes_InternalVal.png.

FIG. 48: Core set analysis: Immune_188GeneSig_CoreGenes_Tothill.png.

FIG. 49: Core set analysis: Immune_188GeneSig_CoreGenes_ICON7_SOC.png.

FIG. 50: Minimum gene set analysis: Immune_188GeneSig_MinGenes_Tothill.png.

FIG. 51: ICON7 SOC: Minimum gene set analysis: Immune_188GeneSig_MinGenes_ICON7 SOC.png.

FIG. 52: ICON7 Immune: Minimum gene set analysis: Immune_188GeneSig_MinGenes_ICON7 Immune.png.

### EXAMPLES

The present invention will be further understood by reference to the following experimental examples.

### Example 1: Tissue Processing, Hierarchical Clustering and Subtype Identification Tumor Material

A cohort of 287 macrodissected epithelial serous ovarian tumor FFPE tissue samples sourced from the NHS Lothian and University of Edinburgh.

Gene Expression Profiling from FFPE

Total RNA was extracted from macrodissected FFPE tissue using the High Pure RNA Paraffin Kit (Roche Diagnostics GmbH, Mannheim, Germany). RNA was converted into complementary deoxyribonucleic acid (cDNA), which was subsequently amplified and converted into single-stranded form using the SPIA® technology of the WT-Ovation™ FFPE RNA Amplification System V2 (NuGEN Technologies Inc., San Carlos, Calif., USA). The amplified single-stranded cDNA was then fragemented and biotin labeled using the FL-Ovation™ cDNA Biotin Module V2 (NuGEN Technologies Inc.). The fragmented and labeled cDNA was then hybridized to the Almac Ovarian Cancer DSA™. Almac's Ovarian Cancer DSA research tool has been optimised for analysis of FFPE tissue samples, enabling the use of valuable archived tissue banks. The Almac Ovarian Cancer DSA™ research tool is an innovative microarray platform that represents the transcriptome in both normal and cancerous ovarian tissues. Consequently, the Ovarian Cancer DSA™ provides a comprehensive representation of the transcriptome within the ovarian disease and tissue setting, not available using generic microarray platforms. Arrays were scanned using the Affymentrix Genechip® Scanner 7G (Affymetrix Inc., Santa Clara, Calif.).

Data Preparation

Quality Control (QC) of profiled samples was carried out using MAS5 pre-processing algorithm. Different technical aspects were addressed: average noise and background homogeneity, percentage of present call (array quality), signal quality, RNA quality and hybridization quality. Distributions and Median Absolute Deviation of corresponding parameters were analyzed and used to identify possible outliers.

Almac's Ovarian Cancer DSA™ contains probes that primarily target the area within 300 nucleotides from the 3′ end. Therefore standard Affymetrix RNA quality measures were adapted—for housekeeping genes intensities of 3′ end probe sets with ratios of 3′ end probe set intensity to the average background intensity were used in addition to usual 3′/5′ ratios. Hybridization controls were checked to ensure that their intensities and present calls conform to the requirements specified by Affymetrix.

Hierarchical Clustering and Functional Analysis

Sample pre-processing was carried out using Robust Multi-Array analysis (RMA) [Irizarry R A, Bolstad B M, Collin F, Cope L M, Hobbs B, Speed T P. Summaries of Affymetrix GeneChip probe level data. Nucleic acids research 2003; 31:015]. The data matrix was sorted by decreasing variance, decreasing intensity and increasing correlation to cDNA yield. Following filtering of probe sets correlated with cDNA yield, incremental subsets of the data matrix were tested for cluster stability: the GAP statistic [Tibshirani R, Walther G, Hastie T. Estimating the number of clusters in a data set via the gap statistic. J Roy Stat Soc B 2001; 63:411-23] was applied to calculate the number of sample and probe set clusters while the stability of cluster composition was assessed using partition comparison methods. The final most variable probe set list was determined based on the smallest and most stable data matrix for the selected number of sample cluster.

Following standardization of the data matrix to the median probe set expression values, agglomerative hierarchical clustering was performed using Euclidean distance and Ward's linkage method [Ward J H. Hierarchical Grouping to Optimize an Objective Function. Journal of the American Statistical Association 1963; 58:236-&.]. The optimal number of sample and probe set clusters was determined using the GAP statistic [Tibshirani R, Walther G, Hastie T. Estimating the number of clusters in a data set via the gap statistic. J Roy Stat Soc B 2001; 63:411-23]. The significance of the distribution of clinical parameter factor levels across sample clusters was assessed using ANOVA (continuous factor) or chi-squared analysis (discrete factor) and corrected for false discovery rate (product of p-value and number of tests performed). A corrected p-value threshold of 0.05 was used as criterion for significance. Ovarian Cancer DSA® probe sets were remapped to genes using an annotation pipeline based on Ensembl v60 [http://oct2012.archive.ensembl.org/]. Functional enrichment analysis was conducted to identify and rank biological entities which were found to be associated with the clustered gene sets using the Gene Ontology biological processes classification [Ashburner M, Ball C A, Blake J A, et al. Gene ontology: tool for the unification of biology. The Gene Ontology Consortium. Nature genetics 2000; 25:25-9]. Entities were ranked according to a statistically derived enrichment score [Cho R J, Huang M X, Campbell M J, et al. Transcriptional regulation and function during the human cell cycle. Nature genetics 2001; 27:48-54] and adjusted for multiple testing [Benjamini Y, Hochberg Y. Controlling the False Discovery Rate—a Practical and Powerful Approach to Multiple Testing. J Roy Stat Soc B Met 1995; 57:289-300]. A corrected p-value of 0.05 was used as significance threshold. The identified enriched processes were summarised into an overall group function for each probe set/gene cluster.

Defining the Core Genes

The core angiogenic and immune genes were defined by evaluating functional enrichment of the 136 immune and 350 angiogeneic probe sets that constitute the immune and angiogenic clusters from the unsupervised analysis of the 265 HGS samples was performed using Almac's Functional Enrichment Tool (FET) v1.1.0. The functions were ordered by p-value and the 100 most significant biological functions were looked at. Of these 100 significant functions the ones directly related to immune processes (immune response, inflamatory response, interferon, antigen processing) or angiogeneic processes (angiogenesis, vasculature development, system development) were kept and the genes involved in each process were kept and remapped to the ovarian array resulting in the 238 core functional genes (77 immune, 161 angiogenesis)

Results

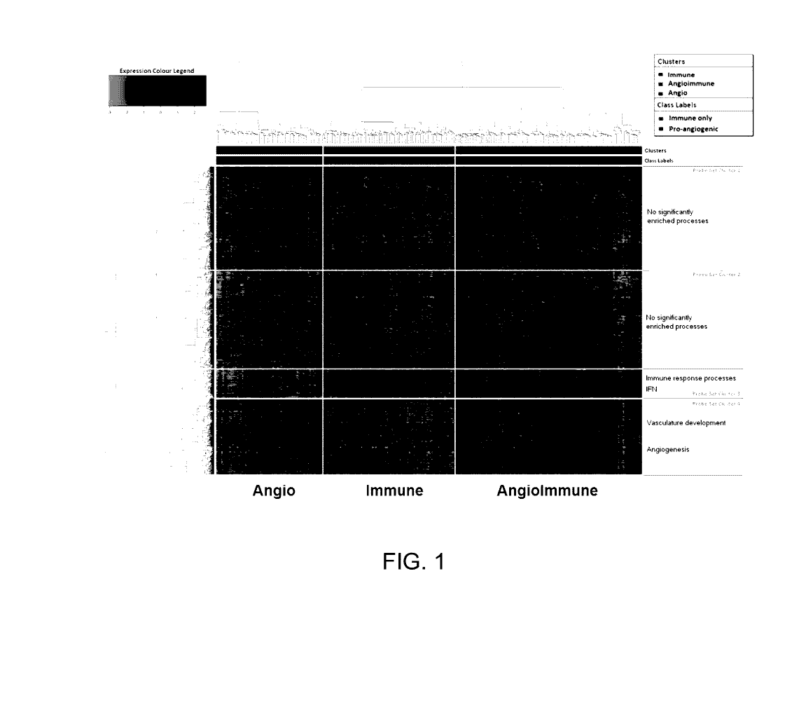

265 HGS tumors passed microarray QC and subsequently underwent unsupervised hierarchical clustering based on 1400 most variable probe sets (corresponding to 1040 genes). Three sample clusters and four gene clusters were identified (FIG. 1). There was no significant association between HGS clusters and clinico-pathological features. Functional analysis (FIG. 1) revealed that cluster HGS3 was characterized by up regulation of genes associated with immune response and angiogenesis/vascular development (cluster referred to as Angioimmune forthwith). Cluster HGS1 was associated with upregulation of angiogenesis/vascular development (although apparently to a lesser extent than cluster HGS3) but without high expression of genes involved in immune response (cluster referred to as Angio forthwith). Cluster HGS2 was characterized by upregulation of genes involved in immune response without upregulation of genes involved in angiogenesis or vascular development (cluster referred to as Immune forthwith).

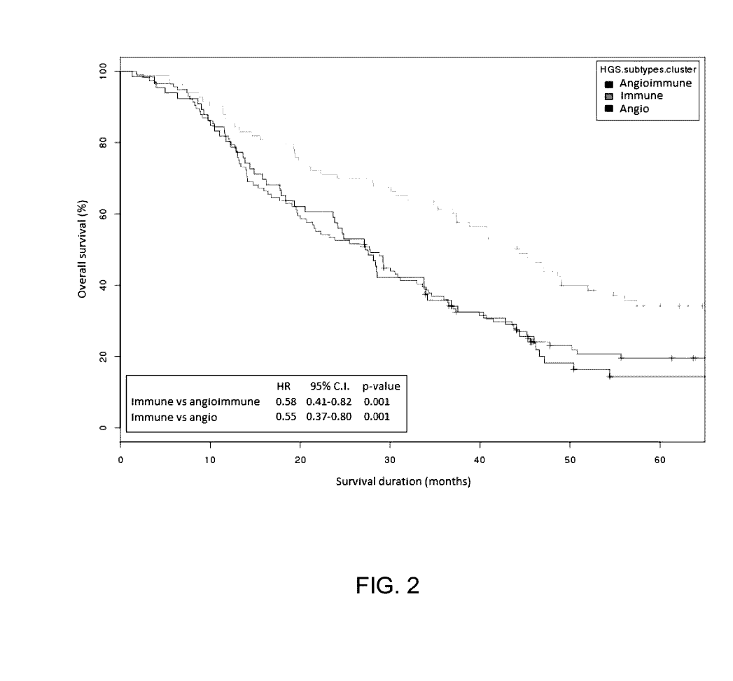

Multivariable survival analysis according to subgroup revealed that the patients in the Immune cluster had significantly prolonged OS compared to both patients in the Angioimmune (HR-0.58 [0.41-0.82], padj=0.001) and Angio clusters (HR-0.55 [0.37−0.80], padj=0.001). Kaplan-Meier curves are shown in FIG. 2 (univariable HR and p-values are shown).

Since patients in the Immune cluster had a significantly better outcome than those in the other clusters we proceeded to develop an assay to prospectively identify these patients in the clinic. In addition, given the low expression of angiogenic genes in the immune cluster, we hypothesized that this assay may identify a population that would not benefit from therapies targeting angiogenesis, although it would require additional datasets to test this theory. For the purpose of signature generation the Angio and Angioimmune clusters were grouped together and labeled as the “pro-angiogenic” group.

### Example 2: Determining the Minimum Number of Core Genes Required to Identify the Subtype

### Methods

The core set of genes to define the “Immune” subtype comprise 161 angiogenesis related probesets and 77 immune related probesets. The general pattern of expression to define the subtype is up-regulation of immune probesets and down-regulation of angiogenesis probesets.

Scoring Method for Predicting the Immune Subtype

A scoring method was derived to enable classification of patients into one of either the Immune or Pro-Angiogenic subtypes. The scoring method is based on the following, using the 265 high grade serous (HGS) samples that were used to discovery the subtype:
Median centre the probeset expression of the RMA (Robust Multi Array) pre-processed data.
To score each sample, calculate the average expression of the 161 angiogenesis probesets subtract from the average expression of the 77 immune probesets.
A score of 0 is used to dichotomise samples into either Immune (greater than 0) or Pro-angiogenic (less than 0).

Minimum Number of Genes Required

The ratio of Immune:Angiogenesis probesets is approximately 2:1, therefore in evaluating the minimum number of probesets required to classify samples into the Immune or Pro-angiogenic subtype, it is assumed that a 2:1 ratio should be maintained.

The minimum number of features considered were 3 (2 angio and 1 immune) increasing by three at each iteration up to 228 (maintaining the 2:1 ratio). At each feature length 1000 random samplings of the probesets was performed, and the 265 HGS samples were scored by the signature as described above.

The performance of the signatures was measured by the following:
The discrimination between the Immune and Pro-angiogenic groups based on the signature scores in the 265 HGS samples, measured using area under the receiver operator characteristic curve (AUC)
The Concordance-index (C-index) in the ICON7 clinical trial control arm samples, measuring the discrimination of overall survival (OS)
The hazard ratio of the treatment effect on OS in the Immune group, as predicted by the signature

Results

Scoring Method for Predicting the Immune Subtype

The scoring method applied to all samples using all core probesets resulted in an AUC performance against the subtype endpoint of 0.89 [0.85−0.93].

Minimum Number of Genes Required

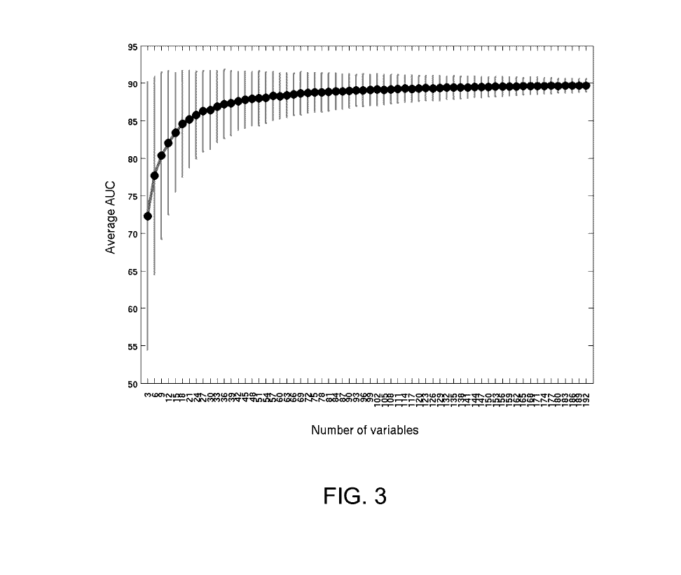

FIG. 3 shows the AUC performance for predicting the subtype using a minimum of 3 probesets up to 228 probesets, where the 2:1 ratio of angiogenesis to immune probesets was maintained across all signatures. At a minimum of 3 probesets, the AUC performance is still significantly greater than 0.5 suggesting that with the use of a minimum of 2 angiogenesis probesets and 1 immune probeset, it is possible to predict the molecular subgroup significantly better than by chance.

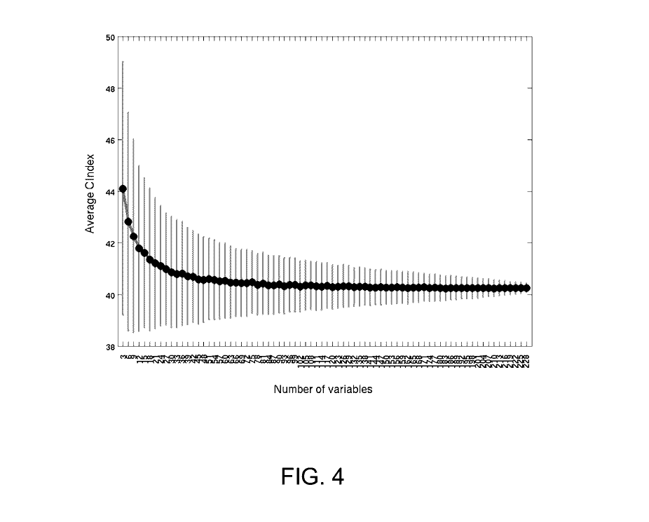

FIG. 4 shows the C-index performance at a range of feature lengths in the ICON7 control samples measured against OS. A C-index that is significantly less than 0.5 is reflective of a survival advantage in patients with higher scores over those with lower scores. The results in FIG. 4 show that with a minimum of 2 angiogenesis probesets and 1 immune probeset the C-index is significantly lower than 0.5, therefore the survival differences in the control arm are evident with a minimum of 3 probesets.

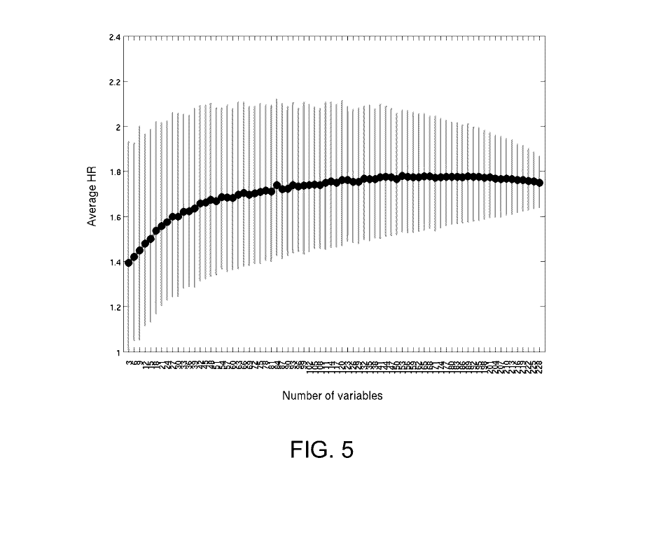

FIG. 5 shows the HR of the treatment effect on OS in the immune group as predicted by the signatures at each feature length. A HR greater than 1.0 is reflective of a survival disadvantage in patients who received the treatment in addition to standard of care. With a minimum of 3 probesets the survival differences are evident between the treated with Avastin and control arm, with a HR significantly greater than 1.0.

### Example Signature 1: Immune 63 Gene Signature

### Samples

Internal training samples: This sample set comprised of 193 High Grade Serous Ovarian samples retrieved from the Edinburgh Ovarian Cancer Database
Tothill samples: This is a publically available dataset, from which 152 High Grade Serous Ovarian samples were used for analysis
ICON7 samples: This sample set comprises of 284 High Grade Serous samples from a phase III randomized trial of carboplatin and paclitaxel with or without bevacizumab first line cancer treatment which were accessed through the MRC (Medical Research Council).
   ICON7 SOC (Standard of Care)—140 samples— refers to patients who did not receive the addition of bevacizumab
   ICON7 Immune group—116 samples: this refers to the ICON7 samples predicted in the Immune group by the Immune 63 gene signature
   ICON7 ProAngio group—168 samples: this refers to the ICON7 samples predicted in the ProAngiogenesis group by the Immune 63 gene signature

Methods:

Signature Development

A balanced sample set of 193 Ovarian HGS samples were used to develop the signature using the PLS¹⁹ (Partial Least Squares) method during 10 repeats of 5-fold cross validation (CV). The following steps were used within signature development:
Probesets mapped to genes and gene expression measured using the log₂ transformation of the median probeset expression for each gene
Within nested CV, quantile normalization was performed following a pre-filtering to remove 75% of genes with low variance, low intensity, and high correlation to cDNA yield
Genes/features were ranking based on correlation adjusted t-scores² and feature reduction involved discarding 10% of the least important genes until 5 genes remained
The 63 gene signature was identified as the feature set for which the hazard ratio (HR) predicting Progression free survival (PFS) under cross-validation was optimal

The following datasets have been evaluated within CV to determine the performance of the 63 gene signature:
Internal training set—193 samples
ICON7 SOC (Standard of Care)—140 samples
ICON7 Immune group—116 samples
ICON7 ProAngio group—168 samples

Core Gene Analysis

The purpose of evaluating the core gene set of the signature is to determine a ranking for the genes based upon their impact on performance when removed from the signature.

This analysis involved 1,000,000 random samplings of 10 signature genes from the original 63 signature gene set. At each iteration, 10 randomly selected signature genes were removed and the performance of the remaining 53 genes was evaluated using the PFS endpoint to determine the impact on HR performance when these 10 genes were removed in the following 3 datasets:
Internal Validation—72 samples
Tothill HGS²¹ (High Grade Serous)—152 samples
ICON7 SOC (Standard of Care)—140 samples

Within each of these 3 datasets, the signature genes were weighted based upon the change in HR performance (Delta HR) based upon their inclusion or exclusion. Genes ranked ‘1’ have the most negative impact on performance when removed and those ranked ‘63’ have the least impact on performance when removed.

Minimum Gene Analysis

The purpose of evaluating the minimum number of genes is to determine if significant performance can be achieved within smaller subsets of the original signature.

This analysis involved 10,000 random samplings of the 63 signature genes starting at 1 gene/feature, up to a maximum of 25 genes/features. For each randomly selected feature length, the signature was redeveloped using the PLS machine learning method under CV and model parameters derived. At each feature length, all randomly selected signatures were applied to calculate signature scores for the following 3 datasets:
Tothill³ HGS (High Grade Serous)—152 samples
ICON7 SOC (Standard of Care)—140 samples
ICON7 Immune group—116 samples

Continuous signature scores were evaluated with PFS (Progression Free Survival) to determine the HR (Hazard Ratio) effect. The HR for all random signatures at each feature length was summarized and figures generated to visualize the performance over CV.

Results

Signature Development

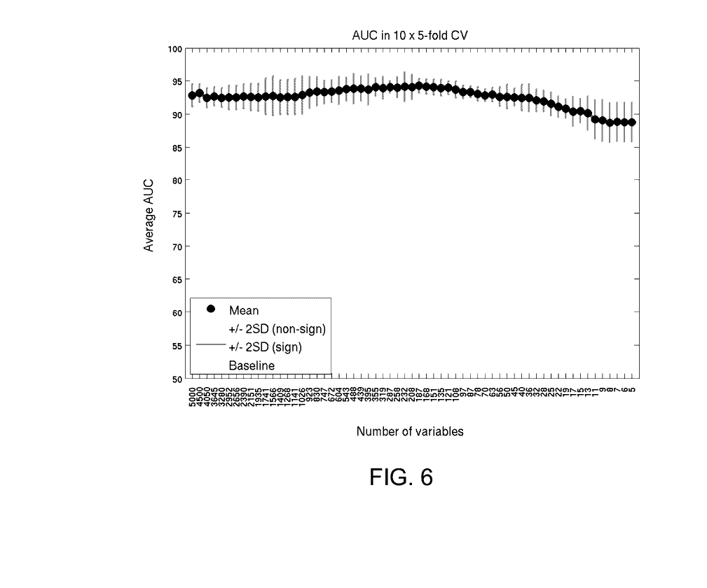

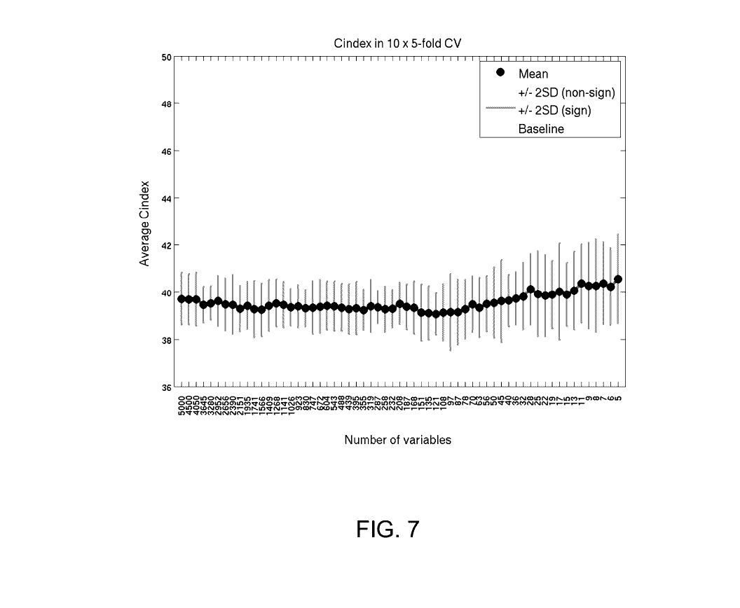

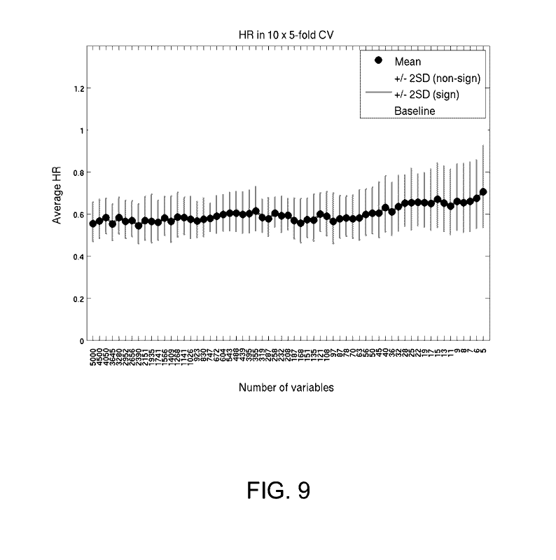

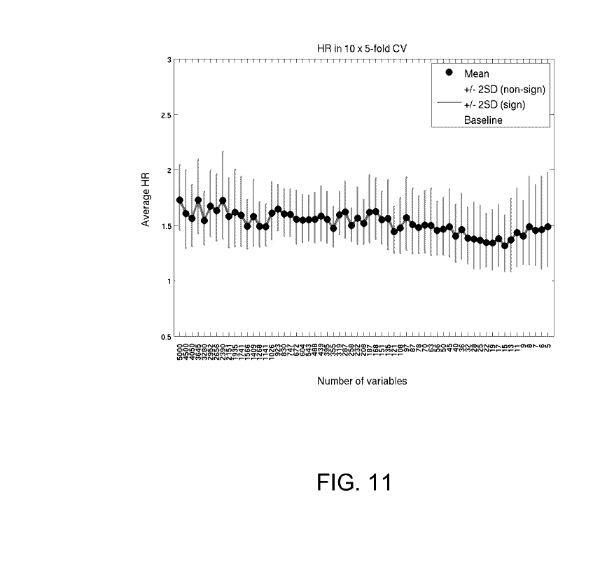

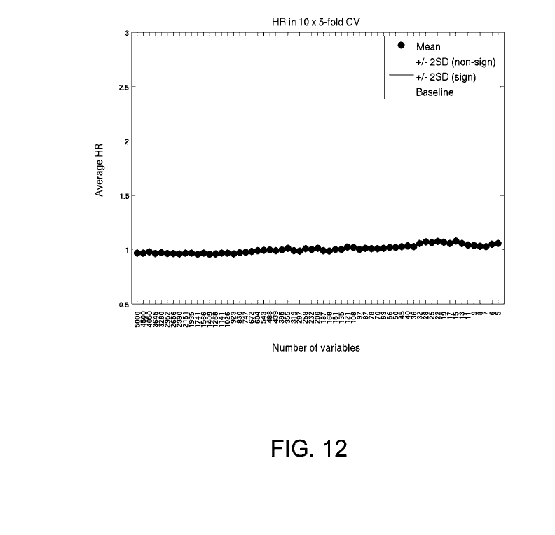

This section presents the results of signature development within CV.
Internal training set: FIGS. 6, 7 & 8 show the AUC (Area under the receiver operating curve), C-Index (Concordance Index) & HR of the training set, from which the 63 gene signature was identified.
ICON7 SOC: FIGS. 9 & 10 show the HR and C-Index of the ICON7 SOC samples under CV.
ICON7 Immune group: FIG. 11 shows the HR of the ICON7 Immune samples (as identified by the 63 gene signature) under CV.
ICON7 ProAngio group: FIG. 12 shows the HR of the ICON7 ProAngio samples (as identified by the 63 gene signature) under CV.

Core Gene Analysis

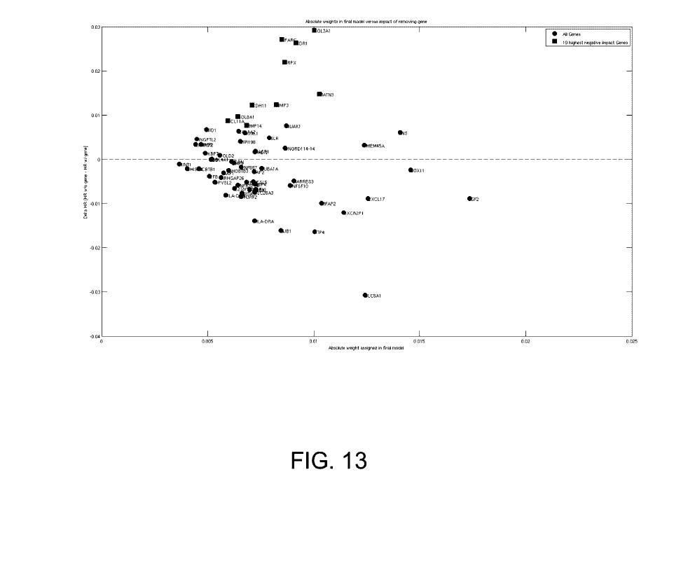

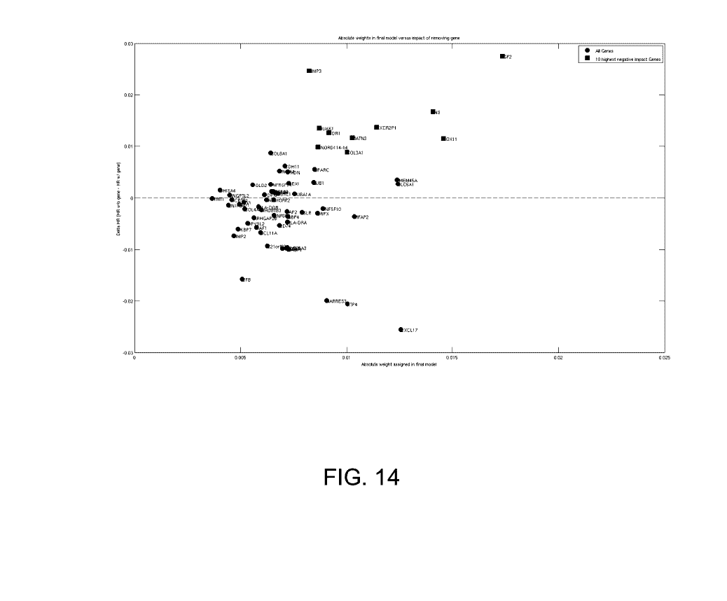

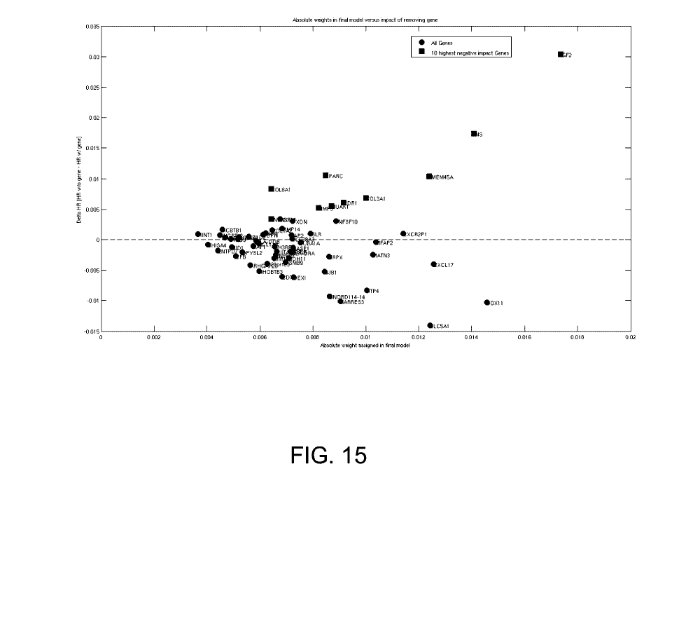

The results for the core gene analysis of the 63 gene signature in 3 datasets is provided in this section.
Internal Validation: Delta HR performance measured in this dataset for the 63 signature genes is shown in FIG. 13. This figure highlights the top 10 ranked genes in the signature which are the most important in retaining a good HR performance within this dataset.
Tothill HGS: Delta HR performance measured in this dataset for the 63 signature genes is shown in FIG. 14. This figure highlights the top 10 ranked genes in the signature which are the most important in retaining a good HR performance within this dataset.
ICON7 SOC: Delta HR performance measured in this dataset for the 63 signature genes is shown in FIG. 15. This figure highlights the top 10 ranked genes in the signature which are the most important in retaining a good HR performance within this dataset.
Delta HR across these 3 datasets was evaluated to obtain a combined gene ranking for each of the signature genes. The ranks assigned to the signature genes based on the core set analysis have been outlined in Immune63GeneSig_CoreGenes_HR.txt.

Minimum Gene Analysis

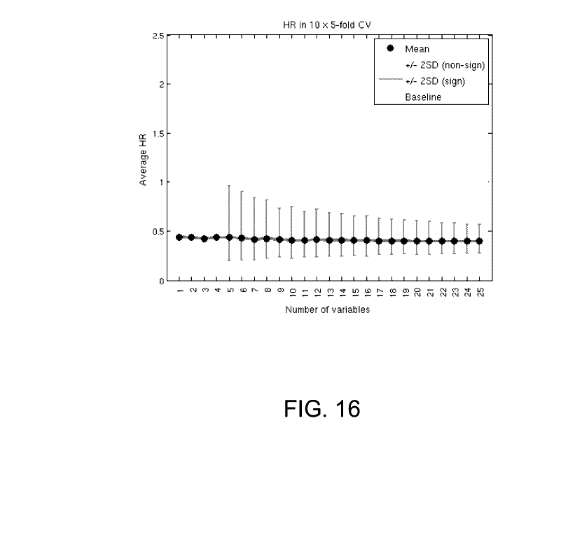

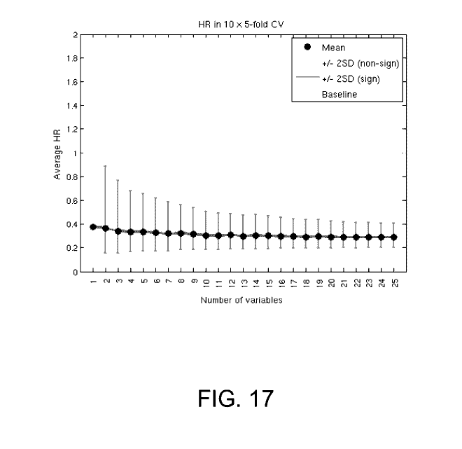

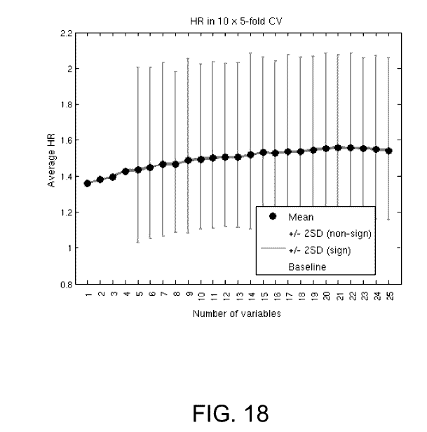

The results for the minimum gene analysis of the 63 gene signature in 3 datasets is provided in this section.
Tothill HGS: The average HR performance measured in this dataset using the random sampling of the signature genes from a feature length of 1.25 is shown in FIG. 16. This figure shows that to retain a significant HR performance (i.e. HR<1) a minimum of 5 of the signature genes must be selected.
ICON7 SOC: The average HR performance measured in this dataset using the random sampling of the signature genes from a feature length of 1.25 is shown in FIG. 17. This figure shows that to retain a significant HR performance (i.e. HR<1) a minimum of 2 of the signature genes must be selected.
ICON7 Immune: The average HR performance measured in this dataset using the random sampling of the signature genes from a feature length of 1.25 is shown in FIG. 18. This figure shows that to retain a significant HR performance (i.e. HR<1) a minimum of 5 of the signature genes must be selected.
In summary, it is recommended that a minimum of at least 5 genes can be used and significant performance will be retained.

### Example Signature 2: Immune 121 Gene Signature

### Samples

Internal training samples: This sample set comprised of 193 High Grade Serous Ovarian samples retrieved from the Edinburgh Ovarian Cancer Database
Tothill samples: This is a publically available dataset, from which 152 High Grade Serous Ovarian samples were used for analysis
ICON7 samples: This sample set comprises of 284 High Grade Serous samples from a phase III randomized trial of carboplatin and paclitaxel with or without bevacizumab first line cancer treatment which were accessed through the MRC (Medical Research Council).
   ICON7 SOC (Standard of Care)—140 samples—refers to patients who did not receive the addition of bevacizumab
   ICON7 Immune group—116 samples: this refers to the ICON7 samples predicted in the Immune group by the Immune 63 gene signature
   ICON7 ProAngio group—168 samples: this refers to the ICON7 samples predicted in the ProAngiogenesis group by the Immune 63 gene signature

Methods:

Signature Development

A balanced sample set of 193 Ovarian HGS samples were used to develop the signature using the PLS¹⁹ (Partial Least Squares) method during 10 repeats of 5-fold cross validation (CV). The following steps were used within signature development:
Probesets mapped to genes and gene expression measured using the log₂ transformation of the median probeset expression for each gene
The Immune 63 signature genes (Example signature 1) were removed from the full set of genes
Within nested CV, quantile normalization was performed following a pre-filtering to remove 75% of genes with low variance, low intensity, and high correlation to cDNA yield
Genes/features were ranking based on correlation adjusted t-scores² and feature reduction involved discarding 10% of the least important genes until 5 genes remained
The 121 gene signature was identified as the smallest feature set for which AUC & C-Index (Concordance Index) for the Progression free survival (PFS) endpoint under cross-validation were optimal.

The following datasets have been evaluated within CV to determine the performance of the 121 gene signature:
Internal training set—193 samples
ICON7 SOC (Standard of Care)—140 samples
ICON7 Immune group—116 samples
ICON7 ProAngio group—168 samples

Core Gene Analysis

The purpose of evaluating the core gene set of the signature is to determine a ranking for the genes based upon their impact on performance when removed from the signature.

This analysis involved 1,000,000 random samplings of 10 signature genes from the original 121 signature gene set. At each iteration, 10 randomly selected signature genes were removed and the performance of the remaining 111 genes was evaluated using the PFS endpoint to determine the impact on HR performance when these 10 genes were removed in the following 3 datasets:
Internal Validation—72 samples
Tothill²¹ HGS (High Grade Serous)—152 samples
ICON7 SOC (Standard of Care)—140 samples

Within each of these 3 datasets, the signature genes were weighted based upon the change in HR performance (Delta HR) based upon their inclusion or exclusion. Genes ranked ‘1’ have the most negative impact on performance when removed and those ranked ‘121’ have the least impact on performance when removed.

Minimum Gene Analysis

The purpose of evaluating the minimum number of genes is to determine if significant performance can be achieved within smaller subsets of the original signature.

This analysis involved 10,000 random samplings of the 121 signature genes starting at 1 gene/feature, up to a maximum of 25 genes/features. For each randomly selected feature length, the signature was redeveloped using the PLS machine learning method under CV and model parameters derived. At each feature length, all randomly selected signatures were applied to calculate signature scores for the following 3 datasets:
Tothill²¹ HGS (High Grade Serous)—152 samples
ICON7 SOC (Standard of Care)—140 samples
ICON7 Immune group—116 samples

Continuous signature scores were evaluated with PFS (Progression Free Survival) to determine the HR (Hazard Ratio) effect. The HR for all random signatures at each feature length was summarized and figures generated to visualize the performance over CV.

Results

Signature Development

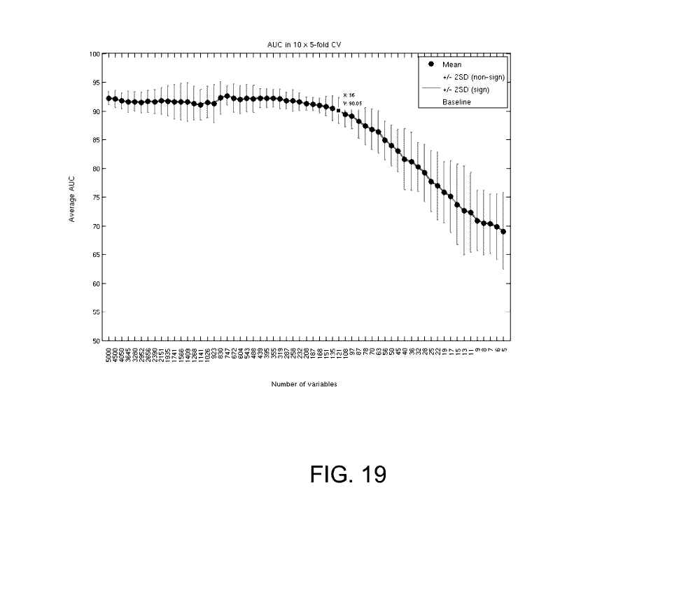

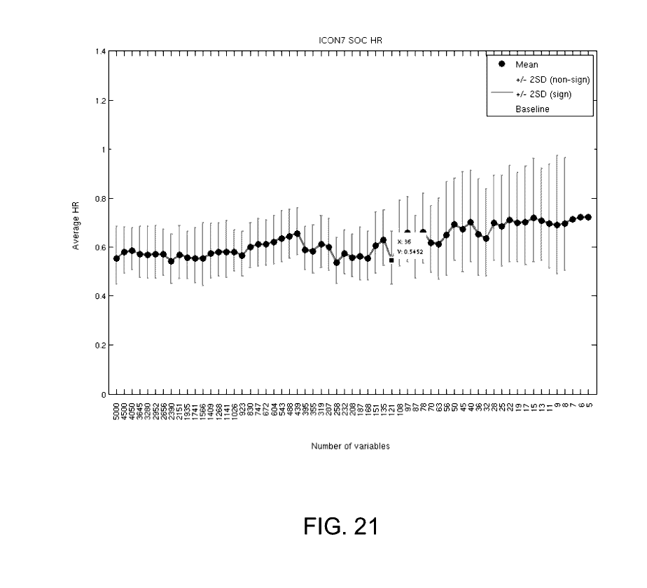

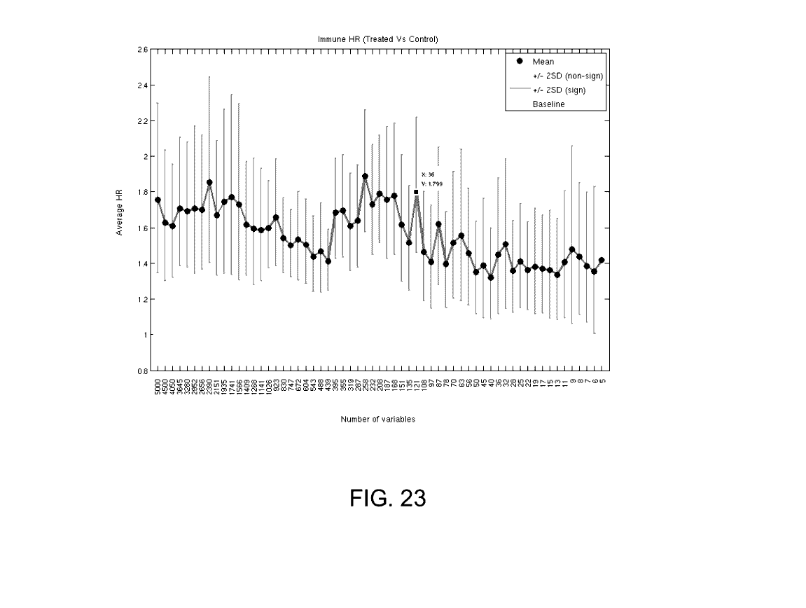

This section presents the results of signature development within CV.
Internal training set: FIGS. 19 & 20 show the AUC (Area under the receiver operating curve), C-Index for the training set, from which the 121 gene signature was identified.
ICON7 SOC: FIGS. 21 & 22 show the HR and C-Index of the ICON7 SOC samples under CV.
ICON7 Immune group: FIG. 23 shows the HR of the ICON7 Immune samples (Immune samples identified by the 63 gene signature) under CV.

Core Gene Analysis

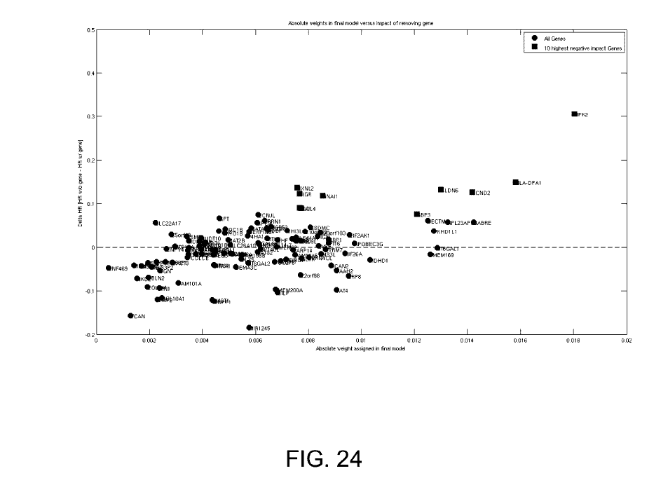

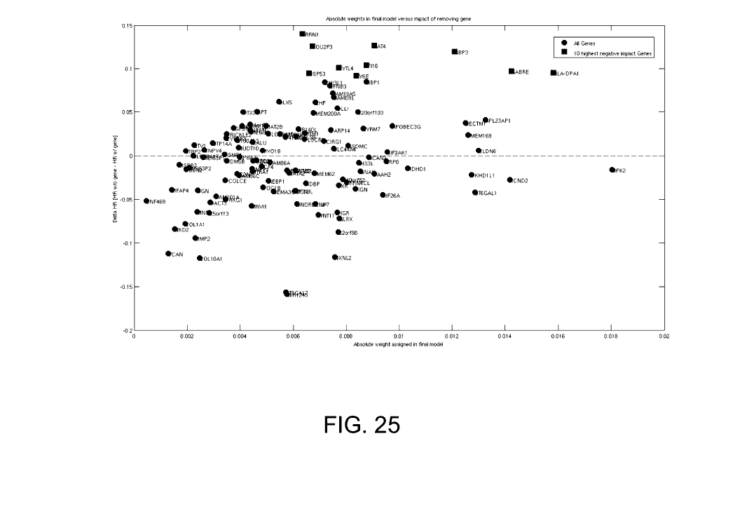

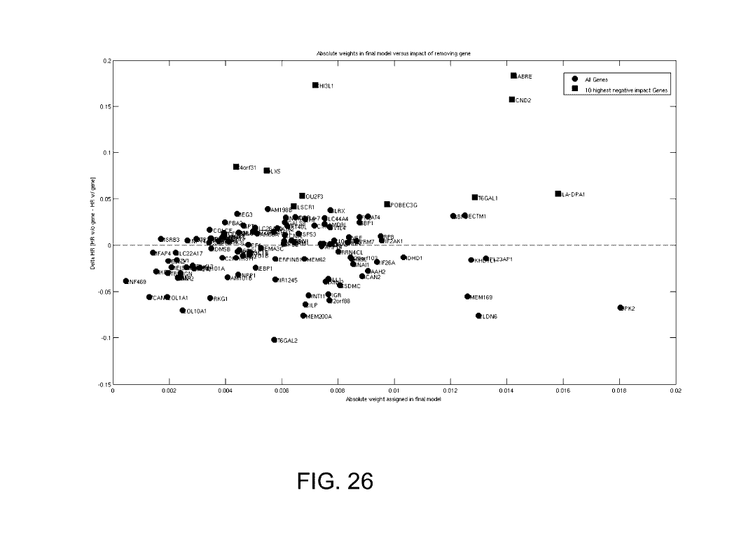

The results for the core gene analysis of the 121 gene signature in 3 datasets are provided in this section.
Internal Validation: Delta HR performance measured in this dataset for the 121 signature genes is shown in FIG. 24. This figure highlights the top 10 ranked genes in the signature which are the most important in retaining a good HR performance within this dataset.
Tothill HGS: Delta HR performance measured in this dataset for the 121 signature genes is shown in FIG. 25. This figure highlights the top 10 ranked genes in the signature which are the most important in retaining a good HR performance within this dataset.
ICON7 SOC: Delta HR performance measured in this dataset for the 121 signature genes is shown in FIG. 26. This figure highlights the top 10 ranked genes in the signature which are the most important in retaining a good HR performance within this dataset.
Delta HR across these 3 datasets was evaluated to obtain a combined gene ranking for each of the signature genes. The ranks assigned to the signature genes based on the core set analysis have been outlined in Immune121GeneSig_CoreGenes_HR.txt.

Minimum Gene Analysis

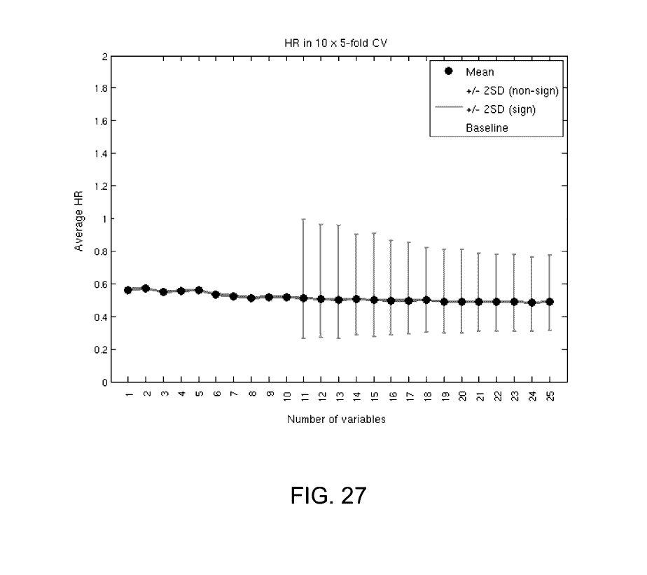

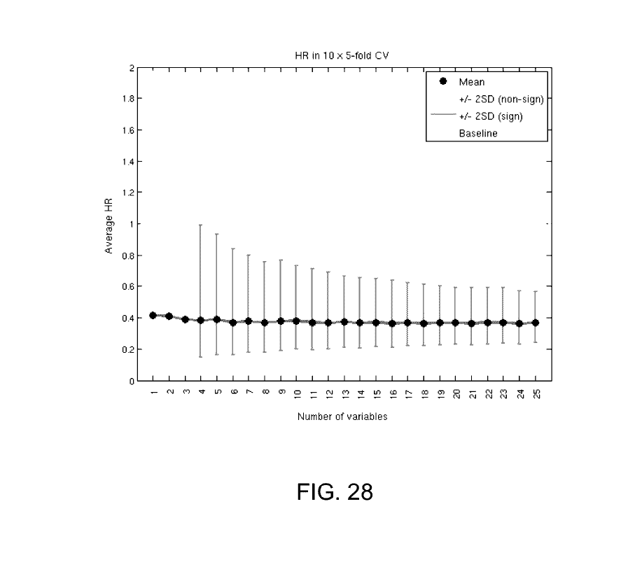

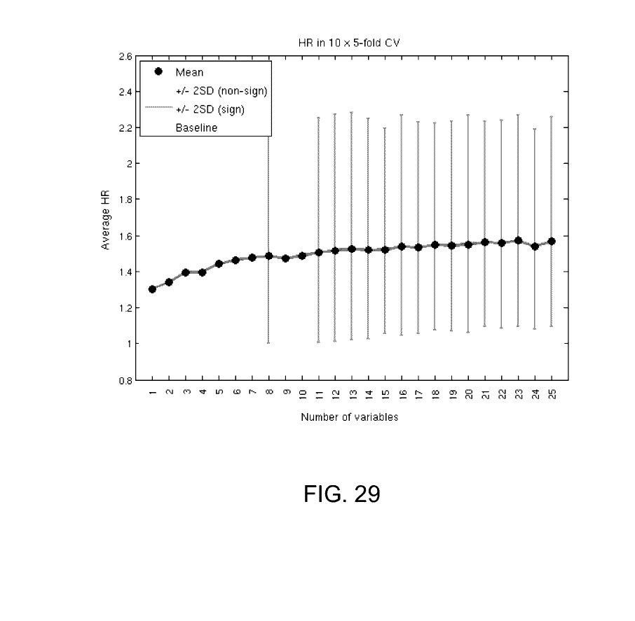

The results for the minimum gene analysis of the 121 gene signature in 3 datasets are provided in this section.
Tothill HGS: The average HR performance measured in this dataset using the random sampling of the signature genes from a feature length of 1.25 is shown in FIG. 27. This figure shows that to retain a significant HR performance (i.e. HR<1) a minimum of 11 of the signature genes must be selected.
ICON7 SOC: The average HR performance measured in this dataset using the random sampling of the signature genes from a feature length of 1.25 is shown in FIG. 28. This figure shows that to retain a significant HR performance (i.e. HR<1) a minimum of 4 of the signature genes must be selected.
ICON7 Immune: The average HR performance measured in this dataset using the random sampling of the signature genes from a feature length of 1.25 is shown in FIG. 29. This figure shows that to retain a significant HR performance (i.e. HR<1) a minimum of 11 of the signature genes must be selected.
In summary, it is recommended that a minimum of at least 11 genes can be used and significant performance will be retained.

### Example Signature 3: Immune 232 Gene Signature

### Samples

Internal training samples: This sample set comprised of 193 High Grade Serous Ovarian samples retrieved from the Edinburgh Ovarian Cancer Database
Tothill samples: This is a publically available dataset, from which 152 High Grade Serous Ovarian samples were used for analysis
ICON7 samples: This sample set comprises of 284 High Grade Serous samples from a phase III randomized trial of carboplatin and paclitaxel with or without bevacizumab first line cancer treatment which were accessed through the MRC (Medical Research Council).
   ICON7 SOC (Standard of Care)—140 samples—refers to patients who did not receive the addition of bevacizumab
   ICON7 Immune group—116 samples: this refers to the ICON7 samples predicted in the Immune group by the Immune 63 gene signature
   ICON7 ProAngio group—168 samples: this refers to the ICON7 samples predicted in the ProAngiogenesis group by the Immune 63 gene signature

Methods:

Signature Development

A balanced sample set of 193 Ovarian HGS samples were used to develop the signature using the PLS¹⁹ (Partial Least Squares) method during 10 repeats of 5-fold cross validation (CV). The following steps were used within signature development:
Probesets mapped to genes and gene expression measured using the log₂ transformation of the median probeset expression for each gene
The Immune 63 (Example signature 1) & 121 (Example signature 2) signature genes were removed from the full set of genes prior to signature development
Within nested CV, quantile normalization was performed following a pre-filtering to remove 75% of genes with low variance, low intensity, and high correlation to cDNA yield
Genes/features were ranking based on correlation adjusted t-scores²⁰ and feature reduction involved discarding 10% of the least important genes until 5 genes remained
The 232 gene signature was identified as a feature set for which AUC & C-Index (Concordance Index) for the Progression free survival (PFS) endpoint under cross-validation were significant

The following datasets have been evaluated within CV to determine the performance of the 232 gene signature:
Internal training set—193 samples
ICON7 SOC (Standard of Care)—140 samples
ICON7 Immune group—116 samples
ICON7 ProAngio group—168 samples

Core Gene Analysis

The purpose of evaluating the core gene set of the signature is to determine a ranking for the genes based upon their impact on performance when removed from the signature.

This analysis involved 1,000,000 random samplings of 10 signature genes from the original 232 signature gene set. At each iteration, 10 randomly selected signature genes were removed and the performance of the remaining 222 genes was evaluated using the PFS endpoint to determine the impact on HR performance when these 10 genes were removed in the following 3 datasets:
Internal Validation—72 samples
Tothill²¹ HGS (High Grade Serous)—152 samples
ICON7 SOC (Standard of Care)—140 samples

Within each of these 3 datasets, the signature genes were weighted based upon the change in HR performance (Delta HR) based upon their inclusion or exclusion. Genes ranked ‘1’ have the most negative impact on performance when removed and those ranked ‘232’ have the least impact on performance when removed.

Minimum Gene Analysis

The purpose of evaluating the minimum number of genes is to determine if significant performance can be achieved within smaller subsets of the original signature.

This analysis involved 10,000 random samplings of the 232 signature genes starting at 1 gene/feature, up to a maximum of 25 genes/features. For each randomly selected feature length, the signature was redeveloped using the PLS machine learning method under CV and model parameters derived. At each feature length, all randomly selected signatures were applied to calculate signature scores for the following 3 datasets:
Tothill²¹ HGS (High Grade Serous)—152 samples
ICON7 SOC (Standard of Care)—140 samples
ICON7 Immune group—116 samples

Continuous signature scores were evaluated with PFS (Progression Free Survival) to determine the HR (Hazard Ratio) effect. The HR for all random signatures at each feature length was summarized and figures generated to visualize the performance over CV.

Results

Signature Development

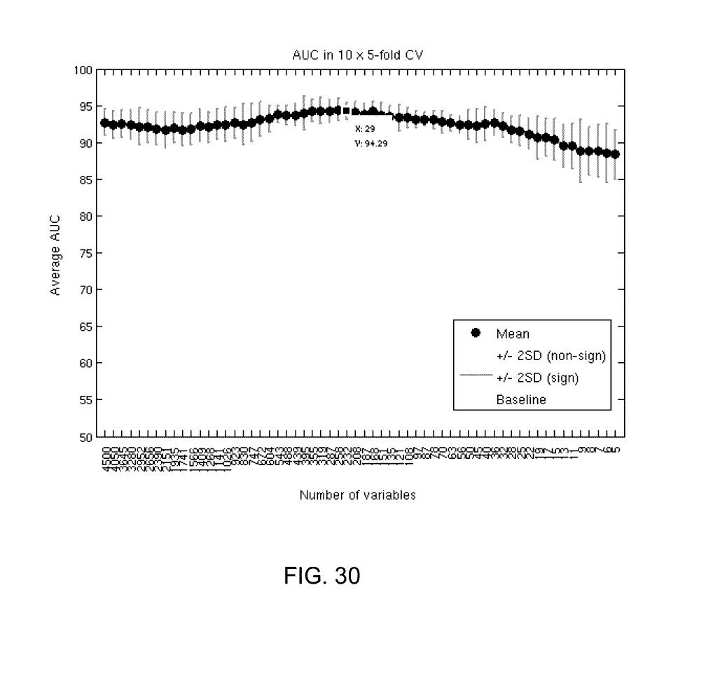

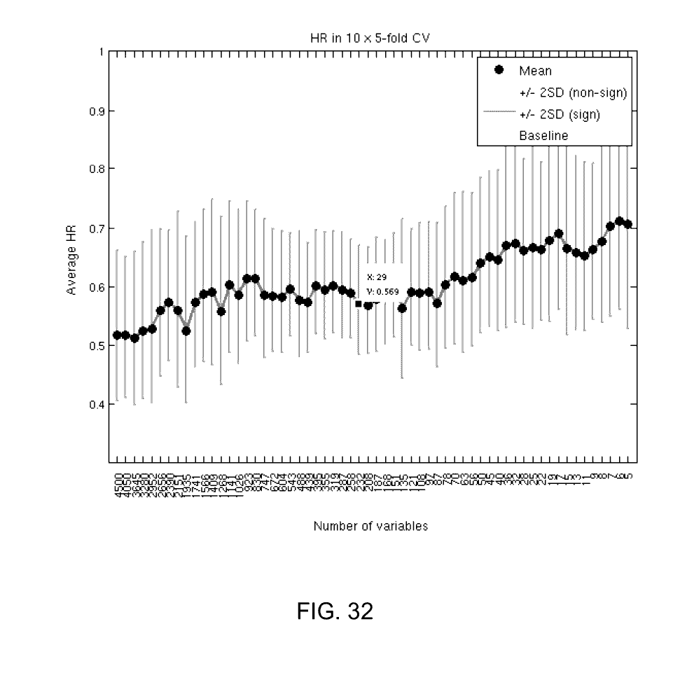

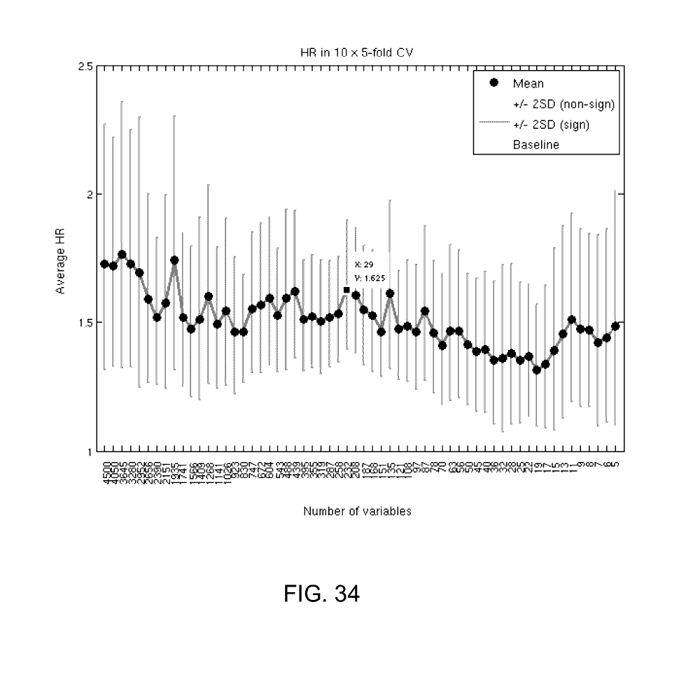

This section presents the results of signature development within CV.
Internal training set: FIGS. 30 & 31 show the AUC (Area under the receiver operating curve), C-Index for the training set, from which the 232 gene signature was identified.
ICON7 SOC: FIGS. 32 & 33 show the HR and C-Index of the ICON7 SOC samples under CV.
ICON7 Immune group: FIG. 34 shows the HR of the ICON7 Immune samples (Immune samples identified by the 63 gene signature) under CV.

Core Gene Analysis

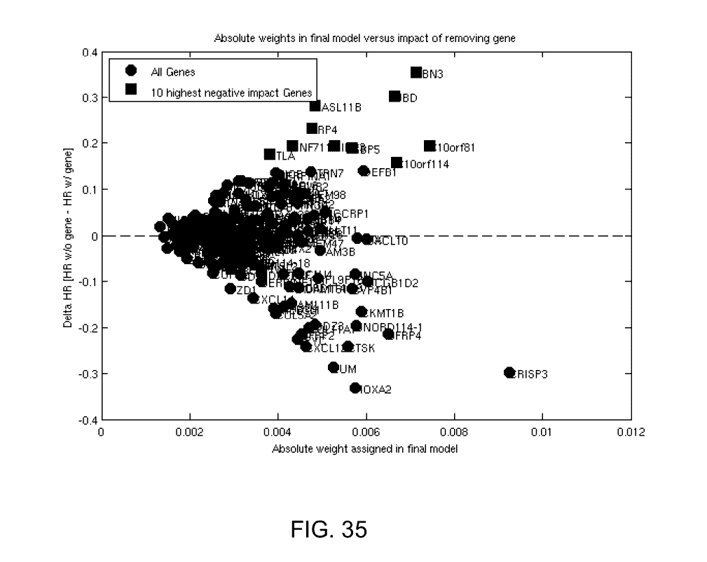

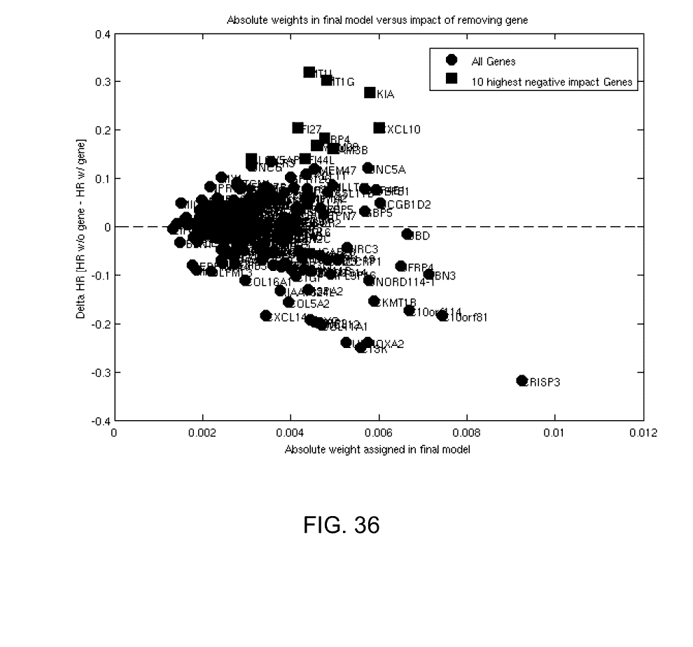

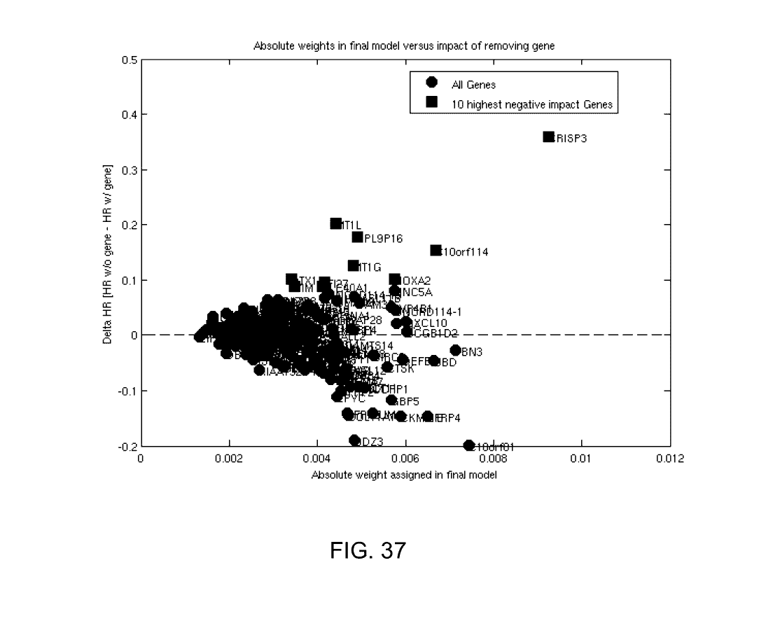

The results for the core gene analysis of the 232 gene signature in 3 datasets are provided in this section.
Internal Validation: Delta HR performance measured in this dataset for the 232 signature genes is shown in FIG. 35. This figure highlights the top 10 ranked genes in the signature which are the most important in retaining a good HR performance within this dataset.
Tothill HGS: Delta HR performance measured in this dataset for the 232 signature genes is shown in FIG. 36. This figure highlights the top 10 ranked genes in the signature which are the most important in retaining a good HR performance within this dataset.
ICON7 SOC: Delta HR performance measured in this dataset for the 232 signature genes is shown in FIG. 37. This figure highlights the top 10 ranked genes in the signature which are the most important in retaining a good HR performance within this dataset.
Delta HR across these 3 datasets was evaluated to obtain a combined gene ranking for each of the signature genes. The ranks assigned to the signature genes based on the core set analysis have been outlined in Immune232GeneSig_CoreGenes_HR.txt.

Minimum Gene Analysis

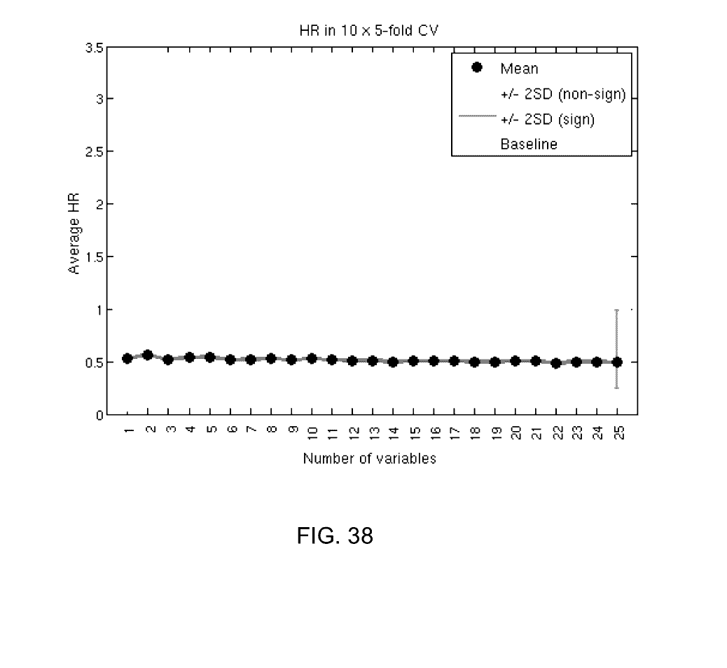

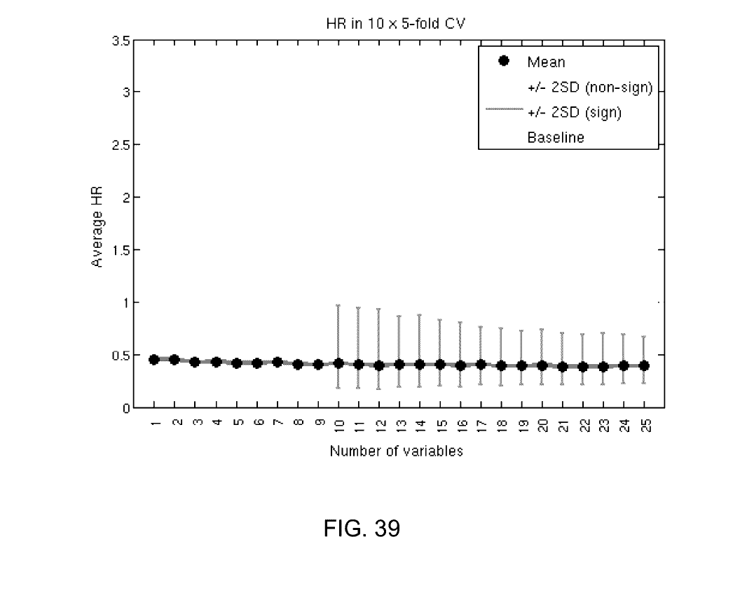

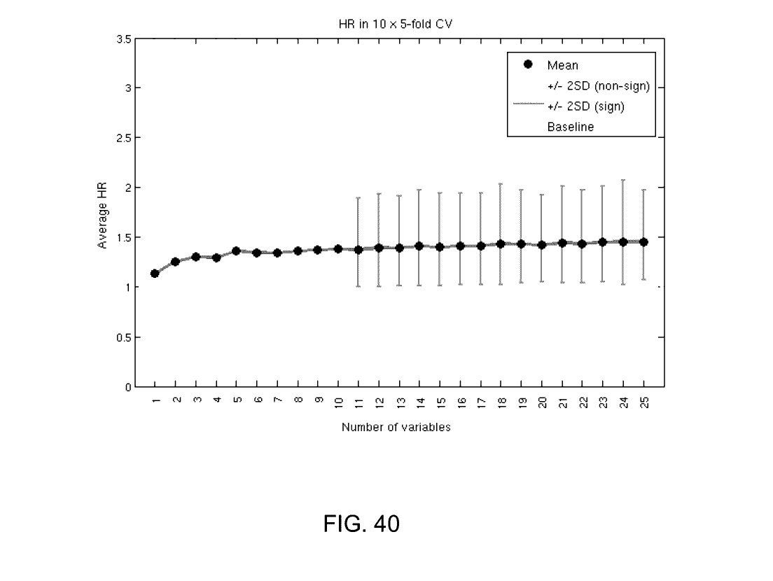

The results for the minimum gene analysis of the 232 gene signature in 3 datasets are provided in this section.
Tothill HGS: The average HR performance measured in this dataset using the random sampling of the signature genes from a feature length of 1.25 is shown in FIG. 38. This figure shows that to retain a significant HR performance (i.e. HR<1) a minimum of 25 signature genes must be selected.
ICON7 SOC: The average HR performance measured in this dataset using the random sampling of the signature genes from a feature length of 1.25 is shown in FIG. 39. This figure shows that to retain a significant HR performance (i.e. HR<1) a minimum of 10 of the signature genes must be selected.
ICON7 Immune: The average HR performance measured in this dataset using the random sampling of the signature genes from a feature length of 1.25 is shown in FIG. 40. This figure shows that to retain a significant HR performance (i.e. HR<1) a minimum of 11 of the signature genes must be selected.
In summary, it is recommended that a minimum of at least 25 genes can be used and significant performance will be retained.

### Example Signature 4: Immune 188 Gene Signature

### Samples

Internal training samples: This sample set comprised of 193 High Grade Serous Ovarian samples retrieved from the Edinburgh Ovarian Cancer Database
Tothill samples: This is a publically available dataset, from which 152 High Grade Serous Ovarian samples were used for analysis
ICON7 samples: This sample set comprises of 284 High Grade Serous samples from a phase III randomized trial of carboplatin and paclitaxel with or without bevacizumab first line cancer treatment which were accessed through the MRC (Medical Research Council).
   ICON7 SOC (Standard of Care)—140 samples—refers to patients who did not receive the addition of bevacizumab
   ICON7 Immune group—116 samples: this refers to the ICON7 samples predicted in the Immune group by the Immune 63 gene signature
   ICON7 ProAngio group—168 samples: this refers to the ICON7 samples predicted in the ProAngiogenesis group by the Immune 63 gene signature

Methods:

Signature Development

A balanced sample set of 193 Ovarian HGS samples were used to develop the signature using the SDA (Ahdesmaki, M. and Strimmer, K. (2010) Feature selection in omics prediction problems using cat scores and false non-discovery rate control Annals of applied statistics 4, 503-519) (Shrinkage Discriminate Analysis) method during 10 repeats of 5-fold cross validation (CV). The following steps were used within signature development:
Probesets mapped to genes and gene expression measured using the log₂ transformation of the median probeset expression for each gene
The Immune 63 signature genes were removed from the full set of genes prior to signature development
Within nested CV, quantile normalization was performed following a pre-filtering to remove 75% of genes with low variance, low intensity, and high correlation to cDNA yield
Recursive Feature Elimination (RFE) was used for feature reduction involved discarding the 10% of the least important genes (based upon their discriminatory ability) until 5 genes remained
The 188 gene signature was identified as a feature set for which AUC & C-Index (Concordance Index) for the Progression free survival (PFS) endpoint under cross-validation were significant

The following datasets have been evaluated within CV to determine the performance of the 188 gene signature:
Internal training set—193 samples
ICON7 SOC (Standard of Care)—140 samples
ICON7 Immune group—116 samples
ICON7 ProAngio group—168 samples

Core Gene Analysis

The purpose of evaluating the core gene set of the signature is to determine a ranking for the genes based upon their impact on performance when removed from the signature.

This analysis involved 1,000,000 random samplings of 10 signature genes from the original 188 signature gene set. At each iteration, 10 randomly selected signature genes were removed and the performance of the remaining 178 genes was evaluated using the PFS endpoint to determine the impact on HR performance when these 10 genes were removed in the following 3 datasets:
Internal Validation—72 samples
Tothill (Tothill R W, Tinker A V, George J, et al. Novel molecular subtypes of serous and endometrioid ovarian cancer linked to clinical outcome. Clin Cancer Res 2008; 14:5198-208) HGS (High Grade Serous)—152 samples
ICON7 SOC (Standard of Care)—140 samples

Within each of these 3 datasets, the signature genes were weighted based upon the change in HR performance (Delta HR) based upon their inclusion or exclusion. Genes ranked ‘1’ have the most negative impact on performance when removed and those ranked ‘188’ have the least impact on performance when removed.

Minimum Gene Analysis

The purpose of evaluating the minimum number of genes is to determine if significant performance can be achieved within smaller subsets of the original signature.

This analysis involved 10,000 random samplings of the 188 signature genes starting at 1 gene/feature, up to a maximum of 25 (or 35 in the case of Tothill dataset) genes/features. For each randomly selected feature length, the signature was redeveloped using the SDA machine learning method under CV and model parameters derived. At each feature length, all randomly selected signatures were applied to calculate signature scores for the following 3 datasets:
Tothill (Tothill R W, Tinker A V, George J, et al. Novel molecular subtypes of serous and endometrioid ovarian cancer linked to clinical outcome. Clin Cancer Res 2008; 14:5198-208) HGS (High Grade Serous)—152 samples
ICON7 SOC (Standard of Care)—140 samples
ICON7 Immune group—116 samples

Continuous signature scores were evaluated with PFS (Progression Free Survival) to determine the HR (Hazard Ratio) effect. The HR for all random signatures at each feature length was summarized and figures generated to visualize the performance over CV.

Results

Signature Development

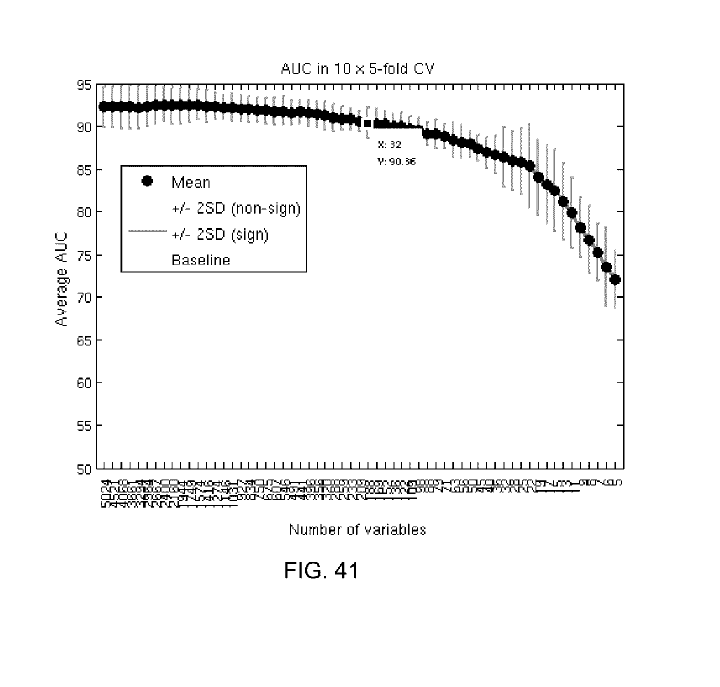

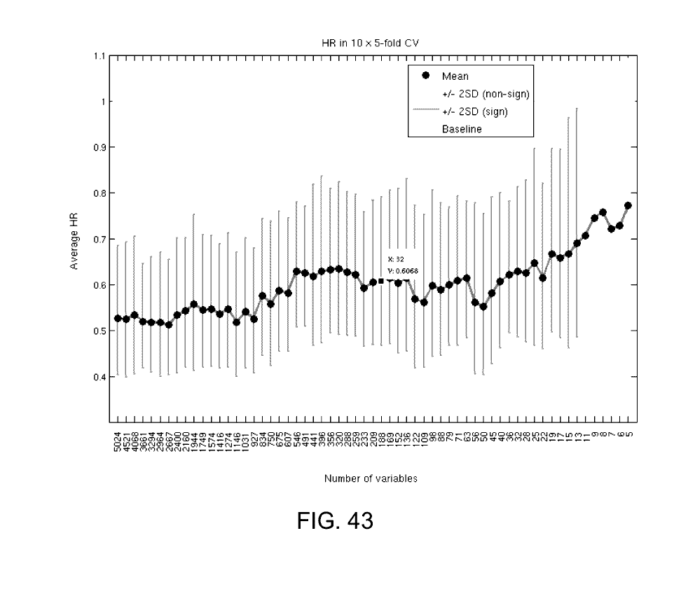

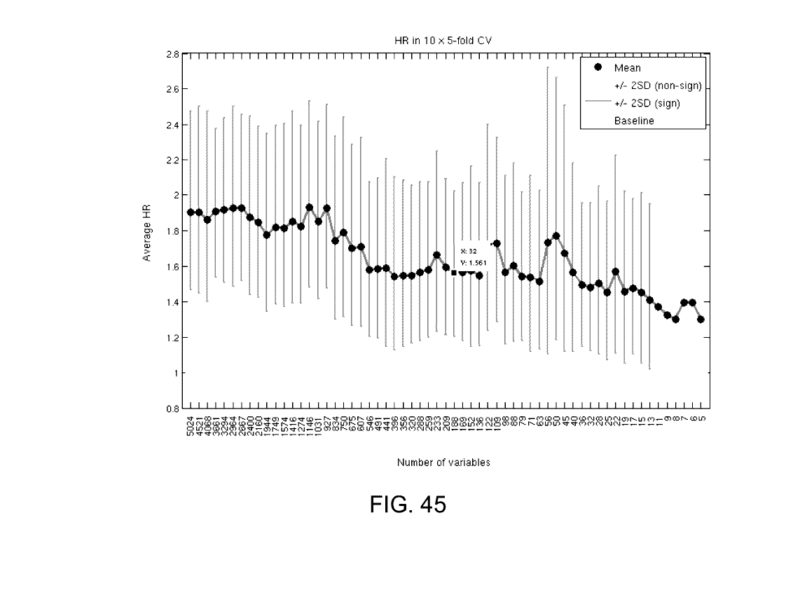

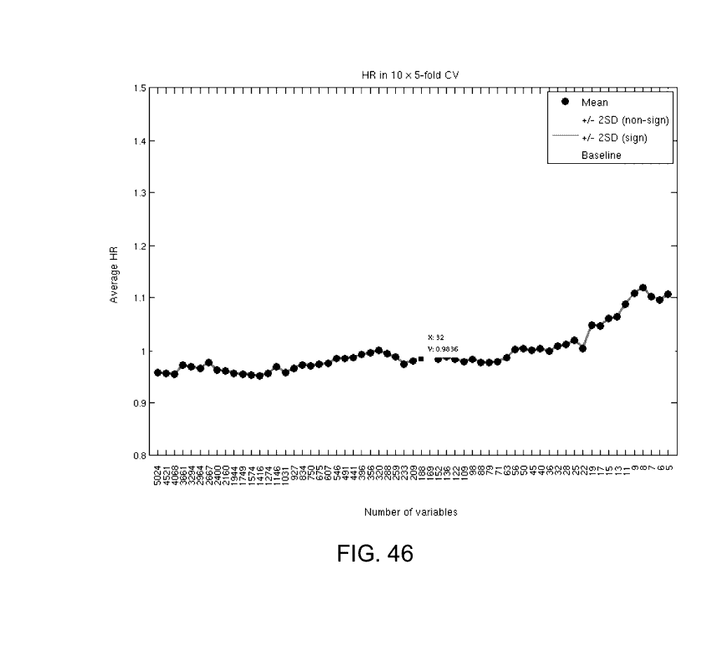

This section presents the results of signature development within CV.
Internal training set: FIGS. 41 & 42 show the AUC (Area under the receiver operating curve), C-Index for the training set, from which the 188 gene signature was identified.
ICON7 SOC: FIGS. 43 & 44 show the HR and C-Index of the ICON7 SOC samples under CV.
ICON7 Immune group: FIG. 45 shows the HR of the ICON7 Immune samples (Immune samples identified by the 63 gene signature) under CV.
ICON7 ProAngio group: FIG. 46 shows the HR of the ICON7 ProAngio samples (ProAngio samples identified by the 63 gene signature) under CV.

Core Gene Analysis

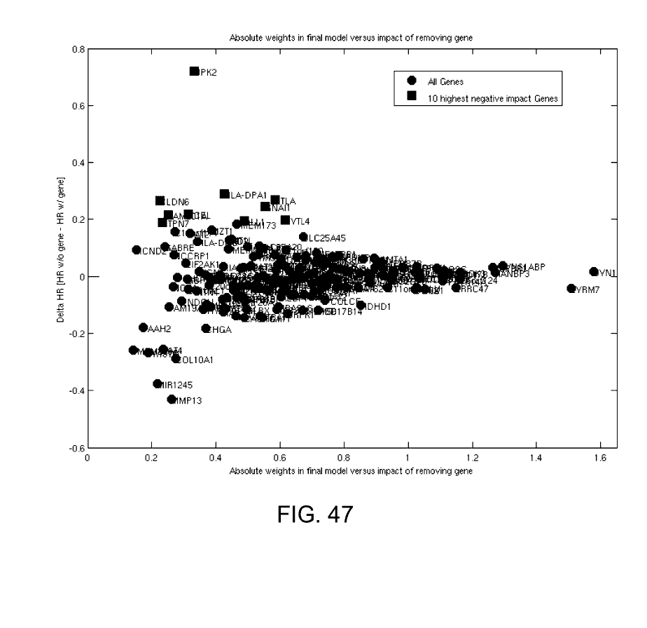

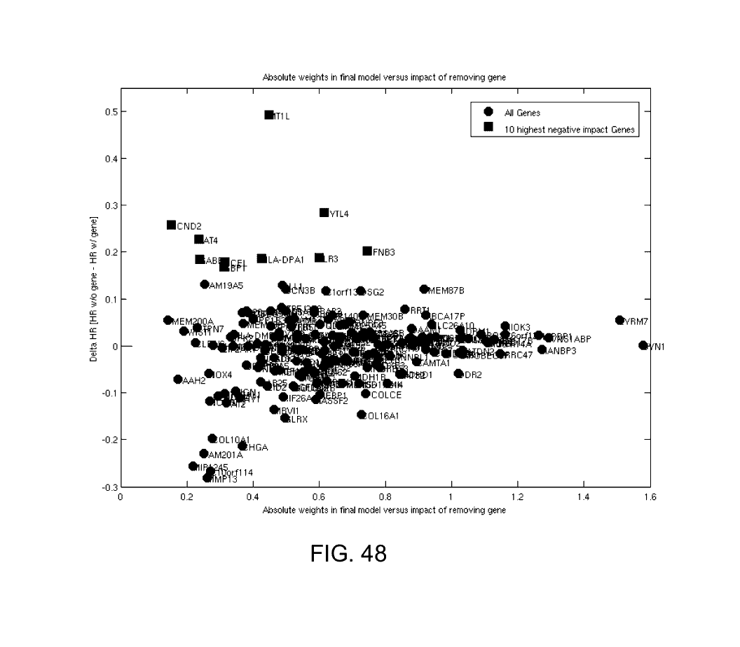

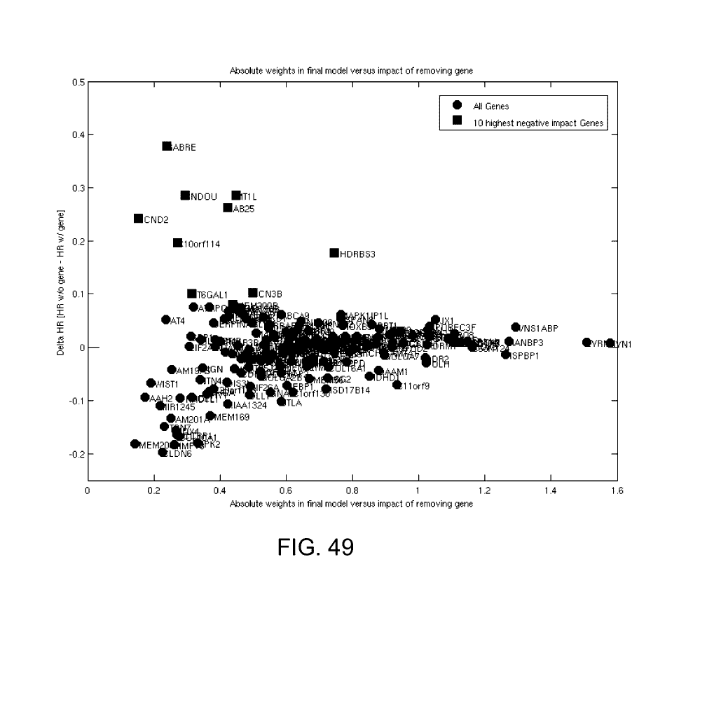

The results for the core gene analysis of the 188 gene signature in 3 datasets is provided in this section.
Internal Validation: Delta HR performance measured in this dataset for the 188 signature genes is shown in FIG. 47. This figure highlights the top 10 ranked genes in the signature which are the most important in retaining a good HR performance within this dataset.
Tothill HGS: Delta HR performance measured in this dataset for the 188 signature genes is shown in FIG. 48. This figure highlights the top 10 ranked genes in the signature which are the most important in retaining a good HR performance within this dataset.
ICON7 SOC: Delta HR performance measured in this dataset for the 188 signature genes is shown in FIG. 49. This figure highlights the top 10 ranked genes in the signature which are the most important in retaining a good HR performance within this dataset.
Delta HR across these 3 datasets was evaluated to obtain a combined gene ranking for each of the signature genes. The ranks assigned to the signature genes based on the core set analysis has been outlined in Immune188GeneSig_CoreGenes_HR.txt.

Minimum Gene Analysis

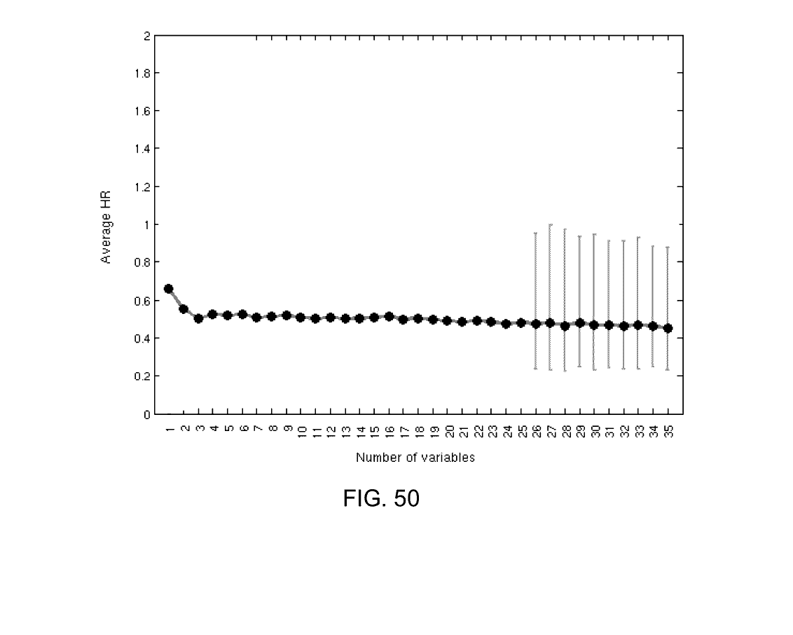

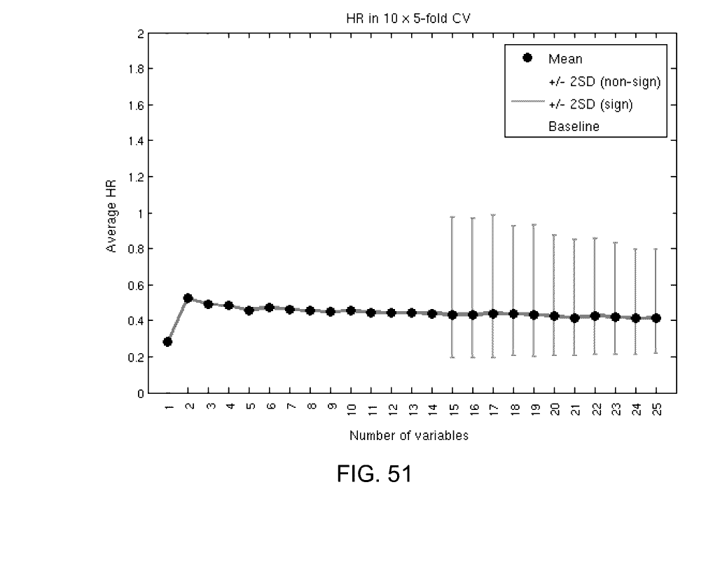

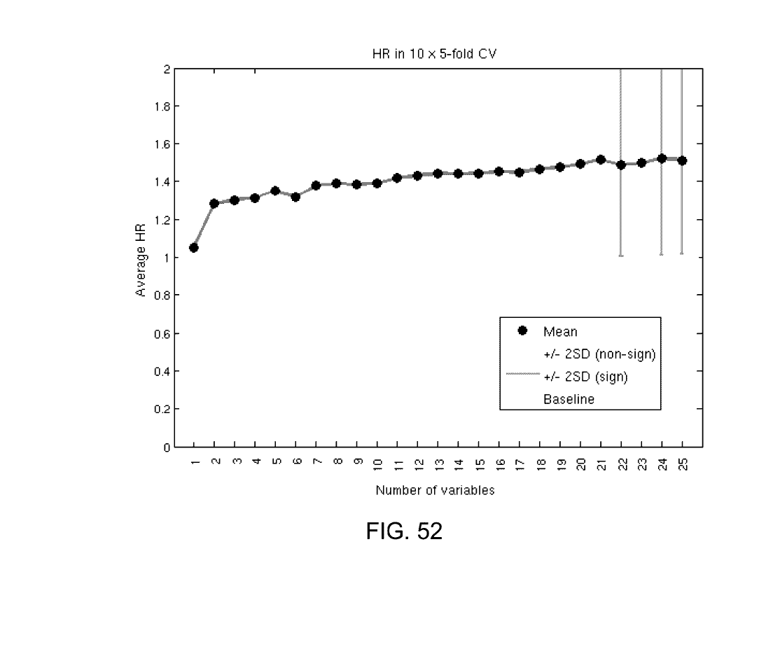

The results for the minimum gene analysis of the 188 gene signature in 3 datasets is provided in this section.
Tothill HGS: The average HR performance measured in this dataset using the random sampling of the signature genes from a feature length of 1.35 is shown in FIG. 50. This figure shows that to retain a significant HR performance (i.e. HR<1) a minimum of 26 signature genes must be selected.
ICON7 SOC: The average HR performance measured in this dataset using the random sampling of the signature genes from a feature length of 1.25 is shown in FIG. 51. This figure shows that to retain a significant HR performance (i.e. HR<1) a minimum of 15 of the signature genes must be selected.
ICON7 Immune: The average HR performance measured in this dataset using the random sampling of the signature genes from a feature length of 1.25 is shown in FIG. 52. This figure shows that to retain a significant HR performance (i.e. HR<1) a minimum of 24 of the signature genes must be selected.
In summary, it is recommended that a minimum of at least 26 genes can be used and significant performance will be retained.

### REFERENCES

1. Friedman H S, Prados M D, Wen P Y, et al. Bevacizumab alone and in combination with irinotecan in recurrent glioblastoma. J Clin Oncol, 27.4733-40 (2009).
2. Hurwitz H, Fehrenbacher L, Novotny W, et al. Bevacizumab plus irinotecan, fluorouracil, and leucovorin for metastatic colorectal cancer. N Engl J Med, 3502335-42 (2004).
3. Rini B I, Halabi S, Rosenberg J E, et al. Bevacizumab plus interferon alfa compared with interferon alfa monotherapy in patients with metastatic renal cell carcinoma: CALGB 90206. J Clin Oncol; 26.5422-8 (2008).
4. Sandler A, Gray R, Perry M C, et al. Paclitaxel-carboplatin alone or with bevacizumab for non-small-cell lung cancer. N Engl J Med; 355: 2542-50 (2006).
5. Wolmark N, Yothers G, O'Connell M J, et al. A phase III trial comparing mFOLFOX6 to mFOLFOX6 plus bevacizumab in stage II or III carcinoma of the colon: results of NSABP protocol C-08. J Clin Oncol; 27.LBA4 (2009).
6. Yang J C, Haworth L, Sherry R M, et al., A randomized trial of bevacizumab, an antivascular endothelial growth factor antibody, for metastatic renal cancer, N Engl J Med 349 427-434 (2003).
7. Willett C G, Boucher Y, di Tomaso E, et al., Direct evidence that the VEGF-specific antibody bevacizumab has antivascular effects in human rectal cancer, Nat. Med. 10, 145-147 (2004).
8. Miller K, Wang M, Gralow J, et al., Paclitaxel plus bevacizumab versus paclitaxel alone for metastatic breast cancer, N Engl J Med 357 2666-2676 (2007).
9. Miller K D, Chap L I, Holmes F A, et al., Randomized phase III trial of capecitabine compared with bevacizumab plus capecitabine in patients with previously treated metastatic breast cancer, J Clin Oncol 23 792-799 (2005).
10. O'Shaughnessy J, Miles D, Gray R J, et al., A meta-analysis of overall survival data from three randomized trials of bevacizumab (BV) and first-line chemotherapy as treatment for patients with metastatic breast cancer (MBC), J Clin Oncol 28 (suppl) (abstr 1005) (2010).
11. Reck M, von Pawel J, Zatloukal P, et al., Phase III trial of cisplatin plus gemcitabine with either placebo or bevacizumab as first-line therapy for nonsquamous non-small-cell lung cancer: AVAil, J Clin Oncol 27, 1227-1234 (2009).
12. Escudier B, Bellmunt J, Negrier S et al., Phase III trial of bevacizumab plus interferon alfa-2a in patients with metastatic renal cell carcinoma (AVOREN): final analysis of overall survival, J Clin Oncol 28, 2144-2150 (2010)
13. Burger R A, Sill M W, Monk B J, et al. Phase II trial of bevacizumab in persistent or recurrent epithelial ovarian cancer or primary peritoneal cancer: a Gynecologic Oncology Group Study. J Clin Oncol, 20; 25(33):5165-71 (2007).
14 Tothill R W et al. Novel molecular subtypes of serous and endometrioid ovarian cancer linked to clinical outcome. Clin Cancer Res. 14(16), 5198-208 (2008).
15. Bagri A, Berry L, Gunter B, et a. (2010) Effects of anti-VEGF treatment duration on tumor growth, tumor regrowth, and treatment efficacy. Clin Cancer Res 16:3887-3900.
16. Ebos J M, Lee C R, Cruz-Munoz W, et al. (2009) Accelerated metastisis after short-term treatment with a potent inhibitor of tumor angiogenesis. Cancer Cell 15:232-239.
17. Grepin R, Guyot M, Jacquin M, Durivault J, Chamorey E. Sudaka A, Serdjebi C, Lacarelle B. Scoazec J Y, Negrier S, Simmonet H, Pages G. Acceleration of clear cell renal cell carcinoma growth in mice following bevacizumab/Avastin treatment: the role of CXCL cytokines. Oncogene 2012 Mar. 29:31 (13).
18. Ma J. Pulfer S, Li S, Chu J, Reed K, Gallo J M. Pharmacodynamic-mediated reducation of temozolomide tumor concentrations by the angiogenesis inhibitor TNP-470. Cancer Res. 2001; 61:5491-5498.
19. Dejong S. Simpis—an Alternative Approach to Partial Least-Squares Regression. Chemometr Intell Lab 1993; 18:251-63.
20. Zuber V, Strimmer K. Gene ranking and biomarker discovery under correlation. Bioinformatics 2009; 25:2700-7.
21. Tothill R W, Tinker A V, George J, et al. Novel molecular subtypes of serous and endometrioid ovarian cancer linked to clinical outcome. Clin Cancer Res 2008; 14:5198-208.

1. A method for selecting whether to administer an anti-angiogenic therapeutic agent to a subject with cancer, comprising:
measuring the expression levels of at least 3 biomarkers in a sample from the subject, wherein at least two of the biomarkers are from Table A and at least one of the biomarkers is from Table B and assessing from the expression levels of the biomarkers whether the cancer belongs to a cancer sub-type
wherein the cancer sub-type is defined by the expression levels of the genes in Tables A and B.
wherein if the cancer belongs to the sub-type an anti-angiogenic therapeutic agent is contraindicated
wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

2. A method for selecting whether to administer an anti-angiogenic therapeutic agent to a subject with cancer, comprising:
measuring the expression levels of at least 3 biomarkers in a sample from the subject, wherein at least two of the biomarkers are from Table A and at least one of the biomarkers is from Table B and assessing from the expression levels of the biomarkers whether the cancer belongs to a cancer sub-type, wherein if the cancer belongs to the subtype the expression levels of the at least two biomarkers from Table A and the at least one biomarker from Table B are increased or decreased as defined for each biomarker in Table A and Table B
wherein the cancer sub-type is defined by the expression levels of the genes in Tables A and B
wherein if the cancer belongs to the sub-type an anti-angiogenic therapeutic agent is contraindicated.

3. A method for predicting the responsiveness of a subject with cancer to an anti-angiogenic therapeutic agent comprising:
allocating the cancer to a cancer sub-type by measuring the expression levels of at least 3 biomarkers in a sample from the subject, wherein at least two of the biomarkers are from Table A and at least one of the biomarkers is from Table B and assessing from the expression levels of the biomarkers whether the cancer belongs to the sub-type
wherein the cancer sub-type is defined by the expression levels of the genes in Tables A and B
classifying the subject as responsive or non-responsive to the anti-angiogenic therapeutic agent on the basis of allocation to the subtype, wherein if the cancer belongs to the sub-type it is predicted to be non-responsive to the anti-angiogenic therapeutic agent
wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

4. A method for predicting the responsiveness of a subject with cancer to an anti-angiogenic therapeutic agent comprising:
allocating the cancer to a cancer sub-type by measuring the expression level of at least 3 biomarkers in a sample from the subject, wherein at least two of the biomarkers are from Table A and at least one of the biomarkers is from Table B and assessing from the expression levels of the biomarkers whether the cancer belongs to the sub-type wherein if the cancer belongs to the subtype the expression levels of the at least two biomarkers from Table A and the at least one biomarker from Table B are increased or decreased as defined for each biomarker in Table A and Table B wherein the cancer sub-type is defined by the expression levels of the genes in Tables A and B
classifying the subject as responsive or non-responsive to the anti-angiogenic therapeutic agent on the basis of allocation to the subtype, wherein if the cancer belongs to the sub-type it is predicted to be non-responsive to the anti-angiogenic therapeutic agent.

5. A method of determining clinical prognosis of a subject with cancer comprising: measuring the expression level of at least 3 biomarkers in a sample from the subject,
wherein at least two of the biomarkers are from Table A and at least one of the biomarkers is from Table B and assessing from the expression levels of the biomarkers whether the cancer belongs to a cancer sub-type
wherein the cancer sub-type is defined by the expression levels of the genes in Tables A and B
classifying the subject as having a good prognosis if the cancer belongs to the sub-type
wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74.

6. A method of determining clinical prognosis of a subject with cancer comprising: measuring the expression level of at least 3 biomarkers in a sample from the subject,
wherein at least two of the biomarkers are from Table A and at least one of the biomarkers is from Table B and assessing from the expression levels of the biomarkers whether the cancer belongs to a cancer sub-type wherein if the cancer belongs to the subtype the expression levels of the at least two biomarkers from Table A and the at least one biomarker from Table B are increased or decreased as defined for each biomarker in Table A and Table B
wherein the cancer sub-type is defined by the expression levels of the genes in Tables A and B
classifying the subject as having a good prognosis if the cancer belongs to the sub-type.

7. A method for selecting whether to administer an anti-angiogenic therapeutic agent to a subject with cancer, comprising:
measuring the expression levels of at least 2 biomarkers in a sample from the subject and assessing from the expression levels of the biomarkers whether the cancer belongs to a cancer sub-type
wherein the cancer sub-type is defined by the expression levels of the genes in Tables A and B
wherein if the cancer belongs to the sub-type an anti-angiogenic therapeutic agent is contraindicated
wherein the at least 2 biomarkers do not consist of from 1 to 63 of the biomarkers shown in Table C.

8. A method for predicting the responsiveness of a subject with cancer to an anti-angiogenic therapeutic agent comprising:
allocating the cancer to a cancer sub-type by measuring the expression levels of at least 2 biomarkers in a sample from the subject and assessing from the expression levels of the biomarkers whether the cancer belongs to the sub-type
wherein the cancer sub-type is defined by the expression levels of the genes in Tables A and B
classifying the subject as responsive or non-responsive to the anti-angiogenic therapeutic agent on the basis of allocation to the subtype, wherein if the cancer belongs to the sub-type it is predicted to be non-responsive to the anti-angiogenic therapeutic agent
wherein the at least 2 biomarkers do not consist of from 1 to 63 of the biomarkers shown in Table C.

9. A method of determining clinical prognosis of a subject with cancer comprising:
measuring the expression levels of at least 2 biomarkers in a sample from the subject and assessing from the expression levels of the biomarkers whether the cancer belongs to a cancer sub-type
wherein the cancer sub-type is defined by the expression levels of the genes in Tables A and B
classifying the subject as having a good prognosis if the cancer belongs to the sub-type
wherein the at least 2 biomarkers do not consist of from 1 to 63 of the biomarkers shown in Table C.

10. The method of claim 5, 6 or 9, wherein the subject is receiving, has received and/or will receive a standard chemotherapeutic treatment for the subject's cancer type and/or will not receive an anti-angiogenic therapeutic agent.

11. The method of claim 5, 6, 9 or 10, wherein good prognosis indicates increased progression free survival and/or overall survival rates and/or decreased likelihood of recurrence or metastasis compared to subjects with cancers that do not belong to the sub-type.

12. A method of treating cancer comprising administering a chemotherapeutic agent to a subject wherein the subject is selected for treatment on the basis of a method as claimed in any previous claim and wherein an anti-angiogenic therapeutic agent is not administered (if the cancer is determined to belong to the subtype).

13. A method of treating cancer comprising administering a chemotherapeutic agent and not administering an anti-angiogenic therapeutic agent to a subject, wherein the subject has a cancer that has been determined to belong to a cancer sub-type,
wherein the cancer sub-type is defined by the expression levels of the genes in Tables A and B, by either:
(i) measuring the expression levels of at least 3 biomarkers in a sample from the subject, wherein at least two of the biomarkers are from Table A and at least one of the biomarkers is from Table B and assessing from the expression levels of the biomarkers whether the cancer belongs to the cancer sub-type
wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74; or
(ii) measuring the expression levels of at least 2 biomarkers in a sample from the subject and assessing from the expression levels of the biomarkers whether the cancer belongs to the cancer sub-type
wherein the at least 2 biomarkers do not consist of from 1 to 63 of the biomarkers shown in Table C.

14. A chemotherapeutic agent for use in treating cancer in a subject wherein the subject is selected for treatment on the basis of a method as claimed in any previous claim and wherein the subject is not treated with an anti-angiogenic therapeutic agent (if the cancer is determined to belong to the subtype).

15. A chemotherapeutic agent for use in treating cancer in a subject wherein the subject has a cancer that has been determined to belong to a cancer sub-type, wherein the cancer sub-type is defined by the expression levels of the genes in Tables A and B, by either:
(i) measuring the expression levels of at least 3 biomarkers in a sample from the subject, wherein at least two of the biomarkers are from Table A and at least one of the biomarkers is from Table B and assessing from the expression levels of the biomarkers whether the cancer belongs to the cancer sub-type
wherein the at least 3 biomarkers do not comprise at least two biomarkers selected from COL1A2, COL3A1, TIMP3, COL4A1, COL8A1, CDH11, TIMP2, ANGPTL2, and MMP14 and at least one biomarker selected from CIITA, XAF1 and CD74; or
(ii) measuring the expression levels of at least 2 biomarkers in a sample from the subject and assessing from the expression levels of the biomarkers whether the cancer belongs to the cancer sub-type
wherein the at least 2 biomarkers do not consist of from 1 to 63 of the biomarkers shown in Table C.
and wherein the subject is not treated with an anti-angiogenic therapeutic agent.

16. A method of treating cancer comprising administering a chemotherapeutic agent to a subject wherein the subject has a cancer that belongs to a cancer sub-type
defined by the expression levels of the genes in Tables A and B and wherein an anti-angiogenic therapeutic agent is not administered.

17. A chemotherapeutic agent for use in treating cancer in a subject wherein the subject has a cancer that belongs to a cancer sub-type defined by the expression levels of the genes in Tables A and B and wherein the subject is not treated with an anti-angiogenic therapeutic agent.

18. The method of any of claims 12, 13, or 16 or chemotherapeutic agent for use of any of claims 14, 15, or 17, wherein the chemotherapeutic agent comprises a platinum-based chemotherapeutic agent, an alkylating agent, an anti-metabolite, an anti-tumor antibiotic, a topoisomerase inhibitor, a mitotic inhibitor, or a combination thereof.

19. The method of any of claims 12, 13, or 16 or chemotherapeutic agent for use of any of claims 14, 15, or 17, wherein the chemotherapeutic agent comprises a platinum based-chemotherapeutic agent, a mitotic inhibitor, or a combination thereof.

20. The method of any of claims 12, 13, or 16 or chemotherapeutic agent for use of any of claims 14, 15, or 17, wherein the chemotherapeutic agent comprises carboplatin and/or paclitaxel.

21. The method of any of claims 1 to 13, 16, or 18 to 20 or chemotherapeutic agent for use of any of claims 14, 15, or 17 to 20 wherein assessing whether the cancer belongs to the sub-type comprises:
determining a sample expression score for the biomarkers;
comparing the sample expression score to a threshold score; and
determining whether the sample expression score is above or
equal to or below the threshold expression score,
wherein if the sample expression score is above or equal to the threshold expression score the cancer belongs to the sub-type.

22. The method of any of claims 1 to 13, 16, or 18 to 21 or chemotherapeutic agent for use of any of claims 14, 15, or 17 to 21 wherein the at least two biomarkers do not comprise any one or more of the 63 biomarkers shown in table C.

23. The method of any of claims 1 to 13, 16, or 18 to 22 or chemotherapeutic agent for use of any of claims 14, 15, or 17 to 22, wherein the cancer sub-type is defined by increased and decreased expression levels of the genes listed in Tables A and B as shown in Tables A and B.

24. The method of any of claims 1 to 13, 16, or 18 to 23 or chemotherapeutic agent for use of any of claims 14, 15, or 17 to 23, wherein the subject is receiving, has received and/or will receive (optionally together with the anti-angiogenic therapeutic agent) treatment with a chemotherapeutic agent.

25. The method of any of claims 1 to 13, 16, or 18 to 24 or chemotherapeutic agent for use of any of claims 14, 15, or 17 to 24 further comprising obtaining a test sample from the subject.

26. The method of any of claims 1 to 13, 16, or 18 to 25 or chemotherapeutic agent for use of any of claims 14, 15, or 17 to 25, wherein the cancer is ovarian cancer, peritoneal cancer or fallopian tube cancer.

27. The method or chemotherapeutic agent for use of claim 26, wherein the ovarian cancer is serous ovarian cancer, optionally high grade serous ovarian cancer.

28. The method of any of claims 1 to 13, 16, or 18 to 27 or chemotherapeutic agent for use of any of claims 14, 15, or 17 to 27, wherein the subject is receiving, has received and/or will receive an anti-angiogenic therapeutic agent.

29. The method of any of claims 1 to 4, 7, 8, 10 to 13 16, or 18 to 28 or chemotherapeutic agent for use of any of claims 14, 15, or 17 to 28, wherein the anti-angiogenic therapeutic agent is a VEGF-pathway-targeted therapeutic agent, an angiopoietin-TIE2 pathway inhibitor, an endogenous angiogenic inhibitor, or an immunomodulatory agent.

30. The method or chemotherapeutic agent for use of claim 29, wherein the VEGF pathway-targeted therapeutic agent is selected from Bevacizumab (Avastin), Aflibercept (VEGF Trap), IMC-1121B (Ramucirumab), Imatinib (Gleevec), Sorafenib (Nexavar), Gefitinib (Iressa), Sunitinib (Sutent), Erlotinib, Tivozinib, Cediranib (Recentin), Pazopanib (Votrient), BIBF 1120 (Vargatef), Dovitinib, Semaxanib (Sugen), Axitinib (AG013736), Vandetanib (Zactima), Nilotinib (Tasigna), Dasatinib (Sprycel), Vatalanib, Motesanib, ABT-869, TKI-258 or a combination thereof.

31. The method or chemotherapeutic agent for use of claim 29, wherein the angiopoietin-TIE2 pathway inhibitor is selected from AMG-386, PF-4856884 CVX-060, CEP-11981, CE-245677, MEDI-3617, CVX-241, Trastuzumab (Herceptin) or a combination thereof.

32. The method or chemotherapeutic agent for use of claim 29, wherein the endogenous angiogenic inhibitor is selected from Thombospondin, Endostatin, Tumstatin, Canstatin, Arrestin, Angiostatin, Vasostatin, Interferon alpha or a combination thereof.

33. The method or chemotherapeutic agent for use of claim 29, wherein the immunomodulatory agent is selected from thalidomide and lenalidomide.

34. The method or chemotherapeutic agent for use of claim 30, wherein the VEGF pathway-targeted therapeutic agent is bevacizumab.

35. A method for selecting whether to administer Bevacizumab to a subject, comprising:
in a test sample obtained from a subject suffering from ovarian cancer, which subject is being, has been and/or will be treated using a platinum-based chemotherapeutic agent and/or a mitotic inhibitor;
measuring expression levels of at least 2 biomarkers;
determining a sample expression score for the 2 or more biomarkers;
comparing the sample expression score to a threshold score;
wherein if the sample expression score is above or equal to the threshold expression score the cancer belongs to a cancer sub-type defined by the expression levels of the genes in Tables A and B
selecting a treatment based on whether the cancer belongs to the sub-type, wherein if the cancer belongs to the sub-type Bevacizumab is contraindicated.

36. The method of claim 35 further comprising obtaining the sample from the subject.

37. The method of claim 35 or 36 wherein the ovarian cancer comprises serous ovarian cancer.

38. The method of claim 37 wherein the serous ovarian cancer is high grade serous ovarian cancer.

39. The method of any one of claims 35 to 38 wherein if Bevacizumab is contraindicated the patient is treated with a platinum-based chemotherapeutic agent and/or a mitotic inhibitor.

40. The method of any one of claims 35 to 38 wherein if the cancer does not belong to the sub-type the patient is treated with a platinum-based chemotherapeutic agent and/or a mitotic inhibitor together with Bevacizumab.

41. The method of any of claims 7 to 13, 16, or 18 to 40 or chemotherapeutic agent for use of any of claims 14, 15, or 17 to 34 comprising measuring the expression level of at least 3 biomarkers in a sample from the subject, wherein at least two of the biomarkers are from Table A and at least one of the biomarkers is from Table B.

42. The method of any of claims 7 to 13, 16, or 18 to 40 or chemotherapeutic agent for use of any of claims 14, 15, or 17 to 34 comprising measuring the expression levels of at least 4 of the biomarkers from Table F.

43. The method of any of claims 7 to 13, 16, 18 to 40 or 42 or chemotherapeutic agent for use of any of claims 14, 15, 17 to 34 or 42 comprising measuring the expression levels of at least one of GABRE, HLA-DPA1, CHI3L1, KCND2, GBP3, UPK2, SYTL4, LRRN1, USP53 and POU2F3.

44. The method of any of claims 7 to 13, 16, 18 to 40 or 42 or chemotherapeutic agent for use of any of claims 14, 15, or 17 to 34 or 42 comprising measuring the expression levels of each of GABRE, HLA-DPA1, CHI3L1, KCND2, GBP3, UPK2, SYTL4, LRRN1, USP53 and POU2F3.

45. The method of any of claims 7 to 13, 16, 18 to 40 or 42 or chemotherapeutic agent for use of any of claims 14, 15, or 17 to 34 or 42 comprising measuring the expression levels of each of the biomarkers from Table F.

46. The method of any of claims 7 to 13, 16, 18 to 40 or 42 to 45 or chemotherapeutic agent for use of any of claims 14, 15, 17 to 34 or 42 to 45 comprising measuring the expression levels of at least 10 of the biomarkers from Table I.

47. The method of any of claims 7 to 13, 16, 18 to 40 or 42 to 46 or chemotherapeutic agent for use of any of claims 14, 15, 17 to 34 or 42 to 46 comprising measuring the expression levels of at least one of MT1L, MT1G, LRP4, RASL11B, IFI27, PKIA, ALOX5AP, UBD, MEX3B, and TMEM98.

48. The method of any of claims 7 to 13, 16, 18 to 40 or 42 to 46 or chemotherapeutic agent for use of any of claims 14, 15, 17 to 34 or 42 to 46 comprising measuring the expression levels of each of MT1L, MT1G, LRP4, RASL11B, IFI27, PKIA, ALOX5AP, UBD, MEX3B, and TMEM98.

49. The method of any of claims 7 to 13, 16, 18 to 40 or 42 to 46 or chemotherapeutic agent for use of any of claims 14, 15, 17 to 34 or 42 to 46, comprising measuring the expression levels of each of the biomarkers listed in Table I.

50. The method of any of claims 7 to 13, 16, 18 to 40 or 42 to 49 or chemotherapeutic agent for use of any of claims 14, 15, 17 to 34 or 42 to 49 comprising measuring the expression levels of at least 15 of the biomarkers from Table L.

51. The method of any of claims 7 to 13, 16, 18 to 40 or 42 to 50 or chemotherapeutic agent for use of any of claims 14, 15, 17 to 34 or 42 to 50 comprising measuring the expression levels of at least one of MTL1, GABRE, KCND2, UPK2, HLA-DPA1, SYTL4, SCEL, MZT1, EFNB3, and DLL1.

52. The method of any of claims 7 to 13, 16, 18 to 40 or 42 to 50 or chemotherapeutic agent for use of any of claims 14, 15, 17 to 34 or 42 to 50 comprising measuring the expression levels of each of MTL1, GABRE, KCND2, UPK2, HLA-DPA1, SYTL4, SCEL, MZT1, EFNB3, and DLL1.

53. The method of any of claims 7 to 13, 16, 18 to 40 or 42 to 50 or chemotherapeutic agent for use of any of claims 14, 15, 17 to 34 or 42 to 50, comprising measuring the expression levels of each of the biomarkers listed in Table L.

54. The method of any of claims 1 to 13, 16, or 18 to 53 or chemotherapeutic agent for use of any of claims 14, 15, 17 to 34 or 41 to 53, wherein the expression score is calculated using a weight value and/or a bias value for each biomarker.

55. The method of any of claims 1 to 13, 16, or 18 to 54 or chemotherapeutic agent for use of any of claims 14, 15, 17 to 34 or 41 to 54 wherein the expression level is determined at the level of RNA.

56. The method or chemotherapeutic agent for use of claim 55 wherein the expression level is determined by microarray, northern blotting, or nucleic acid amplification.

57. The method or chemotherapeutic agent for use of claim 55 or 56, wherein measuring the expression levels of the biomarkers comprises contacting the sample with a set of nucleic acid probes or primers that bind to the biomarkers and detecting binding of the set of nucleic acid probes or primers to the biomarkers by microarray, northern blotting, or nucleic acid amplification.

58. A method of deriving a panel of at least 2 biomarkers, wherein the expression level(s) of the at least 2 biomarkers in a sample from a subject with a cancer allows the cancer to be allocated to a sub-type
wherein the cancer sub-type is defined by the expression levels of the genes in Tables A and B
said method comprising the steps of:
sorting samples from a sample set of known pathology and/or clinical outcome on the basis of allocation to the sub-type obtaining the expression profiles of the samples analysing the expression profiles from the sample set using a mathematical model identifying from the results of the mathematical model one or more biomarkers expressed in the sample set that are most predictive of the cancer sub-type.

59. The method of claim 58, wherein the cancer sub-type is defined by increased and decreased expression levels of the genes listed in Tables A and B as shown in Tables A and B

60. The method of claim 58 or 59, wherein the mathematical model is a parametric, non-parametric or semi-parametric model.

61. The method of any of claims 58 to 60, wherein the mathematical model is Partial Least Squares (PLS), Shrinkage Discriminate Analysis (SDA), or Diagonal SDA (DSDA).

62. The method of any of claims 58 to 61 wherein identifying from the results of the mathematical model one or more biomarkers expressed in the sample set that are most predictive of the cancer sub-type comprises identifying one or more biomarkers for which area under the receiver operator characteristic curve (AUC) and Concordance Index (C-Index) are significant.

63. The method of any of claims 1 to 13, 16, or 18 to 62 or chemotherapeutic agent for use of any of claims 14, 15, 17 to 34 or 41 to 62,
wherein the cancer is allocated to the sub-type based on the expression level of a panel of one or more biomarkers derived using the method of any of claims 58-62 in a sample from the subject.

64. An anti-angiogenic therapeutic agent for use in treating cancer in a subject wherein the subject is selected for treatment on the basis of a method as claimed in any previous claim, wherein allocation of the subject to the subtype contra-indicates the anti-angiogenic therapeutic agent.

65. A method for selecting whether to administer an anti-angiogenic therapeutic agent to a subject having a cancer substantially as herein described.

66. A method for predicting the responsiveness of a subject with cancer to an anti-angiogenic therapeutic agent substantially as herein described.

67. A method of determining clinical prognosis substantially as herein described.

68. A method for selecting whether to administer Bevacizumab to a subject substantially as herein described.

69. A chemotherapeutic agent for use in treating cancer substantially as herein described.

70. A method of treating cancer substantially as herein described.
